(19) United States
(12) Patent Application Publication
(10) Pub. No.: US 2026/0083708 A1
(43) Pub. Date: Mar. 26, 2026

(54) IL-17 MODULATORS AND USES THEREOF

(71) Applicant: NNOCARE PHARMA INC., Wilmington, DE (US)

(72) Inventors: Yucheng PANG, Beijing (CN); Feiyang XU, Beijing (CN); Xiangyang CHEN, Beijing (CN)

(21) Appl. No.: 19/405,049

(22) Filed: Dec. 1, 2025

Related U.S. Application Data
(63) Continuation of application No. PCT/CN2025/086173, filed on Mar. 31, 2025.
(60) Provisional application No. 63/573,653, filed on Apr. 3, 2024.

(51) Int. Cl. A61K 31/4196; A61P 19/02; C07D 401/10

(52) U.S. Cl. CPC A61K 31/4196; A61P 19/02; C07D 401/10

(57) ABSTRACT

The present invention relates to compounds suitable for inhibiting or regulating the activity of IL-17. The present invention is directed to compounds shown in Formula (I) and their pharmaceutically acceptable salts thereof. The present invention further relates to methods for treating and/or preventing various human diseases, including inflammatory and autoimmune disorders. The present invention also provides methods for preparing the compounds of the present invention.

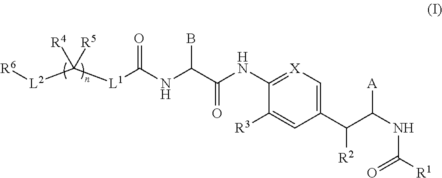

### CROSS-REFERENCE TO RELATED APPLICATIONS

This application is a Continuation application of International Patent Application No. PCT/CN2025/086173, filed on Mar. 31, 2025, which claims the benefit of Provisional Application No. 63/573,653, filed on Apr. 3, 2024, the entire disclosures of which are incorporated herein by reference in their entirety.

### TECHNICAL FIELD

The present invention relates to compounds of Formula (I), or their pharmaceutically acceptable salts thereof, for the regulation of IL-17 biological functions. The present invention also relates to methods for preparing the compounds of Formula (I). The present invention further relates to methods for treating and/or preventing various human diseases, including inflammatory and autoimmune disorders.

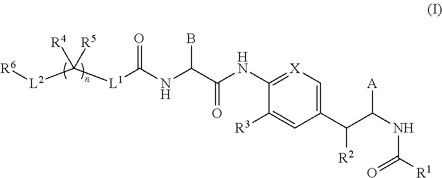

### BACKGROUND

Interleukin-17 (IL-17) is a family of six cytokines (IL-17A through F) that link T cell activation to neutrophil mobilization and activation. Among six members of IL-17, IL-17A (originally named CTLA-8 and also known as IL-17) is a pro-inflammatory cytokine, which is involved in the induction or secretion of IL-6, IL-8, G-GSF, TNF-α, IL-1β, PGE2, and IFN-γ, as well as various chemokines and other effectors. IL-17A and IL-17F share approximately 55% amino acid sequence homology, and they can form homodimers (IL-17AA and IL-17FF) or heterodimers (IL-17AF) by forming disulfide bridges (Aggarwal, S., Gurney, A. L., J. Leukoc. Biol. 2002, 71, 1-8). IL-17A and F signal through the receptors of IL-17RA and IL-17RC, or an IL-17RA/RC 20 receptor complex (Gaffen, S. L., Nat. Rev. Immunol. 2009, 9, 556-567).

IL-17A and IL-17F are mainly expressed by Th17 cells, which are involved in the pathology of inflammation and autoimmunity. Dysregulated expression of IL-17A is implicated in autoimmune disorders and other diseases, including but not limited to psoriasis (Albanesi, C. et.al, Front. Immunol. 2018, 9, 1549), psoriatic arthritis, rheumatoid arthritis, ankylosing spondylitis, nonradiographic axial spondyloarthritis (Amatya, N. et.al, Trends Immunol. 2017, 38, 310-322), bone erosion, intraperitoneal abscesses, inflammatory bowel disease, allograft rejection, angiogenesis, atherosclerosis, asthma, and multiple sclerosis.

Anti-IL-17A monoclonal antibodies (mAbs), such as secukinumab (Krueger, J. G. et.al, J. Allergy Clin. Immunol. 2019, 144, 750-763), ixekizumab (Liu, L. et.al, J. Inflamm. Res. 2016, 9, 39-50) and bimekizumab (Reich, K. et.al, N. Engl. J. Med. 2021, 385, 142-152), have shown good efficacy for the treatment of inflammatory diseases, such as psoriasis, ankylosing spondylitis, and psoriatic arthritis. However, these highly costing treatments require injection to a patient biweekly or monthly of mAbs because they cannot be absorbed through the digestive tract via oral administration.

Small molecule modulators of IL-17, which are convenient for oral administration and have lower cost, have become a better alternative to mAbs. Some patent applications for small molecule modulators of IL-17 have been disclosed, for example, WO2018229079, WO2021055376, WO2020127685, WO2022091056, WO2023166172, and WO2023283453. Moreover, a few small molecule modulators of IL-17 have been approved for clinical trials, such as DC-806, DC-853 and LP0200. However, preliminary clinical results indicated such small molecular IL-17 modulators were not as efficacious as approved biologics. Therefore, there is still a need to develop new compounds with better druggability and efficacy.

### BRIEF DESCRIPTION OF THE DRAWINGS

FIG. 1 shows that compounds 35, 39 and 66 completely suppressed arthritis disease progression at an oral dose of 20 mg/kg, once a day, in the rat collagen induced arthritis (CIA) model.

### DETAILED DESCRIPTION OF THE INVENTION

### Definitions

Unless otherwise stated, the following terms used in this application have the following meanings.

“C_{x-y}” refers to a range of number of carbon atoms, where x and y are both integers, for example, C₃₋₈ cycloalkyl stands for cycloalkyl having 3 to 8 carbon atoms.

“Alkyl” refers to a saturated straight-chain or branched-chain hydrocarbyl substituent containing 1 to 20 carbon atoms, for example, 1 to 8 carbon atoms, 1 to 6 carbon atoms, or 1 to 4 carbon atoms. Unrestricted examples of alkyl include but are not limited to methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl and 2-ethylbutyl.

“alkenyl” and “alkynyl” refer to unsaturated aliphatic groups analogous in length and possible substitution to the alkyl described above, but that contain at least one double or triple bond, respectively. Unrestricted examples of alkenyl include but are not limited to ethenylene, propenylene, butenylene, pentenylene, hexenylene, and any one of which is optionally substituted. An alkenylene chain may have one double bond or more than one double bond in the alkenylene chain. Unrestricted examples of alkynyl include but are not limited to ethynylene, propynylene, butynylene, pentynylene, hexynylene, and any one of which is optionally substituted. An alkynylene chain may have one triple bond or more than one triple bond in the alkynylene chain.

“Alkylene” refers to a saturated straight-chain or branched-chain hydrocarbyl divalent substituent containing 1 to 20 carbon atoms, for example, 1 to 6 carbon atoms or 1 to 4 carbon atoms. Unrestricted examples of alkylene include but are not limited to —CH₂—, —CH(CH₃)—, —CH₂CH₂—, —CH₂CH₂CH₂—, —(CH₃)C(CH₃)—, —CH₂CH₂CH₂CH₂— and —CH₂CH(CH₃)CH₂—.

“Cycloalkyl” refers to a saturated cyclic hydrocarbyl substituent containing 3 to 14 annular carbon atoms. Cycloalkyl can be a mono carbon ring substituent, typically containing 3 to 8, 3 to 7, or 3 to 6 carbon atoms. Unrestricted examples of monocyclic cycloalkyl include but are not limited to cyclopropyl, cyclobutyl, cyclopentyl, cyclohexyl and cycloheptyl. Cycloalkyl can also be a substituent with two or three mono carbon rings that are fused together, such as decahydronaphthyl.

“Heterocyclyl or heterocycle” refers to a saturated or partially unsaturated monocyclic or polycyclic group containing 3 to 20 annular atoms, for example, 3 to 14, 3 to 12, 3 to 10, 3 to 8, 3 to 6, or 5 to 6 annular atoms in which one or more of the annular atoms are selected from N, O and S(O)ₘ (where m is an integer from 0 to 2). Preferably, it can have 3 to 12 annular atoms, 3 to 10 annular atoms, 4 to 7 annular atoms, and 4 to 6 annular atoms, wherein 1 to 4 are heteroatoms, 1 to 3 are heteroatoms, or 1 to 2 are heteroatoms. Unrestricted examples of monocyclic heterocyclyl include but are not limited to pyrrolidinyl, oxetanyl, piperidyl, piperazinyl, tetrahydrofuranyl, tetrahydropyranyl, tetrahydrothiopyranyl, morpholinyl, thiomorpholinyl, homopiperazinyl and azetidinyl. Polycyclic heterocyclyl includes fused, bridged or spiro polycyclic heterocycle, such as octahydrocyclopenta[c]pyrrole, octahydropyrrole[1,2-a]pyrazine, 3,8-diazabicyclo[3.2.1]octane, 5-azaspiro[2.4]heptane and 2-oxa-7-azaspiro[3.5]nonane.

“Aryl or aryl ring” refers to an aromatic monocyclic or fused polycyclic group containing 6 to 14 carbon atoms, preferably 6- to 10-membered, such as phenyl and naphthyl, most preferably phenyl. The aryl ring can be fused with a heteroaryl, heterocyclyl or cycloalkyl ring, and unrestricted examples include but are not limited to:

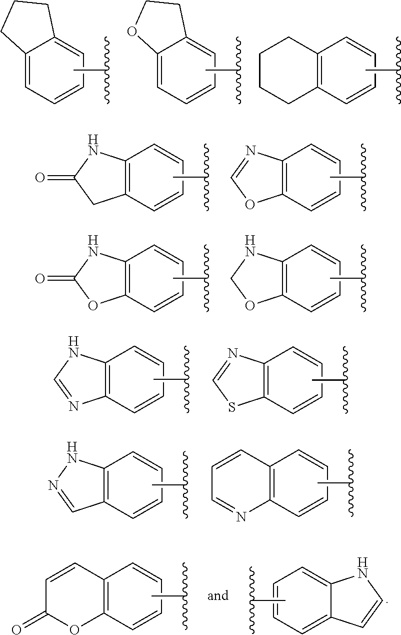

“Heteroaryl or heteroaryl ring” refers to a heteroaromatic system containing 5 to 14 annular atoms, of which 1 to 4 annular atoms are selected from heteroatoms including O, S and N. Heteroaryl preferably is 5- to 10-membered, and more preferably 5- or 6-membered, such as furyl, thienyl, pyridyl, pyrrolyl, pyrimidyl, pyrazinyl, pyrazolyl, imidazolyl, tetrazolyl, oxazolyl, isoxazolyl, thiazolyl, isothiazolyl, quinolinyl, isoquinolinyl, indolyl and isoindolyl.

“Fused heteroaryl ring” refers to a heteroaryl ring fused with an aryl, heterocyclyl or cycloalkyl ring, and unrestricted examples of a fused heteroaryl ring include but are not limited to:

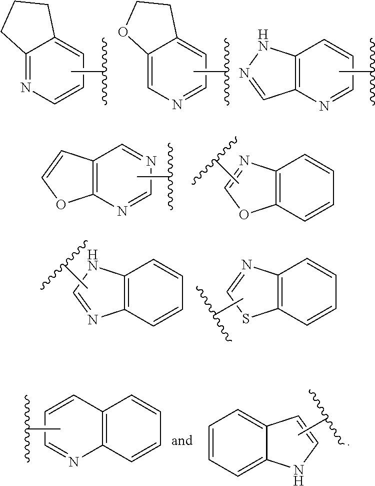

“Halogen” refers to F, Cl, Br, or I.

“Cyano” refers to —CN.

“Oxo” refers to ═O.

“Carbonyl” refers to —C(O)—.

“Sulfonyl” refers to a —S(O)₂—.

“Sulfinyl” refers to a —S(O)—.

“Optional substitution or optionally substituted” refers to that one or more hydrogen atoms in a group, preferably 1-5, for example, 1 to 3 hydrogen atoms, are independently substituted by a corresponding number of substituents. The substituents are located only in the possible chemical positions understood by those skilled in the art. For example, amino or hydroxyl groups with free hydrogen may be unstable when bound to carbon atoms with unsaturated bonds (such as olefinic). The substituents include but are not limited to halogen, hydroxyl, cyano, nitro, oxo, —SF₅, C₁₋₄ alkyl, C₃₋₇ cycloalkyl, etc.

“Isomers” refer to compounds that have the same molecular formula, but their atomic binding position or spatial arrangement is different. Isomers with different arrangement of their atoms in space are called “stereoisomers”. Stereoisomers include optical isomers, geometric isomers, and conformational isomers.

The compounds of the present invention can exist as optical isomers. Optical isomers include enantiomers and diastereomers. An enantiomer is one of two stereoisomers that are mirror images of each other and are non-superimposable. A racemic mixture, or racemate is one that has equal amounts of left- and right-handed enantiomers of a chiral molecule. Diastereomers are stereoisomers that are not mirror images of one another and are non-superimposable on one another. Methods for preparing and separating optical isomers are known in the art. When a compound is a single isomer and its absolute configuration is determined, it is referred as a “R” or “S” isomer according to the configuration of the substituents around the chiral carbon atom. When its absolute configuration is not determined, it is referred as a (+) or (−) isomer according to its measured optical rotation value.

The compounds of the present invention may also have geometric isomers resulting from the distribution of substituents around carbon-carbon double bonds, carbon-nitrogen double bonds, cycloalkyl or heterocyclyl groups. The substituents around carbon-carbon double bond or carbon-nitrogen bond are designated to be in a Z or E configuration, and the substituents around cycloalkyl or heterocycle are designated to be in a cis or trans configuration.

The compounds of the present invention may also show tautomerism, such as keto-enol tautomerism.

The present invention includes any tautomeric or stereoisomeric forms and mixtures thereof and is not limited to any tautomeric or stereoisomeric forms used in the compound nomenclature or chemical structural formulae.

“Isotopes” include all isotopes of the atoms appearing in the compounds of the present invention. Isotopes include those atoms with the same atomic number but in different masses. Examples of isotopes suitable for incorporation into the compounds of the present invention are isotopes of hydrogen, carbon, nitrogen, oxygen, phosphorus, sulfur, fluorine, and chlorine, for example but not limited to ²H (D), ³H, ¹³C, ¹⁴C, ¹⁵N, ¹⁷O, ¹⁸O ³¹P, ³²P, ³⁵S, ¹⁸F and ³⁶Cl. The isotopically labeled compounds of the present invention can generally be prepared by conventional techniques known to those skilled in the art or by methods like those described in the embodiments using appropriate isotopically labeled reagents instead of non-isotopically labeled reagents. Such compounds have various potential uses, for example, as standards and reagents in the determination of biological activities. In the case of stable isotopes such as deuterium ²H (D), ¹³C and ¹⁵N, such compounds have the potential to beneficially alter biological, pharmacological, or pharmacokinetic properties. Deuterium ²H (D) is a preferable isotope of the present invention. For example, the hydrogens of —CH₃ can be substituted by D to —CD₃.

The compounds of the present invention can be administered in form of prodrugs. “Prodrugs” refer to derivatives that are converted into biologically active compounds under the physiological condition in vivo, for example, by oxidation, reduction, and hydrolysis (each of which occurs with or without the participation of enzymes). Examples of a prodrug are a compound of the present invention in which an amino is acylated, alkylated or phosphorylated, for example eicosanoyl amino, alanyl amino and pivaloyloxymethyl amino; a hydroxyl is acylated, alkylated or phosphorylated or converted into borate, for example acetoxy, palmitoyloxy, pivaloyloxy, succinyloxy, fumaroyloxy and alanyloxy; a carbonyl is esterified or amidated; and a thiol forms a disulfide bridge with a carrier molecule that selectively delivers the drug to the target and/or to the cytosol of cells, such as peptide. Prodrugs can be prepared from the compounds of the present invention according to well-known methods.

“Pharmaceutically acceptable salts” refer to salts derived from compounds of the present invention with pharmaceutically acceptable bases or acids, including inorganic alkalis or acids and organic bases or acids, under the condition that the compounds contain one or more acidic or basic groups. Compounds of the present invention that contain acidic groups can exist in form of salts, for example, as alkali metal salts, alkaline earth metal salts, or ammonium salts. For example, such salts include sodium salts, potassium salts, calcium salts, magnesium salts or ammonia or organic amine salts such as salts of ethylamine, ethanolamine, triethanolamine or amino acids. Compounds of the present invention that contain basic groups can exist in form of salts as inorganic or organic acid salts. Examples of suitable acids include hydrochloric acid, hydrobromic acid, phosphoric acid, sulfuric acid, nitric acid, methanesulfonic acid, p-toluenesulfonic acid, naphthalene disulfonic acid, oxalic acid, acetic acid, tartaric acid, lactic acid, salicylic acid, benzoic acid, formic acid, propanoic acid, pivalic acid, malonic acid, succinic acid, pimelic acid, fumaric acid, maleic acid, malic acid, sulfamic acid, phenylpropionic acid, gluconic acid, ascorbic acid, isonicotinic acid, citric acid, adipic acid and other acids known to those skilled in the art. If compounds of the present invention contain both acidic and basic groups in the molecule, the present invention further includes internal salts in addition to the mentioned salt forms. Each salt can be obtained by conventional methods known to those skilled in the art, for example by mixing a compound of the present invention with an organic or inorganic acid or base in a solvent or dispersant, or by anion exchange or cation exchange with another salt.

“Pharmaceutical composition” refers to a composition containing one or more of compounds described herein or pharmaceutically acceptable salts, prodrugs, stable isotope derivatives and isomers thereof, and other components such as pharmaceutically acceptable carriers and excipients.

“Therapeutically effective amount” refers to the amount of the compound of the present invention that can effectively block the function of IL-17, in particular IL-17AA and IL-17AF, and/or treat or prevent the diseases mediated by IL-17.

“Patients” refer to mammals, preferably humans.

The present invention provides compounds useful in inhibiting IL-17 activity. The compounds are shown in Formula (I), or prodrugs, stable isotope derivatives, pharmaceutically acceptable salts and stereoisomers thereof,

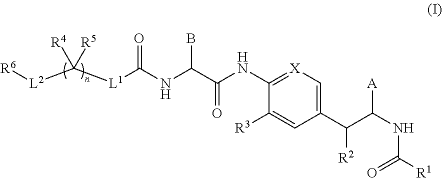

Wherein:
A is 5- to 10-membered heteroaryl or fused heteroaryl, where one or more hydrogens of the heteroaryl and fused heteroaryl are optionally substituted by halogen, C₁₋₆ alkyl, C₃₋₆ cycloalkyl, 4- to 6-membered heterocyclyl, CN, oxo, —OR^{a} or —NR¹¹R¹², where one or more hydrogens of the alkyl, cycloalkyl and heterocyclyl are further optionally substituted by halogen, C₁₋₆ alkyl, C₃₋₆ cycloalkyl, 4- to 6-membered heterocyclyl, CN, oxo, —OR^{b} or —NR¹³R¹⁴, where one or more hydrogens of cycloalkyl or heterocyclyl are further optionally substituted by halogen or C₁₋₆ alkyl;
B is C₁₋₆ alkyl, C₃₋₁₀ cycloalkyl, 4- to 10-membered heterocyclyl, C₆₋₁₀ aryl or 5- to 10-membered heteroaryl, where one or more hydrogens of the alkyl, cycloalkyl, heterocyclyl, aryl and heteroaryl are optionally substituted by C₃₋₁₀ cycloalkyl, halogen, C₁₋₆ alkyl, C₁₋₃ alkenyl, CN or —OR^{c}, where one or more hydrogens of the alkyl, cycloalkyl and alkenyl are further optionally substituted by halogen;
X is CH or N;
R¹ is C₁₋₆ alkyl, C₃₋₆ cycloalkyl, 4- to 6-membered heterocyclyl, H or —NR²¹R²², where one or more hydrogens of the alkyl, cycloalkyl and heterocyclyl are optionally substituted by halogen, D, CN, oxo, C₁₋₆ alkyl, C₃₋₆ cycloalkyl, 4- to 6-membered heterocyclyl, —OR^{d} or —NR¹⁵R¹⁶;
R² is C₁₋₆ alkyl, C₃₋₆ cycloalkyl, H or 4- to 6-membered heterocyclyl, where one or more hydrogens of the alkyl, cycloalkyl and heterocyclyl are optionally substituted by halogen or C₁₋₆ alkyl;
R³ is halogen, H, C₁₋₆ alkyl, C₃₋₆ cycloalkyl or —OR^{e}, where one or more hydrogens of the alkyl and cycloalkyl are optionally substituted by halogen;
L¹ is bond, —O— or —NH—;
L² is bond, —O— or —CH₂O—;
R⁴ and R⁵ are independently selected from H, halogen and C₁₋₆ alkyl;
R⁶ is 5- to 10-membered heteroaryl or C₃₋₁₀ cycloalkyl, where one or more hydrogens of the cycloalkyl and heteroaryl are optionally substituted by C₁₋₆ alkyl, C₃₋₆ cycloalkyl, CN, halogen, 4- to 8-membered heterocyclyl or —OR^{f}, where one or more hydrogens of the alkyl, cycloalkyl and heterocyclyl are further optionally substituted by halogen, D, C₁₋₆ alkyl or C₃₋₁₀ cycloalkyl;
R^{a}, R^{b}, R^{c}, R^{d}, R^{e}, R^{f}, R¹¹, R¹², R¹³, R¹⁴, R¹⁵ R¹⁶, R²¹ and R²² are independently selected from H, C₁₋₆ alkyl, C₃₋₆ cycloalkyl and 4- to 10-member heterocyclyl, where one or more hydrogens of the alkyl, cycloalkyl and heterocyclyl are optionally substituted by halogen, D, C₁₋₆ alkyl or C₃₋₆ cycloalkyl; and
n is 0 or 1.

In one embodiment, A is 5- to 10-membered heteroaryl containing one to four heteroatoms selected from N, O and S, where one or more hydrogens of the heteroaryl are optionally substituted by halogen, C₁₋₆ alkyl, C₃₋₆ cycloalkyl, 4- to 6-membered heterocyclyl or —OR^{a}, where one or more hydrogens of the alkyl, cycloalkyl and heterocyclyl are further optionally substituted by halogen, CN, C₁₋₆ alkyl, C₃₋₆ cycloalkyl, 4- to 6-membered heterocyclyl, —OR^{b} or —NR¹³R¹⁴, where one or more hydrogens of cycloalkyl and heterocyclyl are further optionally substituted by halogen or C₁₋₆ alkyl.

In one embodiment, A is a 5-membered heteroaryl, for example, triazole, imidazole, thiadiazole, or tetrazole.

In one embodiment, A is a 6-membered heteroaryl, for example, pyridine or pyrazine.

In one embodiment, B is C₁₋₆ alkyl or C₃₋₁₀ cycloalkyl, where one or more hydrogens of the alkyl and cycloalkyl are optionally substituted by halogen, C₁₋₆ alkyl, C₁₋₂ alkenyl or C₃₋₁₀ cycloalkyl, where one or more hydrogens of the alkyl, cycloalkyl and alkenyl are further optionally substituted by halogen or C₁₋₆ alkyl.

In one embodiment, R¹ is C₁₋₆ alkyl, where one or more hydrogens of the alkyl are optionally substituted by halogen, D, CN, C₃₋₆ cycloalkyl, 4- to 6-membered heterocyclyl, —OR^{d} or —NR¹⁵R¹⁶.

In one embodiment, R² is C₁₋₃ alkyl or cyclopropyl.

In one embodiment, R³ is H, halogen or —OR^{e}.

In one embodiment, L¹ is bond, L² is bond, n is 0.

In one embodiment, L¹ is bond, L² is bond, n is 1.

In one embodiment, L¹ is bond, L² is —O— or —CH₂O—, n is 1.

In one embodiment, L¹ is —O—, L² is bond, n is 1.

In one embodiment, R⁴ and R⁵ are independently selected from H and F.

In one embodiment, R⁶ is 5-membered heteroaryl containing one to three heteroatoms selected from N, O and S, where one or more hydrogens of the heteroaryl are optionally substituted by C₁₋₆ alkyl, C₃₋₆ cycloalkyl or 4- to 8-membered heterocyclyl, where one or more hydrogens of the alkyl, cycloalkyl and heterocyclyl are further optionally substituted by D, halogen, C₁₋₆ alkyl or C₃₋₁₀ cycloalkyl.

In one embodiment, R^{a}, R^{b}, R^{c}, R^{e}, R^{f}, R¹¹, R¹², R¹³, R¹⁴, R¹⁵ R¹⁶, R²¹ and R²² are independently selected from H, C₁₋₆ alkyl and C₃₋₆ cycloalkyl.

In one embodiment, R^{d} is H, C₁₋₆ alkyl, C₃₋₆ cycloalkyl or 4- to 6-membered heterocyclyl, where one or more hydrogens of the alkyl, cycloalkyl and heterocyclyl are optionally substituted by F, D, C₁₋₆ alkyl or C₃₋₆ cycloalkyl.

In some embodiments, the compounds shown in Formula (I) have the following Formula (II):

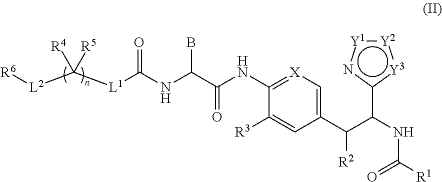

Wherein:
B is C₁₋₆ alkyl or C₃₋₁₀ cycloalkyl, where one or more hydrogens of the alkyl and cycloalkyl are optionally substituted by C₃₋₁₀ cycloalkyl, C₁₋₆ alkyl, C₁₋₂ alkenyl or halogen, where one or more hydrogens of the alkyl, cycloalkyl and alkenyl are further optionally substituted by halogen or C₁₋₆ alkyl;
X is CH or N;
Y¹ is N or CR³¹;
Y² is N, NR³² or CR³¹;
Y³ is N, NR³², O or S;
R³¹ is H, halogen or C₁₋₆ alkyl;
R³² is C₁₋₆ alkyl, C₃₋₆ cycloalkyl or 4- to 6-membered heterocyclyl, where one or more hydrogens of the alkyl, cycloalkyl and heterocyclyl are optionally substituted by C₃₋₆ cycloalkyl, halogen, C₁₋₆ alkyl, 4- to 6-membered heterocyclyl, —OR^{b} or —NR¹³R¹⁴, where one or more hydrogens of cycloalkyl and heterocyclyl are further optionally substituted by halogen or C₁₋₆ alkyl;
R¹ is C₁₋₆ alkyl, C₃₋₆ cycloalkyl or —NR²¹R²², where one or more hydrogens of the alkyl and cycloalkyl are optionally substituted by halogen, D, CN, C₃₋₆ cycloalkyl, 4- to 6-membered heterocyclyl, —OR^{d} or —NR¹⁵R¹⁶;
R² is C₁₋₆ alkyl or C₃₋₆ cycloalkyl;
R³ is H, halogen or —OR^{e};
R⁴ and R⁵ are independently selected from H, halogen and C₁₋₆ alkyl;
L¹ is bond or —O—;
L² is bond, —O— or —CH₂O—;
R⁴ and R⁵ are independently selected from H, halogen and C₁₋₆ alkyl;
R⁶ is C₃₋₁₀ cycloalkyl or 5- to 10-membered heteroaryl, where one or more hydrogens of the cycloalkyl and heteroaryl are optionally substituted by C₁₋₆ alkyl, halogen, CN, C₃₋₆ cycloalkyl, 4- to 8-membered heterocyclyl or —OR^{f}, where one or more hydrogens of the alkyl, cycloalkyl and heterocyclyl are further optionally substituted by D, halogen, C₁₋₆ alkyl or C₃₋₁₀ cycloalkyl;
R^{d} is H, C₁₋₆ alkyl, C₃₋₆ cycloalkyl or 4- to 6-membered heterocyclyl, where one or more hydrogens of the alkyl, cycloalkyl and heterocyclyl are optionally substituted by F, D, C₁₋₆ alkyl or C₃₋₆ cycloalkyl;
R^{b}, R^{e}, R^{f} R¹³, R¹⁴, R¹⁵, R¹⁶, R²¹ and R²² are independently selected from H, C₁₋₆ alkyl and C₃₋₆ cycloalkyl; and
n is 0 or 1.

In one embodiment, B is

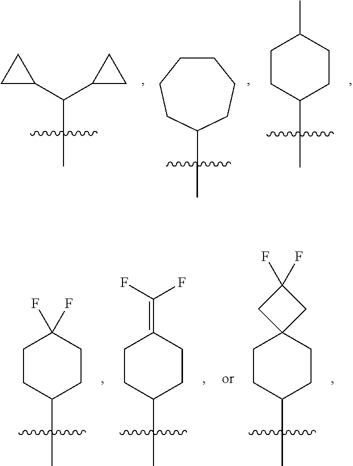

In one embodiment, Y¹ is N, Y² is CR³¹, and Y³ is NR³², O or S.

In one embodiment, Y¹ is CR³¹, Y² is N, and Y³ is NR³².

In one embodiment, Y¹ is CR³¹, Y² is CR³¹, and Y³ is N.

In one embodiment, Y¹ is N, Y² is N, and Y³ is NR³².

In one embodiment, Y¹ is CR³¹, Y² is CR³¹, and Y³ is NR³².

In one embodiment, the 5-membered heteroaryl ring in Formula II is

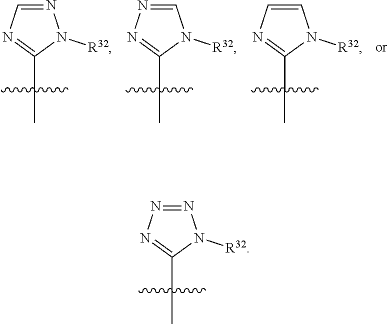

In one embodiment, R¹ is C₁₋₆ alkyl, where one or more hydrogens of the alkyl are optionally substituted by halogen, D, CN, C₃₋₆ cycloalkyl, 4- to 6-membered heterocyclyl, —OR^{d} or —NR¹⁵R¹⁶.

In one embodiment, R² is methyl or cyclopropyl.

In one embodiment, R³ is H, F or —OCH₃.

In one embodiment, L¹ is bond, L² is bond, n is 0.

In one embodiment, L¹ is bond, L² is bond, n is 1.

In one embodiment, L¹ is bond, L² is —O— or —CH₂O—, n is 1.

In one embodiment, L¹ is —O—, L² is bond, n is 1.

In one embodiment, R⁴ and R⁵ are independently selected from H and F.

In one embodiment, R⁶ is pyrazolyl, triazolyl, oxadiazolyl, isoxazolyl or pyridinyl, where one or more hydrogens of pyrazolyl, triazolyl, oxadiazolyl, isoxazolyl and pyridinyl are optionally substituted by F, C₁₋₆ alkyl, fluorinated C₁₋₂ alkyl, deuterated C₁₋₂ alkyl, C₃₋₆ cycloalkyl, —OCH₃ or —OCF₃.

In some embodiments, the compounds shown in Formula (I) have the following Formula (III):

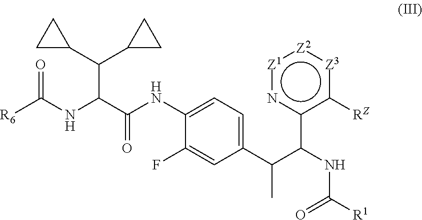

Wherein:
Z¹, Z² and Z³ are independently selected from N and CR⁵¹;
R^{z} is C₁₋₆ alkyl or —OR^{a}, where one or more hydrogens of the alkyl are optionally substituted by F, C₃₋₆ cycloalkyl, 4- to 6-membered heterocyclyl, —OR^{b} or —NR¹³R¹⁴;
R¹ is C₁₋₆ alkyl, where one or more hydrogens of the alkyl are optionally substituted by F, D, CN, 4- to 6-membered heterocyclyl, —OR^{d} or —NR¹⁵R¹⁶;
R⁶ is 5-membered heteroaryl containing one to three heteroatoms selected from N, O and S, where one or more hydrogens of the heteroaryl are optionally substituted by C₁₋₆ alkyl, fluorinated C₁₋₆ alkyl, deuterated C₁₋₆ alkyl or C₃₋₆ cycloalkyl;
R^{d} is H, C₁₋₆ alkyl, C₃₋₆ cycloalkyl or 4- to 6-membered heterocyclyl, where one or more hydrogens of the alkyl, cycloalkyl and heterocyclyl are optionally substituted by F, D, C₁₋₆ alkyl or C₃₋₆ cycloalkyl; and
R^{a}, R^{b}, R¹³, R¹⁴, R¹⁵, R¹⁶ and R⁵¹ are independently selected from H, C₁₋₆ alkyl, fluorinated C₁₋₆ alkyl and C₃₋₆ cycloalkyl.

In one embodiment, the 6-membered heteroaryl ring in Formula III is

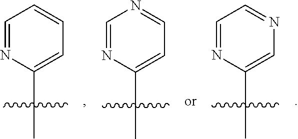

In one embodiment, R⁶ is pyrazolyl, oxadiazolyl or isoxazolyl, where one or more hydrogens of pyrazolyl, oxadiazolyl and isoxazolyl are optionally substituted by F, C₁₋₆ alkyl, fluorinated C₁₋₂ alkyl, deuterated C₁₋₂ alkyl or C₃₋₆ cycloalkyl.

The present invention further relates to the following Compounds 1-88 or their pharmaceutically acceptable salts, prodrugs, stable isotope derivatives, isomers, and mixtures thereof.

| Compound | |
|---|---|
| No. | Compound Structure and Chemical Name |
| 1. | 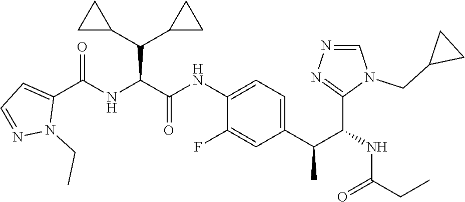 [SMILES: CCC(=O)N[C@@H](c1nncn1CC1CC1)[C@@H](C)c1ccc(NC(=O)[C@@H](NC(=O)c2ccnn2CC)C(C2CC2)C2CC2)c(F)c1] |
| | N-((S)-1,1-dicyclopropyl-3-((4-((1R,2S)-1-(4-(cyclopropylmethyl)-4H-1,2,4- |
| | triazol-3-yl)-1-propionamidopropan-2-yl)-2-fluorophenyl)amino)-3-oxopropan- |
| | 2-yl)-1-ethyl-1H-pyrazole-5-carboxamide |
| 2. | 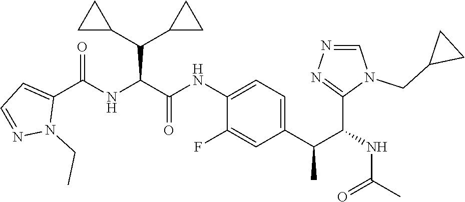 [SMILES: CCn1nccc1C(=O)N[C@H](C(=O)Nc1ccc([C@H](C)[C@@H](NC(C)=O)c2nncn2CC2CC2)cc1F)C(C1CC1)C1CC1] |
| | N-((S)-1-((4-((1R,2S)-1-acetamido-1-(4-(cyclopropylmethyl)-4H-1,2,4-triazol-3- |
| | yl)propan-2-yl)-2-fluorophenyl)amino)-3,3-dicyclopropyl-1-oxopropan-2-yl)-1- |
| | ethyl-1H-pyrazole-5-carboxamide |
| 3. | 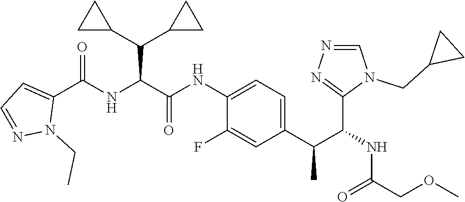 [SMILES: CCn1nccc1C(=O)N[C@H](C(=O)Nc1ccc([C@H](C)[C@@H](NC(=O)COC)c2nncn2CC2CC2)cc1F)C(C1CC1)C1CC1] |
| | N-((S)-1,1-dicyclopropyl-3-((4-((1R,2S)-1-(4-(cyclopropylmethyl)-4H-1,2,4- |
| | triazol-3-yl)-1-(2-methoxyacetamido)propan-2-yl)-2-fluorophenyl)amino)-3- |
| | oxopropan-2-yl)-1-ethyl-1H-pyrazole-5-carboxamide |
| 4. | 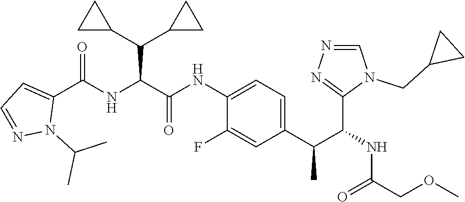 [SMILES: COCC(=O)N[C@@H](c1nncn1CC1CC1)[C@@H](C)c1ccc(NC(=O)[C@@H](NC(=O)c2ccnn2C(C)C)C(C2CC2)C2CC2)c(F)c1] |
| | N-((S)-1,1-dicyclopropyl-3-((4-((1R,2S)-1-(4-(cyclopropylmethyl)-4H-1,2,4- |
| | triazol-3-yl)-1-(2-methoxyacetamido)propan-2-yl)-2-fluorophenyl)amino)-3- |
| | oxopropan-2-yl)-1-isopropyl-1H-pyrazole-5-carboxamide |
| 5. | 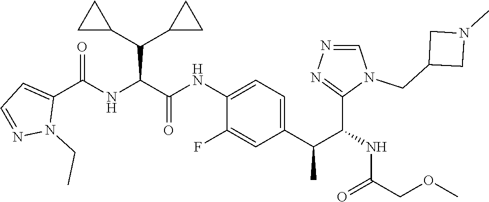 [SMILES: CCn1nccc1C(=O)N[C@H](C(=O)Nc1ccc([C@H](C)[C@@H](NC(=O)COC)c2nncn2CC2CN(C)C2)cc1F)C(C1CC1)C1CC1] |
| | N-((S)-1,1-dicyclopropyl-3-((2-fluoro-4-((1R,2S)-1-(2-methoxyacetamido)-1-(4- |
| | ((1-methylazetidin-3-yl)methyl)-4H-1,2,4-triazol-3-yl)propan-2- |
| | yl)phenyl)amino)-3-oxopropan-2-yl)-1-ethyl-1H-pyrazole-5-carboxamide |
| 6. | 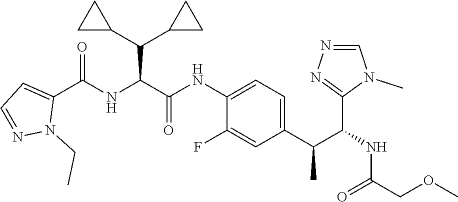 [SMILES: CCn1nccc1C(=O)N[C@H](C(=O)Nc1ccc([C@H](C)[C@@H](NC(=O)COC)c2nncn2C)cc1F)C(C1CC1)C1CC1] |
| | N-((S)-1,1-dicyclopropyl-3-((2-fluoro-4-((1R,2S)-1-(2-methoxyacetamido)-1-(4- |
| | methyl-4H-1,2,4-triazol-3-yl)propan-2-yl)phenyl)amino)-3-oxopropan-2-yl)-1- |
| | ethyl-1H-pyrazole-5-carboxamide |
| 7. | 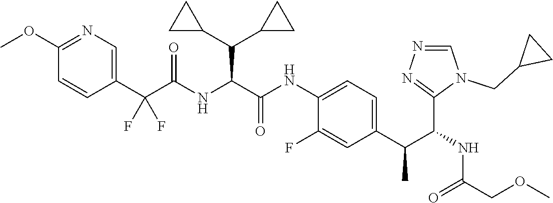 [SMILES: COCC(=O)N[C@@H](c1nncn1CC1CC1)[C@@H](C)c1ccc(NC(=O)[C@@H](NC(=O)C(F)(F)c2ccc(OC)nc2)C(C2CC2)C2CC2)c(F)c1] |
| | (S)-3,3-dicyclopropyl-N-(4-((1R,2S)-1-(4-(cyclopropylmethyl)-4H-1,2,4-triazol- |
| | 3-yl)-1-(2-methoxyacetamido)propan-2-yl)-2-fluorophenyl)-2-(2,2-difluoro-2- |
| | (6-methoxypyridin-3-yl)acetamido)propanamide |
| 8. | 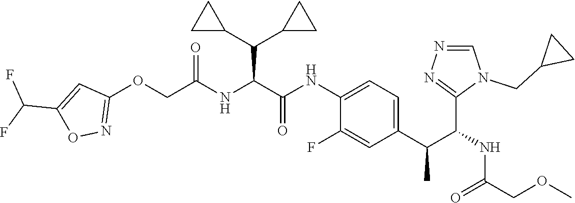 [SMILES: COCC(=O)N[C@@H](c1nncn1CC1CC1)[C@@H](C)c1ccc(NC(=O)[C@@H](NC(=O)COc2cc(C(F)F)on2)C(C2CC2)C2CC2)c(F)c1] |
| | (S)-3,3-dicyclopropyl-N-(4-((1R,2S)-1-(4-(cyclopropylmethyl)-4H-1,2,4-triazol- |
| | 3-yl)-1-(2-methoxyacetamido)propan-2-yl)-2-fluorophenyl)-2-(2-((5- |
| | (difluoromethyl)isoxazol-3-yl)oxy)acetamido)propenamide |
| 9. | 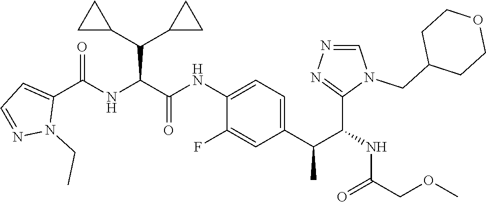 [SMILES: CCn1nccc1C(=O)N[C@H](C(=O)Nc1ccc([C@H](C)[C@@H](NC(=O)COC)c2nncn2CC2CCOCC2)cc1F)C(C1CC1)C1CC1] |
| | N-((S)-1,1-dicyclopropyl-3-((2-fluoro-4-((1R,2S)-1-(2-methoxyacetamido)-1-(4- |
| | ((tetrahydro-2H-pyran-4-yl)methyl)-4H-1,2,4-triazol-3-yl)propan-2- |
| | yl)phenyl)amino)-3-oxopropan-2-yl)-1-ethyl-1H-pyrazole-5-carboxamide |
| 10. | 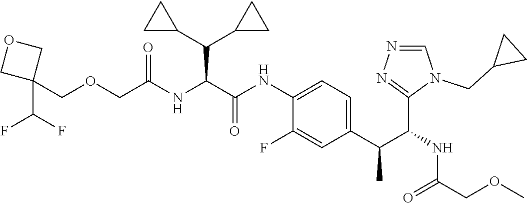 [SMILES: COCC(=O)N[C@@H](c1nncn1CC1CC1)[C@@H](C)c1ccc(NC(=O)[C@@H](NC(=O)COCC2(C(F)F)COC2)C(C2CC2)C2CC2)c(F)c1] |
| | (S)-3,3-dicyclopropyl-N-(4-((1R,2S)-1-(4-(cyclopropylmethyl)-4H-1,2,4-triazol- |
| | 3-yl)-1-(2-methoxyacetamido)propan-2-yl)-2-fluorophenyl)-2-(2-((3- |
| | (difluoromethyl)oxetan-3-yl)methoxy)acetamido)propanamide |
| 11. | 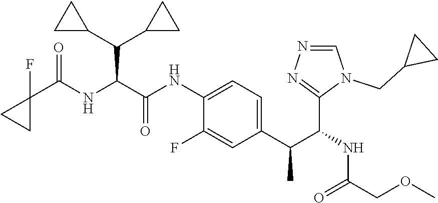 [SMILES: COCC(=O)N[C@@H](c1nncn1CC1CC1)[C@@H](C)c1ccc(NC(=O)[C@@H](NC(=O)C2(F)CC2)C(C2CC2)C2CC2)c(F)c1] |
| | N-((S)-1,1-dicyclopropyl-3-((4-((1R,2S)-1-(4-(cyclopropylmethyl)-4H-1,2,4- |
| | triazol-3-yl)-1-(2-methoxyacetamido)propan-2-yl)-2-fluorophenyl)amino)-3- |
| | oxopropan-2-yl)-1-fluorocyclopropane-1-carboxamide |
| 12. | 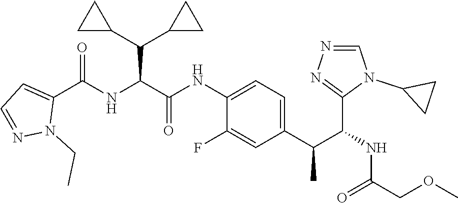 [SMILES: CCn1nccc1C(=O)N[C@H](C(=O)Nc1ccc([C@H](C)[C@@H](NC(=O)COC)c2nncn2C2CC2)cc1F)C(C1CC1)C1CC1] |
| | N-((S)-1,1-dicyclopropyl-3-((4-((1R,2S)-1-(4-cyclopropyl-4H-1,2,4-triazol-3-yl)- |
| | 1-(2-methoxyacetamido)propan-2-yl)-2-fluorophenyl)amino)-3-oxopropan-2- |
| | yl)-1-ethyl-1H-pyrazole-5-carboxamide |
| 13. | 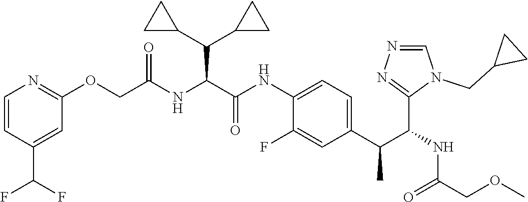 [SMILES: COCC(=O)N[C@@H](c1nncn1CC1CC1)[C@@H](C)c1ccc(NC(=O)[C@@H](NC(=O)COc2cc(C(F)F)ccn2)C(C2CC2)C2CC2)c(F)c1] |
| | (S)-3,3-dicyclopropyl-N-(4-((1R,2S)-1-(4-(cyclopropylmethyl)-4H-1,2,4-triazol- |
| | 3-yl)-1-(2-methoxyacetamido)propan-2-yl)-2-fluorophenyl)-2-(2-((4- |
| | (difluoromethyl)pyridin-2-yl)oxy)acetamido)propanamide |
| 14. | 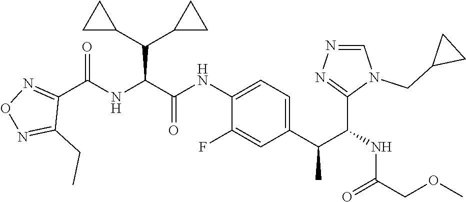 [SMILES: CCc1nonc1C(=O)N[C@H](C(=O)Nc1ccc([C@H](C)[C@@H](NC(=O)COC)c2nncn2CC2CC2)cc1F)C(C1CC1)C1CC1] |
| | N-((S)-1,1-dicyclopropyl-3-((4-((1R,2S)-1-(4-(cyclopropylmethyl)-4H-1,2,4- |
| | triazol-3-yl)-1-(2-methoxyacetamido)propan-2-yl)-2-fluorophenyl)amino)-3- |
| | oxopropan-2-yl)-4-ethyl-1,2,5-oxadiazole-3-carboxamide |
| 15. | 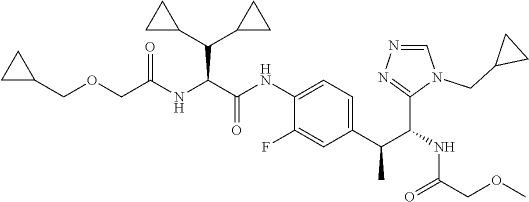 [SMILES: COCC(=O)N[C@@H](c1nncn1CC1CC1)[C@@H](C)c1ccc(NC(=O)[C@@H](NC(=O)COCC2CC2)C(C2CC2)C2CC2)c(F)c1] |
| | (S)-3,3-dicyclopropyl-2-(2-(cyclopropylmethoxy)acetamido)-N-(4-((1R,2S)-1- |
| | (4-(cyclopropylmethyl)-4H-1,2,4-triazol-3-yl)-1-(2-methoxyacetamido)propan- |
| | 2-yl)-2-fluorophenyl)propanamide |
| 16. | 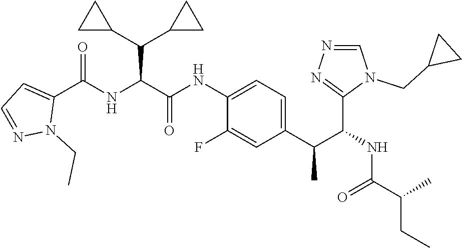 [SMILES: CC[C@@H](C)C(=O)N[C@@H](c1nncn1CC1CC1)[C@@H](C)c1ccc(NC(=O)[C@@H](NC(=O)c2ccnn2CC)C(C2CC2)C2CC2)c(F)c1] |
| | N-((S)-1,1-dicyclopropyl-3-((4-((1R,2S)-1-(4-(cyclopropylmethyl)-4H-1,2,4- |
| | triazol-3-yl)-1-((S)-2-methoxypropanamido)propan-2-yl)-2- |
| | fluorophenyl)amino)-3-oxopropan-2-yl)-1-ethyl-1H-pyrazole-5-carboxamide |
| 17. | 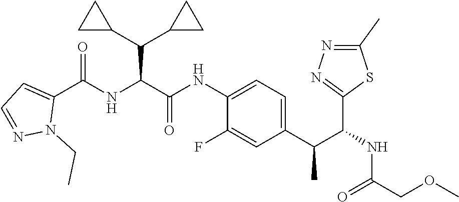 [SMILES: CCn1nccc1C(=O)N[C@H](C(=O)Nc1ccc([C@H](C)[C@@H](NC(=O)COC)c2nnc(C)s2)cc1F)C(C1CC1)C1CC1] |
| | N-((S)-1,1-dicyclopropyl-3-((2-fluoro-4-((1R,2S)-1-(2-methoxyacetamido)-1-(5- |
| | methyl-1,3,4-thiadiazol-2-yl)propan-2-yl)phenyl)amino)-3-oxopropan-2-yl)-1- |
| | ethyl-1H-pyrazole-5-carboxamide |
| 18. | 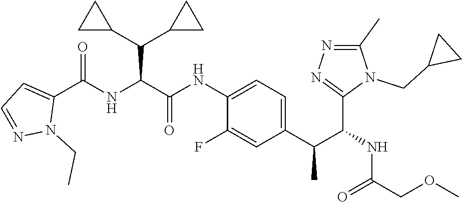 [SMILES: CCn1nccc1C(=O)N[C@H](C(=O)Nc1ccc([C@H](C)[C@@H](NC(=O)COC)c2nnc(C)n2CC2CC2)cc1F)C(C1CC1)C1CC1] |
| | N-((S)-1,1-dicyclopropyl-3-((4-((1R,2S)-1-(4-(cyclopropylmethyl)-5-methyl-4H- |
| | 1,2,4-triazol-3-yl)-1-(2-methoxyacetamido)propan-2-yl)-2-fluorophenyl)amino)- |
| | 3-oxopropan-2-yl)-1-ethyl-1H-pyrazole-5-carboxamide |
| 19. | 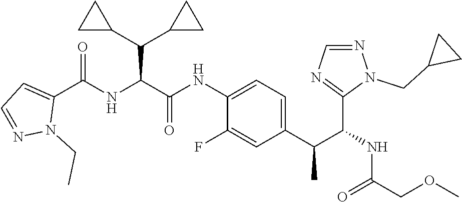 [SMILES: CCn1nccc1C(=O)N[C@H](C(=O)Nc1ccc([C@H](C)[C@@H](NC(=O)COC)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-1-ethyl-1H-pyrazole-5-carboxamide |
| 20. | 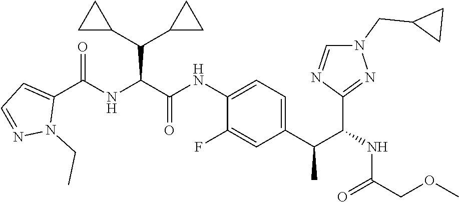 [SMILES: CCn1nccc1C(=O)N[C@H](C(=O)Nc1ccc([C@H](C)[C@@H](NC(=O)COC)c2ncn(CC3CC3)n2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-3-yl)-1-(2-methoxyacetamido)propan-2-yl)-2-fluorophenyl)amino)-3- |
| | oxopropan-2-yl)-1-ethyl-1H-pyrazole-5-carboxamide |
| 21. | 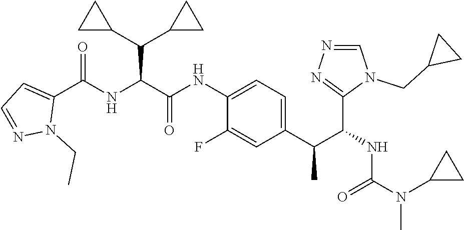 [SMILES: CCn1nccc1C(=O)N[C@H](C(=O)Nc1ccc([C@H](C)[C@@H](NC(=O)N(C)C2CC2)c2nncn2CC2CC2)cc1F)C(C1CC1)C1CC1] |
| | N-((S)-1,1-dicyclopropyl-3-((4-((1R,2S)-1-(3-cyclopropyl-3-methylureido)-1-(4- |
| | (cyclopropylmethyl)-4H-1,2,4-triazol-3-yl)propan-2-yl)-2-fluorophenyl)amino)- |
| | 3-oxopropan-2-yl)-1-ethyl-1H-pyrazole-5-carboxamide |
| 22. | 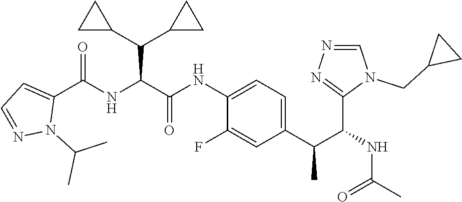 [SMILES: CC(=O)N[C@@H](c1nncn1CC1CC1)[C@@H](C)c1ccc(NC(=O)[C@@H](NC(=O)c2ccnn2C(C)C)C(C2CC2)C2CC2)c(F)c1] |
| | N-((S)-1-((4-((1R,2S)-1-acetamido-1-(4-(cyclopropylmethyl)-4H-1,2,4-triazol-3- |
| | yl)propan-2-yl)-2-fluorophenyl)amino)-3,3-dicyclopropyl-1-oxopropan-2-yl)-1- |
| | isopropyl-1H-pyrazole-5-carboxamide |
| 23. | 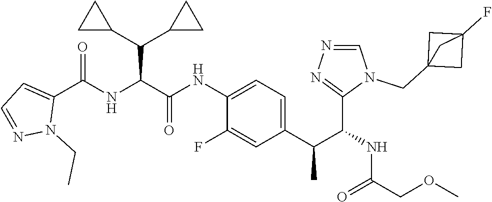 [SMILES: CCn1nccc1C(=O)N[C@H](C(=O)Nc1ccc([C@H](C)[C@@H](NC(=O)COC)c2nncn2CC23CC(F)(C2)C3)cc1F)C(C1CC1)C1CC1] |
| | N-((S)-1,1-dicyclopropyl-3-((2-fluoro-4-((1R,2S)-1-(4-((3- |
| | fluorobicyclo[1.1.1]pentan-1-yl)methyl)-4H-1,2,4-triazol-3-yl)-1-(2- |
| | methoxyacetamido)propan-2-yl)phenyl)amino)-3-oxopropan-2-yl)-1-ethyl-1H- |
| | pyrazole-5-carboxamide |
| 24. | 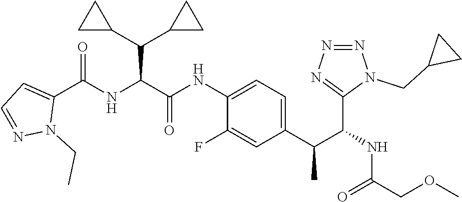 [SMILES: CCn1nccc1C(=O)N[C@H](C(=O)Nc1ccc([C@H](C)[C@@H](NC(=O)COC)c2nnnn2CC2CC2)cc1F)C(C1CC1)C1CC1] |
| | N-((S)-1,1-dicyclopropyl-3-((4-((1R,2S)-1-(1-(cyclopropylmethyl)-1H-tetrazol- |
| | 5-yl)-1-(2-methoxyacetamido)propan-2-yl)-2-fluorophenyl)amino)-3- |
| | oxopropan-2-yl)-1-ethyl-1H-pyrazole-5-carboxamide |
| 25. | 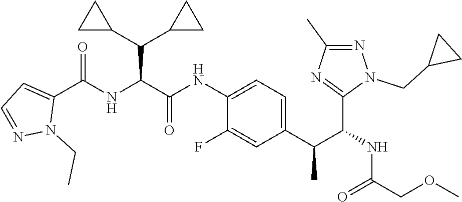 [SMILES: CCn1nccc1C(=O)N[C@H](C(=O)Nc1ccc([C@H](C)[C@@H](NC(=O)COC)c2nc(C)nn2CC2CC2)cc1F)C(C1CC1)C1CC1] |
| | N-((S)-1,1-dicyclopropyl-3-((4-((1R,2S)-1-(1-(cyclopropylmethyl)-3-methyl-1H- |
| | 1,2,4-triazol-5-yl)-1-(2-methoxyacetamido)propan-2-yl)-2-fluorophenyl)amino)- |
| | 3-oxopropan-2-yl)-1-ethyl-1H-pyrazole-5-carboxamide |
| 26. | 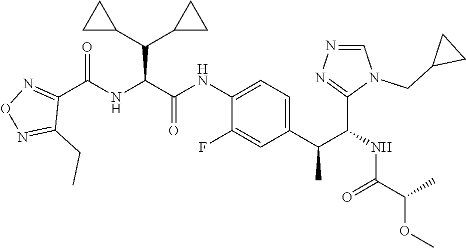 [SMILES: CCc1nonc1C(=O)N[C@H](C(=O)Nc1ccc([C@H](C)[C@@H](NC(=O)[C@H](C)OC)c2nncn2CC2CC2)cc1F)C(C1CC1)C1CC1] |
| | N-((S)-1,1-dicyclopropyl-3-((4-((1R,2S)-1-(4-(cyclopropylmethyl)-4H-1,2,4- |
| | triazol-3-yl)-1-((S)-2-methoxypropanamido)propan-2-yl)-2- |
| | fluorophenyl)amino)-3-oxopropan-2-yl)-4-ethyl-1,2,5-oxadiazole-3- |
| | carboxamide |
| 27. | 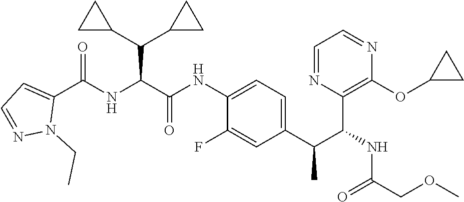 [SMILES: CCn1nccc1C(=O)N[C@H](C(=O)Nc1ccc([C@H](C)[C@@H](NC(=O)COC)c2nccnc2OC2CC2)cc1F)C(C1CC1)C1CC1] |
| | N-((S)-1-((4-((1R,2S)-1-(3-cyclopropoxypyrazin-2-yl)-1-(2- |
| | methoxyacetamido)propan-2-yl)-2-fluorophenyl)amino)-3,3-dicyclopropyl-1- |
| | oxopropan-2-yl)-1-ethyl-1H-pyrazole-5-carboxamide |
| 28. | 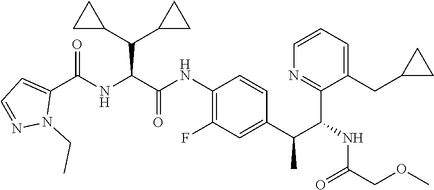 [SMILES: CCn1nccc1C(=O)N[C@H](C(=O)Nc1ccc([C@H](C)[C@@H](NC(=O)COC)c2ncccc2CC2CC2)cc1F)C(C1CC1)C1CC1] |
| | N-((S)-1,1-dicyclopropyl-3-((4-((1R,2S)-1-(3-(cyclopropylmethyl)pyridin-2-yl)- |
| | 1-(2-methoxyacetamido)propan-2-yl)-2-fluorophenyl)amino)-3-oxopropan-2- |
| | yl)-1-ethyl-1H-pyrazole-5-carboxamide |
| 29. | 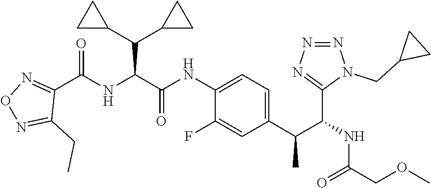 [SMILES: CCc1nonc1C(=O)N[C@H](C(=O)Nc1ccc([C@H](C)[C@@H](NC(=O)COC)c2nnnn2CC2CC2)cc1F)C(C1CC1)C1CC1] |
| | N-((S)-1,1-dicyclopropyl-3-((4-((1R,2S)-1-(1-(cyclopropylmethyl)-1H-tetrazol- |
| | 5-yl)-1-(2-methoxyacetamido)propan-2-yl)-2-fluorophenyl)amino)-3- |
| | oxopropan-2-yl)-4-ethyl-1,2,5-oxadiazole-3-carboxamide |
| 30. | 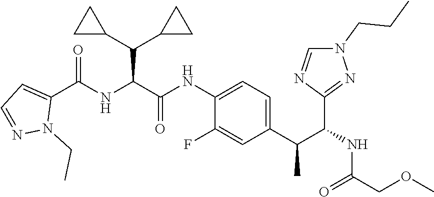 [SMILES: CCCn1cnc([C@H](NC(=O)COC)[C@@H](C)c2ccc(NC(=O)[C@@H](NC(=O)c3ccnn3CC)C(C3CC3)C3CC3)c(F)c2)n1] |
| | N-((S)-1,1-dicyclopropyl-3-((2-fluoro-4-((1R,2S)-1-(2-methoxyacetamido)-1-(1- |
| | propyl-1H-1,2,4-triazol-3-yl)propan-2-yl)phenyl)amino)-3-oxopropan-2-yl)-1- |
| | ethyl-1H-pyrazole-5-carboxamide |
| 31. | 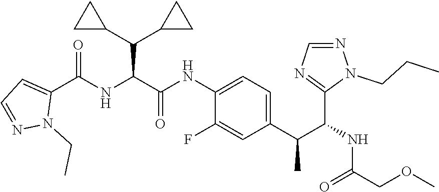 [SMILES: CCCn1ncnc1[C@H](NC(=O)COC)[C@@H](C)c1ccc(NC(=O)[C@@H](NC(=O)c2ccnn2CC)C(C2CC2)C2CC2)c(F)c1] |
| | N-((S)-1,1-dicyclopropyl-3-((2-fluoro-4-((1R,2S)-1-(2-methoxyacetamido)-1-(1- |
| | propyl-1H-1,2,4-triazol-5-yl)propan-2-yl)phenyl)amino)-3-oxopropan-2-yl)-1- |
| | ethyl-1H-pyrazole-5-carboxamide |
| 32. | 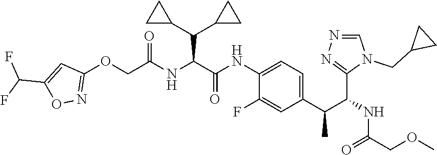 [SMILES: COCC(=O)N[C@@H](c1nncn1CC1CC1)[C@@H](C)c1ccc(NC(=O)[C@@H](NC(=O)COc2cc(C(F)F)on2)C(C2CC2)C2CC2)c(F)c1] |
| | (5-(Difluoromethyl)isoxazol-3-yl)methyl ((S)-1,1-dicyclopropyl-3-((4-((1R,2S)- |
| | 1-(4-(cyclopropylmethyl)-4H-1,2,4-triazol-3-yl)-1-(2- |
| | methoxyacetamido)propan-2-yl)-2-fluorophenyl)amino)-3-oxopropan-2- |
| | yl)carbamate |
| 33. | 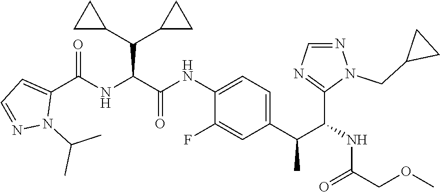 [SMILES: COCC(=O)N[C@@H](c1ncnn1CC1CC1)[C@@H](C)c1ccc(NC(=O)[C@@H](NC(=O)c2ccnn2C(C)C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-1-isopropyl-1H-pyrazole-5-carboxamide |
| 34. | 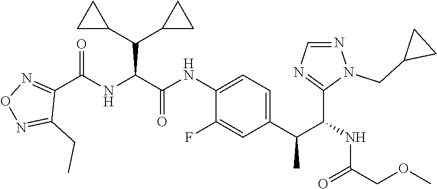 [SMILES: CCc1nonc1C(=O)N[C@H](C(=O)Nc1ccc([C@H](C)[C@@H](NC(=O)COC)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-4-ethyl-1,2,5-oxadiazole-3-carboxamide |
| 35. | 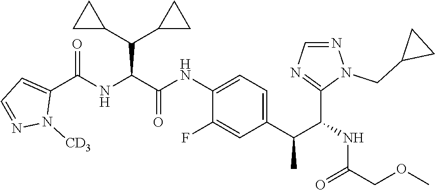 [SMILES: [2H]C([2H])([2H])n1nccc1C(=O)N[C@H](C(=O)Nc1ccc([C@H](C)[C@@H](NC(=O)COC)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-1-(methyl-d₃)-1H-pyrazole-5-carboxamide |
| 36. | 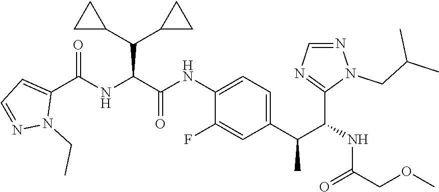 [SMILES: CCn1nccc1C(=O)N[C@H](C(=O)Nc1ccc([C@H](C)[C@@H](NC(=O)COC)c2ncnn2CC(C)C)cc1F)C(C1CC1)C1CC1] |
| | N-((S)-1,1-dicyclopropyl-3-((2-fluoro-4-((1R,2S)-1-(1-isobutyl-1H-1,2,4-triazol- |
| | 5-yl)-1-(2-methoxyacetamido)propan-2-yl)phenyl)amino)-3-oxopropan-2-yl)-1- |
| | ethyl-1H-pyrazole-5-carboxamide |
| 37. | 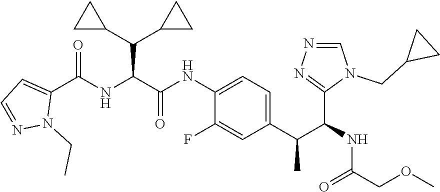 [SMILES: CCn1nccc1C(=O)N[C@H](C(=O)Nc1ccc([C@H](C)[C@H](NC(=O)COC)c2nncn2CC2CC2)cc1F)C(C1CC1)C1CC1] |
| | N-((S)-1,1-dicyclopropyl-3-((4-((1S,2S)-1-(4-(cyclopropylmethyl)-4H-1,2,4- |
| | triazol-3-yl)-1-(2-methoxyacetamido)propan-2-yl)-2-fluorophenyl)amino)-3- |
| | oxopropan-2-yl)-1-ethyl-1H-pyrazole-5-carboxamide |
| 38. | 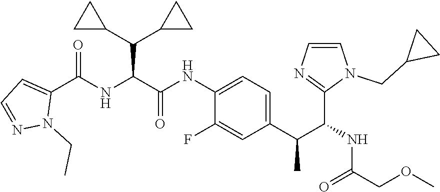 [SMILES: CCn1nccc1C(=O)N[C@H](C(=O)Nc1ccc([C@H](C)[C@@H](NC(=O)COC)c2nccn2CC2CC2)cc1F)C(C1CC1)C1CC1] |
| | N-((S)-1,1-dicyclopropyl-3-((4-((1R,2S)-1-(1-(cyclopropylmethyl)-1H-imidazol- |
| | 2-yl)-1-(2-methoxyacetamido)propan-2-yl)-2-fluorophenyl)amino)-3- |
| | oxopropan-2-yl)-1-ethyl-1H-pyrazole-5-carboxamide |
| 39. | 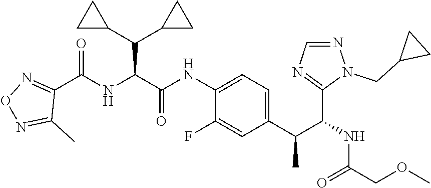 [SMILES: COCC(=O)N[C@@H](c1ncnn1CC1CC1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-4-methyl-1,2,5-oxadiazole-3-carboxamide |
| 40. | 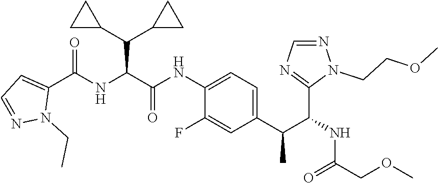 [SMILES: CCn1nccc1C(=O)N[C@H](C(=O)Nc1ccc([C@H](C)[C@@H](NC(=O)COC)c2ncnn2CCOC)cc1F)C(C1CC1)C1CC1] |
| | N-((S)-1,1-dicyclopropyl-3-((2-fluoro-4-((1R,2S)-1-(1-(2-methoxyethyl)-1H- |
| | 1,2,4-triazol-5-yl)-1-propionamidopropan-2-yl)phenyl)amino)-3-oxopropan-2- |
| | yl)-1-ethyl-1H-pyrazole-5-carboxamide |
| 41. | 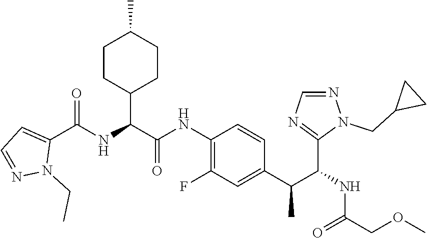 |
| | N-((S)-2-((4-((1R,2S)-1-(1-(cyclopropylmethyl)-1H-1,2,4-triazol-5-yl)-1-(2- |
| | methoxyacetamido)propan-2-yl)-2-fluorophenyl)amino)-1-((1R,4S)-4- |
| | methylcyclohexyl)-2-oxoethyl)-1-ethyl-1H-pyrazole-5-carboxamide |
| 42. | 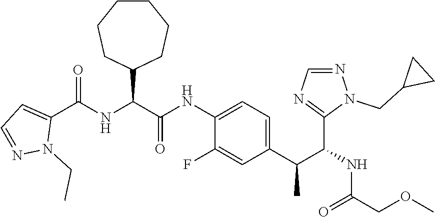 [SMILES: CCn1nccc1C(=O)N[C@H](C(=O)Nc1ccc([C@H](C)[C@@H](NC(=O)COC)c2ncnn2CC2CC2)cc1F)C1CCCCCC1] |
| | N-((S)-1-cycloheptyl-2-((4-((1R,2S)-1-(1-(cyclopropylmethyl)-1H-1,2,4-triazol- |
| | 5-yl)-1-(2-methoxyacetamido)propan-2-yl)-2-fluorophenyl)amino)-2-oxoethyl)- |
| | 1-ethyl-1H-pyrazole-5-carboxamide |
| 43. | 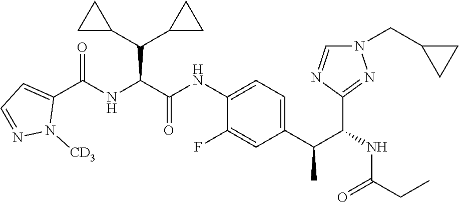 [SMILES: [2H]C([2H])([2H])n1nccc1C(=O)N[C@H](C(=O)Nc1ccc([C@H](C)[C@@H](NC(=O)CC)c2ncn(CC3CC3)n2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-3-yl)-1-propionamidopropan-2-yl)-2-fluorophenyl)amino)-3-oxopropan- |
| | 2-yl)-1-(methyl-d₃)-1H-pyrazole-5-carboxamide |
| 44. | 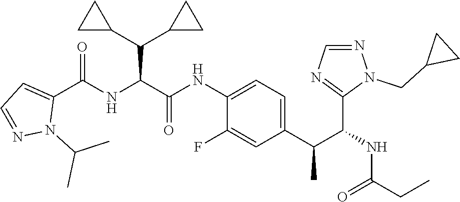 [SMILES: CCC(=O)N[C@@H](c1ncnn1CC1CC1)[C@@H](C)c1ccc(NC(=O)[C@@H](NC(=O)c2ccnn2C(C)C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-1-isopropyl-1H-pyrazole-5-carboxamide |
| 45. | 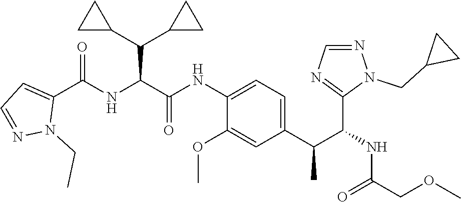 [SMILES: CCn1nccc1C(=O)N[C@H](C(=O)Nc1ccc([C@H](C)[C@@H](NC(=O)COC)c2ncnn2CC2CC2)cc1OC)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-methoxyphenyl)amino)-3- |
| | oxopropan-2-yl)-1-ethyl-1H-pyrazole-5-carboxamide |
| 46. | 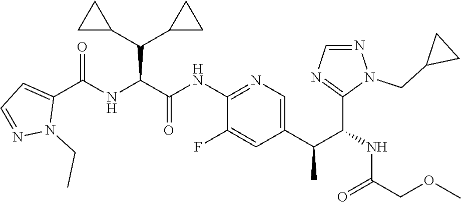 [SMILES: CCn1nccc1C(=O)N[C@H](C(=O)Nc1ncc([C@H](C)[C@@H](NC(=O)COC)c2ncnn2CC2CC2)cc1F)C(C1CC1)C1CC1] |
| | N-((S)-1,1-dicyclopropyl-3-((5-((1R,2S)-1-(1-(cyclopropylmethyl)-1H-1,2,4- |
| | triazol-5-yl)-1-(2-methoxyacetamido)propan-2-yl)-3-fluoropyridin-2-yl)amino)- |
| | 3-oxopropan-2-yl)-1-ethyl-1H-pyrazole-5-carboxamide |
| 47. | 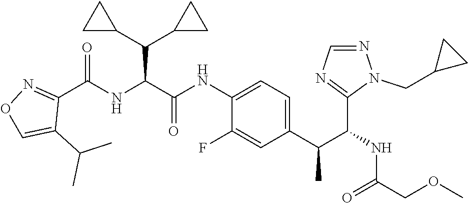 [SMILES: COCC(=O)N[C@@H](c1ncnn1CC1CC1)[C@@H](C)c1ccc(NC(=O)[C@@H](NC(=O)c2nocc2C(C)C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-4-isopropyl-1,2,5-oxadiazole-3-carboxamide |
| 48. | 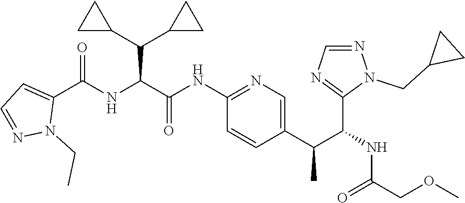 [SMILES: CCn1nccc1C(=O)N[C@H](C(=O)Nc1ccc([C@H](C)[C@@H](NC(=O)COC)c2ncnn2CC2CC2)cn1)C(C1CC1)C1CC1] |
| | N-((S)-1,1-dicyclopropyl-3-((5-((1R,2S)-1-(1-(cyclopropylmethyl)-1H-1,2,4- |
| | triazol-5-yl)-1-(2-methoxyacetamido)propan-2-yl)pyridin-2-yl)amino)-3- |
| | oxopropan-2-yl)-1-ethyl-1H-pyrazole-5-carboxamide |
| 49. | 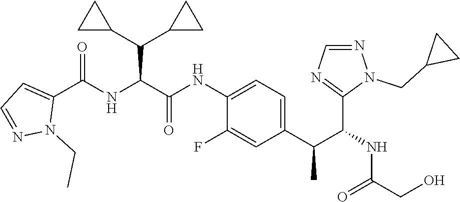 [SMILES: CCn1nccc1C(=O)N[C@H](C(=O)Nc1ccc([C@H](C)[C@@H](NC(=O)CO)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-hydroxyacetamido)propan-2-yl)-2-fluorophenyl)amino)-3- |
| | oxopropan-2-yl)-1-ethyl-1H-pyrazole-5-carboxamide |
| 50. | 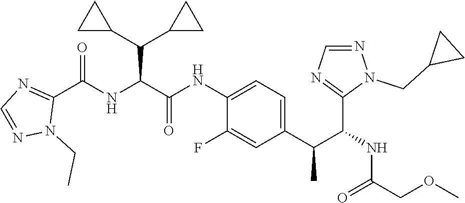 [SMILES: CCn1ncnc1C(=O)N[C@H](C(=O)Nc1ccc([C@H](C)[C@@H](NC(=O)COC)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-1-ethyl-1H-1,2,4-triazole-5-carboxamide |
| 51. | 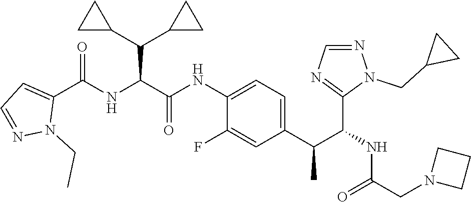 [SMILES: CCn1nccc1C(=O)N[C@H](C(=O)Nc1ccc([C@H](C)[C@@H](NC(=O)CN2CCC2)c2ncnn2CC2CC2)cc1F)C(C1CC1)C1CC1] |
| | N-((S)-1-((4-((1R,2S)-1-(2-(azetidin-1-yl)acetamido)-1-(1-(cyclopropylmethyl)- |
| | 1H-1,2,4-triazol-5-yl)propan-2-yl)-2-fluorophenyl)amino)-3,3-dicyclopropyl-1- |
| | oxopropan-2-yl)-1-ethyl-1H-pyrazole-5-carboxamide |
| 52. | 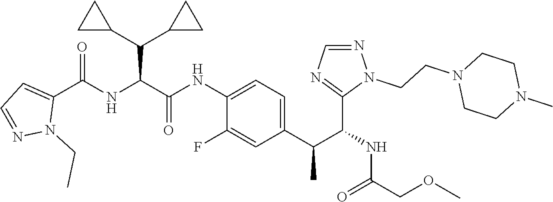 [SMILES: CCn1nccc1C(=O)N[C@H](C(=O)Nc1ccc([C@H](C)[C@@H](NC(=O)COC)c2ncnn2CCN2CCN(C)CC2)cc1F)C(C1CC1)C1CC1] |
| | N-((S)-1,1-dicyclopropyl-3-((2-fluoro-4-((1R,2S)-1-(2-methoxyacetamido)-1-(1- |
| | (2-(4-methylpiperazin-1-yl)ethyl)-1H-1,2,4-triazol-5-yl)propan-2- |
| | yl)phenyl)amino)-3-oxopropan-2-yl)-1-ethyl-1H-pyrazole-5-carboxamide |
| 53. | 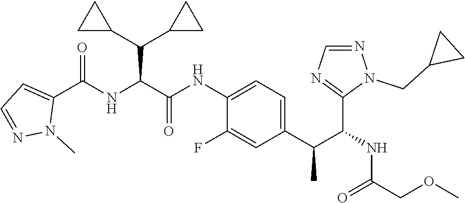 [SMILES: COCC(=O)N[C@@H](c1ncnn1CC1CC1)[C@@H](C)c1ccc(NC(=O)[C@@H](NC(=O)c2ccnn2C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-(2-methoxyacetamido)propan-2-yl)-2-fluorophenyl)amino)-3- |
| | oxopropan-2-yl)-1-methyl-1H-pyrazole-5-carboxamide |
| 54. | 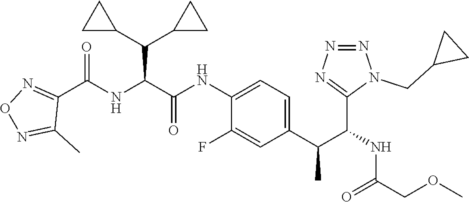 [SMILES: COCC(=O)N[C@@H](c1nnnn1CC1CC1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(1-(cyclopropylmethyl)-1H-tetrazol- |
| | 5-yl)-1-(2-methoxyacetamido)propan-2-yl)-2-fluorophenyl)amino)-3- |
| | oxopropan-2-yl)-4-methyl-1,2,5-oxadiazole-3-carboxamide |
| 55. | 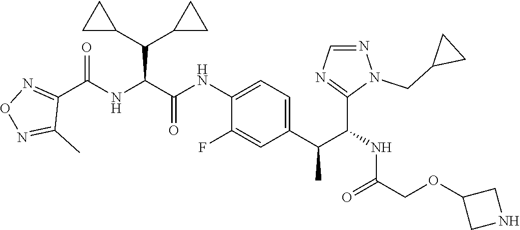 [SMILES: Cc1nonc1C(=O)N[C@H](C(=O)Nc1ccc([C@H](C)[C@@H](NC(=O)COC2CNC2)c2ncnn2CC2CC2)cc1F)C(C1CC1)C1CC1] |
| | N-((S)-1-((4-((1R,2S)-1-(2-(azetidin-3-yloxy)acetamido)-1-(1- |
| | (cyclopropylmethyl)-1H-1,2,4-triazol-5-yl)propan-2-yl)-2-fluorophenyl)amino)- |
| | 3,3-dicyclopropyl-1-oxopropan-2-yl)-4-methyl-1,2,5-oxadiazole-3-carboxamide |
| 56. | 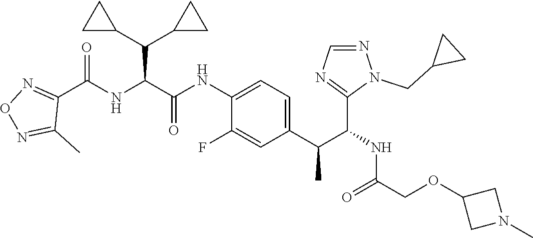 [SMILES: Cc1nonc1C(=O)N[C@H](C(=O)Nc1ccc([C@H](C)[C@@H](NC(=O)COC2CN(C)C2)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-((1-methylazetidin-3-yl)oxy)acetamido)propan-2-yl)-2- |
| | fluorophenyl)amino)-3-oxopropan-2-yl)-4-methyl-1,2,5-oxadiazole-3- |
| | carboxamide |
| 57. | 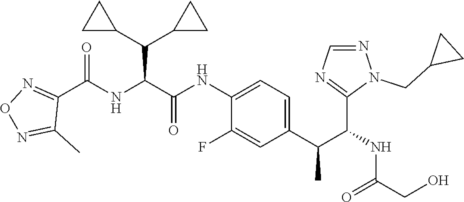 [SMILES: Cc1nonc1C(=O)N[C@H](C(=O)Nc1ccc([C@H](C)[C@@H](NC(=O)CO)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-hydroxyacetamido)propan-2-yl)-2-fluorophenyl)amino)-3- |
| | oxopropan-2-yl)-4-methyl-1,2,5-oxadiazole-3-carboxamide |
| 58. | 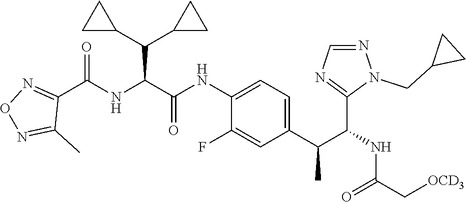 |
| | N-((S)-1,1-dicyclopropyl-3-((4-((1R,2S)-1-(1-(cyclopropylmethyl)-1H-1,2,4- |
| | triazol-5-yl)-1-(2-(methoxy-d₃)acetamido)propan-2-yl)-2-fluorophenyl)amino)- |
| | 3-oxopropan-2-yl)-4-methyl-1,2,5-oxadiazole-3-carboxamide |
| 59. | 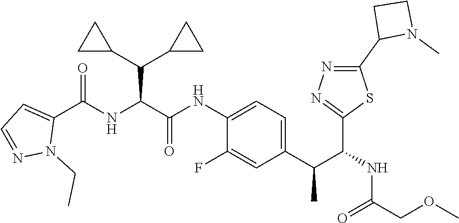 [SMILES: CCn1nccc1C(=O)N[C@H](C(=O)Nc1ccc([C@H](C)[C@@H](NC(=O)COC)c2nnc(C3CCN3C)s2)cc1F)C(C1CC1)C1CC1] |
| | N-((2S)-1,1-dicyclopropyl-3-((2-fluoro-4-((1R,2S)-1-(2-methoxyacetamido)-1- |
| | (5-(1-methylazetidin-2-yl)-1,3,4-thiadiazol-2-yl)propan-2-yl)phenyl)amino)-3- |
| | oxopropan-2-yl)-1-ethyl-1H-pyrazole-5-carboxamide |
| 60. | 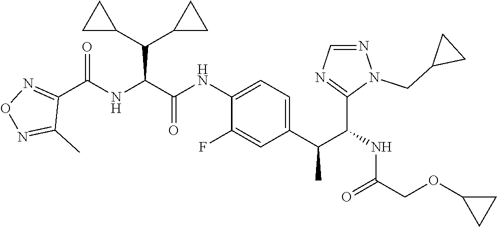 [SMILES: Cc1nonc1C(=O)N[C@H](C(=O)Nc1ccc([C@H](C)[C@@H](NC(=O)COC2CC2)c2ncnn2CC2CC2)cc1F)C(C1CC1)C1CC1] |
| | N-((S)-1-((4-((1R,2S)-1-(2-cyclopropoxyacetamido)-1-(1-(cyclopropylmethyl)- |
| | 1H-1,2,4-triazol-5-yl)propan-2-yl)-2-fluorophenyl)amino)-3,3-dicyclopropyl-1- |
| | oxopropan-2-yl)-4-methyl-1,2,5-oxadiazole-3-carboxamide |
| 61. | 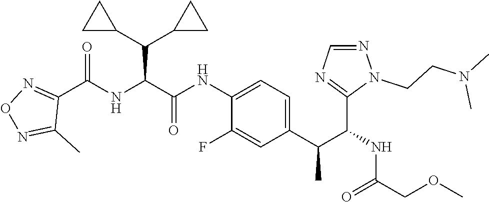 [SMILES: COCC(=O)N[C@@H](c1ncnn1CCN(C)C)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(1-(2-(dimethylamino)ethyl)-1H- |
| | 1,2,4-triazol-5-yl)-1-(2-methoxyacetamido)propan-2-yl)-2-fluorophenyl)amino)- |
| | 3-oxopropan-2-yl)-4-methyl-1,2,5-oxadiazole-3-carboxamide |
| 62. | 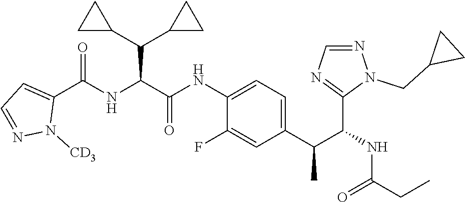 [SMILES: [2H]C([2H])([2H])n1nccc1C(=O)N[C@H](C(=O)Nc1ccc([C@H](C)[C@@H](NC(=O)CC)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-1-(methyl-d₃)-1H-pyrazole-5-carboxamide |
| 63. | 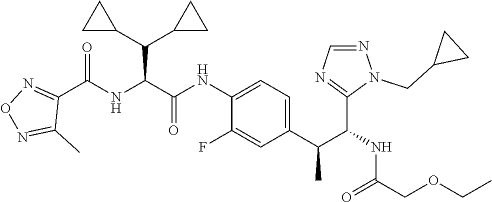 [SMILES: CCOCC(=O)N[C@@H](c1ncnn1CC1CC1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-(2-ethoxyacetamido)propan-2-yl)-2-fluorophenyl)amino)-3- |
| | oxopropan-2-yl)-4-methyl-1,2,5-oxadiazole-3-carboxamide |
| 64. | 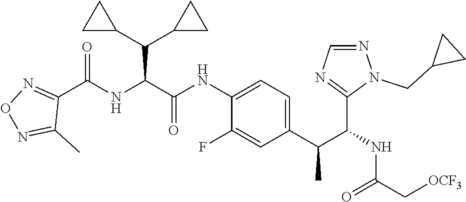 [SMILES: Cc1nonc1C(=O)N[C@H](C(=O)Nc1ccc([C@H](C)[C@@H](NC(=O)COC(F)(F)F)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-(trifluoromethoxy)acetamido)propan-2-yl)-2- |
| | fluorophenyl)amino)-3-oxopropan-2-yl)-4-methyl-1,2,5-oxadiazole-3- |
| | carboxamide |
| 65. | 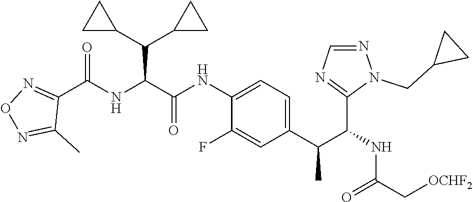 [SMILES: Cc1nonc1C(=O)N[C@H](C(=O)Nc1ccc([C@H](C)[C@@H](NC(=O)COC(F)F)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-(difluoromethoxy)acetamido)propan-2-yl)-2- |
| | fluorophenyl)amino)-3-oxopropan-2-yl)-4-methyl-1,2,5-oxadiazole-3- |
| | carboxamide |
| 66. | 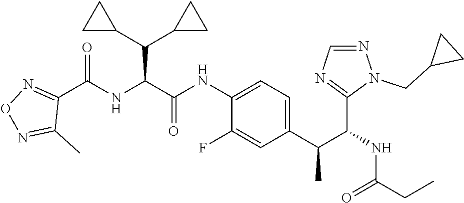 [SMILES: CCC(=O)N[C@@H](c1ncnn1CC1CC1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(cyclopropylmethyl)-1H-1,2,4- |
| | triazol-5-yl)-1-propionamidopropan-2-yl)-2-fluorophenyl)amino)-3-oxopropan- |
| | 2-yl)-4-methyl-1,2,5-oxadiazole-3-carboxamide |
| 67. | 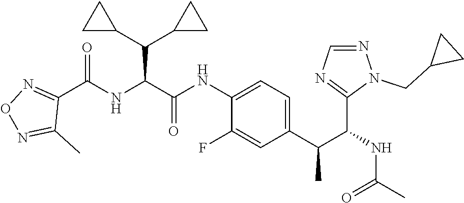 [SMILES: CC(=O)N[C@@H](c1ncnn1CC1CC1)[C@@H](C)c1ccc(NC(=O)[C@@H](NC(=O)c2nonc2C)C(C2CC2)C2CC2)c(F)c1] |
| | N-((S)-1-((4-((1R,2S)-1-acetamido-1-(1-(cyclopropylmethyl)-1H-1,2,4-triazol-5- |
| | yl)propan-2-yl)-2-fluorophenyl)amino)-3,3-dicyclopropyl-1-oxopropan-2-yl)-4- |
| | methyl-1,2,5-oxadiazole-3-carboxamide |
| 68. | 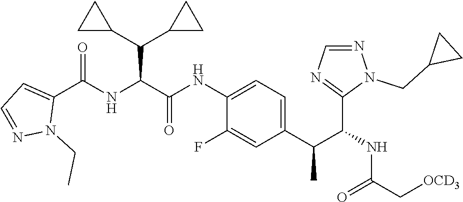 |
| | N-((S)-1,1-dicyclopropyl-3-((4-((1R,2S)-1-(1-(cyclopropylmethyl)-1H-1,2,4- |
| | triazol-5-yl)-1-(2-(methoxy-d₃)acetamido)propan-2-yl)-2-fluorophenyl)amino)- |
| | 3-oxopropan-2-yl)-1-ethyl-1H-pyrazole-5-carboxamide |
| 69. | 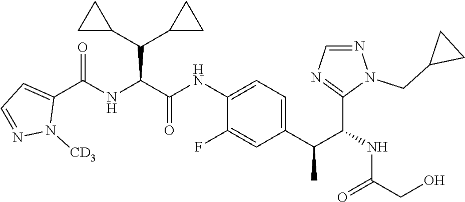 [SMILES: [2H]C([2H])([2H])n1nccc1C(=O)N[C@H](C(=O)Nc1ccc([C@H](C)[C@@H](NC(=O)CO)c2ncnn2CC2CC2)cc1F)C(C1CC1)C1CC1] |
| | N-((S)-1,1-dicyclopropyl-3-((4-((1R,2S)-1-(1-(cyclopropylmethyl)-1H-1,2,4- |
| | triazol-5-yl)-1-(2-hydroxyacetamido)propan-2-yl)-2-fluorophenyl)amino)-3- |
| | oxopropan-2-yl)-1-(methyl-d₃)-1H-pyrazole-5-carboxamide |
| 70. | 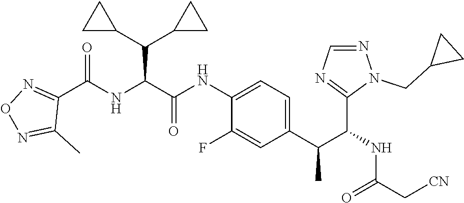 [SMILES: Cc1nonc1C(=O)N[C@H](C(=O)Nc1ccc([C@H](C)[C@@H](NC(=O)CC#N)c2ncnn2CC2CC2)cc1F)C(C1CC1)C1CC1] |
| | N-((S)-1-((4-((1R,2S)-1-(2-cyanoacetamido)-1-(1-(cyclopropylmethyl)-1H-1,2,4- |
| | triazol-5-yl)propan-2-yl)-2-fluorophenyl)amino)-3,3-dicyclopropyl-1- |
| | oxopropan-2-yl)-4-methyl-1,2,5-oxadiazole-3-carboxamide |
| 71. | 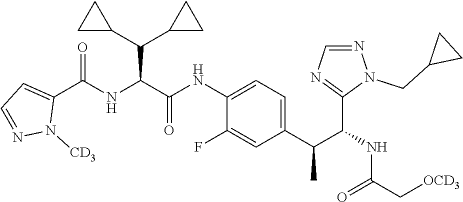 |
| | N-((S)-1,1-dicyclopropyl-3-((4-((1R,2S)-1-(1-(cyclopropylmethyl)-1H-1,2,4- |
| | triazol-5-yl)-1-(2-(methoxy-d₃)acetamido)propan-2-yl)-2-fluorophenyl)amino)- |
| | 3-oxopropan-2-yl)-1-(methyl-d₃)-1H-pyrazole-5-carboxamide |
| 72. | 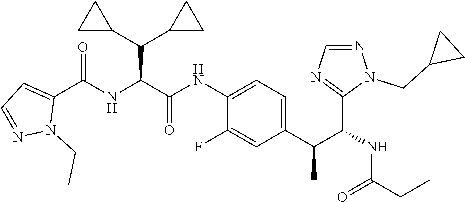 [SMILES: CCC(=O)N[C@@H](c1ncnn1CC1CC1)[C@@H](C)c1ccc(NC(=O)[C@@H](NC(=O)c2ccnn2CC)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-1-ethyl-1H-pyrazole-5-carboxamide |
| 73. | 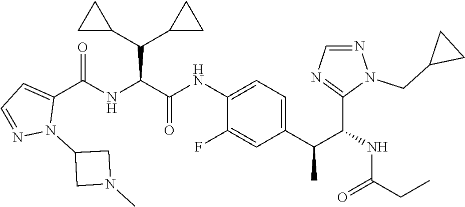 [SMILES: CCC(=O)N[C@@H](c1ncnn1CC1CC1)[C@@H](C)c1ccc(NC(=O)[C@@H](NC(=O)c2ccnn2C2CN(C)C2)C(C2CC2)C2CC2)c(F)c1] |
| | N-((S)-1,1-dicyclopropyl-3-((4-((1R,2S)-1-(cyclopropylmethyl)-1H-1,2,4- |
| | triazol-5-yl)-1-propionamidopropan-2-yl)-2-fluorophenyl)amino)-3-oxopropan- |
| | 2-yl)-1-(1-methylazetidin-3-yl)-1H-pyrazole-5-carboxamide |
| 74. | 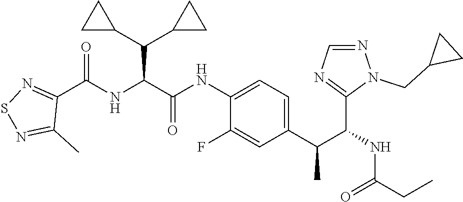 [SMILES: CCC(=O)N[C@@H](c1ncnn1CC1CC1)[C@@H](C)c1ccc(NC(=O)[C@@H](NC(=O)c2nsnc2C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-4-methyl-1,2,5-thiadiazole-3-carboxamide |
| 75. | 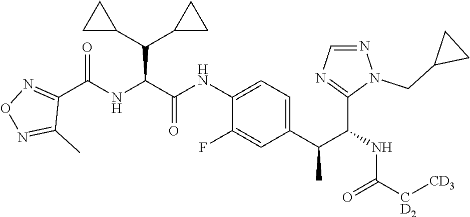 |
| | N-((S)-1,1-dicyclopropyl-3-((4-((1R,2S)-1-(1-(cyclopropylmethyl)-1H-1,2,4- |
| | triazol-5-yl)-1-(propanamido-2,2,3,3,3-d₅)propan-2-yl)-2-fluorophenyl)amino)- |
| | 3-oxopropan-2-yl)-4-methyl-1,2,5-oxadiazole-3-carboxamide |
| 76. | 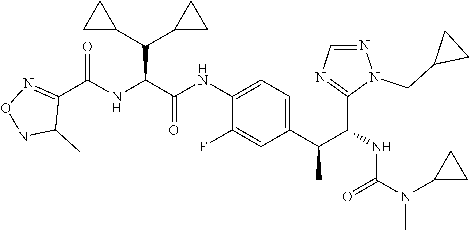 [SMILES: CC1NON=C1C(=O)N[C@H](C(=O)Nc1ccc([C@H](C)[C@@H](NC(=O)N(C)C2CC2)c2ncnn2CC2CC2)cc1F)C(C1CC1)C1CC1] |
| | N-((S)-1,1-dicyclopropyl-3-((4-((1R,2S)-1-(3-cyclopropyl-3-methylureido)-1-(1- |
| | (cyclopropylmethyl)-1H-1,2,4-triazol-5-yl)propan-2-yl)-2-fluorophenyl)amino)- |
| | 3-oxopropan-2-yl)-4-methyl-1,2,5-oxadiazole-3-carboxamide |
| 77. | 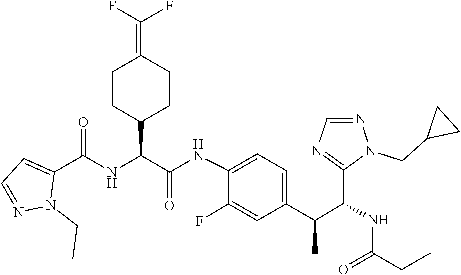 [SMILES: CCC(=O)N[C@@H](c1ncnn1CC1CC1)[C@@H](C)c1ccc(NC(=O)[C@@H](NC(=O)c2ccnn2CC)C2CCC(=C(F)F)CC2)c(F)c1] |
| | N-((S)-2-((4-((1R,2S)-1-(1-(cyclopropylmethyl)-1H-1,2,4-triazol-5-yl)-1- |
| | propionamidopropan-2-yl)-2-fluorophenyl)amino)-1-(4- |
| | (difluoromethylene)cyclohexyl)-2-oxoethyl)-1-ethyl-1H- |
| | pyrazole-5-carboxamide |
| 78. | 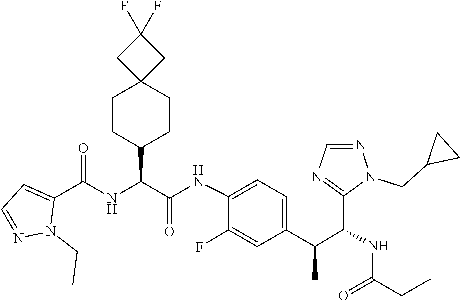 [SMILES: CCC(=O)N[C@@H](c1ncnn1CC1CC1)[C@@H](C)c1ccc(NC(=O)[C@@H](NC(=O)c2ccnn2CC)C2CCC3(CC2)CC(F)(F)C3)c(F)c1] |
| | N-((S)-2-((4-((1R,2S)-1-(1-(cyclopropylmethyl)-1H-1,2,4-triazol-5-yl)-1- |
| | propionamidopropan-2-yl)-2-fluorophenyl)amino)-1-(2,2- |
| | difluorospiro[3.5]nonan-7-yl)-2-oxoethyl)-1-ethyl-1H-pyrazole-5-carboxamide |
| 79. | 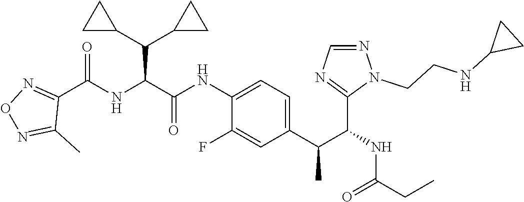 [SMILES: CCC(=O)N[C@@H](c1ncnn1CCNC1CC1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(1-(2-(cyclopropylamino)ethyl)-1H- |
| | 1,2,4-triazol-5-yl)-1-propionamidopropan-2-yl)-2-fluorophenyl)amino)-3- |
| | oxopropan-2-yl)-4-methyl-1,2,5-oxadiazole-3-carboxamide |
| 80. | 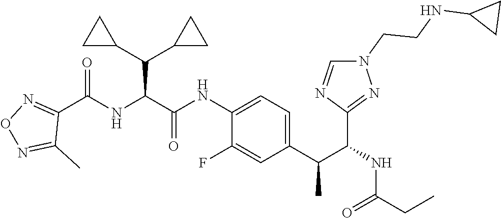 [SMILES: CCC(=O)N[C@@H](c1ncn(CCNC2CC2)n1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(2-(cyclopropylamino)ethyl)-1H- |
| | 1,2,4-triazol-3-yl)-1-propionamidopropan-2-yl)-2-fluorophenyl)amino)-3- |
| | oxopropan-2-yl)-4-methyl-1,2,5-oxadiazole-3-carboxamide |
| 81. | 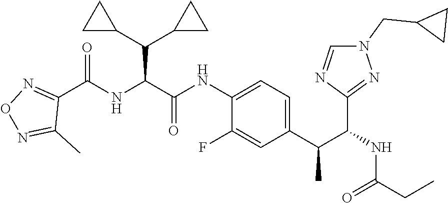 [SMILES: CCC(=O)N[C@@H](c1ncn(CC2CC2)n1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1R,2S)-1-(1-(cyclopropylmethyl)-1H-1,2,4- |
| | triazol-3-yl)-1-propionamidopropan-2-yl)-2-fluorophenyl)amino)-3-oxopropan- |
| | 2-yl)-4-methyl-1,2,5-oxadiazole-3-carboxamide |
| 82. | 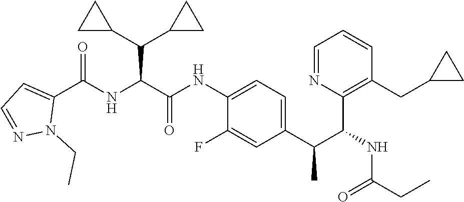 [SMILES: CCC(=O)N[C@@H](c1ncccc1CC1CC1)[C@@H](C)c1ccc(NC(=O)[C@@H](NC(=O)c2ccnn2CC)C(C2CC2)C2CC2)c(F)c1] |
| | N-((S)-1,1-dicyclopropyl-3-((4-((1R,2S)-1-(3-(cyclopropylmethyl)pyridin-2-yl)- |
| | 1-propionamidopropan-2-yl)-2-fluorophenyl)amino)-3-oxopropan-2-yl)-1-ethyl- |
| | 1H-pyrazole-5-carboxamide |
| 83. | 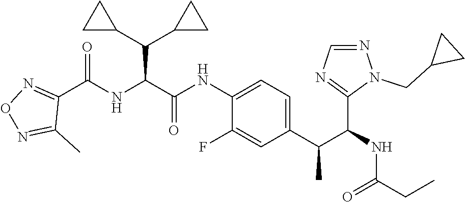 [SMILES: CCC(=O)N[C@H](c1ncnn1CC1CC1)[C@@H](C)c1ccc(NC(=O)[C@@H](NC(=O)c2nonc2C)C(C2CC2)C2CC2)c(F)c1] |
| | N-((S)-1,1-dicyclopropyl-3-((4-((1S,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-4-methyl-1,2,5-oxadiazole-3-carboxamide |
| 84. | 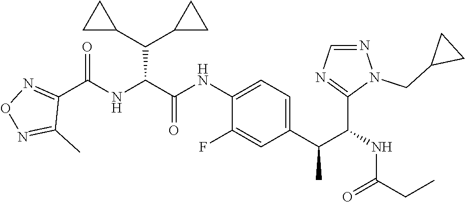 [SMILES: CCC(=O)N[C@@H](c1ncnn1CC1CC1)[C@@H](C)c1ccc(NC(=O)[C@H](NC(=O)c2nonc2C)C(C2CC2)C2CC2)c(F)c1] |
| | N-((R)-1,1-dicyclopropyl-3-((4-((1R,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-4-methyl-1,2,5-oxadiazole-3-carboxamide |
| 85. | 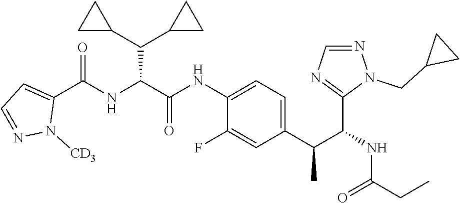 [SMILES: [2H]C([2H])([2H])n1nccc1C(=O)N[C@@H](C(=O)Nc1ccc([C@H](C)[C@@H](NC(=O)CC)c2ncnn2CC2CC2)cc1F)C(C1CC1)C1CC1] |
| | N-((R)-1,1-dicyclopropyl-3-((4-((1R,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-1-(methyl-d₃)-1H-pyrazole-5-carboxamide |
| 86. | 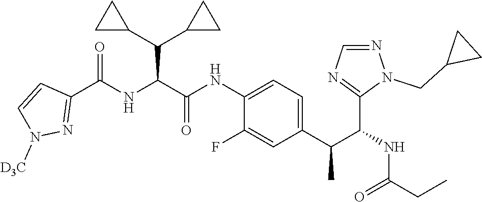 [SMILES: [2H]C([2H])([2H])n1ccc(C(=O)N[C@H](C(=O)Nc2ccc([C@H](C)[C@@H](NC(=O)CC)c3ncnn3CC3CC3)cc2F)C(C2CC2)C2CC2)n1] |
| | N-((S)-1,1-dicyclopropyl-3-((4-((1R,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-1-(methyl-d₃)-1H-pyrazole-5-carboxamide |
| 87. | 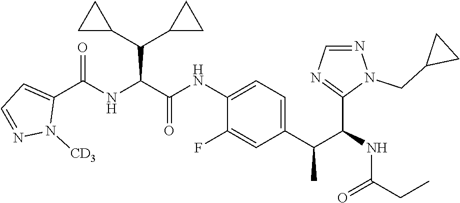 [SMILES: [2H]C([2H])([2H])n1nccc1C(=O)N[C@H](C(=O)Nc1ccc([C@H](C)[C@H](NC(=O)CC)c2ncnn2CC2CC2)cc1F)C(C1CC1)C1CC1] |
| | N-((S)-1,1-dicyclopropyl-3-((4-((1S,2S)-1-(1-(cyclopropylmethyl)-1H-1,2,4- |
| | triazol-5-yl)-1-propionamidopropan-2-yl)-2-fluorophenyl)amino)-3-oxopropan- |
| | 2-yl)-1-(methyl-d₃)-1H-pyrazole-5-carboxamide |
| 88. | 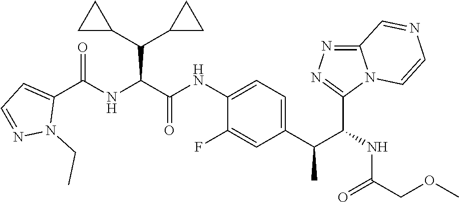 [SMILES: CCn1nccc1C(=O)N[C@H](C(=O)Nc1ccc([C@H](C)[C@@H](NC(=O)COC)c2nnc3cnccn23)cc1F)C(C1CC1)C1CC1] |
| | N-((S)-1-((4-((1R,2S)-1-([1,2,4]triazolo[4,3-a]pyrazin-3-yl)-1-(2- |
| | methoxyacetamido)propan-2-yl)-2-fluorophenyl)amino)-3,3-dicyclopropyl-1- |
| | oxopropan-2-yl)-1-ethyl-1H-pyrazole-5-carboxamide |

The compounds of the present invention effectively prevent IL-17AA binding to its receptors, having an IC₅₀ of less than 500 nM, preferably an IC₅₀ of less than 50 nM. The compounds of the present invention have a significant inhibitory effect on IL-17AA induced IL-17 signaling pathway in HEK-Blue cells, having an IC₅₀ of less than 1000 nM, preferably an IC₅₀ of less than 50 nM. The compounds of the present invention also have a significant inhibitory effect on IL-17AF induced IL-17 signaling pathway in HEK-Blue cells, having an IC₅₀ of less than 1000 nM, preferably an IC₅₀ of less than 100 nM. The compounds of the present invention further demonstrate effectiveness in suppression of arthritis disease progression a rat CIA model.

The present invention further relates to pharmaceutical compositions comprising compounds of Formula (I) or pharmaceutically acceptable salts, prodrugs, stable isotope derivatives, or isomers thereof, and pharmaceutically acceptable carriers or excipients. The pharmaceutical compositions are useful for the treatment or prevention of IL-17, in particular IL-17AA and IL-17AF mediated diseases, including but not limited to autoimmune or inflammatory disorders, for example, psoriasis, psoriatic arthritis, rheumatoid arthritis, spondylarthritis, ankylosing spondylitis, hidradenitis suppurativa, etc.

The present invention further provides a method for treating or preventing diseases mediated by IL-17, particularly by IL-17AA and IL-17AF. The method comprises administering to a patient in need thereof a therapeutically effective amount of the compounds shown in Formula (I) or pharmaceutically acceptable salts, prodrugs, stable isotope derivatives and isomers thereof. The diseases include but are not limited to autoimmune and inflammatory diseases.

According to the present invention, the pharmaceuticals can be in any dosage form, including but not limited to tablets, capsules, a solution, a freeze-drying preparation and injectable.

The pharmaceutical formulation of the present invention can be administered in form of a dosage unit containing a predetermined amount of active ingredient. Such a unit may contain 1 mg to 2 g, preferably 10 mg to 1 g of a compound of the present invention, depending on the disease being treatment, the method of administration, as well as age, weight, and condition of the patients. The pharmaceutical formulation can be prepared using methods well-known in the pharmaceutical field, for example, by formulating the active ingredient with one or more excipients or one or more adjuvants.

The pharmaceutical formulation of the present invention is suitable for administration by any appropriate method, for example by oral (including buccal or sublingual) or parenteral (including subcutaneous, intramuscular, intravenous, or intradermal).

The present invention further provides methods for preparing the compounds. The preparation of compounds of the present invention can be accomplished by the following exemplary methods and embodiments, but these methods and embodiments should not be considered as limitations to the scope of the present invention. Alternatively, compounds of the present invention can be synthesized by methods known to those skilled in the art, or by methods described in the present invention. The starting materials and chemical reagents used for synthesis can be conventionally made based on literature (for example, SciFinder) or purchased.

The compounds shown in Formula (I) of the present invention can be synthesized according to the route shown below: substitution of the leaving group X (such as halide) of Int2 by Int1 using the Buchwald amination or the Ullmann coupling to afford the desired product.

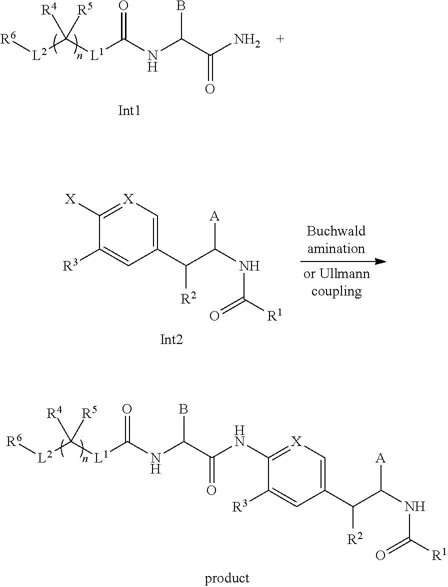

Int1 can be synthesized according to the route shown below: 1) condensation of the aldehyde A1 with sulfinamide to give A2; 2) cyanation of A2 to give A3; 3) deprotection of the sulfinamide A3, followed by amide coupling with carboxylic acid to give A4; 4) hydrolysis of the nitrile group of A4 under a basic condition to provide Int1.

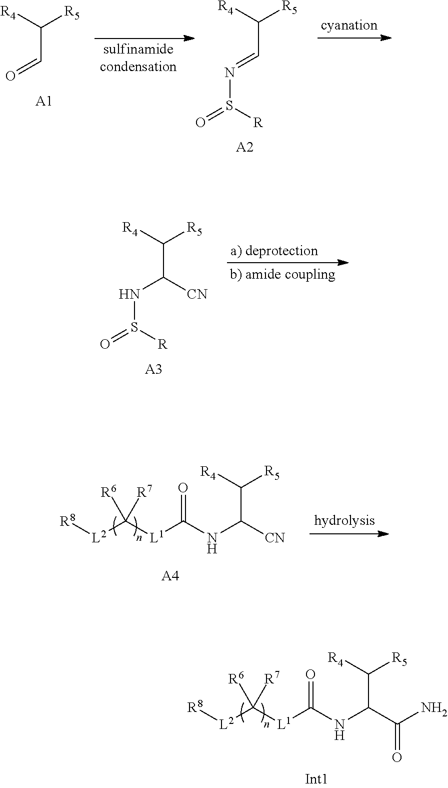

Int2 can be synthesized according to the route shown below: 1) protection of the amino acid B1 with phthalic anhydride, followed by amidation of carboxylic acid to give B2; 2) palladium-catalyzed C—H arylation of B2, followed by double deprotection to give the amino acid B3; 3) esterification of carboxylic acid of B3 and consequent amide coupling of amine to give B4; 4) hydrazinolysis of the ester B4, followed by condensation with DMF-DMA to give B5; 5) final cyclization with amine to provide Int2.

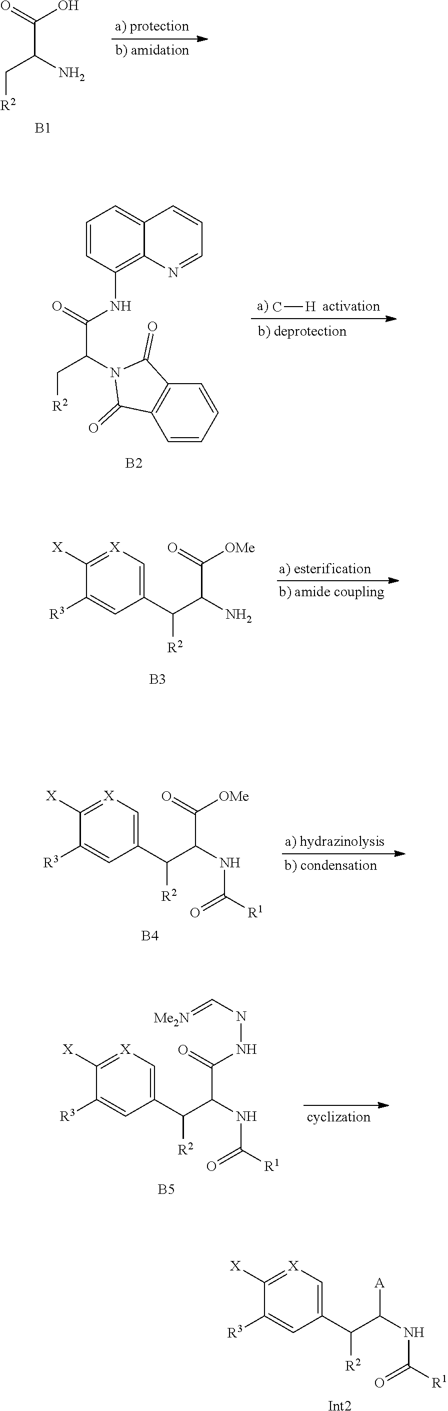

Int2 can also be synthesized according to the route shown below: 1) hydrolysis of the ester B4 under a basic condition, followed by amide coupling with NH₄Cl to give the primary amide C1; 2) condensation of C1 with DMF-DMA, followed by cyclization with alkyl hydrazine to provide Int2.

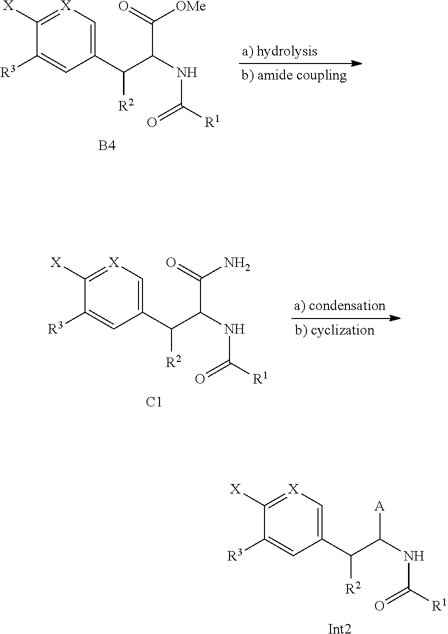

The compounds shown in Formula (I) of the present invention can also be synthesized according to the route shown below: 1) deprotection of the sulfinamide A3, followed by Boc protection and hydrolysis of nitrile to give D1; 2) cross-coupling of D1 with Int2 to give E2; 3) removal of Boc protecting group, followed by amide coupling with carboxylic acid to afford the desired product.

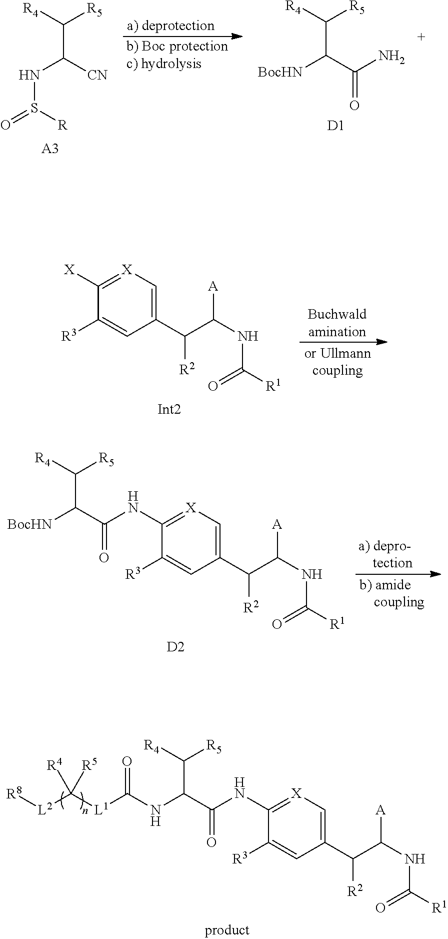

### EXAMPLES

The structure of a compound was determined by nuclear magnetic resonance (NMR) or mass spectrometry (MS). NMR determination used a Bruker ASCEND-400 NMR spectrometer. The solvent for the determination was deuterated dimethyl sulfoxide (DMSO-d₆), deuterated chloroform (CDCl₃), or deuterated methanol (CD₃OD). The internal standard was tetramethylsilane (TMS), and the chemical shift was given in a unit of 10⁻⁶ (ppm). MS determination used an Agilent SQD (ESI) mass spectrometer (Agilent 6120).

HPLC determination used Agilent 1260 DAD high pressure liquid chromatograph (column: Poroshell120 EC-C18, 50×3.0 mm, 2.7 μm) or Waters Arc high pressure liquid chromatograph (column: Sunfire C18, 150×4.6 mm, 5 μm).

Thin layer chromatography (TLC) used GF254 silica gel plates from Qingdao Haiyang Chemical Co., Ltd. with a thickness of 0.15 to 0.2 mm. Separation/purification of products by thin layer chromatography used silica plates with a thickness 0.4 to 0.5 mm.

Column chromatography generally used 200 to 300 mesh silica gel from Qingdao Haiyang Chemical Co., Ltd.

Known starting materials in the present invention were synthesized according to the methods known in the art, or purchased from ABCR GmbH&Co. KG, Acros Organics, Aldrich Chemical Company, Accela ChemBio Inc., Beijing Ouhe Technology Co., Ltd., etc.

Unless otherwise stated in the embodiments, the reactions were carried out under an atmosphere of argon or nitrogen using a balloon with a volume of about 1 L.

Hydrogenation was carried out under an atmosphere of hydrogen using a balloon with a volume of about 1 L that was attached to the reaction vessel after being vacuumed and filled with hydrogen repeatedly for 3 times.

The microwave reaction used a CEM Discover-SP microwave reactor.

Unless otherwise stated in the embodiments, the reaction was run at room temperature.

The reaction was monitored using Agilent LCMS (1260/6120) or thin layer chromatography. The solvent eluting systems for column chromatography and TLC included a) dichloromethane/methanol, b) petroleum ether/ethyl acetate, or other systems as indicated. The ratio of the solvents was adjusted according to polarity of compound, and further adjusted by addition of a small amount of TEA, or an acidic or alkaline reagent as needed. The compound purification was alternatively done using Waters' MS-guided automated preparation system (abbreviated as prep-HPLC) with a MS detector (SQD2), eluting at a flow rate of 20 mL/min with an appropriate acetonitrile/water (containing 0.1% TFA or formic acid) or acetonitrile/water (containing 0.05% of 25-28% ammonium hydroxide) gradient (XBridge-C18, 19×150 mm, 5 μm).

The abbreviation PE refers to petroleum ether.

The abbreviation DMF refers to N,N-dimethylformamide.

The abbreviation DMA refers to N,N-dimethylacetamide.

The abbreviation DMF-DMA refers to N,N-dimethylformamide dimethyl acetal.

The abbreviation THF refers to tetrahydrofuran.

The abbreviation DMSO refers to dimethyl sulfoxide.

The abbreviation DIPEA refers to N,N-diisopropylethylamine.

The abbreviation TEA refers to triethylamine.

The abbreviation TFA refers to trifluoroacetic acid.

The abbreviation DMAP refers to 4-dimethylaminopyridine.

The abbreviation DAST refers to diethylaminosulfur trifluoride.

The abbreviation IBX refers to 2-iodoxybenzoic acid.

The abbreviation EDCI refers to 1-ethyl-3-(3-dimethylaminopropyl)carbodiimide hydrochloride.

The abbreviation DMTMM·BF₄ refers to 4-(4,6-dimethoxy-1,3,5-triazin-2-yl)-4-methylmorpholinium tetrafluoroborate.

The abbreviation HATU refers to 2-(7-azabenzotriazol-1-yl)-N,N,N′,N′-tetramethyluronium hexafluorophosphate.

The abbreviation DMEDA refers to N,N′-dimethylethylenediamine.

The abbreviation DEAD refers to diethyl azodicarboxylate.

The abbreviation DIAD refers to diisopropyl azodicarboxylate.

The abbreviation KHMDS refers to potassium bis(trimethylsilyl)amide.

BrettPhos refers to dicyclohexyl(2′,4′,6′-triisopropyl-3,6-dimethoxy-[1,1′-biphenyl]-2-yl)phosphine.

NiCl₂(dtbpy) refers to bis(1,1-dimethylethyl)-2,2′-bipyridine]nickel(II) dichloride.

XantPhos refers to 4,5-bis(diphenylphosphino)-9,9-dimethylxanthene.

Pd₂(dba)₃ refers to tris(dibenzylideneacetone)dipalladium.

Pd(dppf)Cl₂ refers to [1,1′-bis(diphenylphosphino)ferrocene]dichloropalladium(II).

BrettPhos Pd G3 refers to [(2-di-cyclohexylphosphino-3,6-dimethoxy-2′,4′,6′-triisopropyl-1,1′-biphenyl)-2-(2′-amino-1,1′-biphenyl)]palladium(II) methanesulfonate.

CDI refers to N,N′-carbonyl diimidazole.

CbzCl refers to benzyl chloroformate.

EA, also EtOAc, refers to ethyl acetate.

### Example 1. N—((S)-1,1-dicyclopropyl-3-((4-((1R,2S)-1-(4-(cyclopropylmethyl)-4H-1,2,4-triazol-3-yl)-1-propionamidopropan-2-yl)-2-fluorophenyl)amino)-3-oxopropan-2-yl)-1-ethyl-1H-pyrazole-5-carboxamide (Compound 1)

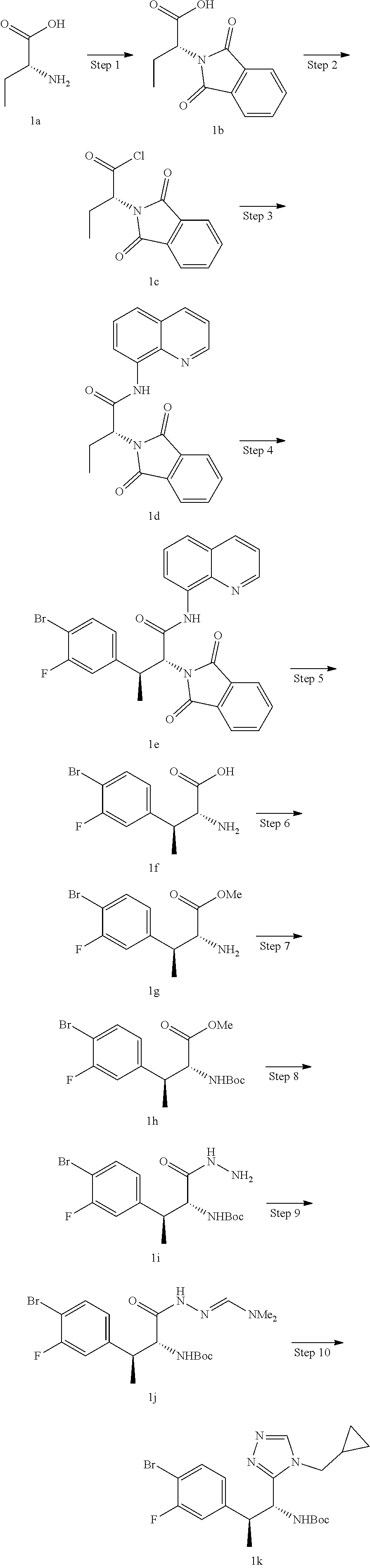

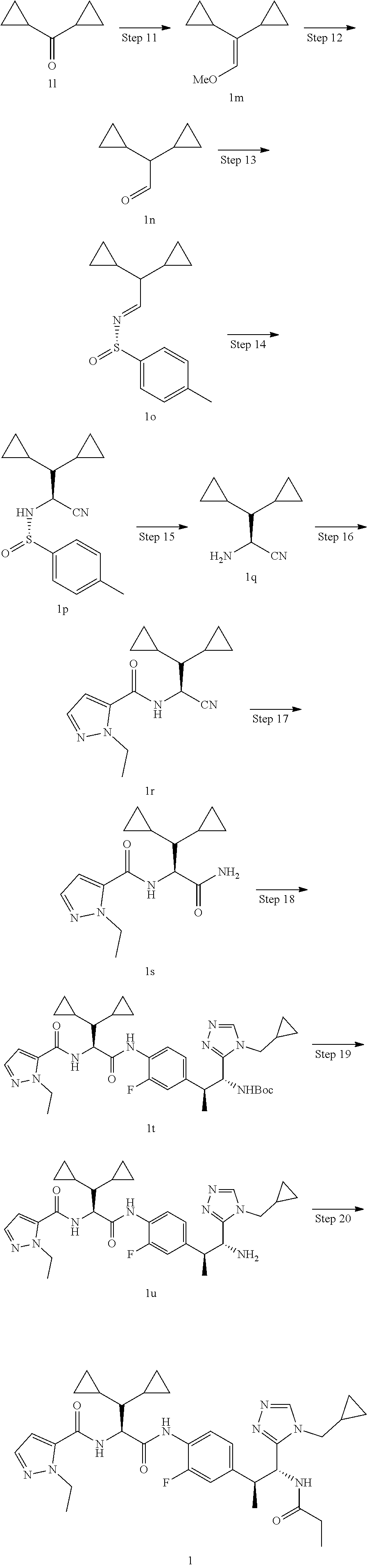

### Step 1. (R)-2-(1,3-dioxoisoindolin-2-yl)butanoic acid (1b)

To a solution of (R)-2-aminobutanoic acid 1a (100 g, 971 mmol) in toluene (500 mL) were added phthalic anhydride (158 g, 1068 mmol) and TEA (29.4 g, 291 mmol), which was then heated to 130° C. and stirred for 12 h. After cooling to room temperature, the reaction mixture was concentrated to dryness. The residue was added with dilute hydrochloric acid (1 N, 200 mL) and extracted with EtOAc (3×200 mL). The combined organic phase was dried over anhydrous Na₂SO₄ and filtered. The filtrate was concentrated to dryness to give the title compound 1b (220 g, 97%). The crude product was directly used in the next step without further purification.

MS m/z (ESI): 234 [M+1]

### Step 2. (R)-2-(1,3-dioxoisoindolin-2-yl)butanoyl chloride (1c)

To a solution of 1b (220 g, 944 mmol) in toluene (300 mL) was added thionyl chloride (337 g, 2832 mmol), which was then heated to 80° C. and stirred for 1 h. After cooling to room temperature, the reaction mixture was concentrated to dryness to give the title compound 1c (230 g, crude). The crude product was directly used in the next step without further purification.

### Step 3. (R)-2-(1,3-dioxoisoindolin-2-yl)-N-(quinolin-8-yl)butanamide (1d)

To a mixture of 8-aminoquinoline (132 g, 916 mmol), DIPEA (320 mL, 1832 mmol) and dichloromethane (1000 mL) at 0° C. was slowly added 1c (230 g, 916 mmol), which was then stirred at 0° C. for 12 h. The reaction mixture was concentrated to dryness. The residue was added with dilute hydrochloric acid (1 N, 500 mL) and extracted with EtOAc (3×500 mL). The combined organic phase was dried over anhydrous Na₂SO₄ and filtered. The filtrate was concentrated to dryness to give the title compound 1d (297 g, 91%). The crude product was directly used in the next step without further purification.

MS m/z (ESI): 360 [M+1]

### Step 4. (2R,3S)-3-(4-bromo-3-fluorophenyl)-2-(1,3-dioxoisoindolin-2-yl)-N-(quinolin-8-yl)butanamide (1e)

A mixture of 1d (297 g, 827 mmol), 1-bromo-2-fluoro-4-iodobenzene (496 g, 1654 mmol), silver acetate (207 g, 1241 mmol), Pd(OAc)₂ (18.6 g, 83 mmol) and toluene (1500 mL) was heated to 110° C. and stirred for 12 h. The reaction mixture was directly filtered without cooling and the filtrate was concentrated to dryness. The residue was purified by trituration from EtOAc (1000 mL) to give the title compound 1e (210 g, 48%). The crude product was directly used in the next step without further purification.

MS m/z (ESI): 532, 534 [M+1]

### Step 5. (2R,3S)-2-amino-3-(4-bromo-3-fluorophenyl)butanoic acid (1f)

A mixture of 1e (210 g, 585 mmol) and concentrated hydrochloric acid (1000 mL) was heated to 130° C. and stirred for 12 h. After cooling to room temperature, the reaction mixture was concentrated to dryness. The residue was added with dilute hydrochloric acid (1 N, 500 mL) and washed with EtOAc (3×500 mL). The combined aqueous phase was added with NaOH until pH=4, then washed with EtOAc (3×500 mL) and concentrated to dryness to give the title compound 1f (105 g, 97%). The crude product was directly used in the next step without further purification.

MS m/z (ESI): 276, 278 [M+1]

### Step 6. Methyl (2R,3S)-2-amino-3-(4-bromo-3-fluorophenyl)butanoate (1g)

A mixture of 1f (55 g, 200 mmol), concentrated sulfuric acid (5 mL) and methanol (300 mL) was heated to 100° C. and stirred for 12 h. After cooling to room temperature, the reaction mixture was concentrated to dryness. The residue was added with a saturated aqueous NaHCO₃ solution (500 mL) and extracted with EtOAc (3×500 mL). The combined organic phase was dried over anhydrous Na₂SO₄ and filtered. The filtrate was concentrated to dryness to give the title compound 1g (51 g, 88%). The crude product was directly used in the next step without further purification.

MS m/z (ESI): 290, 292 [M+1]

### Step 7. Methyl (2R,3S)-3-(4-bromo-3-fluorophenyl)-2-((tert-butoxycarbonyl)amino)butanoate (1h)

To a mixture of 1g (2.90 g, 10 mmol), Boc₂O (2.8 mL, 12 mmol) and dichloromethane (50 mL) at 0° C. was added TEA (6.9 mL, 50 mmol), which was then warmed to room temperature and stirred overnight. The reaction mixture was concentrated to dryness to give the title compound 1h (3.90 g, crude). The crude product was directly used in the next step without further purification.

MS m/z (ESI): 390, 392 [M+1]

### Step 8. Tert-butyl ((2R,3S)-3-(4-bromo-3-fluorophenyl)-1-hydrazineyl-1-oxobutan-2-yl)carbamate (1i)

To a solution of 1h (3.90 g, crude, 10 mmol) in methanol (50 mL) was added hydrazine hydrate (2.4 mL, 50 mmol), which was then heated to reflux and stirred overnight. After cooling to room temperature, the reaction mixture was concentrated to dryness. The residue was added dichloromethane (100 mL), dried over anhydrous Na₂SO₄ and filtered. The filtrate was concentrated to dryness to give the title compound 1i (3.90 g, crude). The crude product was directly used in the next step without further purification.

MS m/z (ESI): 390, 392 [M+1]

### Step 9. Tert-butyl ((2R,3S)-3-(4-bromo-3-fluorophenyl)-1-(2-((E)-(dimethylamino)methylene)hydrazineyl)-1-oxobutan-2-yl)carbamate (1j)

To a solution of 1i (3.90 g, crude, 10 mmol) in acetonitrile (20 mL) was added DMF-DMA (2.0 mL, 15 mmol), which was then heated to 50° C. and stirred for 2 h. The crude reaction mixture was directly used in the next step without further purification.

MS m/z (ESI): 445, 447 [M+1]

### Step 10. Tert-butyl ((1R,2S)-2-(4-bromo-3-fluorophenyl)-1-(4-(cyclopropylmethyl)-4H-1,2,4-triazol-3-yl)propyl)carbamate (1k)

To a reaction mixture of the previous step was added acetic acid (10 mL) and (aminomethyl)cyclopropane (3.5 mL, 50 mmol), which was then heated to 100° C. and stirred for 3 h. After cooling to room temperature, the reaction mixture was added with a saturated aqueous NaHCO₃ solution (100 mL) and extracted with dichloromethane (100 mL). The organic phase was concentrated to dryness and the residue was purified by silica gel column chromatography (dichloromethane/methanol=100/0 to 9/1) to give the title compound 1k (1.43 g, 32%).

MS m/z (ESI): 453, 455 [M+1]

### Step 11. (2-Methoxyethene-1,1-diyl)dicyclopropane (1m)

To a suspension of (methoxymethyl)triphenylphosphonium chloride (446 g, 1300 mmol) in THF (1500 mL) at 0° C. was added potassium tert-butoxide (146 g, 1300 mmol), which was then stirred at 0° C. for 1 h. The resulting mixture was added with dicyclopropyl ketone 1l (110 g, 1000 mmol), which was then warmed to room temperature and stirred for 1 h. The reaction mixture was added with a saturated aqueous NH₄Cl solution (1000 mL) and extracted with EtOAc (1000 mL). The organic phase was dried over anhydrous Na₂SO₄ and filtered. The filtrate was concentrated to dryness. The residue was added with PE (1000 mL), which was then stirred for 30 min and filtered. The filtrate was concentrated to dryness to give the title compound 1m (138 g, crude). The crude product was directly used in the next step without further purification.

### Step 12. 2,2-Dicyclopropylacetaldehyde (1n)

A mixture of 1m (138 g, crude, 1000 mmol), THF (1000 mL) and dilute hydrochloric acid (6 N, 1000 mL) was stirred for 1 h. The reaction mixture was concentrated to remove THF. The residue was added with a saturated aqueous NaHCO₃ solution (1000 mL) and extracted with EtOAc (1000 mL). The organic phase was dried over anhydrous Na₂SO₄ and filtered. The filtrate was concentrated to dryness to give the title compound in (115 g, crude). The crude product was directly used in the next step without further purification.

### Step 13. (S,E)-N-(2,2-dicyclopropylethylidene)-4-methylbenzenesulfinamide (1o)

To a mixture of in (112 g, crude, 900 mmol), (S)-4-methylbenzenesulfenamide (140 g, 900 mmol) and THF (2000 mL) at 0° C. was added Ti(OEt)₄ (471 mL, 2250 mmol), which was then warmed to room temperature and stirred overnight. The reaction mixture was added with saturated brine (2000 mL) and filtered. The filtrate was extracted with EtOAc (2000 mL). The organic phase was concentrated to dryness and the residue was purified by silica gel column chromatography (PE/EtOAc=100/0 to 4/1) to give the title compound to (150 g, 64%).

MS m/z (ESI): 262 [M+1]

### Step 14. (S)—N—((S)-1-cyano-2,2-dicyclopropylethyl)-4-methylbenzenesulfinamide (1p)

To a solution of to (149 g, 570 mmol) in hexanes (3500 mL) at −40° C. were added trimethylsilyl cyanide (471 mL, 855 mmol) and CsF (130 g, 855 mmol), which was then stirred at −40° C. overnight. The reaction mixture was added with water (2000 mL) and extracted with EtOAc (2000 mL). The organic phase was dried over anhydrous Na₂SO₄ and filtered. The filtrate was concentrated to dryness and the residue was recrystallized from PE/EtOAc to give the title compound 1p (100 g, 61%).

MS m/z (ESI): 289 [M+1]

### Step 15. (S)-2-amino-3,3-dicyclopropylpropanenitrile hydrochloride (1q)

A solution of 1p (1.15 g, 4 mmol) in a mixture of hydrochloric acid in 1,4-dioxane (4 M, 10 mL) and methanol (10 mL) was stirred for 1 h. The reaction mixture was concentrated to dryness to give the title compound 1q (746 mg, crude). The crude product was directly used in the next step without further purification.

MS m/z (ESI): 151 [M+1]

### Step 16. (S)—N-(1-cyano-2,2-dicyclopropylethyl)-1-ethyl-1H-pyrazole-5-carboxamide (1r)

To a mixture of 1q (300 mg, 2 mmol), 1-ethyl-1H-pyrazole-5-carboxylic acid (280 mg, 2 mmol), HATU (913 mg, 2.4 mmol) and DMF (10 mL) was added DIPEA (1 mL, 6 mmol), which was then stirred for 1 h. The reaction mixture was added with EtOAc (100 mL) and then washed sequentially with a saturated aqueous NaHCO₃ solution (100 mL), dilute hydrochloric acid (1 N, 100 mL) and saturated brine (100 mL). The organic phase was concentrated to dryness and the residue was purified by silica gel column chromatography (PE/EtOAc=100/0 to 3/2) to give the title compound 1r (350 mg, 64%).

MS m/z (ESI): 273 [M+1]

### Step 17. (S)—N-(1-amino-3,3-dicyclopropyl-1-oxopropan-2-yl)-1-ethyl-1H-pyrazole-5-carboxamide (Is)

To a mixture of 1r (327 mg, 1.2 mmol), hydrogen peroxide (30% aqueous solution, 1.5 mL) and DMSO (3 mL) at 0° C. was added NaOH (8 mg, 0.2 mmol), which was then stirred for 1 h. The reaction mixture was added with water (20 mL) and filtered to give the title compound is (290 mg, 83%). The crude product was directly used in the next step without further purification.

MS m/z (ESI): 291 [M+1]

### Step 18. Tert-butyl ((1R,2S)-1-(4-(cyclopropylmethyl)-4H-1,2,4-triazol-3-yl)-2-(4-((S)-3,3-dicyclopropyl-2-(1-ethyl-1H-pyrazole-5-carboxamido)propanamido)-3-fluorophenyl)propyl)carbamate (1t)

A mixture of 1k (453 mg, 1 mmol), is (348 mg, 1 mmol), BrettPhos Pd G3 (91 mg, 0.1 mmol), Cs₂CO₃ (652 mg, 2 mmol) and 1,4-dioxane (4 mL) was heated to 110° C. and stirred overnight. After cooling to room temperature, the reaction mixture was quenched with saturated brine (10 mL) and extracted with EtOAc (10 mL). The organic phase was concentrated to dryness and the residue was purified by silica gel column chromatography (dichloromethane/methanol=100/0 to 9/1) to give the title compound it (663 mg, 99%).

MS m/z (ESI): 663 [M+1]

### Step 19. N—((S)-1-((4-((1R,2S)-1-amino-1-(4-(cyclopropylmethyl)-4H-1,2,4-triazol-3-yl)propan-2-yl)-2-fluorophenyl)amino)-3,3-dicyclopropyl-1-oxopropan-2-yl)-1-ethyl-1H-pyrazole-5-carboxamide hydrochloride (1u)

A solution of it (663 mg, 1 mmol) in a mixture of hydrochloric acid in 1,4-dioxane (4 M, 3 mL) and methanol (3 mL) was stirred for 1 h. The reaction mixture was concentrated to dryness to give the title compound 1u (563 mg, crude). The crude product was directly used in the next step without further purification.

MS m/z (ESI): 563 [M+1]

### Step 20. N—((S)-1,1-dicyclopropyl-3-((4-((1R,2S)-1-(4-(cyclopropylmethyl)-4H-1,2,4-triazol-3-yl)-1-propionamidopropan-2-yl)-2-fluorophenyl)amino)-3-oxopropan-2-yl)-1-ethyl-1H-pyrazole-5-carboxamide (1)

To a mixture of 1u (169 mg, 0.3 mmol), propionyl chloride (39 uL, 0.45 mmol) and DMF (1.5 mL) at 0° C. was added TEA (208 uL, 1.5 mmol), which was then warmed to room temperature and stirred for 1 h. The reaction mixture was added with EtOAc (10 mL) and washed with a saturated aqueous NaHCO₃ solution (10 mL). The organic phase was concentrated to dryness and the residue was purified by silica gel column chromatography (dichloromethane/methanol=100/0 to 9/1) then by prep-HPLC to give the title compound 1 (9.3 mg, 64%).

MS m/z (ESI): 619 [M+1]

¹H NMR (400 MHz, CD₃OD) δ 8.33 (s, 1H), 7.73 (t, J=6.9 Hz, 1H), 7.49 (d, J=2.1 Hz, 1H), 7.01 (t, J=11.3 Hz, 2H), 6.83 (d, J=2.1 Hz, 1H), 5.22 (d, J=11.0 Hz, 1H), 4.92 (d, J=6.9 Hz, 2H), 4.53 (dd, J=14.0, 6.8 Hz, 3H), 3.70 (dd, J=14.3, 7.4 Hz, 1H), 3.56 (dd, J=13.3, 6.3 Hz, 2H), 2.37-2.16 (m, 1H), 2.02 (s, 1H), 1.48-1.42 (m, 3H), 1.37 (dd, J=8.5, 5.8 Hz, 3H), 1.14-1.08 (m, 3H), 0.89 (dd, J=22.8, 12.8 Hz, 3H), 0.81-0.71 (m, 2H), 0.53 (s, 3H), 0.42 (s, 1H), 0.33 (s, 1H), 0.26 (s, 3H), 0.21-0.16 (in, 1H).

The following intermediates were prepared according to steps 1 to 5 of Example 1, except that different reagents were used instead of 1-bromo-2-fluoro-4-iodobenzene.

| | Reagent replacing 1- | |
|---|---|---|
| | bromo-2-fluoro-4- | MS m/z |
| Intermediate | iodobenzene | (ESI) |
| (2R,3S)-2-amino-3-(6-bromo-5- | 2-Bromo-3-fluoro-5- | 276, 278 |
| fluoropyridin-3-yl)butanoic acid | iodopyridine | [M + 1] |
| (2R,3S)-2-amino-3-(6-bromopyridin- | 2-Bromo-5- | 259, 261 |
| 3-yl)butanoic acid | iodopyridine | [M + 1] |
| (2R,3S)-2-amino-3-(4-bromo-3- | 1-Bromo-4-iodo-2- | 288, 290 |
| methoxyphenyl)butanoic acid | methoxybenzene | [M + 1] |

The following intermediate was prepared according to steps 11 to 15 of Example 1, except that a different reagent was used instead of (S)-4-methylbenzenesulfenamide.

| | | MS |
|---|---|---|
| | Reagent replacing (S)-4- | m/z |
| Intermediate | methylbenzenesulfenamide | (ESI) |
| (R)-2-amino-3,3- | (R)-4- | 151 |
| dicyclopropylpropanenitrile | methylbenzenesulfenamide | [M + 1] |
| hydrochloride | | |

The following intermediates were prepared according to steps 11 to 17 of Example 1, except that different reagents were used instead of 1-ethyl-1H-pyrazole-5-carboxylic acid.

| | Reagent replacing 1- | MS |
|---|---|---|
| | ethyl-1H-pyrazole-5- | m/z |
| Intermediate | carboxylic acid | (ESI) |
| (S)-N-(1-amino-3,3-dicyclopropyl-1-oxopropan-2-yl)-1- | 1-Fluorocyclopropane- | 255 |
| fluorocyclopropane-1-carboxamide | 1-carboxylic acid | [M + 1] |
| (S)-N-(1-amino-3,3-dicyclopropyl-1-oxopropan-2-yl)-4- | 4-Ethyl-1,2,5- | 293 |
| ethyl-1,2,5-oxadiazole-3-carboxamide | oxadiazole-3-carboxylic | [M + 1] |
| | acid (15g) | |
| (S)-N-(1-amino-3,3-dicyclopropyl-1-oxopropan-2-yl)-4- | 4-Methyl-1,2,5- | 279 |
| methyl-1,2,5-oxadiazole-3-carboxamide | oxadiazole-3-carboxylic | [M + 1] |
| | acid | |
| (S)-N-(1-amino-3,3-dicyclopropyl-1-oxopropan-2-yl)-4- | 4-Isopropyl-1,2,5- | 307 |
| isopropyl-1,2,5-oxadiazole-3-carboxamide | oxadiazole-3-carboxylic | [M + 1] |
| | acid | |
| (S)-N-(1-amino-3,3-dicyclopropyl-1-oxopropan-2-yl)-1- | 1-Methyl-1H-pyrazole- | 277 |
| methyl-1H-pyrazole-5-carboxamide | 5-carboxylic acid | [M + 1] |

### Example 2. N—((S)-1-((4-((1R,2S)-1-acetamido-1-(4-(cyclopropylmethyl)-4H-1,2,4-triazol-3-yl)propan-2-yl)-2-fluorophenyl)amino)-3,3-dicyclopropyl-1-oxopropan-2-yl)-1-ethyl-1H-pyrazole-5-carboxamide (Compound 2)

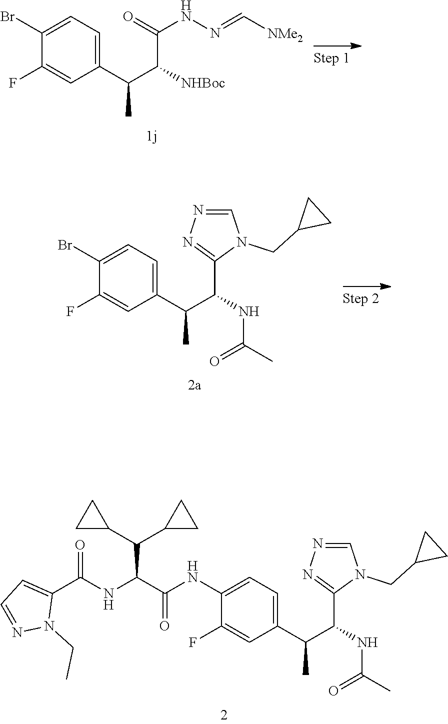 [SMILES: C[C@@H](c1ccc(Br)c(F)c1)[C@@H](NC(=O)OC(C)(C)C)C(=O)N/N=C/N(C)C ; CC(=O)N[C@@H](c1nncn1CC1CC1)[C@@H](C)c1ccc(Br)c(F)c1 ; CCn1nccc1C(=O)N[C@H](C(=O)Nc1ccc([C@H](C)[C@@H](NC(C)=O)c2nncn2CC2CC2)cc1F)C(C1CC1)C1CC1]

### Step 1. N-((1R,2S)-2-(4-bromo-3-fluorophenyl)-1-(4-(cyclopropylmethyl)-4H-1,2,4-triazol-3-yl)propyl)acetamide (2a)

To a solution of 1j (668 mg, 1.5 mmol) in acetic acid (10 mL) was added (aminomethyl)cyclopropane (1.5 mL, 21 mmol), which was then heated to 120° C. and stirred for 4 h. After cooling to room temperature, the reaction mixture was added with a saturated aqueous NaHCO₃ solution (100 mL) and extracted with dichloromethane (100 mL). The organic phase was concentrated to dryness and the residue was purified by prep-HPLC to give the title compound 2a (220 mg, 32%).

MS m/z (ESI): 395, 397 [M+1]

### Step 2. N—((S)-1-((4-((1R,2S)-1-acetamido-1-(4-(cyclopropylmethyl)-4H-1,2,4-triazol-3-yl)propan-2-yl)-2-fluorophenyl)amino)-3,3-dicyclopropyl-1-oxopropan-2-yl)-1-ethyl-1H-pyrazole-5-carboxamide (2)

A mixture of 2a (50 mg, 0.13 mmol), is (48 mg, 0.16 mmol), Cs₂CO₃ (123 mg, 0.38 mmol), CuI (20 mg, 0.11 mmol), DMEDA (20 mg, 0.23 mmol) and DMF (2 mL) was heated to 110° C. and stirred for 10 h. After cooling to room temperature, the reaction mixture was directly purified by prep-HPLC to give the title compound 2 (6.4 mg, 8%).

MS m/z (ESI): 605 [M+1]

¹H NMR (400 MHz, CD₃OD) δ 8.33 (s, 1H), 7.73 (t, J=8.2 Hz, 1H), 7.49 (d, J=2.1 Hz, 1H), 7.01 (t, J=10.2 Hz, 2H), 6.83 (d, J=2.1 Hz, 1H), 5.21 (d, J=11.0 Hz, 1H), 4.91 (d, J=7.1 Hz, 1H), 4.53 (q, J=7.2 Hz, 2H), 3.69 (dd, J=14.3, 7.4 Hz, 1H), 3.53 (ddd, J=18.4, 12.9, 7.2 Hz, 2H), 2.01 (d, J=14.1 Hz, 3H), 1.46 (t, J=7.0 Hz, 3H), 1.38 (t, J=7.2 Hz, 3H), 0.98-0.73 (m, 4H), 0.60-0.14 (m, 12H).

### Example 3. N—((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-1-ethyl-1H-pyrazole-5-carboxamide (Compound 3)

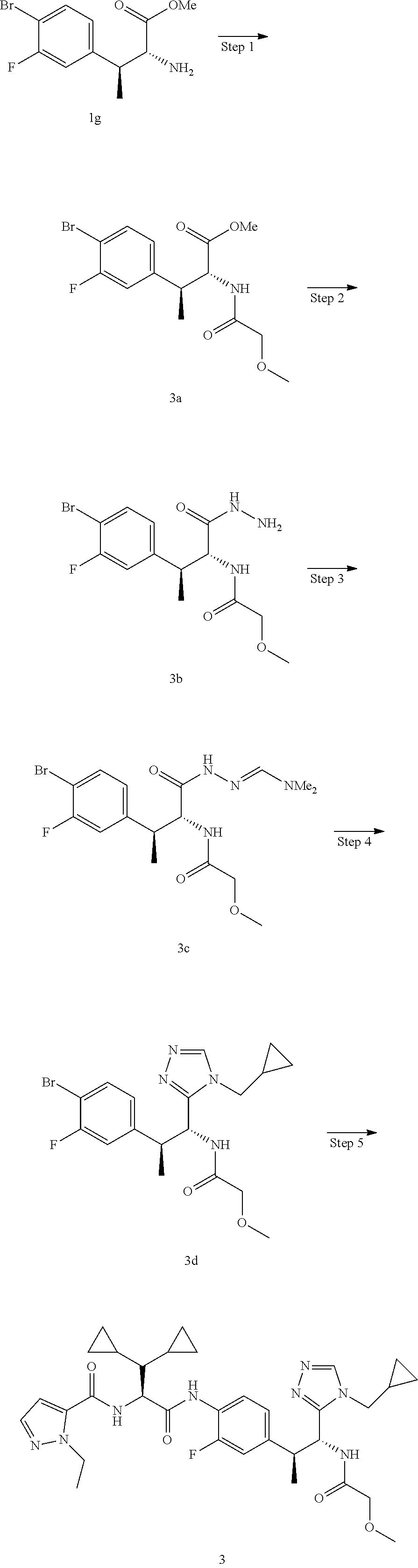 [SMILES: COC(=O)[C@H](N)[C@@H](C)c1ccc(Br)c(F)c1 ; COCC(=O)N[C@@H](C(=O)OC)[C@@H](C)c1ccc(Br)c(F)c1 ; COCC(=O)N[C@@H](C(=O)NN)[C@@H](C)c1ccc(Br)c(F)c1 ; COCC(=O)N[C@@H](C(=O)N/N=C/N(C)C)[C@@H](C)c1ccc(Br)c(F)c1 ; COCC(=O)N[C@@H](c1nncn1CC1CC1)[C@@H](C)c1ccc(Br)c(F)c1 ; CCn1nccc1C(=O)N[C@H](C(=O)Nc1ccc([C@H](C)[C@@H](NC(=O)COC)c2nncn2CC2CC2)cc1F)C(C1CC1)C1CC1]

### Step 1. Methyl (2R,3S)-3-(4-bromo-3-fluorophenyl)-2-(2-methoxyacetamido)butanoate (3a)

To a mixture of 1g (580 mg, 2 mmol), 2-methoxyacetyl chloride (201 uL, 2.4 mmol) and dichloromethane (10 mL) at 0° C. was added TEA (554 uL, 4 mmol), which was then warmed to room temperature and stirred for 1 h. The reaction mixture was added with a saturated aqueous NaHCO₃ solution (50 mL) and extracted with dichloromethane (50 mL). The organic phase was concentrated to dryness and the residue was purified by silica gel column chromatography (dichloromethane/methanol=100/0 to 9/1) to give the title compound 3a (724 mg, 99%).

MS m/z (ESI): 362, 364 [M+1]

### Step 2. N-((2R,3S)-3-(4-bromo-3-fluorophenyl)-1-hydrazineyl-1-oxobutan-2-yl)-2-methoxyacetamide (3b)

To a solution of 3a (724 mg, 2 mmol) in methanol (10 mL) was added hydrazine hydrate (486 uL, 10 mmol), which was then heated to reflux and stirred overnight. After cooling to room temperature, the reaction mixture was concentrated to dryness. The residue was dissolved in dichloromethane (100 mL) and the resulting solution was dried over anhydrous Na₂SO₄ and filtered. The filtrate was concentrated to dryness to give the title compound 3b (724 mg, crude). The crude product was directly used in the next step without further purification.

MS m/z (ESI): 362, 364 [M+1]

### Step 3. N-((2R,3S)-3-(4-bromo-3-fluorophenyl)-1-(2-((E)-(dimethylamino)methylene)hydrazineyl)-1-oxobutan-2-yl)-2-methoxyacetamide (3c)

To a solution of 3b (724 mg, crude, 2 mmol) in acetonitrile (5 mL) was added DMF-DMA (402 uL, 3 mmol), which was then heated to 50° C. and stirred for 1 h. The crude reaction mixture was directly used in the next step without further purification.

MS m/z (ESI): 417, 419 [M+1]

### Step 4. N-((1R,2S)-2-(4-bromo-3-fluorophenyl)-1-(4-(cyclopropylmethyl)-4H-1,2,4-triazol-3-yl)propyl)-2-methoxyacetamide (3d)

To a mixture from the previous step were added acetic acid (7 mL) and (aminomethyl)cyclopropane (2.4 mL, 35 mmol), which was then heated to 100° C. and stirred overnight. After cooling to room temperature, the reaction mixture was added with a saturated aqueous NaHCO₃ solution (100 mL) and extracted with dichloromethane (100 mL). The organic phase was concentrated to dryness, then the residue was purified by silica gel column chromatography (dichloromethane/methanol=100/0 to 9/1) and prep-HPLC in sequence to give the title compound 3d (1.31 g, 45%).

MS m/z (ESI): 425, 427 [M+1]

### Step 5. N—((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-1-ethyl-1H-pyrazole-5-carboxamide (3)

A mixture of 3d (70 mg, 0.16 mmol), is (62 mg, 0.21 mmol), Cs₂CO₃ (160 mg, 0.49 mmol), CuI (30 mg, 0.16 mmol), DMEDA (30 mg, 0.34 mmol) and DMF (2 mL) was heated to 110° C. and stirred for 10 h. After cooling to room temperature, the reaction mixture was purified by prep-HPLC to give the title compound 3 (30.7 mg, 30%).

MS m/z (ESI): 635 [M+1]

¹H NMR (400 MHz, CD₃OD) δ 8.33 (s, 1H), 7.74 (t, J=8.2 Hz, 1H), 7.50 (d, J=2.1 Hz, 1H), 7.05-6.96 (m, 2H), 6.84 (t, J=4.0 Hz, 1H), 5.30 (d, J=11.0 Hz, 1H), 4.92 (d, J=7.1 Hz, 1H), 4.53 (q, J=7.1 Hz, 2H), 3.95 (q, J=15.2 Hz, 2H), 3.68 (dd, J=14.3, 7.4 Hz, 1H), 3.55 (dt, J=14.3, 5.3 Hz, 2H), 3.42 (s, 3H), 1.45 (d, J=7.0 Hz, 3H), 1.38 (t, J=7.2 Hz, 3H), 0.96-0.75 (m, 4H), 0.60-0.14 (in, 12H).

Compounds 9, 12 and 23 were prepared according to the procedures of Example 3, except that different reagents were used instead of (aminomethyl)cyclopropane.

| | | MS |
|---|---|---|
| | Reagent replacing | m/z |
| Compound | (aminomethyl)cyclopropane | (ESI) |
| N-((S)-1,1-dicyclopropyl-3-((2-fluoro-4-((1R,2S)-1-(2- | 4- | 679 |
| methoxyacetamido)-1-(4-((tetrahydro-2H-pyran-4-yl)methyl)- | Aminomethyltetrahydropyran | [M + 1] |
| 4H-1,2,4-triazol-3-yl)propan-2-yl)phenyl)amino)-3- | | |
| oxopropan-2-yl)-1-ethyl-1H-pyrazole-5-carboxamide (9) | | |
| N-((S)-1,1-dicyclopropyl-3-((4-((1R,2S)-1-(4-cyclopropyl-4H- | Cyclopropylamine | 621 |
| 1,2,4-triazol-3-yl)-1-(2-methoxyacetamido)propan-2-yl)-2- | | [M + 1] |
| fluorophenyl)amino)-3-oxopropan-2-yl)-1-ethyl-1H-pyrazole- | | |
| 5-carboxamide (12) | | |
| N-((S)-1,1-dicyclopropyl-3-((2-fluoro-4-((1R,2S)-1-(4-((3- | (3-Fluoro-1- | 679 |
| fluorobicyclo[1.1.1]pentan-1-yl)methyl)-4H-1,2,4-triazol-3- | bicyclo[1.1.1]pentanyl)methanamine | [M + 1] |
| yl)-1-(2-methoxyacetamido)propan-2-yl)phenyl)amino)-3- | hydrochloride | |
| oxopropan-2-yl)-1-ethyl-1H-pyrazole-5-carboxamide (23) | | |

Compound 11 was prepared according to the procedures of Example 3, except that a different reagent was used instead of is.

| | | MS |
|---|---|---|
| | | m/z |
| Compound | Reagent replacing 1s | (ESI) |
| N-((S)-1,1-dicyclopropyl-3-((4-((1R,2S)-1-(4- | (S)-N-(1-amino-3,3- | 599 |
| (cyclopropylmethyl)-4H-1,2,4-triazol-3-yl)-1-(2- | dicyclopropyl-1- | [M + 1] |
| methoxyacetamido)propan-2-yl)-2- | oxopropan-2-yl)-1- | |
| fluorophenyl)amino)-3-oxopropan-2-yl)-1- | fluorocyclopropane-1- | |
| fluorocyclopropane-1-carboxamide (11) | carboxamide | |

Compound 16 was prepared according to the procedures of Example 3, except that a different reagent was used instead of 2-methoxyacetic acid.

| | Reagent | MS |
|---|---|---|
| | replacing 2- | m/z |
| Compound | methoxyacetic acid | (ESI) |
| N-((S)-1,1-dicyclopropyl-3-((4-((1R,2S)- | (S)-2- | 649 |
| 1-(4-(cyclopropylmethyl)-4H-1,2,4-triazol- | methoxypropanoic | [M + |
| 3-yl)-1-((S)-2-methoxypropanamido)propan- | acid | 1] |
| 2-yl)-2-fluorophenyl)amino)-3-oxopropan-2- | | |
| yl)-1-ethyl-1H-pyrazole-5-carboxamide (16) | | |

Compound 18 was prepared according to the procedures of Example 3, except that a different reagent used instead of DMF-DMA.

| | Reagent replacing | MS m/z |
|---|---|---|
| Compound | DMF-DMA | (ESI) |
| N-((S)-1,1-dicyclopropyl-3-((4-((1R,2S)- | N,N- | 649 |
| 1-(4-(cyclopropylmethyl)-5-methyl-4H- | dimethylacetamide | [M + 1] |
| 1,2,4-triazol-3-yl)-1-(2-methoxyacetamido)propan- | dimethyl acetal | |
| 2-yl)-2-fluorophenyl)amino)-3-oxopropan-2- | | |
| yl)-1-ethyl-1H-pyrazole-5-carboxamide (18) | | |

¹H NMR data of Compounds 9, 11, 12, 16, 18 and 23 are shown below:

| Compound # | ¹H NMR |
|---|---|
| 9 | ¹H NMR (400 MHz, CD₃OD) δ 8.26 (s, 1H), 7.76 (t, J = 8.2 Hz, 1H), 7.49 (d, |
| | J = 2.1 Hz, 1H), 7.14-6.98 (m, 2H), 6.83 (d, J = 2.1 Hz, 1H), 5.29 (d, J = 11.1 |
| | Hz, 1H), 4.92 (d, J = 7.1 Hz, 1H), 4.53 (d, J = 7.4 Hz, 2H), 3.96 (d, J = 11.6 |
| | Hz, 2H), 3.89-3.74 (m, 3H), 3.69-3.58 (m, 2H), 3.42 (s, 3H), 3.25 (d, J = 11.5 |
| | Hz, 1H), 3.16 (dd, J = 12.8, 10.8 Hz, 1H), 1.66 (s, 1H), 1.45 (d, J = 7.0 Hz, |
| | 3H), 1.37 (t, J = 7.2 Hz, 3H), 1.28 (d, J = 13.5 Hz, 2H), 1.17 (dd, J = 19.8, 9.3 |
| | Hz, 2H), 1.05 (d, J = 12.7 Hz, 1H), 0.93-0.76 (m, 3H), 0.57-0.40 (m, 3H), 0.27 (s, 4H). |
| 11 | ¹H NMR (400 MHZ, DMSO-d₆) δ 9.82 (s, 1H), 8.48 (d, J = 8.9 Hz, 1H), 8.31 |
| | (s, 1H), 7.89 (d, J = 7.3 Hz, 1H), 7.64 (t, J = 8.3 Hz, 1H), 7.06 (d, J = 12.2 Hz, |
| | 1H), 6.98 (d, J = 8.3 Hz, 1H), 5.23 (t, J = 9.6 Hz, 1H), 4.83 (dd, J = 8.4, 6.3 |
| | Hz, 1H), 3.87 (s, 2H), 3.65 (dd, J = 14.3, 7.2 Hz, 1H), 3.57 (dd, J = 14.5, 7.3 |
| | Hz, 2H), 3.29 (s, 3H), 1.40-1.30 (m, 5H), 1.20 (ddd, J = 19.4, 9.5, 5.1 Hz, |
| | 4H), 1.00-0.90 (m, 1H), 0.74 (t, J = 12.5 Hz, 3H), 0.49-0.32 (m, 4H), 0.27 |
| | (dd, J = 12.6, 4.5 Hz, 3H), 0.20 (dd, J = 7.8, 4.2 Hz, 2H), 0.15-0.09 (m, 1H). |
| 12 | ¹H NMR (400 MHZ, DMSO-d₆) δ 9.84 (s, 1H), 8.38 (t, J = 8.6 Hz, 2H), 8.21 |
| | (s, 1H), 7.70 (t, J = 8.3 Hz, 1H), 7.47 (d, J = 2.0 Hz, 1H), 7.05-6.93 (m, 3H), |
| | 5.36 (dd, J = 16.6, 7.2 Hz, 1H), 4.99-4.84 (m, 1H), 4.51-4.43 (m, 2H), 3.86 |
| | (s, 2H), 3.56-3.48 (m, 1H), 3.29 (s, 3H), 2.90 (dt, J = 11.1, 3.7 Hz, 1H), 2.00 |
| | (dd, J = 14.6, 6.8 Hz, 1H), 1.35-1.23 (m, 10H), 0.83-0.68 (m, 2H), 0.25 (dd, |
| | J = 49.3, 24.6 Hz, 8H). |
| 16 | ¹H NMR (400 MHZ, CD₃OD) δ 8.18 (d, J = 8.7 Hz, 1H), 7.74 (t, J = 8.1 Hz, |
| | 1H), 7.49 (d, J = 2.0 Hz, 1H), 7.00 (s, 2H), 6.83 (d, J = 2.0 Hz, 1H), 5.29-5.21 |
| | (m, 1H), 4.92 (dd, J = 10.7, 5.0 Hz, 1H), 4.53 (d, J = 7.2 Hz, 2H), 3.84 (d, J = |
| | 6.7 Hz, 1H), 3.69 (dd, J = 14.3, 7.4 Hz, 1H), 3.61-3.48 (m, 2H), 3.37 (s, 3H), |
| | 1.46 (d, J = 6.9 Hz, 3H), 1.38 (t, J = 7.2 Hz, 3H), 1.27 (d, J = 6.7 Hz, 3H), 0.95 |
| | (ddd, J = 12.8, 7.8, 5.0 Hz, 1H), 0.85 (ddd, J = 13.4, 10.9, 6.5 Hz, 2H), 0.76 (dd, |
| | J = 17.1, 8.4 Hz, 1H), 0.61-0.46 (m, 4H), 0.45-0.40 (m, 1H), 0.40-0.14 (m, 7H). |
| 18 | ¹H NMR (400 MHZ, CD₃OD) δ 7.71 (t, J = 8.2 Hz, 1H), 7.49 (d, J = 2.1 Hz, |
| | 1H), 7.09-6.98 (m, 2H), 6.84 (d, J = 2.1 Hz, 1H), 5.32 (d, J = 10.8 Hz, 1H), |
| | 4.92 (d, J = 7.0 Hz, 1H), 4.53 (q, J = 7.2 Hz, 2H), 3.96 (q, J = 15.2 Hz, 2H), |
| | 3.72 (dd, J = 15.2, 6.9 Hz, 1H), 3.61-3.49 (m, 2H), 3.42 (s, 3H), 2.31 (s, 3H), |
| | 1.49-1.31 (m, 6H), 0.91-0.73 (m, 4H), 0.57-0.17 (m, 12H). |
| 23 | ¹H NMR (400 MHZ, DMSO-d₆) δ 9.83 (s, 1H), 8.39 (s, 1H), 8.36 (s, 1H), 8.25 |
| | (d, J = 2.3 Hz, 1H), 7.71 (s, 1H), 7.47 (d, J = 2.0 Hz, 1H), 7.10 (d, J = 12.2 Hz, |
| | 1H), 7.00 (d, J = 8.3 Hz, 1H), 6.97 (d, J = 1.9 Hz, 1H), 5.20 (s, 1H), 4.92 (s, |
| | 1H), 4.47 (dd, J = 7.1, 2.4 Hz, 2H), 4.19 (s, 2H), 3.88 (s, 2H), 3.67-3.52 (m, |
| | 1H), 3.29 (s, 3H), 1.82 (d, J = 9.0 Hz, 3H), 1.76 (d, J = 9.2 Hz, 3H), 1.31-1.28 |
| | (m, 3H), 1.28-1.22 (m, 3H), 0.95-0.85 (m, 1H), 0.85-0.77 (m, 1H), 0.72 |
| | (dd, J = 17.3, 9.0 Hz, 1H), 0.43 (dd, J = 7.9, 5.5 Hz, 1H), 0.38-0.32 (m, 1H), |
| | 0.29 (s, 2H), 0.23-0.15 (m, 3H), 0.15-0.06 (m, 1H). |

### Example 4. N—((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-1-isopropyl-1H-pyrazole-5-carboxamide (Compound 4)

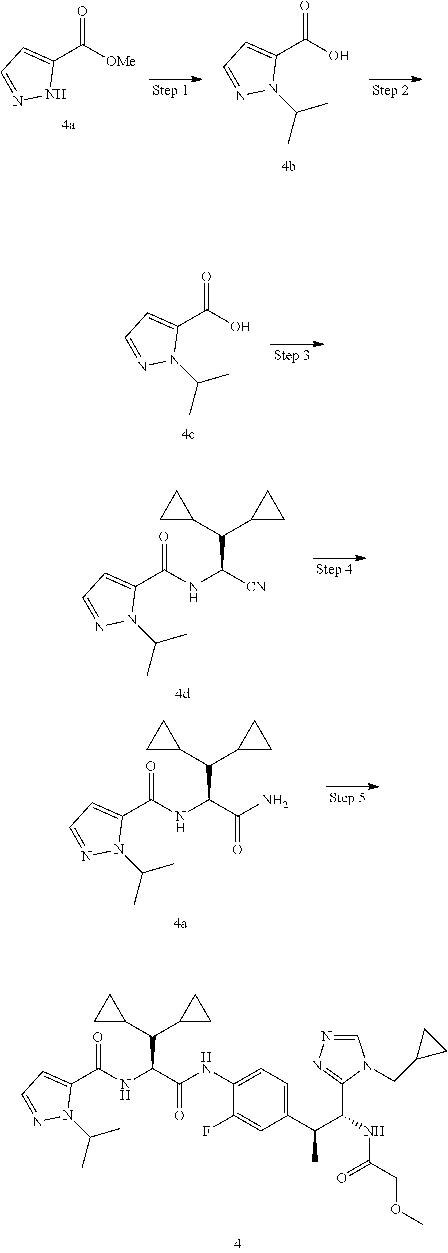 [SMILES: COC(=O)c1ccn[nH]1 ; CC(C)n1nccc1C(=O)O ; CC(C)n1nccc1C(=O)O ; CC(C)n1nccc1C(=O)N[C@H](C#N)C(C1CC1)C1CC1 ; CC(C)n1nccc1C(=O)N[C@H](C(N)=O)C(C1CC1)C1CC1 ; COCC(=O)N[C@@H](c1nncn1CC1CC1)[C@@H](C)c1ccc(NC(=O)[C@@H](NC(=O)c2ccnn2C(C)C)C(C2CC2)C2CC2)c(F)c1]

### Step 1. Methyl 1-isopropyl-1H-pyrazole-5-carboxylate (4b)

To a mixture of methyl 1H-pyrazole-5-carboxylate 4a (5.00 g, 40 mmol), isopropanol (2.86 g, 48 mmol), PPh₃ (12.48 g, 48 mmol) and THF (100 mL) at 0° C. was added DEAD (26 mL, 150 mmol), which was then stirred at 0° C. for 1 h. The reaction mixture was concentrated to dryness and the residue was purified by silica gel column chromatography (PE/EtOAc=100/0 to 7/3) to give the title compound 4b (5.90 g, 90%).

MS m/z (ESI): 169 [M+1]

### Step 2. 1-Isopropyl-1H-pyrazole-5-carboxylic acid (4c)

To a solution of 4b (5.90 g, 35 mmol) in THF (50 mL) and water (50 mL) was added LiOH·H₂O (3.40 g, 140 mmol), which was then stirred overnight. The reaction mixture was added with dilute hydrochloric acid (1 N, 200 mL) and extracted with EtOAc (200 mL). The organic phase was dried over anhydrous Na₂SO₄ and filtered. The filtrate was concentrated to dryness to give the title compound 4c (1.75 g, 32%). The crude product was directly used in the next step without further purification.

MS m/z (ESI): 155 [M+1]

### Step 3. (S)—N-(1-cyano-2,2-dicyclopropylethyl)-1-isopropyl-1H-pyrazole-5-carboxamide (4d)

To a mixture of 1q (7.50 g, 50 mmol), 4c (7.52 g, 50 mmol), HATU (20.91 g, 55 mmol) and DMF (200 mL) was added DIPEA (26 mL, 150 mmol), which was then stirred for 1 h. The reaction mixture was added with EtOAc (800 mL) and washed sequentially with a saturated aqueous NaHCO₃ solution (500 mL), dilute hydrochloric acid (1 N, 500 mL) and saturated brine (3×500 mL). The organic phase was concentrated to dryness and the residue was purified by silica gel column chromatography (PE/EtOAc=100/0 to 7/3) to give the title compound 4d (14.32 g, 99%).

MS m/z (ESI): 287 [M+1]

### Step 4. (S)—N-(1-amino-3,3-dicyclopropyl-1-oxopropan-2-yl)-1-isopropyl-1H-pyrazole-5-carboxamide (4e)

To a mixture of 4d (14.32 g, 50 mmol), hydrogen peroxide (30% aqueous solution, 50 mL) and DMSO (100 mL) at 0° C. was added NaOH (400 mg, 10 mmol), which was then stirred for 1 h. The reaction mixture was added with water (500 mL) and filtered to give the title compound 4e (15.21 g, 99%). The crude product was directly used in the next step without further purification.

MS m/z (ESI): 305 [M+1]

### Step 5. N—((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-1-isopropyl-1H-pyrazole-5-carboxamide (4)

A mixture of 3d (70 mg, 0.16 mmol), 4e (65 mg, 0.21 mmol), Cs₂CO₃ (160 mg, 0.49 mmol), CuI (30 mg, 0.16 mmol), DMEDA (30 mg, 0.34 mmol) and DMF (2 mL) was heated to 110° C. and stirred for 10 h. After cooling to room temperature, the reaction mixture was purified by prep-HPLC to give the title compound 4 (25.2 mg, 24%).

MS m/z (ESI): 649 [M+1]

¹H NMR (400 MHz, CD₃OD) δ 8.34 (s, 1H), 7.74 (dd, J=15.3, 7.2 Hz, 1H), 7.51 (d, J=2.0 Hz, 1H), 7.06-6.96 (m, 2H), 6.77 (dd, J=8.2, 2.0 Hz, 1H), 5.38 (dt, J=13.4, 6.7 Hz, 1H), 5.30 (d, J=10.9 Hz, 1H), 4.91 (d, J=7.2 Hz, 1H), 3.95 (q, J=15.2 Hz, 2H), 3.68 (dd, J=14.3, 7.4 Hz, 1H), 3.60-3.50 (m, 2H), 3.42 (s, 3H), 1.47 (ddd, J=17.2, 6.6, 5.0 Hz, 9H), 0.97-0.74 (m, 4H), 0.57-0.19 (m, 12H).

The following intermediate was prepared according to steps 1 to 2 of Example 4, except that a different reagent was used instead of isopropanol.

| | Reagent replacing | MS m/z |
|---|---|---|
| Intermediate | isopropanol | (ESI) |
| 1-(Methyl-d₃)-1H-pyrazole-5-carboxylic acid | CD₃OD | 130 |
| | | [M + 1] |

The following intermediates were prepared according to steps 1 to 4 of Example 4, except that different reagents were used instead of isopropanol.

| | Reagent | |
|---|---|---|
| | replacing | MS m/z |
| Intermediate | isopropanol | (ESI) |
| (S)-N-(1-amino-3,3-dicyclopropyl-1-oxopropan- | CD₃OD | 280 |
| 2-yl)-1-(methyl-d₃)-1H-pyrazole-5-carboxamide | | [M + 1] |
| (S)-N-(1-amino-3,3-dicyclopropyl-1-oxopropan- | CD₃OD | 280 |
| 2-yl)-1-(methyl-d₃)-1H-pyrazole-3-carboxamide | | [M + 1] |

Compound 22 was prepared according to the procedures of Example 4, except that a different reagent was used instead of 3d.

| | | MS m/z |
|---|---|---|
| Compound | Reagent replacing 3d | (ESI) |
| N-((S)-1-((4-((1R,2S)-1-acetamido-1-(4- | N-((1R,2S)-2-(4-bromo- | 619 |
| (cyclopropylmethyl)-4H-1,2,4-triazol-3-yl)propan-2-yl)-2- | 3-fluorophenyl)-1-(4- | [M + 1] |
| fluorophenyl)amino)-3,3-dicyclopropyl-1-oxopropan-2- | (cyclopropylmethyl)- | |
| yl)-1-isopropyl-1H-pyrazole-5-carboxamide (22) | 4H-1,2,4-triazol-3- | |
| | yl)propyl)acetamide (2a) | |

¹H NMR data of Compound 22 are shown below:

| Compound # | ¹H NMR |
|---|---|
| 22 | ¹H NMR (400 MHz, CD₃OD) δ 8.33 (s, 1H), 7.73 (t, J = 8.2 Hz, 1H), |
| | 7.49 (d, J = 2.1 Hz, 1H), 7.01 (t, J = 10.2 Hz, 2H), 6.83 (d, J = 2.1 Hz, |
| | 1H), 5.21 (d, J = 11.0 Hz, 1H), 4.91 (d, J = 7.1 Hz, 1H), 4.53 (q, J = 7.2 |
| | Hz, 2H), 3.69 (dd, J = 14.3, 7.4 Hz, 1H), 3.53 (ddd, J = 18.4, 12.9, 7.2 |
| | Hz, 2H), 2.01 (d, J = 14.1 Hz, 3H), 1.46 (t, J = 7.0 Hz, 3H), 1.38 (t, J = |
| | 7.2 Hz, 3H), 0.98-0.73 (m, 4H) 0.60-0.13 (m, 14H). |

### Example 5. N—((S)-1,1-dicyclopropyl-3-((2-fluoro-4-((1R,2S)-1-(2-methoxyacetamido)-1-(4-((1-methylazetidin-3-yl)methyl)-4H-1,2,4-triazol-3-yl)propan-2-yl)phenyl)amino)-3-oxopropan-2-yl)-1-ethyl-1H-pyrazole-5-carboxamide (Compound 5)

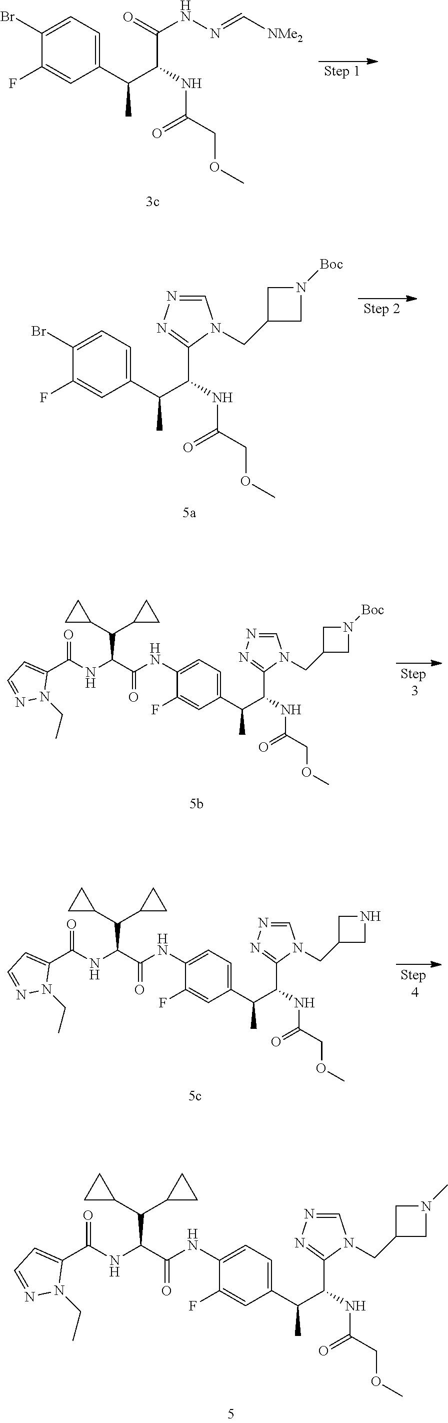 [SMILES: COCC(=O)N[C@@H](C(=O)N/N=C/N(C)C)[C@@H](C)c1ccc(Br)c(F)c1 ; COCC(=O)N[C@@H](c1nncn1CC1CN(C(=O)OC(C)(C)C)C1)[C@@H](C)c1ccc(Br)c(F)c1 ; CCn1nccc1C(=O)N[C@H](C(=O)Nc1ccc([C@H](C)[C@@H](NC(=O)COC)c2nncn2CC2CN(C(=O)OC(C)(C)C)C2)cc1F)C(C1CC1)C1CC1 ; CCn1nccc1C(=O)N[C@H](C(=O)Nc1ccc([C@H](C)[C@@H](NC(=O)COC)c2nncn2CC2CNC2)cc1F)C(C1CC1)C1CC1 ; CCn1nccc1C(=O)N[C@H](C(=O)Nc1ccc([C@H](C)[C@@H](NC(=O)COC)c2nncn2CC2CN(C)C2)cc1F)C(C1CC1)C1CC1]

### Step 1. Tert-butyl 3-((3-((1R,2S)-2-(4-bromo-3-fluorophenyl)-1-(2-methoxyacetamido)propyl)-4H-1,2,4-triazol-4-yl)methyl)azetidine-1-carboxylate (5a)

To a solution of 3c (530 mg, 1.46 mmol) in acetic acid (10 mL) was added tert-butyl 3-(aminomethyl)azetidine-1-carboxylate (1.36 g, 7.31 mmol), which was then heated to 120° C. and stirred for 30 min. After cooling to room temperature, the reaction mixture was purified by prep-HPLC to give the title compound 5a (320 mg, 41%).

MS m/z (ESI): 540, 542 [M+1]

### Step 2. Tert-butyl 3-((3-((1R,2S)-2-(4-((S)-3,3-dicyclopropyl-2-(1-ethyl-1H-pyrazole-5-carboxamido)propanamido)-3-fluorophenyl)-1-(2-methoxyacetamido)propyl)-4H-1,2,4-triazol-4-yl)methyl)azetidine-1-carboxylate (5b)

A mixture of 5a (100 mg, 0.19 mmol), is (54 mg, 0.19 mmol), Cs₂CO₃ (121 mg, 0.37 mmol), CuI (30 mg, 0.16 mmol), DMEDA (30 mg, 0.34 mmol) and DMF (4 mL) was heated to 110° C. and stirred for 10 h. After cooling to room temperature, the reaction mixture was purified by prep-HPLC to give the title compound 5b (46 mg, 52%).

MS m/z (ESI): 750 [M+1]

### Step 3. N—((S)-1-((4-((1R,2S)-1-(4-(azetidin-3-ylmethyl)-4H-1,2,4-triazol-3-yl)-1-(2-methoxyacetamido)propan-2-yl)-2-fluorophenyl)amino)-3,3-dicyclopropyl-1-oxopropan-2-yl)-1-ethyl-1H-pyrazole-5-carboxamide hydrochloride (5c)

A mixture of 5b (46 mg, 0.06 mmol), hydrochloric acid in 1,4-dioxane (4 M, 2 mL) and methanol (2 mL) was stirred for 3 h and then concentrated to dryness to give the title compound 5c (45 mg, crude). The crude product was directly used in the next step without further purification.

MS m/z (ESI): 650 [M+1]

### Step 4. N—((S)-1,1-dicyclopropyl-3-((2-fluoro-4-((1R,2S)-1-(2-methoxyacetamido)-1-(4-((1-methylazetidin-3-yl)methyl)-4H-1,2,4-triazol-3-yl)propan-2-yl)phenyl)amino)-3-oxopropan-2-yl)-1-ethyl-1H-pyrazole-5-carboxamide formate (5)

To a mixture of 5c (45 mg, 0.06 mmol), formaldehyde (40% aqueous solution, 5 mL) and methanol (10 mL) was added NaBH₃CN (15 mg, 0.24 mmol), which was then stirred for 1 h. The reaction mixture was purified by prep-HPLC to give the title compound 5 (8.2 mg, formic acid salt, 52%).

MS m/z (ESI): 664 [M+1]

¹H NMR (400 MHz, CD₃OD) δ 8.49 (d, J=26.2 Hz, 1H), 8.25 (s, 1H), 7.69 (dt, J=16.5, 8.2 Hz, 1H), 7.50 (d, J=2.1 Hz, 1H), 7.08-7.01 (m, 1H), 6.97 (d, J=8.2 Hz, 1H), 6.85 (d, J=2.0 Hz, 1H), 5.19 (d, J=11.1 Hz, 1H), 4.90 (dd, J=6.7, 3.2 Hz, 1H), 4.54 (dt, J=8.1, 6.6 Hz, 2H), 4.12 (dd, J=14.5, 8.2 Hz, 1H), 4.04-3.88 (m, 4H), 3.85-3.55 (m, 4H), 3.45 (d, J=20.0 Hz, 3H), 3.00-2.89 (m, 1H), 2.82-2.70 (m, 3H), 1.48 (d, J=6.9 Hz, 3H), 1.38 (t, J=7.2 Hz, 3H), 0.94-0.77 (m, 3H), 0.60-0.19 (m, 8H).

### Example 6. N—((S)-1,1-dicyclopropyl-3-((2-fluoro-4-((1R,2S)-1-(2-methoxyacetamido)-1-(4-methyl-4H-1,2,4-triazol-3-yl)propan-2-yl)phenyl)amino)-3-oxopropan-2-yl)-1-ethyl-1H-pyrazole-5-carboxamide (Compound 6)

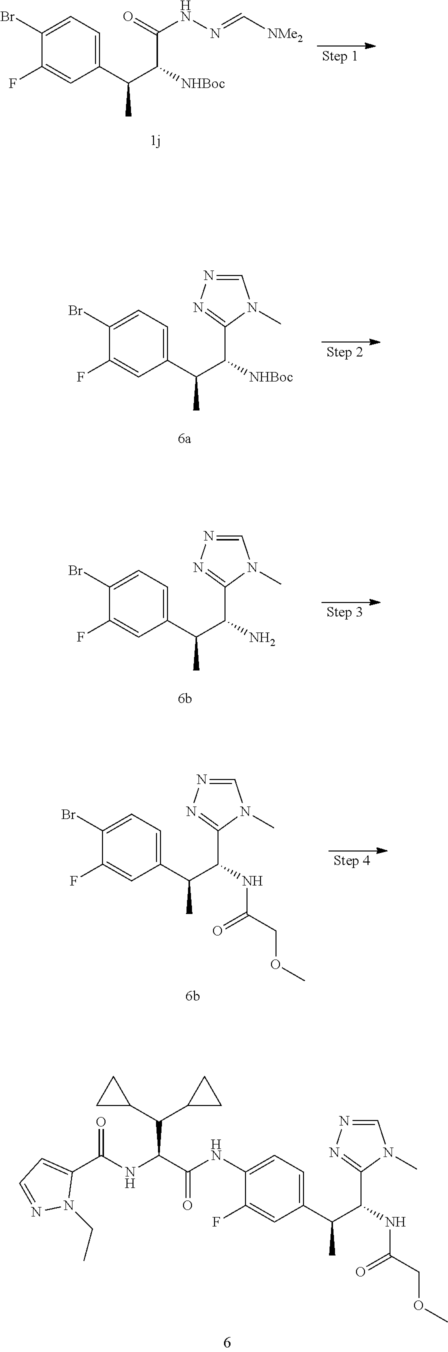 [SMILES: C[C@@H](c1ccc(Br)c(F)c1)[C@@H](NC(=O)OC(C)(C)C)C(=O)N/N=C/N(C)C ; C[C@@H](c1ccc(Br)c(F)c1)[C@@H](NC(=O)OC(C)(C)C)c1nncn1C ; C[C@@H](c1ccc(Br)c(F)c1)[C@@H](N)c1nncn1C ; COCC(=O)N[C@@H](c1nncn1C)[C@@H](C)c1ccc(Br)c(F)c1 ; CCn1nccc1C(=O)N[C@H](C(=O)Nc1ccc([C@H](C)[C@@H](NC(=O)COC)c2nncn2C)cc1F)C(C1CC1)C1CC1]

### Step 1. Tert-butyl ((1R,2S)-2-(4-bromo-3-fluorophenyl)-1-(4-methyl-4H-1,2,4-triazol-3-yl)propyl)carbamate (6a)

To a solution of 1j (500 mg, 1.28 mmol) in acetic acid (7 mL) was added methylamine (40% aqueous solution, 3 mL), which was then heated to 120° C. and stirred for 15 min. After cooling to room temperature, the reaction mixture was concentrated to dryness and the residue was purified by prep-HPLC to give the title compound 6a (230 mg, 43%).

MS m/z (ESI): 413, 415 [M+1]

### Step 2. (1R,2S)-2-(4-bromo-3-fluorophenyl)-1-(4-methyl-4H-1,2,4-triazol-3-yl)propan-1-amine hydrochloride (6b)

A mixture of 6a (230 mg, 0.56 mmol), methanol (5 mL) and hydrochloric acid in 1,4-dioxane (4 M, 5 mL) was stirred for 5 h and then concentrated to dryness to give the title compound 6b (230 mg, crude). The crude product was directly used in the next step without further purification.

MS m/z (ESI): 313, 315 [M+1]

### Step 3. N-((1R,2S)-2-(4-bromo-3-fluorophenyl)-1-(4-methyl-4H-1,2,4-triazol-3-yl)propyl)-2-methoxyacetamide (6c)

To a mixture of 6b (230 mg, crude, 0.56 mmol), 2-methoxyacetic acid (151 mg, 1.68 mmol), HATU (319 mg, 0.84 mmol) and DMF (5 mL) was added DIPEA (289 mg, 2.24 mmol), which was then stirred for 30 min. The reaction mixture was purified by prep-HPLC to give the title compound 6c (170 mg, 79%).

MS m/z (ESI): 385, 387 [M+1]

### Step 4. N—((S)-1,1-dicyclopropyl-3-((2-fluoro-4-((1R,2S)-1-(2-methoxyacetamido)-1-(4-methyl-4H-1,2,4-triazol-3-yl)propan-2-yl)phenyl)amino)-3-oxopropan-2-yl)-1-ethyl-1H-pyrazole-5-carboxamide (6)

A mixture of 6c (100 mg, 0.26 mmol), is (75 mg, 0.26 mmol), Cs₂CO₃ (169 mg, 0.52 mmol), CuI (30 mg, 0.16 mmol), DMEDA (30 mg, 0.34 mmol) and DMF (5 mL) was heated to 110° C. and stirred for 10 h. After cooling to room temperature, the reaction mixture was purified by prep-HPLC to give the title compound 6 (33.6 mg, 22%).

MS m/z (ESI): 595 [M+1]

¹H NMR (400 MHz, CD₃OD) δ 8.15 (s, 1H), 7.76 (t, J=8.2 Hz, 1H), 7.50 (d, J=2.1 Hz, 1H), 7.09-6.95 (m, 2H), 6.84 (d, J=2.1 Hz, 1H), 5.29 (d, J=10.8 Hz, 1H), 4.93 (d, J=7.0 Hz, 1H), 4.53 (q, J=7.2 Hz, 2H), 3.95 (q, J=15.2 Hz, 2H), 3.52 (dd, J=10.8, 6.9 Hz, 1H), 3.43 (d, J=3.4 Hz, 6H), 1.45 (d, J=6.9 Hz, 3H), 1.38 (t, J=7.2 Hz, 3H), 0.91-0.73 (m, 3H), 0.57-0.18 (m, 8H).

### Example 7. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2,2-difluoro-2-(6-methoxypyridin-3-yl)acetamido)propanamide (Compound 7)

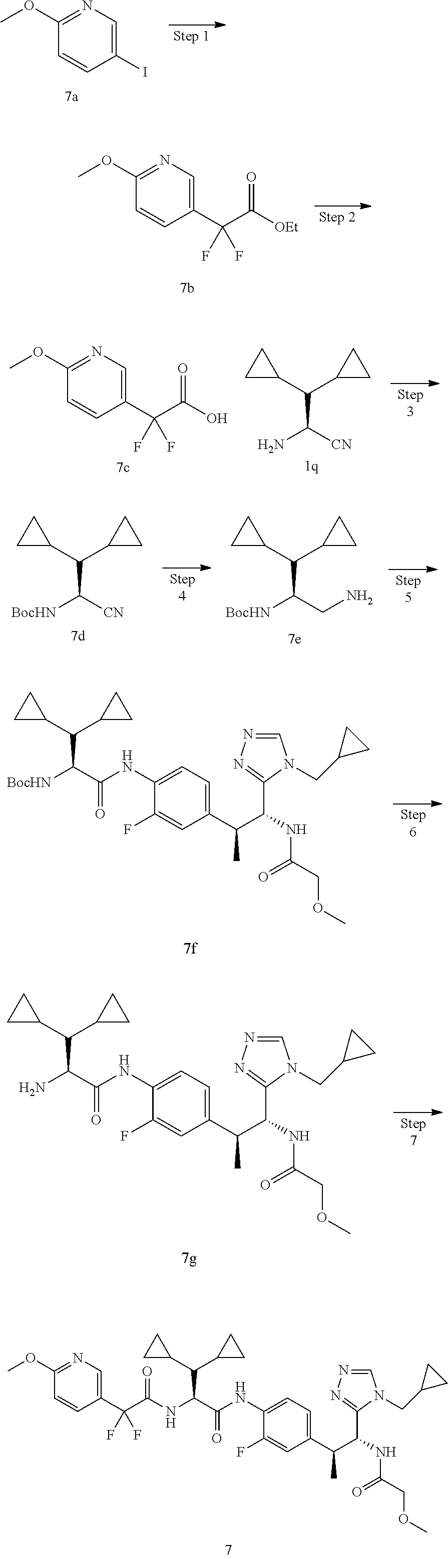

### Step 1. Ethyl 2,2-difluoro-2-(6-methoxypyridin-3-yl)acetate (7b)

To a mixture of 5-iodo-2-methoxypyridine 7a (10.00 g, 42.6 mmol), ethyl bromodifluoroacetate (17.27 g, 85.1 mmol) and DMSO (120 mL) was added copper powder (5.41 g, 85.1 mmol), which was then heated to 60° C. and stirred for 16 h. After cooling to room temperature, the reaction mixture was added with water (100 mL) and extracted with EtOAc (3×100 mL). The combined organic phase was dried over anhydrous Na₂SO₄ and filtered. The filtrate was concentrated to dryness to give the title compound 7b (9.83 g, crude). The crude product was directly used in the next step without further purification.

MS m/z (ESI): 232 [M+1]

### Step 2. 2,2-Difluoro-2-(6-methoxypyridin-3-yl)acetic acid (7c)

To a solution of 7b (9.83 g, crude, 42.5 mmol) in THF (50 mL) and water (50 mL) was added LiOH·H₂O (1.78 g, 50.5 mmol), which was then stirred overnight. The reaction mixture was added with dilute hydrochloric acid (1 N, 100 mL) and extracted with EtOAc (3×100 mL). The combined organic phase was dried over anhydrous Na₂SO₄ and filtered. The filtrate was concentrated to dryness to give the title compound 7c (5.40 g, 63%). The crude product was directly used in the next step without further purification.

MS m/z (ESI): 204 [M+1]

### Step 3. Tert-butyl (S)-(1-cyano-2,2-dicyclopropylethyl)carbamate (7d)

To a mixture of 1q (1.50 g, 10 mmol), Boc₂O (4.3 mL, 15 mmol), THF (20 mL) and water (20 mL) was added K₂CO₃ (4.15 g, 30 mmol), which was then stirred for 6 h. The reaction mixture was added EtOAc (100 mL) and washed with dilute hydrochloric acid (1 N, 100 mL) and saturated brine (100 mL). The organic phase was dried over anhydrous Na₂SO₄ and filtered. The filtrate was concentrated to dryness to give the title compound 7d (2.50 g, crude). The crude product was directly used in the next step without further purification.

MS m/z (ESI): 251 [M+1]

### Step 4. Tert-butyl (S)-(1-amino-3,3-dicyclopropyl-1-oxopropan-2-yl)carbamate (7e)

To a solution of 7d (2.50 g, crude, 10 mmol) in hydrogen peroxide (30% aqueous solution, 10 mL) and DMSO (20 mL) at 0° C. was added NaOH (80 mg, 2 mmol), which was then stirred for 1 h. The reaction mixture was added with water (200 mL) and filtered to give the title compound 7e (2.03 g, 76%). The crude product was directly used in the next step without further purification.

MS m/z (ESI): 269 [M+1]

### Step 5. Tert-butyl ((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)carbamate (7f)

A mixture of 7e (966 mg, 3.6 mmol), 3d (1.28 g, 3.0 mmol), BrettPhos Pd G3 (272 mg, 0.3 mmol), Cs₂CO₃ (1.95 g, 6.0 mmol) and 1,4-dioxane (15 mL) was heated to 110° C. and stirred for 10 h. After cooling to room temperature, the reaction mixture was added EtOAc (100 mL) and washed with saturated brine (100 mL). The organic phase was concentrated to dryness and the residue was purified by silica gel column chromatography (dichloromethane/methanol=100/0 to 9/1) to give the title compound 7f (1.53 g, 83%).

MS m/z (ESI): 613 [M+1]

### Step 6. (S)-2-amino-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)propanamide hydrochloride (7g)

A solution of 7f (1.22 g, 2 mmol) in hydrochloric acid in 1,4-dioxane (4 M, 5 mL) and methanol (5 mL) was stirred for 5 h. The reaction mixture was concentrated to dryness to give the title compound 7g (1.03 g, crude). The crude product was directly used in the next step without further purification.

MS m/z (ESI): 513 [M+1]

### Step 7. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2,2-difluoro-2-(6-methoxypyridin-3-yl)acetamido)propanamide (7)

To a mixture of 7c (61 mg, 0.3 mmol), 7g (100 mg, 0.2 mmol), HATU (114 mg, 0.3 mmol) and DMF (5 mL) was added DIPEA (129 mg, 1.0 mmol), which was then stirred for 1 h. The reaction mixture was purified by prep-HPLC to give the title compound 7 (33.7 mg, 25%).

MS m/z (ESI): 698 [M+1]

¹H NMR (400 MHz, CD₃OD) δ 8.41 (s, 1H), 8.39 (s, 1H), 7.90 (dd, J=8.8, 2.5 Hz, 1H), 7.72 (t, J=8.2 Hz, 1H), 7.06-6.93 (m, 2H), 6.87 (d, J=8.7 Hz, 1H), 5.29 (d, J=11.0 Hz, 1H), 4.04-3.86 (m, 5H), 3.69 (dd, J=14.3, 7.4 Hz, 1H), 3.60-3.49 (m, 2H), 3.42 (s, 3H), 1.45 (d, J=6.9 Hz, 3H), 1.04-0.82 (m, 2H), 0.81-0.68 (m, 3H), 0.58-0.38 (m, 4H), 0.35-0.07 (m, 8H).

The following intermediate was prepared according to steps 3 to 6 of Example 7, except that a different reagent was used instead of 2-methoxyacetic acid.

| | Reagent replacing 2- | MS m/z |
|---|---|---|
| Intermediate | methoxyacetic acid | (ESI) |
| (S)-2-amino-3,3-dicyclopropyl-N-(4-((1R,2S)-1-(4- | (S)-2- | 527 |
| (cyclopropylmethyl)-4H-1,2,4-triazol-3-yl)-1-((S)-2- | methoxypropanoic | [M + 1] |
| methoxypropanamido)propan-2-yl)-2- | acid | |
| fluorophenyl)propanamide hydrochloride | | |

### Example 8. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2-((5-(difluoromethyl)isoxazol-3-yl)oxy)acetamido)propanamide (Compound 8)

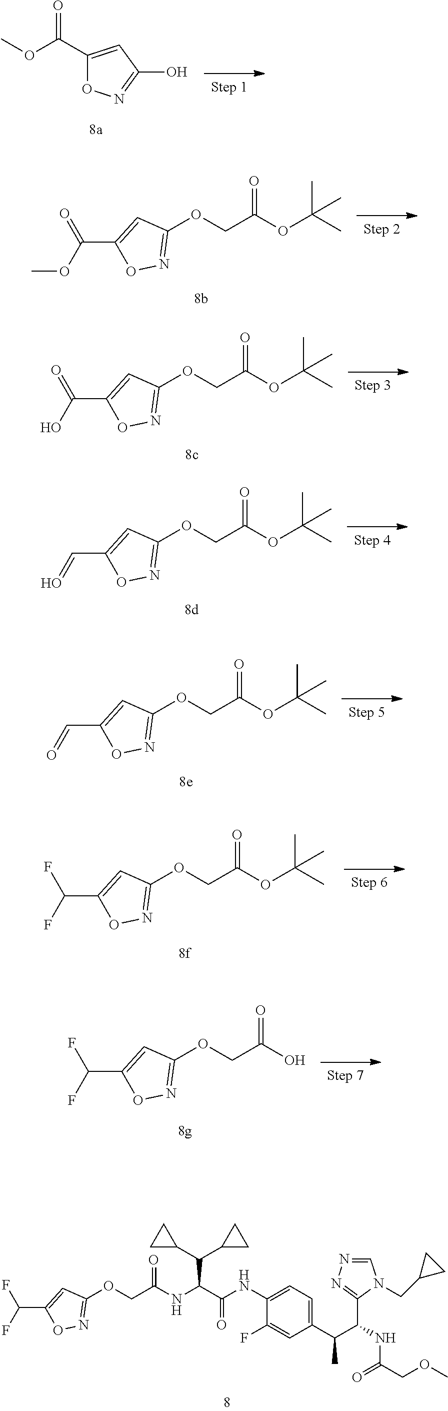

### Step 1. Methyl 3-(2-(tert-butoxy)-2-oxoethoxy)isoxazole-5-carboxylate (8b)

To a mixture of methyl 3-hydroxyisoxazole-5-carboxylate 8a (2.86 g, 20 mmol), K₂CO₃ (5.52 g, 40 mmol) and DMF (15 mL) was added tert-butyl bromoacetate (5.85 g, 30 mmol), which was then heated to 70° C. and stirred for 1 h. After cooling to room temperature, the reaction mixture was added with water (150 mL) and extracted with EtOAc (2×200 mL). The combined organic phase was washed with saturated brine (150 mL) and concentrated to dryness. The residue was purified by silica gel column chromatography (PE/EtOAc=100/1 to 10/1) to give the title compound 8b (4.20 g, 82%).

¹H NMR (400 MHz, CDCl₃) δ 6.63 (s, 1H), 4.75 (s, 2H), 3.95 (s, 3H), 1.49 (s, 9H).

### Step 2. 3-(2-(Tert-butoxy)-2-oxoethoxy)isoxazole-5-carboxylic acid (8c)

To a solution of 8b (4.2 g, 16.3 mmol) in THF (15 mL) and methanol (5 mL) was added an aqueous LiOH solution (1 M, 24.5 mL), which was then stirred for 4 h. The reaction mixture was added with acetic acid (2.2 g) and the resulting mixture was concentrated to dryness. The residue was purified by prep-HPLC to give the title compound 8c (3.0 g, 76%).

MS m/z (ESI): 242 [M−1]

### Step 3. Tert-butyl 2-((5-(hydroxymethyl)isoxazol-3-yl)oxy)acetate (8d)

To a solution of 8c (2.5 g, 10.3 mmol) in THF (60 mL) was added a solution of borane in THF (2 M, 51.5 mL), which was then stirred for 4 h. The reaction mixture was added with methanol (100 mL) and the resulting mixture was concentrated to dryness. The residue was purified by prep-HPLC to give the title compound 8d (1.5 g, 64%).

MS m/z (ESI): 252 [M+Na]

### Step 4. Tert-butyl 2-((5-formylisoxazol-3-yl)oxy)acetate (8e)

To a solution of 8d (115 mg, 0.5 mmol) in acetonitrile (5 mL) was added IBX (210 mg, 0.75 mmol), which was then heated to 80° C. and stirred for 4 h. After cooling to room temperature, the reaction mixture was purified by silica gel column chromatography (PE/EtOAc=50/1 to 3/1) to give the title compound 8e (70 mg, 62%).

¹H NMR (400 MHz, CDCl₃) δ 9.84 (s, 1H), 6.67 (s, 1H), 4.77 (s, 2H), 1.50 (s, 9H).

### Step 5. Tert-butyl 2-((5-(difluoromethyl)isoxazol-3-yl)oxy)acetate (8f)

To a solution of 8e (1.00 g, 4.4 mmol) in dichloromethane (30 mL) was added DAST (1.42 g, 8.8 mmol), which was then stirred for 4 h. The reaction mixture was added with dichloromethane (100 mL) and washed with a saturated aqueous NaHCO₃ solution (50 mL). The organic phase was concentrated to dryness and the residue was purified by silica gel column chromatography (PE/EtOAc=50/1 to 3/1) to give the title compound 8f (800 mg, 73%).

MS m/z (ESI): 250 [M+1]

### Step 6. 2-((5-(Difluoromethyl)isoxazol-3-yl)oxy)acetic acid (8g)

A solution of 8f (500 mg, 2 mmol) in dichloromethane (5 mL) and TFA (3 mL) was stirred for 5 h. The reaction mixture was concentrated to dryness and the residue was purified by prep-HPLC to give the title compound 8g (330 mg, 85%).

MS m/z (ESI): 194 [M+1]

### Step 7. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2-((5-(difluoromethyl)isoxazol-3-yl)oxy)acetamido)propanamide (8)

To a mixture of 8g (58 mg, 0.30 mmol), 7g (128 mg, 0.25 mmol), HATU (143 mg, 0.38 mmol) and DMF (3 mL) was added DIPEA (97 mg, 0.75 mmol), which was then stirred for 1 h. The reaction mixture was purified by prep-HPLC to give the title compound 8 (23.5 mg, 11%).

MS m/z (ESI): 688 [M+1]

¹H NMR (400 MHz, CD₃OD) δ 8.52 (s, 1H), 7.67 (dd, J=13.9, 5.8 Hz, 1H), 7.06-6.73 (m, 3H), 6.56 (t, J=1.4 Hz, 1H), 5.31 (d, J=11.0 Hz, 1H), 4.86 (d, J=6.0 Hz, 2H), 4.84 (s, 1H), 3.95 (q, J=15.2 Hz, 2H), 3.73 (dd, J=14.3, 7.4 Hz, 1H), 3.64-3.51 (m, 2H), 3.45 (d, J=20.9 Hz, 3H), 1.45 (d, J=6.9 Hz, 3H), 1.30 (ddd, J=13.7, 7.6, 4.8 Hz, 1H), 0.97 (ddd, J=12.5, 7.8, 4.8 Hz, 1H), 0.84-0.14 (m, 14H).

### Example 9. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2-((3-(difluoromethyl)oxetan-3-yl)methoxy)acetamido)propanamide (Compound 10)

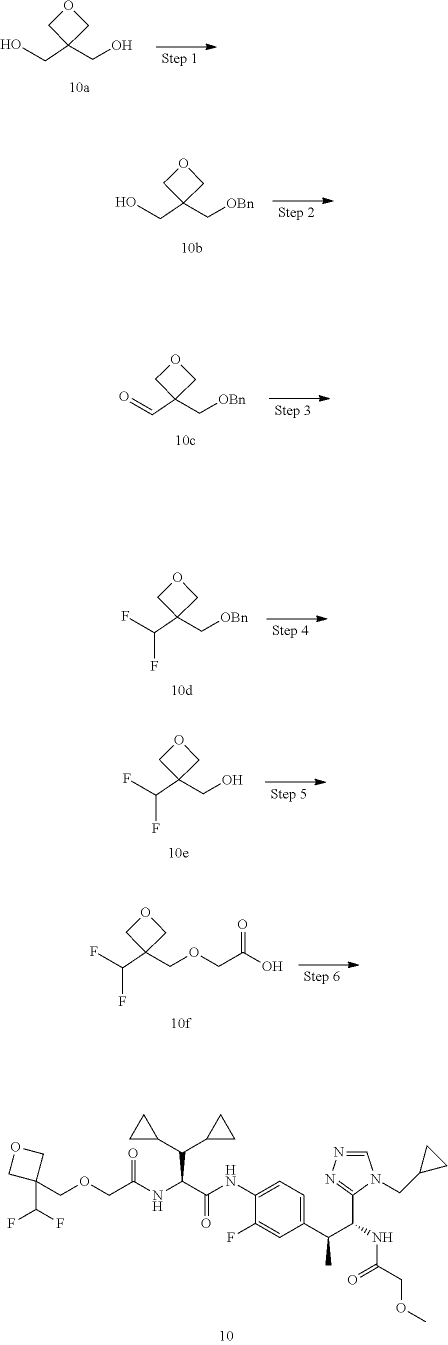

### Step 1. (3-((Benzyloxy)methyl)oxetan-3-yl)methanol (10b)

To a mixture of oxetane-3,3-diyldimethanol 10a (4.96 g, 42 mmol), tetrabutylammonium bromide (2.58 g, 8 mmol) and THF (100 mL) at 0° C. was added NaH (60% in mineral oil, 1.68 g, 42 mmol), which was then stirred for 1 h. The mixture was added with benzyl bromide (7.18 g, 42 mmol), warmed to room temperature and stirred overnight. The mixture was quenched with water (100 mL) and extracted with EtOAc (3×100 mL). The combined organic phase was concentrated to dryness and the residue was purified by silica gel column chromatography (PE/EtOAc=100/0 to 0/100) to give the title compound 10b (2.00 g, 23%).

¹H NMR (400 MHz, CDCl₃) δ 7.52-7.28 (m, 5H), 4.56 (s, 2H), 4.49 (d, J=6.2 Hz, 2H), 4.43 (d, J=6.2 Hz, 2H), 3.93 (s, 2H), 3.80 (s, 2H).

### Step 2. 3-((Benzyloxy)methyl)oxetane-3-carbaldehyde (10c)

To a solution of 10b (1.04 g, 5 mmol) in dichloromethane (20 mL) was added Dess-Martin periodinane (8.48 g, 20 mmol), which was then stirred for 2 h. The reaction mixture was filtered and the filtrate was concentrated to dryness to give the title compound 10c (1.00 g, crude). The crude product was directly used in the next step without further purification.

### Step 3. 3-((Benzyloxy)methyl)-3-(difluoromethyl)oxetane (10d)

To a solution of 10c (1.0 g, crude, 5 mmol) in dichloromethane (20 mL) at 0° C. was added DAST (3.22 g, 20 mmol), which was then warmed to room temperature and stirred overnight. The reaction mixture was added with water (100 mL) and extracted with dichloromethane (3×100 mL). The combined organic phase was concentrated to dryness and the residue was purified by silica gel column chromatography (PE/EtOAc=100/0 to 1/2) to give the title compound 10d (200 mg, 18%).

¹H NMR (400 MHz, CDCl₃) δ 7.42-7.25 (m, 5H), 6.01 (t, J=56.5 Hz, 1H), 4.71 (d, J=6.5 Hz, 2H), 4.55 (s, 2H), 4.42 (dt, J=6.5, 1.7 Hz, 2H), 3.78 (s, 2H).

### Step 4. (3-(Difluoromethyl)oxetan-3-yl)methanol (10e)

To a solution of 10d (200 mg, 0.87 mmol) in methanol (2 mL) and formic acid (2 mL) was added Pd(OH)₂/C (10%, 400 mg), which was then heated to reflux and stirred overnight under hydrogen atmosphere. After cooling to room temperature, the reaction mixture was filtered, and the filtrate was concentrated to dryness to give the title compound 10e (120 mg, 99%). The crude product was directly used in the next step without further purification.

### Step 5. 2-((3-(Difluoromethyl)oxetan-3-yl)methoxy)acetic acid (10f)

To a solution of 10e (120 mg, 0.87 mmol) in THF (4 mL) at 0° C. was added NaH (60% in mineral oil, 160 mg, 4.0 mmol), which was then stirred for 1 h. The mixture was added with bromoacetic acid (276 mg, 3.0 mmol), warmed to room temperature and stirred overnight. The mixture was added with dilute hydrochloric acid (1 N, 10 mL) and concentrated to dryness. The residue was purified by prep-HPLC to give the title compound 10f (18 mg, 13%).

MS m/z (ESI): 195 [M−1]

### Step 6. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2-((3-(difluoromethyl)oxetan-3-yl)methoxy)acetamido)propanamide (10)

To a mixture of 10f (18 mg, 0.09 mmol), 7g (51 mg, 0.09 mmol), HATU (76 mg, 0.20 mmol) and DMF (4 mL) was added DIPEA (65 mg, 0.50 mmol), which was then stirred for 1 h. The reaction mixture was purified by prep-HPLC to give the title compound 10 (11.3 mg, 16%).

MS m/z (ESI): 691 [M+1]

¹H NMR (400 MHz, CD₃OD) δ 8.30 (s, 1H), 7.74 (d, J=9.1 Hz, 1H), 7.58 (t, J=8.3 Hz, 1H), 7.05 (d, J=12.2 Hz, 1H), 6.97 (d, J=8.3 Hz, 1H), 6.30 (t, J=56.1 Hz, 1H), 5.25-5.13 (m, 1H), 4.80 (dd, J=8.9, 6.4 Hz, 1H), 4.54 (d, J=6.5 Hz, 2H), 4.50-4.43 (m, 2H), 4.06 (s, 2H), 3.86 (s, 2H), 3.80 (d, J=10.2 Hz, 2H), 3.60 (ddd, J=33.7, 14.4, 7.4 Hz, 3H), 3.28 (s, 3H), 2.10-1.81 (m, 1H), 1.32 (d, J=6.9 Hz, 3H), 0.93 (s, 1H), 0.64 (dd, J=32.4, 23.6 Hz, 2H), 0.50-0.09 (m, 11H).

### Example 10. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2-((4-(difluoromethyl)pyridin-2-yl)oxy)acetamido)propanamide (Compound 13)

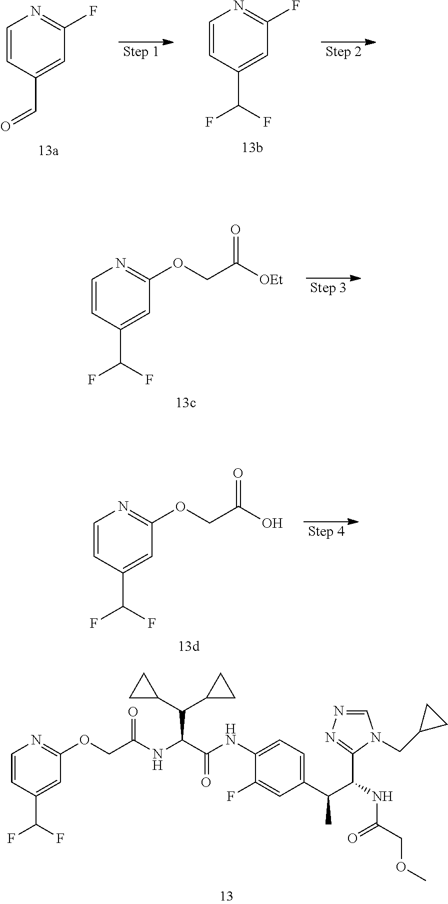 [SMILES: O=Cc1ccnc(F)c1 ; Fc1cc(C(F)F)ccn1 ; CCOC(=O)COc1cc(C(F)F)ccn1 ; O=C(O)COc1cc(C(F)F)ccn1 ; COCC(=O)N[C@@H](c1nncn1CC1CC1)[C@@H](C)c1ccc(NC(=O)[C@@H](NC(=O)COc2cc(C(F)F)ccn2)C(C2CC2)C2CC2)c(F)c1]

### Step 1. 4-(Difluoromethyl)-2-fluoropyridine (13b)

To a solution of 2-fluoroisonicotinaldehyde 13a (4.0 g, 32 mmol) in dichloromethane (50 mL) at 0° C. was added DAST (12.9 g, 80 mmol), which was then warmed to room temperature and stirred for 12 h. The reaction mixture was added with water (100 mL) and extracted with dichloromethane (200 mL). The organic phase was concentrated to dryness and the residue was purified by silica gel column chromatography (PE/EtOAc=100/0 to 2/1) to give the title compound 13d (3.7 g, 78%).

MS m/z (ESI): 148 [M+1]

### Step 2. Ethyl 2-((4-(difluoromethyl)pyridin-2-yl)oxy)acetate (13c)

To a mixture of 13b (2.0 g, 13.6 mmol), ethyl glycolate (2.8 g, 27.2 mmol) and DMF (20 mL) was added Cs₂CO₃ (13.3 g, 40.8 mmol), which was then heated to 60° C. and stirred for 12 h. After cooling to room temperature, the reaction mixture was added with water (50 mL) and extracted with EtOAc (3×50 mL). The combined organic phase was concentrated to dryness and the residue was purified by silica gel column chromatography (PE/EtOAc=100/0 to 3/1) to give the title compound 13c (2.5 g, 77%).

MS m/z (ESI): 232 [M+1]

### Step 3. 2-((4-(Difluoromethyl)pyridin-2-yl)oxy)acetic acid (13d)

To a solution of 13c (2.5 g, 10.8 mmol) in THF (10 mL) and water (2 mL) was added LiOH·H₂O (9.0 g, 21.6 mmol), which was then stirred for 1 h. The reaction mixture was added with dilute hydrochloric acid (2 N, 20 mL) and extracted with EtOAc (3×20 mL). The combined organic phase was dried over anhydrous Na₂SO₄ and filtered. The filtrate was concentrated to dryness to give the title compound 13d (1.5 g, 68%). The crude product was directly used in the next step without further purification.

MS m/z (ESI): 204 [M+1]

### Step 4. (S)-3,3-dicyclopropyl-N-(4-((1R,2S)-1-(4-(cyclopropylmethyl)-4H-1,2,4-triazol-3-yl)-1-(2-methoxyacetamido)propan-2-yl)-2-fluorophenyl)-2-(2-((4-(difluoromethyl)pyridin-2-yl)oxy)acetamido)propanamide (13)

To a mixture of 13d (42 mg, 0.21 mmol), 7g (70 mg, 0.14 mmol), HATU (78 mg, 0.23 mmol) and DMF (10 mL) was added DIPEA (88 mg, 0.69 mmol), which was then stirred for 1 h. The reaction mixture was purified by prep-HPLC to give the title compound 13 (15.6 mg, 16%).

MS m/z (ESI): 691 [M+1]

¹H NMR (400 MHz, DMSO-d₆) δ 9.69 (s, 1H), 8.47 (d, J=9.0 Hz, 1H), 8.30 (s, 1H), 8.23 (d, J=5.1 Hz, 1H), 8.10 (d, J=9.1 Hz, 1H), 7.59 (t, J=8.3 Hz, 1H), 7.22-6.88 (m, 5H), 5.27-5.14 (m, 1H), 4.88 (d, J=3.2 Hz, 2H), 4.81-4.73 (m, 1H), 3.86 (s, 2H), 3.60 (ddd, J=18.7, 14.4, 7.3 Hz, 3H), 3.28 (s, 3H), 2.54 (s, 1H), 1.32 (d, J=6.9 Hz, 3H), 0.65 (d, J=63.8 Hz, 3H), 0.48-0.05 (m, 12H).

### Example 11. N—((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-4-ethyl-1,2,5-oxadiazole-3-carboxamide (Compound 14)

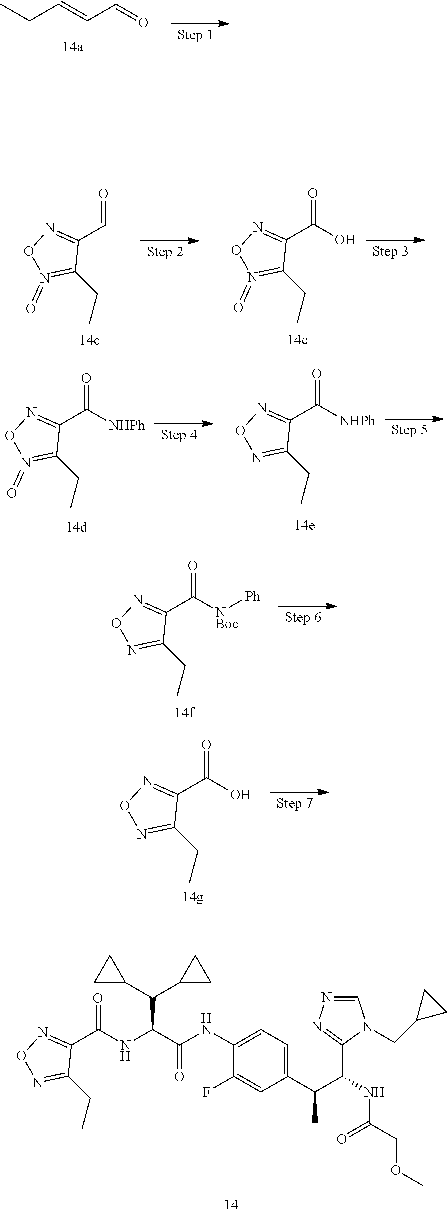

### Step 1. 3-Ethyl-4-formyl-1,2,5-oxadiazole 2-oxide (14b)

To a solution of (E)-pent-2-enal 14a (9.8 mL, 100 mmol) in acetic acid (60 mL) at 0° C. was added a solution of NaNO₂ (2.42 g, 350 mmol) in water (60 mL) slowly, which was then warmed to room temperature and stirred for 12 h. The reaction mixture was added with a saturated aqueous NaHCO₃ solution (500 mL) and extracted with EtOAc (3×200 mL). The combined organic phase was dried over anhydrous Na₂SO₄ and filtered. The filtrate was concentrated to dryness to give the title compound 14b (7.3 g, 51%). The crude product was directly used in the next step without further purification.

### Step 2. 4-Carboxy-3-ethyl-1,2,5-oxadiazole 2-oxide (14c)

To a solution of 14b (7.3 g, 51.4 mmol) in acetic acid (30 mL) was added hydrogen peroxide (30% aqueous solution, 20 mL), which was then stirred for 12 h. The reaction mixture was added with a saturated aqueous Na₂S₂O₃ solution (100 mL) and extracted with dichloromethane (3×100 mL). The combined organic phase was dried over anhydrous Na₂SO₄ and filtered. The filtrate was concentrated to dryness to give the title compound 14c (8.1 g, 99%). The crude product was directly used in the next step without further purification.

### Step 3. 3-Ethyl-4-(phenylcarbamoyl)-1,2,5-oxadiazole 2-oxide (14d)

To a mixture of 14c (8.1 g, 51.3 mmol), aniline (14 g, 154 mmol), HATU (58.5 g, 154 mmol) and DMF (200 mL) was added TEA (26 g, 257 mmol), which was then stirred for 2 h. The reaction mixture was added with water (200 mL) and extracted with EtOAc (3×200 mL).

The combined organic phase was concentrated to dryness and the residue was purified by silica gel column chromatography (PE/EtOAc=7/1) to give the title compound 14d (11.9 g, 99%).

MS m/z (ESI): 234 [M+1]

### Step 4. 4-Ethyl-N-phenyl-1,2,5-oxadiazole-3-carboxamide (14e)

To a solution of 14d (11.9 g, 50.9 mmol) in toluene (100 mL) was added trimethyl phosphite (50 mL), which was then heated to 120° C. and stirred for 12 h. After cooling to room temperature, the reaction mixture was concentrated to dryness. The residue was added with dilute hydrochloric acid (1 N, 200 mL) and extracted with EtOAc (3×100 mL). The combined organic phase was concentrated to dryness and the residue was purified by silica gel column chromatography (PE/EtOAc=3/1) to give the title compound 14e (3.0 g, 27%).

MS m/z (ESI): 218 [M+1]

### Step 5. Tert-butyl (4-ethyl-1,2,5-oxadiazole-3-carbonyl)(phenyl)carbamate (14f)

To a solution of 14e (3.0 g, 13.8 mmol) in dichloromethane (20 mL) were added Boc₂O (3.6 g, 16.6 mmol) and DMAP (168 mg, 1.38 mmol), which was then stirred for 2 h. The reaction mixture was concentrated to dryness and the residue was purified by silica gel column chromatography (PE/EtOAc=5/1) to give the title compound 14f (4.2 g, 97%).

MS m/z (ESI): 318 [M+1]

### Step 6. 4-Ethyl-1,2,5-oxadiazole-3-carboxylic acid (14g)

To a solution of 14f (4.2 g, 13.2 mmol) in THF (20 mL) and water (5 mL) was added LiOH·H₂O (1.66 g, 39.6 mmol), which was then stirred for 2 h. The reaction mixture was added with dilute hydrochloric acid (2 N, 20 mL) and extracted with EtOAc (3×30 mL). The combined organic phase was dried over anhydrous Na₂SO₄ and filtered. The filtrate was concentrated to dryness to give the title compound 14g (1.6 g, 88%). The crude product was directly used in the next step without further purification.

MS m/z (ESI): 141 [M−1]

### Step 7. N—((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-4-ethyl-1,2,5-oxadiazole-3-carboxamide (14)

To a mixture of 14g (30 mg, 0.21 mmol), 7g (70 mg, 0.14 mmol), HATU (78 mg, 0.23 mmol) and DMF (1 mL) was added DIPEA (88 mg, 0.69 mmol), which was then stirred for 1 h. The mixture was purified by prep-HPLC to give the title compound 14 (15.2 mg, 17%).

MS m/z (ESI): 637 [M+1]

¹H NMR (400 MHz, DMSO-d₆) δ 9.86 (s, 1H), 9.05 (d, J=9.0 Hz, 1H), 8.48 (d, J=8.9 Hz, 1H), 8.33 (s, 1H), 7.65 (t, J=8.3 Hz, 1H), 7.02 (dd, J=31.9, 9.2 Hz, 2H), 5.35-5.17 (m, 1H), 4.97 (dd, J=8.8, 6.8 Hz, 1H), 3.86 (s, 2H), 3.68-3.53 (m, 3H), 3.28 (s, 3H), 2.90 (q, J=7.5 Hz, 2H), 2.54 (s, 1H), 1.33 (d, J=6.9 Hz, 3H), 1.23 (d, J=7.5 Hz, 3H), 0.89-0.67 (m, 3H), 0.52-0.12 (in, 12H).

The following intermediate was prepared according to steps 1 to 6 of Example 11, except that a different reagent was used instead of 14a.

| | | MS m/z |
|---|---|---|
| Intermediate | Reagent replacing 14a | (ESI) |
| 4-Isopropyl-1,2,5-oxadiazole- | (E)-4-methylpent-2-enal | 155 |
| 3-carboxylic acid | | [M − 1] |

Compound 26 was prepared according to the procedures of Example 11, except that a different reagent was used instead of 7g.

| | | MS m/z |
|---|---|---|
| Compound | Reagent replacing 7g | (ESI) |
| N-((S)-1,1-dicyclopropyl-3-((4-((1R,2S)-1-(4- | (S)-2-amino-3,3-dicyclopropyl-N- | 651 |
| (cyclopropylmethyl)-4H-1,2,4-triazol-3-yl)-1- | (4-((1R,2S)-1-(4- | [M + 1] |
| ((S)-2-methoxypropanamido)propan-2-yl)-2- | (cyclopropylmethyl)-4H-1,2,4- | |
| fluorophenyl)amino)-3-oxopropan-2-yl)-4- | triazol-3-yl)-1-((S)-2- | |
| ethyl-1,2,5-oxadiazole-3-carboxamide (26) | methoxypropanamido)propan-2- | |
| | yl)-2-fluorophenyl)propanamide | |
| | hydrochloride | |

¹H NMR data of Compound 26 are shown below:

| Compound # | ¹H NMR |
|---|---|
| 26 | ¹H NMR (400 MHz, CD₃OD) δ 8.60 (d, J = 8.7 Hz, 1H), 8.42 (s, 1H), 7.73 |
| | (t, J = 8.1 Hz, 1H), 7.01 (dd, J = 12.7, 10.3 Hz, 2H), 5.25 (d, J = 11.0 Hz, |
| | 1H), 5.01-4.96 (m, 1H), 3.84 (d, J = 6.7 Hz, 1H), 3.72 (dd, J = 14.3, 7.4 |
| | Hz, 1H), 3.62-3.50 (m, 2H), 3.37 (s, 3H), 3.00 (q, J = 7.5 Hz, 2H), 1.46 (d, |
| | J = 6.9 Hz, 3H), 1.32 (t, J = 7.5 Hz, 3H), 1.27 (d, J = 6.8 Hz, 3H), 0.96 (s, |
| | 1H), 0.92-0.74 (m, 3H), 0.61-0.46 (m, 3H), 0.45-0.41 (m, 1H), 0.39-0.35 |
| | (m, 2H), 0.29 (ddd, J = 16.1, 10.9, 6.2 Hz, 4H), 0.22 (d, J = 4.6 Hz, 1H). |

### Example 12. (S)-3,3-dicyclopropyl-2-(2-(cyclopropylmethoxy)acetamido)-N-(4-((1R,2S)-1-(4-(cyclopropylmethyl)-4H-1,2,4-triazol-3-yl)-1-(2-methoxyacetamido)propan-2-yl)-2-fluorophenyl)propanamide (Compound 15)

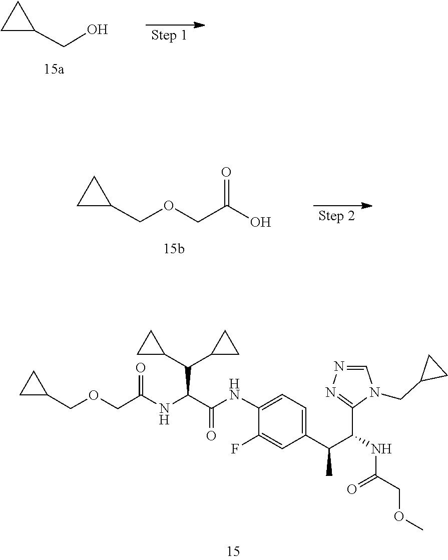 [SMILES: OCC1CC1 ; O=C(O)COCC1CC1 ; COCC(=O)N[C@@H](c1nncn1CC1CC1)[C@@H](C)c1ccc(NC(=O)[C@@H](NC(=O)COCC2CC2)C(C2CC2)C2CC2)c(F)c1]

### Step 1. 2-(Cyclopropylmethoxy)acetic acid (15b)

To a solution of cyclopropylmethanol 15a (3.0 g, 42 mmol) in DMF (20 mL) at 0° C. was added NaH (60% in mineral oil, 1.68 g, 42 mmol), which was then stirred for 30 min. The mixture was then added with bromoacetic acid (1.7 g, 13 mmol) and warmed to room temperature. After stirring for 2 h, the mixture was added with dilute hydrochloric acid (2 N, 10 mL) and the resulting mixture was concentrated to dryness. The residue was purified by prep-HPLC to give the title compound 15b (1.0 g, 59%).

MS m/z (ESI): 129 [M−1]

### Step 2. (S)-3,3-dicyclopropyl-2-(2-(cyclopropylmethoxy)acetamido)-N-(4-((1R,2S)-1-(4-(cyclopropylmethyl)-4H-1,2,4-triazol-3-yl)-1-(2-methoxyacetamido)propan-2-yl)-2-fluorophenyl)propanamide (15)

To a mixture of 15b (27 mg, 0.21 mmol), 7g (70 mg, 0.14 mmol), HATU (88 mg, 0.23 mmol) and DMF (1 mL) was added DIPEA (88 mg, 0.69 mmol), which was then stirred for 1 h. The reaction mixture was purified by prep-HPLC to give the title compound 15 (14.8 mg, 17%).

MS m/z (ESI): 625 [M+1]

¹H NMR (400 MHz, DMSO-d₆) δ 9.78 (s, 1H), 8.47 (d, J=9.0 Hz, 1H), 8.30 (s, 1H), 7.57 (s, 2H), 7.05 (d, J=12.1 Hz, 1H), 6.97 (d, J=8.3 Hz, 1H), 5.26-5.15 (m, 1H), 4.80 (dd, J=9.1, 6.0 Hz, 1H), 3.93 (d, J=3.8 Hz, 2H), 3.86 (s, 2H), 3.60 (ddd, J=33.7, 14.4, 7.5 Hz, 3H), 3.35-3.32 (m, 2H), 3.28 (s, 3H), 1.32 (d, J=6.9 Hz, 3H), 1.04 (t, J=7.5 Hz, 1H), 0.93 (s, 1H), 0.70 (d, J=4.8 Hz, 2H), 0.62 (dd, J=16.6, 7.5 Hz, 1H), 0.51-0.45 (m, 2H), 0.44-0.30 (m, 5H), 0.24 (s, 3H), 0.20 (s, 6H).

The following intermediates were prepared according to Step 1 of Example 12, except that different reagents were used instead of cyclopropylmethanol.

| | Reagent replacing | MS m/z |
|---|---|---|
| Intermediate | cyclopropylmethanol | (ESI) |
| 2-((1-(Tert-butoxycarbonyl)azetidin-3- | Tert-butyl 3-hydroxyazetidine- | 232 |
| yl)oxy)acetic acid | 1-carboxylate | [M + 1] |
| 2-(Methoxy-d₃)acetic acid | CD₃OD | 92 |
| | | [M − 1] |
| 2-Cyclopropoxyacetic acid | Cyclopropanol | 115 |
| | | [M − 1] |

### Example 13. N—((S)-1,1-dicyclopropyl-3-((2-fluoro-4-((1R,2S)-1-(2-methoxyacetamido)-1-(5-methyl-1,3,4-thiadiazol-2-yl)propan-2-yl)phenyl)amino)-3-oxopropan-2-yl)-1-ethyl-1H-pyrazole-5-carboxamide (Compound 17)

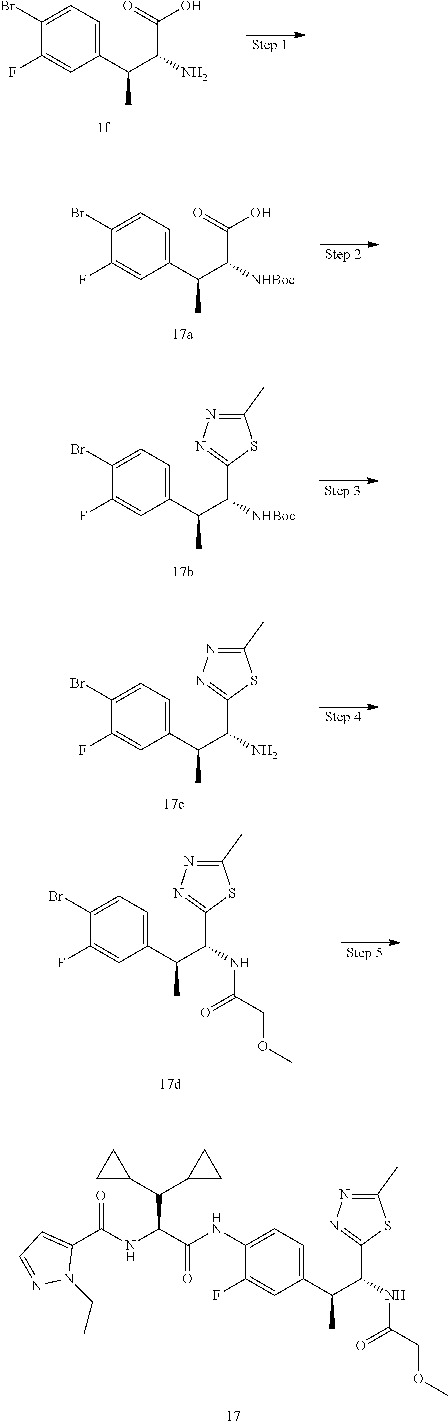 [SMILES: C[C@@H](c1ccc(Br)c(F)c1)[C@@H](N)C(=O)O ; C[C@@H](c1ccc(Br)c(F)c1)[C@@H](NC(=O)OC(C)(C)C)C(=O)O ; Cc1nnc([C@H](NC(=O)OC(C)(C)C)[C@@H](C)c2ccc(Br)c(F)c2)s1 ; Cc1nnc([C@H](N)[C@@H](C)c2ccc(Br)c(F)c2)s1 ; COCC(=O)N[C@@H](c1nnc(C)s1)[C@@H](C)c1ccc(Br)c(F)c1 ; CCn1nccc1C(=O)N[C@H](C(=O)Nc1ccc([C@H](C)[C@@H](NC(=O)COC)c2nnc(C)s2)cc1F)C(C1CC1)C1CC1]

### Step 1. (2R,3S)-3-(4-bromo-3-fluorophenyl)-2-((tert-butoxycarbonyl)amino)butanoic acid (17a)

To a mixture of 1f (1.30 g, 4.7 mmol), Boc₂O (1.13 g, 5.2 mmol), THF (20 mL) and water (20 mL) was added NaOH (414 mg, 10.4 mmol), which was then stirred overnight. The reaction mixture was added with dilute hydrochloric acid (2 N, 20 mL) and extracted with EtOAc (3×30 mL). The combined organic phase was dried over anhydrous Na₂SO₄ and filtered. The filtrate was concentrated to dryness to give the title compound 17a (950 mg, 54%). The crude product was directly used in the next step without further purification.

MS m/z (ESI): 376, 378 [M+1]

### Step 2. Tert-butyl ((1R,2S)-2-(4-bromo-3-fluorophenyl)-1-(5-methyl-1,3,4-thiadiazol-2-yl)propyl)carbamate (17b)

To a mixture of 17a (100 mg, 0.27 mmol), acetohydrazide (20 mg, 0.27 mmol), a solution of propylphosphonic anhydride in EtOAc (50%, 152 mg, 0.48 mmol), Lawesson's reagent (193 mg, 0.48 mmol) and EtOAc (3 mL) was added DIPEA (86 mg, 0.66 mmol), which was then heated to 80° C. and stirred for 16 h. After cooling to room temperature, the reaction mixture was added with EtOAc (50 mL) and washed with a saturated aqueous NaHCO₃ solution (50 mL). The organic phase was concentrated to dryness and the residue was purified by silica gel column chromatography (dichloromethane/methanol=20/1) to give the title compound 17b (42 mg, 37%).

MS m/z (ESI): 430, 432 [M+1]

### Step 3. (1R,2S)-2-(4-bromo-3-fluorophenyl)-1-(5-methyl-1,3,4-thiadiazol-2-yl)propan-1-amine hydrochloride (17c)

A mixture of 17b (42 mg, 0.1 mmol) and a solution of hydrochloric acid in 1,4-dioxane (4 M, 3 mL) was stirred for 2 h and then concentrated to dryness to give the title compound 17c (32 mg, 99%). The crude product was directly used in the next step without further purification.

MS m/z (ESI): 330, 332 [M+1]

### Step 4. N-((1R,2S)-2-(4-bromo-3-fluorophenyl)-1-(5-methyl-1,3,4-thiadiazol-2-yl)propyl)-2-methoxyacetamide (17d)

To a mixture of 17c (32 mg, 0.10 mmol), 2-methoxyacetyl chloride (13 mg, 0.12 mmol) and THF (3 mL) was added TEA (29 mg, 0.30 mmol), which was then stirred for 2 h. The reaction mixture was added with EtOAc (10 mL) and washed with water (10 mL). The organic phase was concentrated to dryness and the residue was purified by silica gel column chromatography (dichloromethane/methanol=20/1) to give the title compound 17d (15 mg, 39%).

MS m/z (ESI): 385, 387 [M+1]

### Step 5. N—((S)-1,1-dicyclopropyl-3-((2-fluoro-4-((1R,2S)-1-(2-methoxyacetamido)-1-(5-methyl-1,3,4-thiadiazol-2-yl)propan-2-yl)phenyl)amino)-3-oxopropan-2-yl)-1-ethyl-1H-pyrazole-5-carboxamide (17)

A mixture of 17d (15 mg, 0.037 mmol), is (16 mg, 0.06 mmol), Cs₂CO₃ (36 mg, 0.11 mmol), Pd₂(dba)₃ (7 mg, 0.007 mmol), XantPhos (9 mg, 0.015 mmol) and 1,4-dioxane (500 uL) was heated to 100° C. and stirred for 2 h. After cooling to room temperature, the reaction mixture was purified by silica gel column chromatography (dichloromethane/methanol=8/1) and prep-HPLC in sequence to give the title compound 17 (4.0 mg, 17%).

MS m/z (ESI): 612 [M+1]

¹H NMR (400 MHz, CD₃OD) δ 7.79 (t, J=8.2 Hz, 1H), 7.50 (d, J=2.0 Hz, 1H), 7.07 (d, J=11.9 Hz, 1H), 7.00 (d, J=8.4 Hz, 1H), 6.84 (d, J=2.1 Hz, 1H), 5.58 (d, J=9.9 Hz, 1H), 4.94 (d, J=7.0 Hz, 1H), 4.54 (dd, J=14.3, 7.1 Hz, 2H), 3.93 (t, J=10.4 Hz, 3H), 3.56-3.46 (m, 2H), 3.42 (s, 3H), 2.63 (s, 3H), 1.38 (s, 1H), 0.90-0.84 (m, 4H), 0.55-0.37 (m, 7H), 0.35-0.20 (m, 3H).

The following intermediates were prepared according to Step 1 of Example 13, except that different reagents were used instead of 1f.

| | | MS m/z |
|---|---|---|
| Intermediate | Reagent replacing 1f | (ESI) |
| (2R,3S)-3-(6-bromo-5-fluoropyridin-3-yl)-2- | (2R,3S)-2-amino-3-(6-bromo-5- | 376, 378 |
| ((tert-butoxycarbonyl)amino)butanoic acid | fluoropyridin-3-yl)butanoic acid | [M + 1] |
| (2R,3S)-3-(6-bromopyridin-3-yl)-2-((tert- | (2R,3S)-2-amino-3-(6- | 359, 361 |
| butoxycarbonyl)amino)butanoic acid | bromopyridin-3-yl)butanoic acid | [M + 1] |
| (2R,3S)-3-(4-bromo-3-methoxyphenyl)-2- | (2R,3S)-2-amino-3-(4-bromo-3- | 388, 390 |
| ((tert-butoxycarbonyl)amino)butanoic acid | methoxyphenyl)butanoic acid | [M + 1] |

### Example 14. N—((S)-1,1-dicyclopropyl-3-((4-((1R,2S)-1-(1-(cyclopropylmethyl)-1H-1,2,4-triazol-5-yl)-1-(2-methoxyacetamido)propan-2-yl)-2-fluorophenyl)amino)-3-oxopropan-2-yl)-1-ethyl-1H-pyrazole-5-carboxamide (Compound 19)

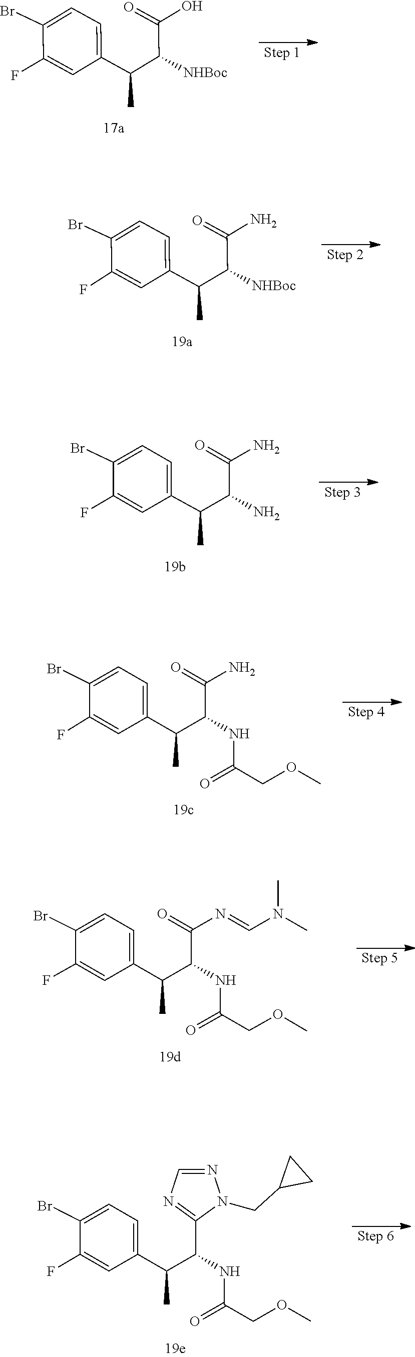 [SMILES: C[C@@H](c1ccc(Br)c(F)c1)[C@@H](NC(=O)OC(C)(C)C)C(=O)O ; C[C@@H](c1ccc(Br)c(F)c1)[C@@H](NC(=O)OC(C)(C)C)C(N)=O ; C[C@@H](c1ccc(Br)c(F)c1)[C@@H](N)C(N)=O ; COCC(=O)N[C@@H](C(N)=O)[C@@H](C)c1ccc(Br)c(F)c1 ; COCC(=O)N[C@@H](C(=O)/N=C/N(C)C)[C@@H](C)c1ccc(Br)c(F)c1 ; COCC(=O)N[C@@H](c1ncnn1CC1CC1)[C@@H](C)c1ccc(Br)c(F)c1]

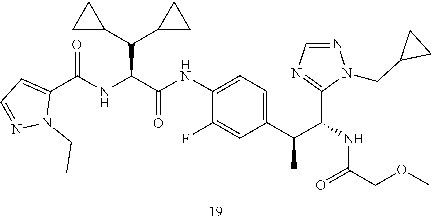 [SMILES: CCn1nccc1C(=O)N[C@H](C(=O)Nc1ccc([C@H](C)[C@@H](NC(=O)COC)c2ncnn2CC2CC2)cc1F)C(C1CC1)C1CC1]

### Step 1. Tert-butyl ((2R,3S)-1-amino-3-(4-bromo-3-fluorophenyl)-1-oxobutan-2-yl)carbamate (19a)

To a mixture of 17a (37.62 g, 100 mmol), NH₄Cl (26.75 g, 500 mmol), DMTMM·BF₄ (39.37 g, 120 mmol) and acetonitrile (500 mL) was added TEA (69 mL, 500 mmol), which was then stirred for 1 h. The reaction mixture was concentrated to dryness. The residue was added EtOAc (200 mL) and washed with dilute hydrochloric acid (2 N, 200 mL) and saturated brine (200 mL). The organic phase was dried over anhydrous Na₂SO₄ and filtered. The filtrate was concentrated to dryness to give the title compound 19a (37.52 g, 99%). The crude product was directly used in the next step without further purification.

MS m/z (ESI): 375, 377 [M+1]

### Step 2. (2R,3S)-2-amino-3-(4-bromo-3-fluorophenyl)butanamide hydrochloride (19b)

A solution of 19a (37.52 g, 100 mmol) in hydrochloric acid in 1,4-dioxane (4M, 200 mL) and methanol (200 mL) was stirred for 1 h. The reaction mixture was concentrated to dryness to give the title compound 19b (27.51 g, crude). The crude product was directly used in the next step without further purification.

MS m/z (ESI): 275, 277 [M+1]

### Step 3. (2R,3S)-3-(4-bromo-3-fluorophenyl)-2-(2-methoxyacetamido)butanamide (19c)

To a mixture of 19b (27.51 g, 100 mmol), 2-methoxyacetic acid (9.2 mL, 120 mmol), DMTMM·BF₄ (39.37 g, 120 mmol) and acetonitrile (500 mL) was added TEA (69 mL, 500 mmol), which was then stirred for 1 h. The reaction mixture was concentrated to dryness. The residue was added EtOAc (200 mL), washed with dilute hydrochloric acid (2 N, 200 mL) and saturated brine (200 mL). The organic phase was dried over anhydrous Na₂SO₄ and filtered. The filtrate was concentrated to dryness to give the title compound 19c (27.35 g, 79%). The crude product was directly used in the next step without further purification.

MS m/z (ESI): 347, 349 [M+1]

### Step 4. (2R,3S)-3-(4-bromo-3-fluorophenyl)-N-((E)-(dimethylamino)methylene)-2-(2-methoxyacetamido)butanamide (19d)

To a solution of 19c (530 mg, 1.5 mmol) in acetonitrile (20 mL) was added DMF-DMA (273 mg, 2.3 mmol), which was then heated to 70° C. and stirred for 1.5 h. After cooling to room temperature, the reaction mixture was concentrated to dryness to give the title compound 19d (600 mg, crude). The crude product was directly used in the next step without further purification.

MS m/z (ESI): 402, 404 [M+1]

### Step 5. N-((1R,2S)-2-(4-bromo-3-fluorophenyl)-1-(1-(cyclopropylmethyl)-1H-1,2,4-triazol-5-yl)propyl)-2-methoxyacetamide (19e)

To a solution of 19d (600 mg, 1.5 mmol) in acetic acid (20 mL) was added (cyclopropylmethyl)hydrazine dihydrochloride (1.21 g, 7.7 mmol), which was then heated to 110° C. and stirred for 8 h. After cooling to room temperature, the reaction mixture was concentrated to dryness and the residue was purified by prep-HPLC to give the title compound 19e (130 mg, 20%).

MS m/z (ESI): 425, 427 [M+1]

### Step 6. N—((S)-1,1-dicyclopropyl-3-((4-((1R,2S)-1-(1-(cyclopropylmethyl)-1H-1,2,4-triazol-5-yl)-1-(2-methoxyacetamido)propan-2-yl)-2-fluorophenyl)amino)-3-oxopropan-2-yl)-1-ethyl-1H-pyrazole-5-carboxamide (19)

A mixture of 19e (70 mg, 0.16 mmol), is (72 mg, 0.25 mmol), Cs₂CO₃ (108 mg, 0.33 mmol), Pd₂(dba)₃ (30 mg, 0.033 mmol), XantPhos (38 mg, 0.066 mmol) and 1,4-dioxane (3 mL) was heated to 105° C. under microwave and stirred for 2.5 h. After cooling to room temperature, the reaction mixture was concentrated to dryness and the residue was purified by prep-HPLC to give the title compound 19 (11.5 mg, 11%).

MS m/z (ESI): 635 [M+1]

¹H NMR (400 MHz, CD₃OD) δ 7.83 (s, 1H), 7.75 (t, J=8.3 Hz, 1H), 7.49 (d, J=2.1 Hz, 1H), 7.03-6.93 (m, 2H), 6.83 (d, J=2.1 Hz, 1H), 5.35 (d, J=11.0 Hz, 1H), 4.92 (d, J=7.1 Hz, 1H), 4.53 (q, J=7.2 Hz, 2H), 3.95 (q, J=15.2 Hz, 2H), 3.73 (ddd, J=39.5, 14.4, 7.2 Hz, 2H), 3.50-3.38 (m, 4H), 1.43 (d, J=7.0 Hz, 3H), 1.38 (t, J=7.2 Hz, 3H), 0.98-0.73 (m, 4H), 0.57-0.17 (m, 12H).

The following intermediate was prepared according to steps 1 to 3 of Example 14, except that a different reagent was used instead of 2-methoxyacetic acid.

| | Reagent replacing 2- | MS m/z |
|---|---|---|
| Intermediate | methoxyacetic acid | (ESI) |
| (2R,3S)-3-(4-bromo-3-fluorophenyl)-2- | Propionic acid | 331, 333 |
| propionamidobutanamide | | [M + 1] |

The following intermediate was prepared according to steps 1 to 4 of Example 14, except that a different reagent was used instead of 2-methoxyacetic acid.

| | Reagent replacing 2- | MS m/z |
|---|---|---|
| Intermediate | methoxyacetic acid | (ESI) |
| (2R,3S)-3-(4-bromo-3-fluorophenyl)-N-((E)- | Propionic acid | 386, 388 |
| (dimethylamino)methylene)-2-propionamidobutanamide | | [M + 1] |

Compounds 31 and 36 were prepared according to the procedures of Example 14, except that different reagents were used instead of (cyclopropylmethyl)hydrazine dihydrochloride.

| | Reagent replacing | MS |
|---|---|---|
| | (cyclopropylmethyl)hydrazine | m/z |
| Compound | dihydrochloride | (ESI) |
| N-((S)-1,1-dicyclopropyl-3-((2-fluoro-4-((1R,2S)- | Propylhydrazine | 623 |
| 1-(2-methoxyacetamido)-1-(1-propyl-1H-1,2,4- | dihydrochloride | [M + 1] |
| triazol-5-yl)propan-2-yl)phenyl)amino)-3- | | |
| oxopropan-2-yl)-1-ethyl-1H-pyrazole-5- | | |
| carboxamide (31) | | |
| N-((S)-1,1-dicyclopropyl-3-((2-fluoro-4-((1R,2S)- | Isobutylhydrazine | 637 |
| 1-(1-isobutyl-1H-1,2,4-triazol-5-yl)-1-(2- | dihydrochloride | [M + 1] |
| methoxyacetamido)propan-2-yl)phenyl)amino)-3- | | |
| oxopropan-2-yl)-1-ethyl-1H-pyrazole-5- | | |
| carboxamide (36) | | |

Compounds 33, 34, 35, 39, 47 and 53 were prepared according to the procedures of Example 14, except that different reagents were used instead of Is.

| | | MS m/z |
|---|---|---|
| Compound | Reagent replacing 1s | (ESI) |
| N-((S)-1,1-dicyclopropyl-3-((4-((1R,2S)-1-(1- | (S)-N-(1-amino-3,3- | 649 |
| (cyclopropylmethyl)-1H-1,2,4-triazol-5-yl)-1-(2- | dicyclopropyl-1-oxopropan- | [M + 1] |
| methoxyacetamido)propan-2-yl)-2- | 2-yl)-1-isopropyl-1H- | |
| fluorophenyl)amino)-3-oxopropan-2-yl)-1- | pyrazole-5-carboxamide (4e) | |
| isopropyl-1H-pyrazole-5-carboxamide (33) | | |
| N-((S)-1,1-dicyclopropyl-3-((4-((1R,2S)-1-(1- | (S)-N-(1-amino-3,3- | 637 |
| (cyclopropylmethyl)-1H-1,2,4-triazol-5-yl)-1-(2- | dicyclopropyl-1-oxopropan- | [M + 1] |
| methoxyacetamido)propan-2-yl)-2- | 2-yl)-4-ethyl-1,2,5- | |
| fluorophenyl)amino)-3-oxopropan-2-yl)-4-ethyl- | oxadiazole-3-carboxamide | |
| 1,2,5-oxadiazole-3-carboxamide (34) | | |
| N-((S)-1,1-dicyclopropyl-3-((4-((1R,2S)-1-(1- | (S)-N-(1-amino-3,3- | 624 |
| (cyclopropylmethyl)-1H-1,2,4-triazol-5-yl)-1-(2- | dicyclopropyl-1-oxopropan- | [M + 1] |
| methoxyacetamido)propan-2-yl)-2- | 2-yl)-1-(methyl-d₃)-1H- | |
| fluorophenyl)amino)-3-oxopropan-2-yl)-1- | pyrazole-5-carboxamide | |
| (methyl-d₃)-1H-pyrazole-5-carboxamide (35) | | |
| N-((S)-1,1-dicyclopropyl-3-((4-((1R,2S)-1-(1- | (S)-N-(1-amino-3,3- | 623 |
| (cyclopropylmethyl)-1H-1,2,4-triazol-5-yl)-1-(2- | dicyclopropyl-1-oxopropan- | [M + 1] |
| methoxyacetamido)propan-2-yl)-2- | 2-yl)-4-methyl-1,2,5- | |
| fluorophenyl)amino)-3-oxopropan-2-yl)-4-methyl- | oxadiazole-3-carboxamide | |
| 1,2,5-oxadiazole-3-carboxamide (39) | | |
| N-((S)-1,1-dicyclopropyl-3-((4-((1R,2S)-1-(1- | (S)-N-(1-amino-3,3- | 651 |
| (cyclopropylmethyl)-1H-1,2,4-triazol-5-yl)-1-(2- | dicyclopropyl-1-oxopropan- | [M + 1] |
| methoxyacetamido)propan-2-yl)-2- | 2-yl)-4-isopropyl-1,2,5- | |
| fluorophenyl)amino)-3-oxopropan-2-yl)-4- | oxadiazole-3-carboxamide | |
| isopropyl-1,2,5-oxadiazole-3-carboxamide (47) | | |
| N-((S)-1,1-dicyclopropyl-3-((4-((1R,2S)-1-(1- | (S)-N-(1-amino-3,3- | 621 |
| (cyclopropylmethyl)-1H-1,2,4-triazol-5-yl)-1-(2- | dicyclopropyl-1-oxopropan- | [M + 1] |
| methoxyacetamido)propan-2-yl)-2- | 2-yl)-1-methyl-1H-pyrazole- | |
| fluorophenyl)amino)-3-oxopropan-2-yl)-1-methyl- | 5-carboxamide | |
| 1H-pyrazole-5-carboxamide (53) | | |

Compounds 45, 46 and 48 were prepared according to the procedures for Example 14, except that different reagents were used instead of 18a.

| | | MS m/z |
|---|---|---|
| Compound | Reagent replacing 18a | (ESI) |
| N-((S)-1,1-dicyclopropyl-3-((4-((1R,2S)-1-(1- | (2R,3S)-3-(4-bromo-3- | 647 |
| (cyclopropylmethyl)-1H-1,2,4-triazol-5-yl)-1-(2- | methoxyphenyl)-2-((tert- | [M + 1] |
| methoxyacetamido)propan-2-yl)-2- | butoxycarbonyl)amino)butanoic | |
| methoxyphenyl)amino)-3-oxopropan-2-yl)-1- | acid | |
| ethyl-1H-pyrazole-5-carboxamide (45) | | |
| N-((S)-1,1-dicyclopropyl-3-((5-((1R,2S)-1-(1- | (2R,3S)-3-(6-bromo-5- | 636 |
| (cyclopropylmethyl)-1H-1,2,4-triazol-5-yl)-1-(2- | fluoropyridin-3-yl)-2-((tert- | [M + 1] |
| methoxyacetamido)propan-2-yl)-3-fluoropyridin- | butoxycarbonyl)amino)butanoic | |
| 2-yl)amino)-3-oxopropan-2-yl)-1-ethyl-1H- | acid | |
| pyrazole-5-carboxamide (46) | | |
| N-((S)-1,1-dicyclopropyl-3-((5-((1R,2S)-1-(1- | (2R,3S)-3-(6-bromopyridin-3- | 618 |
| (cyclopropylmethyl)-1H-1,2,4-triazol-5-yl)-1-(2- | yl)-2-((tert- | [M + 1] |
| methoxyacetamido)propan-2-yl)pyridin-2- | butoxycarbonyl)amino)butanoic | |
| yl)amino)-3-oxopropan-2-yl)-1-ethyl-1H- | acid | |
| pyrazole-5-carboxamide (48) | | |

Compounds 49, 68 and 72 were prepared according to the procedures for Example 14, except that different reagents were used instead of 2-methoxyacetic acid.

| | Reagent replacing 2- | MS m/z |
|---|---|---|
| Compound | methoxyacetic acid | (ESI) |
| N-((S)-1,1-dicyclopropyl-3-((4-((1R,2S)-1-(1- | 2-Acetoxyacetic acid | 621 |
| (cyclopropylmethyl)-1H-1,2,4-triazol-5-yl)-1-(2- | | [M + 1] |
| hydroxyacetamido)propan-2-yl)-2- | | |
| fluorophenyl)amino)-3-oxopropan-2-yl)-1-ethyl- | | |
| 1H-pyrazole-5-carboxamide (49) | | |
| N-((S)-1,1-dicyclopropyl-3-((4-((1R,2S)-1-(1- | 2-(Methoxy-d₃)acetic acid | 638 |
| (cyclopropylmethyl)-1H-1,2,4-triazol-5-yl)-1-(2- | | [M + 1] |
| (methoxy-d₃)acetamido)propan-2-yl)-2- | | |
| fluorophenyl)amino)-3-oxopropan-2-yl)-1-ethyl- | | |
| 1H-pyrazole-5-carboxamide (68) | | |
| N-((S)-1,1-dicyclopropyl-3-((4-((1R,2S)-1-(1- | Propionic acid | 619 |
| (cyclopropylmethyl)-1H-1,2,4-triazol-5-yl)-1- | | [M + 1] |
| propionamidopropan-2-yl)-2- | | |
| fluorophenyl)amino)-3-oxopropan-2-yl)-1-ethyl- | | |
| 1H-pyrazole-5-carboxamide (72) | | |

¹H NMR data of compounds 31, 33, 34, 35, 36, 39, 45, 46, 47, 48, 49, 53, 68 and 72 are shown below:

| Compound # | ¹H NMR |
|---|---|
| 31 | ¹H NMR (400 MHz, DMSO-d₆) δ 9.80 (s, 1H), 8.49-8.30 (m, 2H), 7.79 (s, |
| | 1H), 7.67 (dd, J = 19.2, 8.4 Hz, 1H), 7.47 (d, J = 1.9 Hz, 1H), 7.11-6.99 |
| | (m, 1H), 6.97 (d, J = 1.7 Hz, 1H), 6.95-6.89 (m, 1H), 5.24 (t, J = 9.8 Hz, |
| | 1H), 4.91 (t, J = 8.2 Hz, 1H), 4.53-4.40 (m, 2H), 3.86 (s, 2H), 3.82 (d, J = |
| | 7.9 Hz, 1H), 3.45 (dd, J = 10.4, 7.1 Hz, 1H), 3.28 (s, 3H), 1.57-1.46 (m, |
| | 1H), 1.33 (d, J = 6.9 Hz, 3H), 1.27 (dd, J = 15.4, 8.2 Hz, 5H), 0.95-0.84 |
| | (m, 1H), 0.82 (d, J = 15.3 Hz, 1H), 0.76-0.69 (m, 1H), 0.66 (d, J = 2.4 Hz, |
| | 3H), 0.50-0.40 (m, 1H), 0.35 (dd, J = 8.6, 5.1 Hz, 1H), 0.31-0.23 (m, |
| | 2H), 0.24 (s, 3H), 0.11 (s, 1H). |
| 33 | ¹H NMR (400 MHz, DMSO-d₆) δ 9.79 (s, 1H), 8.42 (d, J = 8.8 Hz, 1H), |
| | 8.36 (d, J = 8.8 Hz, 1H), 7.79 (s, 1H), 7.66 (s, 1H), 7.49 (d, J = 1.7 Hz, 1H), |
| | 7.04 (d, J = 12.4 Hz, 1H), 6.94 (s, 1H), 6.89 (d, J = 1.9 Hz, 1H), 5.38 (s, |
| | 1H), 5.25 (s, 1H), 4.89 (s, 1H), 3.86 (s, 2H), 3.74 (d, J = 7.0 Hz, 2H), 3.45 |
| | (s, 1H), 3.29 (d, J = 3.6 Hz, 3H), 1.37 (d, J = 6.6 Hz, 3H), 1.36-1.30 (m, |
| | 6H), 0.98-0.85 (m, 2H), 0.82 (d, J = 14.8 Hz, 1H), 0.71 (dd, J = 17.2, 9.0 |
| | Hz, 1H), 0.43 (d, J = 5.4 Hz, 1H), 0.36 (d, J = 8.2 Hz, 2H), 0.31-0.26 (m, |
| | 2H), 0.24 (dd, J = 9.3, 5.5 Hz, 3H), 0.20 (d, J = 5.7 Hz, 2H), 0.14-0.06 (m, |
| | 2H). |
| 34 | ¹H NMR (400 MHz, DMSO-d₆) δ 9.61 (s, 1H), 8.37-8.07 (m, 2H), 7.62 |
| | (d, J = 3.8 Hz, 1H), 7.56-7.40 (m, 1H), 7.36-7.22 (m, 1H), 6.93-6.67 |
| | (m, 1H), 5.05 (t, J = 7.7 Hz, 1H), 4.71 (d, J = 4.6 Hz, 1H), 4.35-4.09 (m, |
| | 2H), 3.67 (d, J = 3.6 Hz, 2H), 3.56-3.38 (m, 2H), 3.28 (s, 1H), 3.10-3.03 |
| | (m, 3H), 1.66 (d, J = 6.3 Hz, 1H), 1.18-1.05 (m, 6H), 0.70 (s, 1H), 0.61 (s, |
| | 1H), 0.53 (d, J = 5.5 Hz, 1H), 0.50-0.39 (m, 4H), 0.25 (s, 1H), 0.15 (d, J = |
| | 8.4 Hz, 1H), 0.09 (s, 2H), −0.00 (s, 3H), −0.08 (s, 1H). |
| 35 | ¹H NMR (400 MHz, DMSO-d₆) δ 9.82 (s, 1H), 8.42 (d, J = 8.4 Hz, 1H), |
| | 8.36 (d, J = 7.8 Hz, 1H), 7.80 (d, J = 4.5 Hz, 1H), 7.65 (t, J = 8.3 Hz, 1H), |
| | 7.46 (d, J = 1.9 Hz, 1H), 7.04 (d, J = 12.3 Hz, 2H), 6.95 (d, J = 8.1 Hz, 1H), |
| | 5.26 (s, 1H), 4.91 (t, J = 8.2 Hz, 1H), 3.86 (s, 2H), 3.74 (d, J = 7.0 Hz, 2H), |
| | 3.45 (s, 1H), 3.29 (s, 3H), 1.33 (d, J = 6.9 Hz, 3H), 0.94 (s, 1H), 0.90-0.83 |
| | (m, 1H), 0.79 (s, 1H), 0.71 (d, J = 9.0 Hz, 1H), 0.44 (d, J = 7.8 Hz, 1H), |
| | 0.39-0.30 (m, 3H), 0.29 (d, J = 4.5 Hz, 1H), 0.24 (d, J = 4.4 Hz, 3H), |
| | 0.23-0.14 (m, 3H), 0.11 (s, 1H). |
| 36 | ¹H NMR (400 MHz, DMSO-d₆) δ 9.61 (s, 1H), 8.37-8.07 (m, 2H), 7.62 |
| | (d, J = 3.8 Hz, 1H), 7.56-7.40 (m, 1H), 7.36-7.22 (m, 1H), 6.93-6.67 |
| | (m, 3H), 5.05 (t, J = 7.7 Hz, 1H), 4.71 (d, J = 4.6 Hz, 1H), 4.35-4.09 (m, |
| | 2H), 3.67 (d, J = 3.6 Hz, 2H), 3.56-3.38 (m, 2H), 3.28 (s, 1H), 3.10-3.03 |
| | (m, 3H), 1.66 (d, J = 6.3 Hz, 1H), 1.18-1.05 (m, 6H), 0.70 (s, 1H), 0.61 (s, |
| | 1H), 0.53 (d, J = 5.5 Hz, 1H), 0.50-0.39 (m, 6H), 0.25 (s, 1H), 0.15 (d, J = |
| | 8.4 Hz, 1H), 0.09 (s, 2H), −0.00 (s, 3H), −0.08 (s, 1H). |
| 39 | ¹H NMR (400 MHz, DMSO-d₆) δ 9.62 (s, 1H), 8.74 (dd, J = 8.9, 4.0 Hz, |
| | 1H), 8.19 (d, J = 8.7 Hz, 1H), 7.55 (s, 1H), 7.40 (dd, J = 14.6, 6.3 Hz, 1H), |
| | 6.81 (dd, J = 12.1, 1.6 Hz, 1H), 6.72 (d, J = 8.3 Hz, 1H), 5.07-4.97 (m, |
| | 1H), 4.73 (dd, J = 8.9, 6.7 Hz, 1H), 3.62 (s, 2H), 3.50 (d, J = 7.1 Hz, 2H), |
| | 3.21 (dd, J = 10.4, 6.9 Hz, 1H), 3.05 (s, 3H), 2.23 (s, 3H), 1.09 (d, J = 6.9 |
| | Hz, 3H), 0.75-0.44 (m, 4H), 0.30-−0.17 (m, 12H). |
| 45 | ¹H NMR (400 MHz, CD₃OD) δ 7.93-7.80 (m, 2H), 7.51 (t, J = 2.9 Hz, |
| | 1H), 6.86 (dd, J = 7.1, 2.1 Hz, 1H), 6.81-6.75 (m, 1H), 6.65 (dd, J = 11.2, |
| | 1.6 Hz, 1H), 5.30 (d, J = 11.1 Hz, 1H), 4.88 (d, J = 6.2 Hz, 1H), 4.53 (q, J = |
| | 7.1 Hz, 2H), 4.02-3.88 (m, 2H), 3.74 (d, J = 4.3 Hz, 3H), 3.70-3.63 (m, |
| | 1H), 3.56 (dd, J = 14.5, 7.7 Hz, 1H), 3.48-3.35 (m, 4H), 1.46 (d, J = 7.0 |
| | Hz, 3H), 1.38 (t, J = 7.2 Hz, 3H), 0.84 (ddd, J = 21.6, 16.8, 13.6 Hz, 4H), |
| | 0.55-0.18 (m, 12H). |
| 46 | ¹H NMR (400 MHz, CD₃OD) δ 8.00 (d, J = 1.7 Hz, 1H), 7.83 (s, 1H), 7.64 |
| | (dd, J = 10.9, 1.8 Hz, 1H), 7.49 (d, J = 2.1 Hz, 1H), 6.84 (d, J = 2.1 Hz, 1H), |
| | 5.44 (d, J = 10.8 Hz, 1H), 4.98 (s, 1H), 4.53 (q, J = 7.2 Hz, 2H), 4.01-3.87 |
| | (m, 3H), 3.80 (dd, J = 14.4, 7.4 Hz, 1H), 3.63 (dd, J = 10.6, 7.1 Hz, 1H), |
| | 3.42 (d, J = 3.7 Hz, 3H), 1.47 (d, J = 7.0 Hz, 3H), 1.41-1.35 (m, 3H), |
| | 1.00-0.78 (m, 4H), 0.58-0.21 (m, 12H). |
| 47 | ¹H NMR (400 MHz, DMSO-d₆) δ 9.85 (s, 1H), 9.14 (d, J = 8.9 Hz, 1H), |
| | 8.43 (d, J = 8.7 Hz, 1H), 7.79 (s, 1H), 7.64 (t, J = 8.3 Hz, 1H), 7.00 (dd, J = |
| | 31.3, 9.5 Hz, 2H), 5.25 (m, 1H), 4.97 (dd, J = 8.9, 6.7 Hz, 1H), 3.86 (s, 2H), |
| | 3.74 (d, J = 6.7 Hz, 2H), 3.45 (dd, J = 10.1, 7.2 Hz, 1H), 3.36 (d, J = 6.9 Hz, |
| | 2H), 3.29 (s, 3H), 1.30 (m, 9H), 0.76 (ddd, J = 25.3, 22.6, 8.2 Hz, 3H), 0.30 |
| | (m, 12H). |
| 48 | ¹H NMR (400 MHz, CD₃OD) δ 8.07 (d, J = 7.6 Hz, 1H), 7.86 (dd, J = 11.5, |
| | 4.4 Hz, 3H), 7.50 (d, J = 2.1 Hz, 1H), 6.86 (d, J = 2.1 Hz, 1H), 5.42 (d, J = |
| | 10.7 Hz, 1H), 4.90 (m, 2H), 4.52 (q, J = 7.2 Hz, 2H), 3.96 (q, J = 15.2 Hz, |
| | 2H), 3.88-3.70 (m, 2H), 3.67-3.52 (m, 1H), 3.42 (s, 3H), 1.47 (d, J = 7.0 |
| | Hz, 3H), 1.37 (t, J = 7.2 Hz, 3H), 0.88 (ddd, J = 41.7, 19.5, 7.7 Hz, 4H), |
| | 0.64-0.11 (m, 11H). |
| 49 | ¹H NMR (400 MHz, DMSO-d₆) δ 9.80 (s, 1H), 8.36 (dd, J = 8.9, 2.0 Hz, |
| | 1H), 8.15 (d, J = 8.9 Hz, 1H), 7.79 (s, 1H), 7.64 (dd, J = 17.6, 8.5 Hz, 1H), |
| | 7.47 (d, J = 2.0 Hz, 1H), 7.10-7.01 (m, 1H), 6.97 (d, J = 2.0 Hz, 1H), 6.94 |
| | (d, J = 6.8 Hz, 1H), 5.47 (t, J = 5.8 Hz, 1H), 5.32-5.18 (m, 1H), 4.90 (t, J = |
| | 7.5 Hz, 1H), 4.54-4.34 (m, 2H), 3.86 (qd, J = 16.0, 5.7 Hz, 2H), 3.80-3.67 |
| | (m, 2H), 3.51-3.37 (m, 1H), 1.32 (d, J = 6.9 Hz, 3H), 1.28 (t, J = 7.1 |
| | Hz, 4H), 1.00-0.65 (m, 4H), 0.52-0.05 (m, 11H). |
| 53 | ¹H NMR (400 MHz, DMSO-d₆) δ 9.82 (s, 1H), 8.42 (d, J = 8.8 Hz, 1H), |
| | 8.36 (d, J = 8.9 Hz, 1H), 7.79 (s, 1H), 7.65 (t, J = 8.3 Hz, 1H), 7.46 (d, J = |
| | 2.0 Hz, 1H), 7.05 (d, J = 1.7 Hz, 1H), 7.02 (d, J = 2.0 Hz, 1H), 6.94 (t, J = |
| | 7.3 Hz, 1H), 5.31-5.21 (m, 1H), 4.91 (t, J = 8.2 Hz, 1H), 4.03 (s, 3H), 3.86 |
| | (s, 2H), 3.74 (d, J = 7.1 Hz, 2H), 3.45 (dd, J = 10.5, 7.0 Hz, 1H), 3.29 (s, |
| | 3H), 1.33 (d, J = 6.9 Hz, 3H), 1.00-0.66 (m, 4H), 0.48-0.05 (m, 12H). |
| 68 | ¹H NMR (400 MHz, DMSO-d₆) δ 9.80 (s, 1H), 8.42 (d, J = 8.7 Hz, 1H), |
| | 8.36 (d, J = 8.9 Hz, 1H), 7.79 (s, 1H), 7.65 (t, J = 8.3 Hz, 1H), 7.47 (d, J = |
| | 2.0 Hz, 1H), 7.04 (dd, J = 12.2, 1.7 Hz, 1H), 6.96 (dd, J = 7.5, 5.3 Hz, 2H), |
| | 5.29-5.18 (m, 1H), 4.95-4.86 (m, 1H), 4.54-4.40 (m, 2H), 3.86 (s, 2H), |
| | 3.74 (d, J = 7.1 Hz, 2H), 3.45 (dq, J = 13.8, 7.0 Hz, 1H), 1.33 (d, J = 6.9 Hz, |
| | 3H), 1.28 (t, J = 7.1 Hz, 3H), 0.98-0.67 (m, 4H), 0.50-0.06 (m, 12H). |
| 72 | ¹H NMR (400 MHz, DMSO-d₆) δ 9.80 (s, 1H), 8.58 (d, J = 8.6 Hz, 1H), |
| | 8.36 (d, J = 8.8 Hz, 1H), 7.78 (s, 1H), 7.65 (t, J = 8.3 Hz, 1H), 7.47 (d, J = |
| | 2.0 Hz, 1H), 7.03 (d, J = 12.1 Hz, 1H), 6.96 (t, J = 5.6 Hz, 2H), 5.28-5.11 |
| | (m, 1H), 4.90 (t, J = 8.2 Hz, 1H), 4.56-4.36 (m, 2H), 3.76 (qd, J = 14.4, |
| | 7.1 Hz, 2H), 3.44-3.37 (m, 1H), 2.18 (dt, J = 15.2, 7.5 Hz, 1H), 2.09 (dd, |
| | J = 14.8, 7.5 Hz, 1H), 1.33 (d, J = 6.9 Hz, 3H), 1.28 (t, J = 7.1 Hz, 3H), 0.98 |
| | (t, J = 7.6 Hz, 3H), 0.95-0.84 (m, 2H), 0.80 (d, J = 4.9 Hz, 1H), 0.77-0.66 |
| | (m, 1H), 0.50-0.41 (m, 1H), 0.40-0.32 (m, 2H), 0.32-0.25 (m, 3H), |
| | 0.25-0.20 (m, 3H), 0.20-0.15 (m, 2H), 0.11 (dd, J = 10.3, 5.4 Hz, 1H). |

### Example 15. N—((S)-1,1-dicyclopropyl-3-((4-((1R,2S)-1-(1-(cyclopropylmethyl)-1H-1,2,4-triazol-3-yl)-1-(2-methoxyacetamido)propan-2-yl)-2-fluorophenyl)amino)-3-oxopropan-2-yl)-1-ethyl-1H-pyrazole-5-carboxamide (Compound 20)

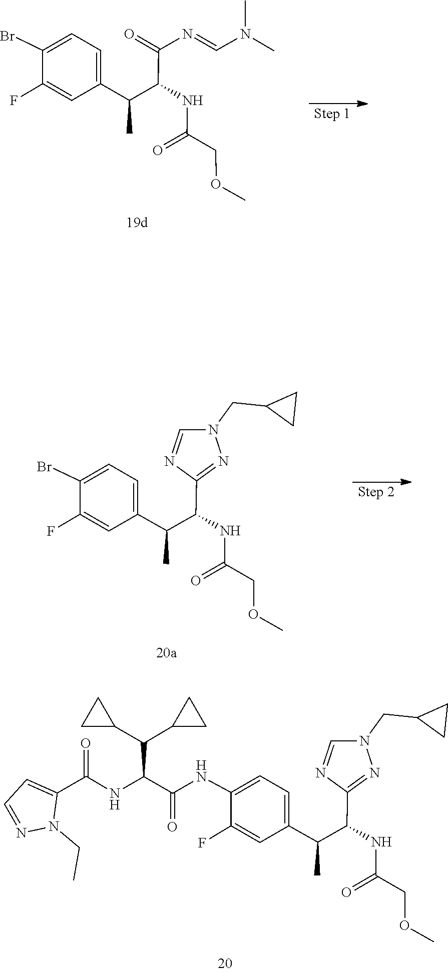 [SMILES: COCC(=O)N[C@@H](C(=O)/N=C/N(C)C)[C@@H](C)c1ccc(Br)c(F)c1 ; COCC(=O)N[C@@H](c1ncn(CC2CC2)n1)[C@@H](C)c1ccc(Br)c(F)c1 ; CCn1nccc1C(=O)N[C@H](C(=O)Nc1ccc([C@H](C)[C@@H](NC(=O)COC)c2ncn(CC3CC3)n2)cc1F)C(C1CC1)C1CC1]

### Step 1. N-((1R,2S)-2-(4-bromo-3-fluorophenyl)-1-(1-(cyclopropylmethyl)-1H-1,2,4-triazol-3-yl)propyl)-2-methoxyacetamide (20a)

To a solution of 19d (600 mg, 1.5 mmol) in acetic acid (20 mL) was added (cyclopropylmethyl)hydrazine dihydrochloride (1.21 g, 7.7 mmol), which was then heated to 110° C. and stirred for 8 h. After cooling to room temperature, the reaction mixture was concentrated to dryness and the residue was purified by prep-HPLC to give the title compound 20a (70 mg, 11%).

MS m/z (ESI): 425, 427 [M+1]

### Step 2. N—((S)-1,1-dicyclopropyl-3-((4-((1R,2S)-1-(1-(cyclopropylmethyl)-1H-1,2,4-triazol-3-yl)-1-(2-methoxyacetamido)propan-2-yl)-2-fluorophenyl)amino)-3-oxopropan-2-yl)-1-ethyl-1H-pyrazole-5-carboxamide (20)

A mixture of 20a (40 mg, 0.09 mmol), is (41 mg, 0.14 mmol), Cs₂CO₃ (61 mg, 0.19 mmol), Pd₂(dba)₃ (17 mg, 0.019 mmol), XantPhos (22 mg, 0.038 mmol) and 1,4-dioxane (3 mL) was heated to 105° C. under microwave and stirred for 2.5 h. After cooling to room temperature, the reaction mixture was concentrated to dryness and the residue was purified by prep-HPLC to give the title compound 20 (10.8 mg, 18%).

MS m/z (ESI): 635 [M+1]

¹H NMR (400 MHz, CD₃OD) δ 8.29 (s, 1H), 7.73 (t, J=8.2 Hz, 1H), 7.50 (d, J=2.1 Hz, 1H), 6.96-6.85 (m, 2H), 6.84 (d, J=2.1 Hz, 1H), 5.32 (dt, J=9.1, 4.6 Hz, 1H), 4.93 (d, J=7.0 Hz, 1H), 4.53 (q, J=7.2 Hz, 2H), 4.01-3.84 (m, 4H), 3.46-3.33 (m, 4H), 1.46-1.33 (m, 6H), 1.13 (ddd, J=12.3, 7.7, 4.8 Hz, 1H), 0.91-0.73 (m, 3H), 0.57-0.17 (m, 12H).

Compound 30 was prepared according to the procedures of Example 15, except that a different reagent was used instead of (cyclopropylmethyl)hydrazine dihydrochloride.

| | Reagent replacing | MS |
|---|---|---|
| | (cyclopropylmethyl)hydrazine | m/z |
| Compound | dihydrochloride | (ESI) |
| N-((S)-1,1-dicyclopropyl-3-((2-fluoro-4-((1R,2S)- | Propylhydrazine | 623 |
| 1-(2-methoxyacetamido)-1-(1-propyl-1H-1,2,4- | dihydrochloride | [M + 1] |
| triazol-3-yl)propan-2-yl)phenyl)amino)-3- | | |
| oxopropan-2-yl)-1-ethyl-1H-pyrazole-5- | | |
| carboxamide (30) | | |

¹H NMR data of Compound 30 are shown below:

| Compound # | ¹H NMR |
|---|---|
| 30 | ¹H NMR (400 MHz, DMSO-d₆) δ 9.79 (s, 1H), 8.38 (d, J = 8.6 Hz, 1H), 8.32 |
| | (s, 1H), 7.80 (d, J = 8.9 Hz, 1H), 7.67 (d, J = 5.8 Hz, 1H), 7.47 (s, 1H), 6.97 (s, |
| | 1H), 6.90-6.73 (m, 2H), 5.13 (s, 1H), 4.92 (s, 1H), 4.54-4.37 (m, 2H), 3.96 |
| | (t, J = 6.4 Hz, 2H), 3.85 (d, J = 7.4 Hz, 2H), 3.29 (s, 3H), 1.60 (dd, J = 13.9, |
| | 7.0 Hz, 2H), 1.30 (s, 1H), 1.25 (d, J = 7.7 Hz, 6H), 0.89 (s, 1H), 0.81 (s, 1H), |
| | 0.73 (d, J = 8.7 Hz, 1H), 0.63 (t, J = 7.2 Hz, 3H), 0.44 (s, 1H), 0.40-0.26 (m, |
| | 3H), 0.22 (dd, J = 12.3, 6.1 Hz, 3H), 0.13 (d, J = 5.4 Hz, 1H). |

### Example 16. N—((S)-1,1-dicyclopropyl-3-((4-((1R,2S)-1-(3-cyclopropyl-3-methylureido)-1-(4-(cyclopropylmethyl)-4H-1,2,4-triazol-3-yl)propan-2-yl)-2-fluorophenyl)amino)-3-oxopropan-2-yl)-1-ethyl-1H-pyrazole-5-carboxamide (Compound 21)

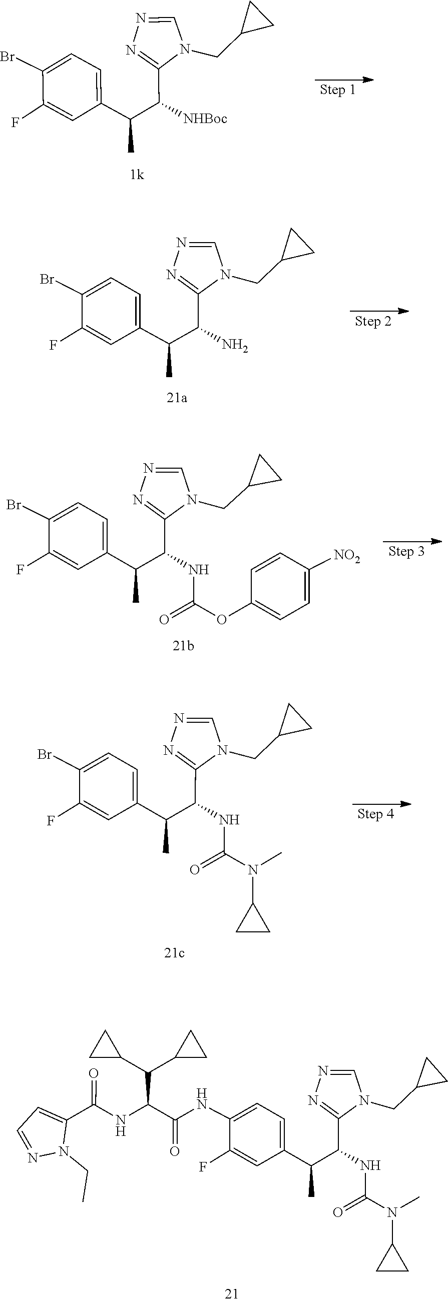 [SMILES: C[C@@H](c1ccc(Br)c(F)c1)[C@@H](NC(=O)OC(C)(C)C)c1nncn1CC1CC1 ; C[C@@H](c1ccc(Br)c(F)c1)[C@@H](N)c1nncn1CC1CC1 ; C[C@@H](c1ccc(Br)c(F)c1)[C@@H](NC(=O)Oc1ccc([N+](=O)[O-])cc1)c1nncn1CC1CC1 ; C[C@@H](c1ccc(Br)c(F)c1)[C@@H](NC(=O)N(C)C1CC1)c1nncn1CC1CC1 ; CCn1nccc1C(=O)N[C@H](C(=O)Nc1ccc([C@H](C)[C@@H](NC(=O)N(C)C2CC2)c2nncn2CC2CC2)cc1F)C(C1CC1)C1CC1]

### Step 1. (1R,2S)-2-(4-bromo-3-fluorophenyl)-1-(4-(cyclopropylmethyl)-4H-1,2,4-triazol-3-yl)propan-1-amine hydrochloride (21a)

A mixture of 1k (453 mg, 1 mmol), hydrochloric acid in 1,4-dioxane (4M, 2 mL) and methanol (2 mL) was stirred for 1 h and then concentrated to dryness to give the title compound 21a (353 mg, crude). The crude product was directly used in the next step without further purification.

MS m/z (ESI): 353, 355 [M+1]

### Step 2. 4-Nitrophenyl ((1R,2S)-2-(4-bromo-3-fluorophenyl)-1-(4-(cyclopropylmethyl)-4H-1,2,4-triazol-3-yl)propyl)carbamate (21b)

To a solution of 21a (106 mg, 0.3 mmol) in dichloromethane (1.5 mL) at 0° C. were added 4-nitrophenyl chloroformate (91 mg, 0.45 mmol) and TEA (208 uL, 1.5 mmol), which was then stirred at 0° C. for 30 min. The crude reaction mixture was directly used in the next step without further purification.

MS m/z (ESI): 518, 520 [M+1]

### Step 3. 3-((1R,2S)-2-(4-bromo-3-fluorophenyl)-1-(4-(cyclopropylmethyl)-4H-1,2,4-triazol-3-yl)propyl)-1-cyclopropyl-1-methylurea (21c)

To a mixture of 21b (135 mg, 0.3 mmol), TEA (208 uL, 1.5 mmol) and dichloromethane (1.5 mL) was added 1-methyl-1-cyclopropylamine hydrochloride (121 mg, 0.6 mmol), which was then stirred for 2 h. The reaction mixture was added with a saturated aqueous NaHCO₃ solution (20 mL) and extracted with dichloromethane (20 mL). The organic phase was concentrated to dryness and the residue was purified by silica gel column chromatography (dichloromethane/methanol=100/0 to 9/1) to give the title compound 21c (90 mg, 66%).

MS m/z (ESI): 450, 452 [M+1]

### Step 4. N—((S)-1,1-dicyclopropyl-3-((4-((1R,2S)-1-(3-cyclopropyl-3-methylureido)-1-(4-(cyclopropylmethyl)-4H-1,2,4-triazol-3-yl)propan-2-yl)-2-fluorophenyl)amino)-3-oxopropan-2-yl)-1-ethyl-1H-pyrazole-5-carboxamide (21)

A mixture of 21c (90 mg, 0.2 mmol), is (87 mg, 0.3 mmol), Cs₂CO₃ (198 mg, 0.6 mmol), BrettPhos Pd G3 (18 mg, 0.02 mmol) and 1,4-dioxane (1 mL) was heated to 110° C. and stirred overnight. After cooling to room temperature, the reaction mixture was added with a saturated aqueous NaHCO₃ solution (20 mL) and extracted with EtOAc (20 mL). The organic phase was concentrated to dryness and the residue was purified by silica gel column chromatography (dichloromethane/methanol=100/0 to 9/1) and prep-HPLC in sequence to give the title compound 21 (18.5 mg, 13%).

MS m/z (ESI): 660 [M+1]

¹H NMR (400 MHz, DMSO-d₆) δ 9.82 (s, 1H), 8.38 (d, J=8.5 Hz, 1H), 8.29 (s, 1H), 7.75-7.60 (m, 1H), 7.47 (d, J=2.1 Hz, 1H), 7.02 (d, J=12.3 Hz, 1H), 6.97 (s, 2H), 6.53 (d, J=5.8 Hz, 1H), 5.05 (t, J=7.7 Hz, 1H), 4.91 (t, J=6.3 Hz, 1H), 4.56-4.36 (m, 2H), 3.57 (d, J=15.5 Hz, 3H), 2.74 (d, J=4.1 Hz, 3H), 1.40-1.32 (m, 3H), 1.28 (td, J=7.1, 4.1 Hz, 4H), 1.24 (s, 1H), 0.86 (s, 3H), 0.78 (d, J=14.1 Hz, 2H), 0.72 (d, J=8.8 Hz, 1H), 0.58 (s, 2H), 0.40 (dd, J=20.9, 10.2 Hz, 4H), 0.28 (d, J=4.6 Hz, 1H), 0.21 (d, J=10.5 Hz, 5H), 0.11 (s, 1H).

The following intermediate was prepared according to steps 2 to 3 of Example 16, except that a different reagent was used instead of 21a.

| | Reagent replacing | MS m/z |
|---|---|---|
| Intermediate | 21a | (ESI) |
| 3-((1R,2S)-2-(4-bromo-3-fluorophenyl)-1-(1- | (1R,2S)-2-(4-bromo- | 450, 452 |
| (cyclopropylmethyl)-1H-1,2,4-triazol-5-yl)propyl)-1- | 3-fluorophenyl)-1-(1- | [M + 1] |
| cyclopropyl-1-methylurea | (cyclopropylmethyl)- | |
| | 1H-1,2,4-triazol-5- | |
| | yl)propan-1-amine | |
| | (55a) | |

### Example 17. N—((S)-1,1-dicyclopropyl-3-((4-((1R,2S)-1-(1-(cyclopropylmethyl)-1H-tetrazol-5-yl)-1-(2-methoxyacetamido)propan-2-yl)-2-fluorophenyl)amino)-3-oxopropan-2-yl)-1-ethyl-1H-pyrazole-5-carboxamide (Compound 24)

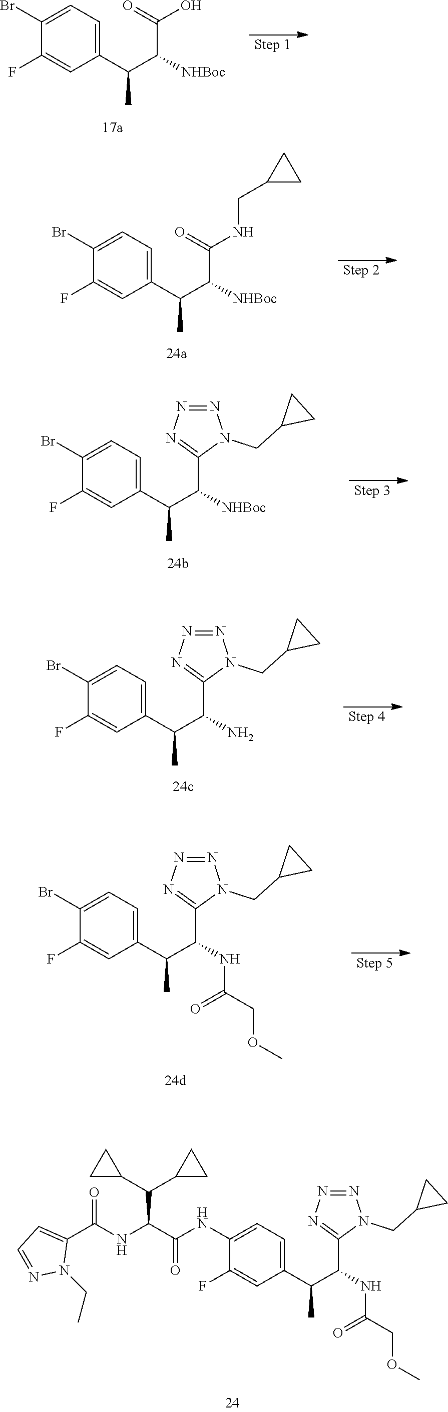 [SMILES: C[C@@H](c1ccc(Br)c(F)c1)[C@@H](NC(=O)OC(C)(C)C)C(=O)O ; C[C@@H](c1ccc(Br)c(F)c1)[C@@H](NC(=O)OC(C)(C)C)C(=O)NCC1CC1 ; C[C@@H](c1ccc(Br)c(F)c1)[C@@H](NC(=O)OC(C)(C)C)c1nnnn1CC1CC1 ; C[C@@H](c1ccc(Br)c(F)c1)[C@@H](N)c1nnnn1CC1CC1 ; COCC(=O)N[C@@H](c1nnnn1CC1CC1)[C@@H](C)c1ccc(Br)c(F)c1 ; CCn1nccc1C(=O)N[C@H](C(=O)Nc1ccc([C@H](C)[C@@H](NC(=O)COC)c2nnnn2CC2CC2)cc1F)C(C1CC1)C1CC1]

### Step 1. Tert-butyl ((2R,3S)-3-(4-bromo-3-fluorophenyl)-1-((cyclopropylmethyl)amino)-1-oxobutan-2-yl)carbamate (24a)

To a mixture of 17a (1.01 g, 2.69 mmol), (aminomethyl)cyclopropane (191 mg, 2.69 mmol), DMTMM·BF₄ (791 mg, 2.69 mmol) and acetonitrile (20 mL) was added TEA (272 mg, 2.69 mmol), which was then stirred for 2 h. The reaction mixture was concentrated to dryness. The residue was added with EtOAc (100 mL), washed with dilute hydrochloric acid (2 N, 50 mL) and saturated brine (50 mL). The organic phase was concentrated to dryness and the residue was purified by silica gel column chromatography (PE/EtOAc=100/0 to 5/1) to give the title compound 24a (890 mg, 77%).

MS m/z (ESI): 347, 349 [M+1]

### Step 2. Tert-butyl ((1R,2S)-2-(4-bromo-3-fluorophenyl)-1-(1-(cyclopropylmethyl)-1H-tetrazol-5-yl)propyl)carbamate (24b)

To a mixture of 24a (890 mg, 1.9 mmol), trimethylsilyl azide (4.45 g, 38.6 mmol), PPh₃ (10.10 g, 38.6 mmol) and THF (50 mL) at 0° C. was added DEAD (6.72 g, 38.6 mmol), which was then warmed to room temperature and stirred for 5 days. The reaction mixture was concentrated to dryness and the residue was purified by silica gel column chromatography (PE/EtOAc=100/0 to 10/1) to give the title compound 24b (570 mg, 65%).

MS m/z (ESI): 454, 456 [M+1]

### Step 3. (1R,2S)-2-(4-bromo-3-fluorophenyl)-1-(1-(cyclopropylmethyl)-1H-tetrazol-5-yl)propan-1-amine hydrochloride (24c)

A mixture of 24b (570 mg, 1.25 mmol), hydrochloric acid in 1,4-dioxane (4 M, 3 mL) and methanol (3 mL) was stirred for 2 h. The reaction mixture was concentrated to dryness to give the title compound 24c (488 mg, 99%). The crude product was directly used in the next step without further purification.

MS m/z (ESI): 354, 356 [M+1]

### Step 4. N-((1R,2S)-2-(4-bromo-3-fluorophenyl)-1-(1-(cyclopropylmethyl)-1H-tetrazol-5-yl)propyl)-2-methoxyacetamide (24d)

To a solution of 24c (488 mg, 1.25 mmol) in THF (20 mL) were added TEA (632 mg, 6.25 mmol) and 2-methoxyacetyl chloride (204 mg, 1.88 mmol), which was then stirred for 1 h. The reaction mixture was added EtOAc (50 mL) and washed with saturated brine (50 mL). The organic phase was concentrated to dryness and the residue was purified by prep-HPLC to give the title compound 24d (280 mg, 53%).

MS m/z (ESI): 426, 428 [M+1]

### Step 5. N—((S)-1,1-dicyclopropyl-3-((4-((1R,2S)-1-(1-(cyclopropylmethyl)-1H-tetrazol-5-yl)-1-(2-methoxyacetamido)propan-2-yl)-2-fluorophenyl)amino)-3-oxopropan-2-yl)-1-ethyl-1H-pyrazole-5-carboxamide (24)

A mixture of 24d (88 mg, 0.21 mmol), is (67 mg, 0.23 mmol), Cs₂CO₃ (205 mg, 0.63 mmol), Pd₂(dba)₃ (29 mg, 0.032 mmol), XantPhos (37 mg, 0.064 mmol) and 1,4-dioxane (3 mL) was heated to 100° C. under microwave and stirred for 1.5 h. After cooling to room temperature, the reaction mixture was filtered. The filtrate was concentrated to dryness and the residue was purified by silica gel column chromatography (dichloromethane/methanol=100/0 to 40/1) then by prep-HPLC to give the title compound 24 (30 mg, 23%).

MS m/z (ESI): 636 [M+1]

¹H NMR (400 MHz, DMSO-d₆) δ 9.82 (s, 1H), 8.81 (d, J=8.2 Hz, 1H), 8.37 (d, J=8.8 Hz, 1H), 7.64 (dd, J=11.0, 5.6 Hz, 1H), 7.47 (d, J=2.0 Hz, 1H), 7.12 (dd, J=12.1, 1.5 Hz, 1H), 6.97 (d, J=1.9 Hz, 1H), 6.95 (s, 1H), 5.38 (dd, J=10.7, 8.3 Hz, 1H), 4.90 (t, J=8.2 Hz, 1H), 4.53-4.39 (m, 2H), 4.05 (p, J=7.1 Hz, 2H), 3.87 (s, 2H), 3.57 (dd, J=10.7, 6.9 Hz, 1H), 3.28 (s, 3H), 1.38 (d, J=6.8 Hz, 3H), 1.28 (t, J=7.1 Hz, 3H), 1.14-0.98 (m, 1H), 0.96-0.65 (m, 3H), 0.49-0.05 (m, 12H).

Compounds 29 and 54 were prepared according to the procedures of Example 17, except that different reagents were used instead of is.

| | | MS m/z |
|---|---|---|
| Compound | Reagent replacing 1s | (ESI) |
| N-((S)-1,1-dicyclopropyl-3-((4-((1R,2S)-1-(1- | (S)-N-(1-amino-3,3- | 638 |
| (cyclopropylmethyl)-1H-tetrazol-5-yl)-1-(2- | dicyclopropyl-1- | [M + 1] |
| methoxyacetamido)propan-2-yl)-2- | oxopropan-2-yl)-4-ethyl- | |
| fluorophenyl)amino)-3-oxopropan-2-yl)-4-ethyl- | 1,2,5-oxadiazole-3- | |
| 1,2,5-oxadiazole-3-carboxamide (29) | carboxamide | |
| N-((S)-1,1-dicyclopropyl-3-((4-((1R,2S)-1-(1- | (S)-N-(1-amino-3,3- | 624 |
| (cyclopropylmethyl)-1H-tetrazol-5-yl)-1-(2- | dicyclopropyl-1- | [M + 1] |
| methoxyacetamido)propan-2-yl)-2- | oxopropan-2-yl)-4- | |
| fluorophenyl)amino)-3-oxopropan-2-yl)-4-methyl- | methyl-1,2,5-oxadiazole- | |
| 1,2,5-oxadiazole-3-carboxamide (54) | 3-carboxamide | |

¹H NMR data of compounds 29 and 54 are shown below:

| Compound # | ¹H NMR |
|---|---|
| 29 | ¹H NMR (400 MHz, DMSO-d₆) δ 9.86 (s, 1H), 9.04 (d, J = 8.9 Hz, 1H), |
| | 8.81 (d, J = 8.1 Hz, 1H), 7.62 (q, J = 8.4 Hz, 1H), 7.12 (d, J = 12.1 Hz, 1H), |
| | 6.96 (t, J = 6.6 Hz, 1H), 5.45-5.31 (m, 1H), 5.04-4.89 (m, 1H), 4.12-3.98 |
| | (m, 2H), 3.87 (s, 2H), 3.57 (td, J = 13.5, 6.6 Hz, 1H), 3.28 (s, 3H), 2.89 |
| | (q, J = 7.5 Hz, 2H), 1.38 (d, J = 6.8 Hz, 3H), 1.23 (t, J = 7.5 Hz, 3H), |
| | 1.09-1.00 (m, 1H), 0.94-0.66 (m, 3H), 0.54-0.13 (m, 12H). |
| 54 | ¹H NMR (400 MHz, DMSO-d₆) δ 9.87 (s, 1H), 9.05-8.94 (m, 1H), 8.81 |
| | (d, J = 8.2 Hz, 1H), 7.62 (dd, J = 16.9, 8.4 Hz, 1H), 7.13 (d, J = 12.2 Hz, |
| | 1H), 6.96 (t, J = 7.1 Hz, 1H), 5.39 (dd, J = 10.6, 8.3 Hz, 1H), 5.01-4.93 |
| | (m, 1H), 4.12-3.98 (m, 2H), 3.92-3.82 (m, 2H), 3.57 (dd, J = 10.7, 7.0 |
| | Hz, 1H), 3.28 (s, 3H), 2.47 (s, 3H), 1.38 (d, J = 6.9 Hz, 3H), 1.06 (dd, J = |
| | 10.1, 5.3 Hz, 1H), 0.92-0.68 (m, 3H), 0.54-0.07 (m, 12H). |

### Example 18. N—((S)-1,1-dicyclopropyl-3-((4-((1R,2S)-1-(1-(cyclopropylmethyl)-3-methyl-1H-1,2,4-triazol-5-yl)-1-(2-methoxyacetamido)propan-2-yl)-2-fluorophenyl)amino)-3-oxopropan-2-yl)-1-ethyl-1H-pyrazole-5-carboxamide (Compound 25)

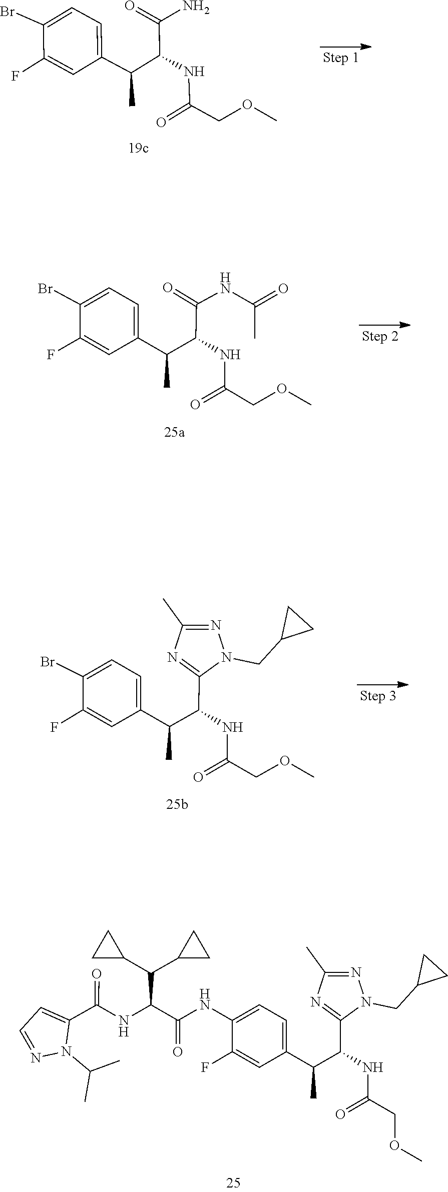 [SMILES: COCC(=O)N[C@@H](C(N)=O)[C@@H](C)c1ccc(Br)c(F)c1 ; COCC(=O)N[C@@H](C(=O)NC(C)=O)[C@@H](C)c1ccc(Br)c(F)c1 ; COCC(=O)N[C@@H](c1nc(C)nn1CC1CC1)[C@@H](C)c1ccc(Br)c(F)c1 ; COCC(=O)N[C@@H](c1nc(C)nn1CC1CC1)[C@@H](C)c1ccc(NC(=O)[C@@H](NC(=O)c2ccnn2C(C)C)C(C2CC2)C2CC2)c(F)c1]

### Step 1. (2R,3S)—N-acetyl-3-(4-bromo-3-fluorophenyl)-2-(2-methoxyacetamido)butanamide (25a)

To a solution of 19c (69 mg, 0.2 mmol) in acetic anhydride (2 mL) at 0° C. was added a drop of concentrated sulfuric acid, which was then stirred for 4 h. The reaction mixture was added with a saturated aqueous NaHCO₃ solution (50 mL) and extracted with EtOAc (2×75 mL). The combined organic phase was concentrated to dryness and the residue was purified by silica gel column chromatography (PE/EtOAc=50/1 to 1/1) to give the title compound 25a (50 mg, 64%).

MS m/z (ESI): 389, 391 [M+1]

### Step 2. N-((1R,2S)-2-(4-bromo-3-fluorophenyl)-1-(1-(cyclopropylmethyl)-3-methyl-1H-1,2,4-triazol-5-yl)propyl)-2-methoxyacetamide (25b)

To a solution of 25a (400 mg, 1.4 mmol) in pyridine (10 mL) was added (cyclopropylmethyl)hydrazine dihydrochloride (1.09 g, 6.9 mmol), which was then heated to 150° C. under microwave and stirred for 1 h. After cooling to room temperature, the reaction mixture was concentrated to dryness and the residue was purified by prep-HPLC to give the title compound 25b (120 mg, 20%).

MS m/z (ESI): 439, 441 [M+1]

### Step 3. N—((S)-1,1-dicyclopropyl-3-((4-((1R,2S)-1-(1-(cyclopropylmethyl)-3-methyl-1H-1,2,4-triazol-5-yl)-1-(2-methoxyacetamido)propan-2-yl)-2-fluorophenyl)amino)-3-oxopropan-2-yl)-1-ethyl-1H-pyrazole-5-carboxamide (25)

A mixture of 25b (100 mg, 0.23 mmol), is (100 mg, 0.35 mmol), Cs₂CO₃ (150 mg, 0.46 mmol), BrettPhos Pd G3 (42 mg, 0.046 mmol) and 1,4-dioxane (3 mL) was heated to 110° C. under microwave and stirred for 3 h. After cooling to room temperature, the reaction mixture was concentrated to dryness and the residue was purified by prep-HPLC to give the title compound 25 (21 mg, 14%).

MS m/z (ESI): 649 [M+1]

¹H NMR (400 MHz, CD₃OD) δ 7.81-7.70 (m, 1H), 7.49 (d, J=2.1 Hz, 1H), 7.04-6.93 (m, 2H), 6.84 (d, J=2.1 Hz, 1H), 5.28 (d, J=11.0 Hz, 1H), 4.92 (dd, J=10.6, 5.2 Hz, 1H), 4.53 (q, J=7.2 Hz, 2H), 4.02-3.88 (m, 2H), 3.68 (dd, J=14.5, 6.7 Hz, 1H), 3.57 (dd, J=14.5, 7.6 Hz, 1H), 3.47-3.34 (m, 4H), 2.27 (s, 3H), 1.49-1.34 (m, 6H), 0.83 (ddt, J=25.4, 18.3, 8.4 Hz, 4H), 0.56-0.18 (m, 12H).

### Example 19. N—((S)-1-((4-((1R,2S)-1-(3-cyclopropoxypyrazin-2-yl)-1-(2-methoxyacetamido)propan-2-yl)-2-fluorophenyl)amino)-3,3-dicyclopropyl-1-oxopropan-2-yl)-1-ethyl-1H-pyrazole-5-carboxamide (Compound 27)

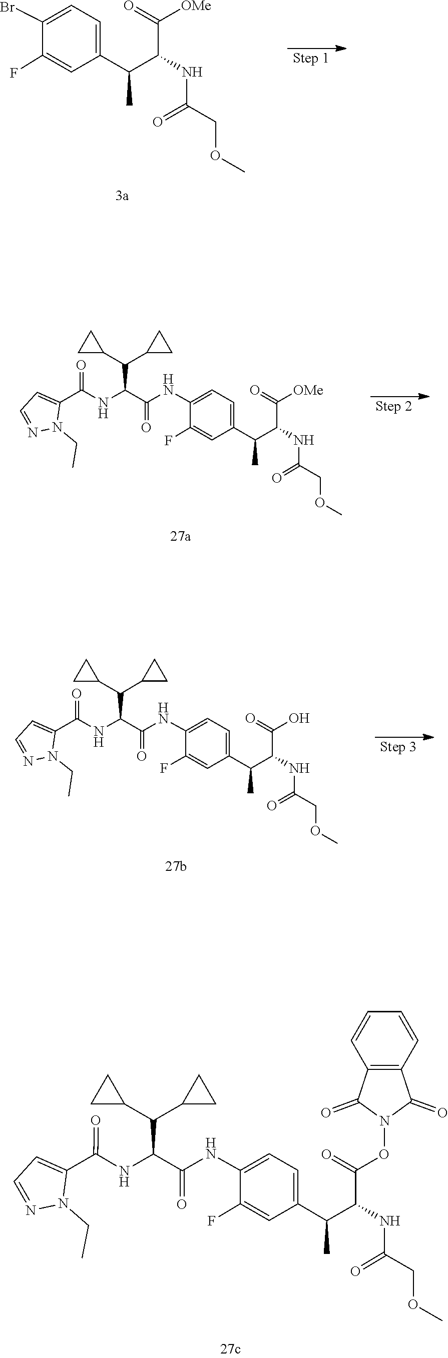 [SMILES: COCC(=O)N[C@@H](C(=O)OC)[C@@H](C)c1ccc(Br)c(F)c1 ; CCn1nccc1C(=O)N[C@H](C(=O)Nc1ccc([C@H](C)[C@@H](NC(=O)COC)C(=O)OC)cc1F)C(C1CC1)C1CC1 ; CCn1nccc1C(=O)N[C@H](C(=O)Nc1ccc([C@H](C)[C@@H](NC(=O)COC)C(=O)O)cc1F)C(C1CC1)C1CC1 ; CCn1nccc1C(=O)N[C@H](C(=O)Nc1ccc([C@H](C)[C@@H](NC(=O)COC)C(=O)ON2C(=O)c3ccccc3C2=O)cc1F)C(C1CC1)C1CC1]

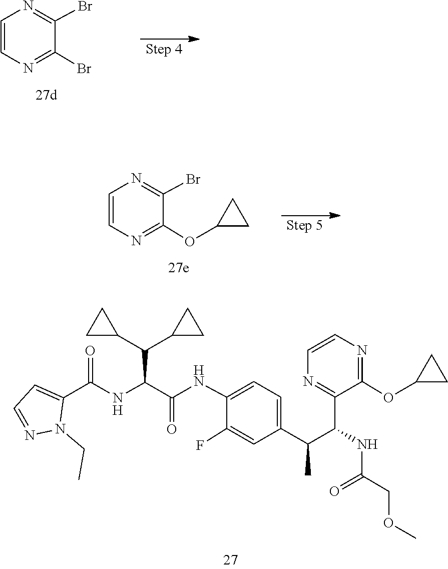 [SMILES: Brc1nccnc1Br ; Brc1nccnc1OC1CC1 ; CCn1nccc1C(=O)N[C@H](C(=O)Nc1ccc([C@H](C)[C@@H](NC(=O)COC)c2nccnc2OC2CC2)cc1F)C(C1CC1)C1CC1]

### Step 1. Methyl (2R,3S)-3-(4-((S)-3,3-dicyclopropyl-2-(1-ethyl-1H-pyrazole-5-carboxamido)propanamido)-3-fluorophenyl)-2-(2-methoxyacetamido)butanoate (27a)

A mixture of 3a (543 mg, 1.5 mmol), is (523 mg, 1.8 mmol), Cs₂CO₃ (977 mg, 3.0 mmol), BrettPhos Pd G3 (136 mg, 0.15 mmol) and 1,4-dioxane (4 mL) was stirred overnight at 110° C. After cooling to room temperature, the reaction mixture was added with dilute hydrochloric acid (2 N, 20 mL) and extracted with EtOAc (3×30 mL). The combined organic phase was dried over anhydrous Na₂SO₄ and filtered. The filtrate was concentrated to dryness to give the title compound 27a (857 mg, crude). The crude product was directly used in the next step without further purification.

MS m/z (ESI): 572 [M+1]

### Step 2. (2R,3S)-3-(4-((S)-3,3-dicyclopropyl-2-(1-ethyl-1H-pyrazole-5-carboxamido)propanamido)-3-fluorophenyl)-2-(2-methoxyacetamido)butanoic acid (27b)

To a mixture of 27a (857 mg, crude, 1.5 mmol), THF (5 mL) and water (5 mL) was added LiOH·H₂O (315 mg, 7.5 mmol), which was then stirred for 1 h. The reaction mixture was added with dilute hydrochloric acid (1 N, 20 mL) and extracted with EtOAc (3×20 mL). The combined organic phase was dried over anhydrous Na₂SO₄ and filtered. The filtrate was concentrated to dryness to give the title compound 27b (836 mg, crude). The crude product was directly used in the next step without further purification.

MS m/z (ESI): 558 [M+1]

### Step 3. 1,3-Dioxoisoindolin-2-yl (2R,3S)-3-(4-((S)-3,3-dicyclopropyl-2-(1-ethyl-1H-pyrazole-5-carboxamido)propanamido)-3-fluorophenyl)-2-(2-methoxyacetamido)butanoate (27c)

To a mixture of 27b (836 mg, crude, 1.5 mmol), 2-hydroxyisoindoline-1,3-dione (294 mg, 1.8 mmol) and dichloromethane (10 mL) were added EDCI (345 mg, 1.8 mmol) and DMAP (37 mg, 0.3 mmol), which was then stirred for 2 h. The reaction mixture was added with dilute hydrochloric acid (1 N, 20 mL) and extracted with EtOAc (3×20 mL). The combined organic phase was concentrated to dryness and the residue was purified by silica gel column chromatography (dichloromethane/methanol=100/0 to 9/1) to give the title compound 27c (1.05 g, 99%).

MS m/z (ESI): 703 [M+1]

### Step 4. 2-Bromo-3-cyclopropoxypyrazine (27e)

To a solution of cyclopropanol (947 uL, 15 mmol) in THF (25 mL) at 0° C. was added NaH (60% in mineral oil, 600 mg, 15 mmol), which was then stirred for 1 h. The mixture was added 2,3-dibromopyrazine 27d (1.19 g, 5 mmol), warmed to room temperature and stirred for 1 h. The reaction mixture was added with a saturated aqueous NaHCO₃ solution (100 mL) and extracted with EtOAc (100 mL). The combined organic phase was concentrated to dryness and the residue was purified by silica gel column chromatography (PE/EtOAc=100/0 to 7/3) to give the title compound 27e (1.05 g, 97%).

MS m/z (ESI): 215, 217 [M+1]

### Step 5. N—((S)-1-((4-((1R,2S)-1-(3-cyclopropoxypyrazin-2-yl)-1-(2-methoxyacetamido)propan-2-yl)-2-fluorophenyl)amino)-3,3-dicyclopropyl-1-oxopropan-2-yl)-1-ethyl-1H-pyrazole-5-carboxamide (27)

A mixture of 27c (1.05 g, 1.5 mmol), 27e (387 mg, 1.8 mmol), NiCl₂(dtbpy) (205 mg, 0.63 mmol), zinc dust (196 mg, 3.0 mmol) and DMA (7.5 mL) was stirred overnight. The reaction mixture was added with dilute hydrochloric acid (1 N, 50 mL) and extracted with EtOAc (3×50 mL). The combined organic phase was concentrated to dryness and the residue was purified by prep-HPLC to give the title compound 27 (2.5 mg, 1%).

MS m/z (ESI): 648 [M+1]

¹H NMR (400 MHz, DMSO-d₆) δ 9.81 (s, 1H), 8.37 (d, J=8.6 Hz, 1H), 8.18 (d, J=2.6 Hz, 1H), 8.06 (d, J=2.6 Hz, 1H), 7.89 (d, J=9.3 Hz, 1H), 7.63 (dd, J=18.3, 9.9 Hz, 1H), 7.47 (d, J=1.9 Hz, 1H), 6.97 (d, J=1.9 Hz, 1H), 6.85 (d, J=11.9 Hz, 1H), 6.74 (d, J=8.9 Hz, 1H), 5.35 (t, J=9.3 Hz, 1H), 4.92 (s, 1H), 4.47 (d, J=6.9 Hz, 2H), 4.04 (s, 1H), 3.84 (d, J=11.0 Hz, 2H), 3.30 (s, 3H), 3.20-3.15 (m, 1H), 2.00 (dd, J=14.4, 6.9 Hz, 1H), 1.29 (d, J=7.2 Hz, 3H), 1.25 (s, 2H), 0.85 (s, 3H), 0.72 (d, J=6.1 Hz, 2H), 0.64 (d, J=7.1 Hz, 1H), 0.44 (s, 1H), 0.34 (d, J=5.8 Hz, 1H), 0.28 (d, J=6.3 Hz, 3H), 0.20 (dd, J=9.6, 4.7 Hz, 3H), 0.13 (s, 1H).

### Example 20. N—((S)-1,1-dicyclopropyl-3-((4-((1R,2S)-1-(3-(cyclopropylmethyl)pyridin-2-yl)-1-(2-methoxyacetamido)propan-2-yl)-2-fluorophenyl)amino)-3-oxopropan-2-yl)-1-ethyl-1H-pyrazole-5-carboxamide (Compound 28)

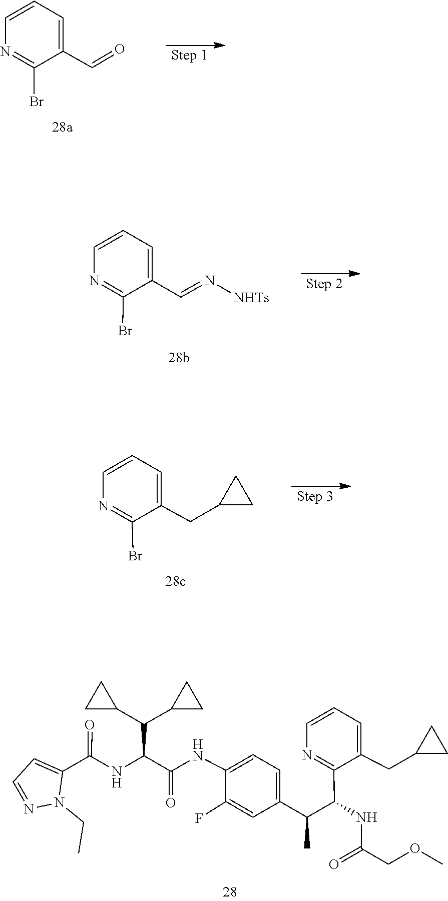

### Step 1. (E′-N′-((2-bromopyridin-3-yl)methylene)-4-methylbenzenesulfonohydrazide (28b)

To a solution of 2-bromonicotinaldehyde 28a (1.0 g, 5.4 mmol) in methanol (10 mL) was added p-toluenesulfonyl hydrazide (1.00 g, 5.4 mmol), which was then stirred for 2 h. The reaction mixture was concentrated to dryness. The residue was added EtOAc (50 mL), washed with water (50 mL) and saturated brine (50 mL). The organic phase was dried over anhydrous Na₂SO₄ and filtered. The filtrate was concentrated to dryness to give the title compound 28b (1.78 g, 93%). The crude product was directly used in the next step without further purification.

MS m/z (ESI): 354, 356 [M+1]

### Step 2. 2-Bromo-3-(cyclopropylmethyl)pyridine (28c)

To a solution of 28b (1.78 g, 5 mmol) in 1,4-dioxane (30 mL) were added cyclopropylboronic acid (430 mg, 5 mmol) and Cs₂CO₃ (3.20 g, 10 mmol), which was then heated to reflux and stirred for 2 h. After cooling to room temperature, the reaction mixture was filtered. The filtrate was concentrated to dryness and the residue was purified by silica gel column chromatography (PE/EtOAc=100/0 to 8/1) to give the title compound 28c (400 mg, 40%).

MS m/z (ESI): 212, 214 [M+1]

### Step 3. N—((S)-1,1-dicyclopropyl-3-((4-((1R,2S)-1-(3-(cyclopropylmethyl)pyridin-2-yl)-1-(2-methoxyacetamido)propan-2-yl)-2-fluorophenyl)amino)-3-oxopropan-2-yl)-1-ethyl-1H-pyrazole-5-carboxamide (28)

A mixture of 27c (1.41 g, 2 mmol), 28c (200 mg, 1 mmol), NiCl₂(dtbpy) (80 mg, 0.2 mmol), zinc dust (130 mg, 2 mmol) and DMA (5 mL) was stirred overnight. The reaction mixture was added with water (50 mL) and extracted with EtOAc (3×50 mL). The combined organic phase was concentrated to dryness and the residue was purified by prep-HPLC to give the title compound 28 (2.5 mg, 1%).

MS m/z (ESI): 645 [M+1]

¹H NMR (400 MHz, DMSO-d₆) δ 9.76 (s, 1H), 8.46 (d, J=4.7 Hz, 1H), 8.35 (d, J=8.7 Hz, 1H), 7.81 (d, J=9.4 Hz, 1H), 7.61 (t, J=8.3 Hz, 1H), 7.49 (dd, J=13.6, 5.4 Hz, 2H), 7.26-7.13 (m, 2H), 6.78 (t, J=8.4 Hz, 2H), 5.33 (dd, J=10.3, 7.0 Hz, 1H), 4.89 (t, J=8.1 Hz, 1H), 4.46 (d, J=8.9 Hz, 1H), 3.85 (q, J=15.0 Hz, 2H), 3.17 (s, 5H), 2.22-1.76 (m, 3H), 1.40-1.16 (m, 6H), 0.70 (d, J=7.8 Hz, 3H), 0.50-−0.01 (m, 12H).

### Example 21. (5-(Difluoromethyl)isoxazol-3-yl)methyl ((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)carbamate (Compound 32)

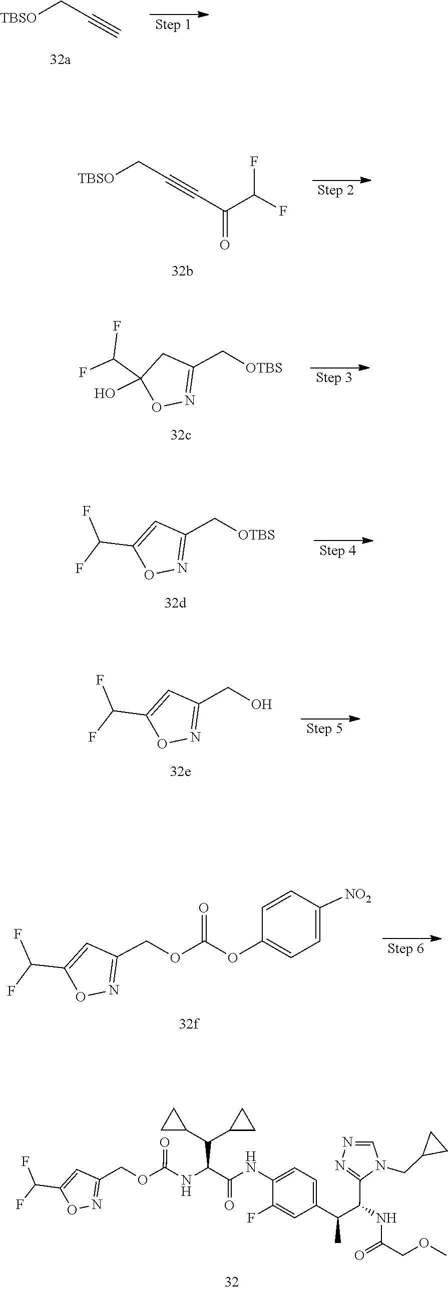

### Step 1. 5-((Tert-butyldimethylsilyl)oxy)-1,1-difluoropent-3-yn-2-one (32b)

To a solution of tert-butyldimethyl(prop-2-yn-1-yloxy)silane 32a (5.1 g, 30 mmol) in THF (60 mL) at −78° C. was added a solution of n-BuLi in hexanes (2.5 M, 12 mL), which was then warmed to −15° C. and stirred for 30 min. The reaction mixture was added with 2,2-difluoroethyl acetate (4.1 g, 33 mmol) and BF₃ Et₂O (4.7 g, 33 mmol) at −78° C., which was then warmed to room temperature and stirred for 15 h. The reaction mixture was added with a saturated aqueous NH₄Cl solution (100 mL) and extracted with EtOAc (2×200 mL). The combined organic phase was washed with saturated brine (150 mL) and concentrated to dryness. The residue was purified by silica gel column chromatography (PE/EtOAc=100/1 to 20/1) to give the title compound 32b (3.9 g, 52%).

¹H NMR (400 MHz, CDCl₃) δ 5.62 (t, J=54.0 Hz, 1H), 4.41 (s, 2H), 0.83-0.74 (m, 9H), 0.04-−0.05 (m, 6H).

### Step 2. 3-(((Tert-butyldimethylsilyl)oxy)methyl)-5-(difluoromethyl)-4,5-dihydroisoxazol-5-ol (32c)

To a solution of hydroxylamine hydrochloride (1.55 g, 22 mmol) in water (20 mL) at 0° C. was added NaHCO₃ (1.87 g, 22 mmol), which was then warmed to room temperature and stirred for 10 min. The mixture was added a solution of 32b (3.7 g, 15 mmol) in THF (20 mL) and stirred for 15 h. The reaction mixture was extracted with EtOAc (2×100 mL). The combined organic phase was washed with saturated brine (150 mL), dried over anhydrous Na₂SO₄ and filtered. The filtrate was concentrated to dryness to give the title compound 32c (3.90 g, 93%). The crude product was directly used in the next step without further purification.

MS m/z (ESI): 282 [M+1]

### Step 3. 3-(((Tert-butyldimethylsilyl)oxy)methyl)-5-(difluoromethyl)isoxazole (32d)

To a solution of 32c (3.90 g, 13.9 mmol) in dichloromethane (60 mL) was added CDI (6.74 g, 41.6 mmol), which was then stirred for 24 h. The reaction mixture was concentrated to dryness and the residue was purified by silica gel column chromatography (PE/dichloromethane=100/1 to 10/1) to give the title compound 32d (1.70 g, 47%).

MS m/z (ESI): 264 [M+1]

### Step 4. (5-(Difluoromethyl)isoxazol-3-yl)methanol (32e)

To a solution of 32d (1.7 g, 6.5 mmol) in THF (9 mL) and water (3 mL) was added TFA (3 mL), which was then stirred for 2 h. The reaction mixture was concentrated to dryness and the residue was purified by prep-HPLC to give the title compound 32e (360 mg, 37%).

MS m/z (ESI): 150 [M+1]

### Step 5. (5-(Difluoromethyl)isoxazol-3-yl)methyl (4-nitrophenyl) carbonate (32f)

To a solution of 32e (110 mg, 0.74 mmol) in dichloromethane (5 mL) at 0° C. were added 4-nitrophenyl chloroformate (193 mg, 0.96 mmol) and TEA (225 mg, 2.22 mmol), which was then warmed to room temperature and stirred for 1 h. The reaction mixture was concentrated to dryness and the residue was purified by silica gel column chromatography (PE/EtOAc=50/1 to 5/1) to give the title compound 32f (150 mg, 65%).

MS m/z (ESI): 315 [M+1]

### Step 6. (5-(Difluoromethyl)isoxazol-3-yl)methyl ((S)-1,1-dicyclopropyl-3-((4-((1R,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)carbamate (32)

To a mixture of 32f (55 mg, 0.18 mmol), 7g (30 mg, 0.06 mmol) and DMF (4 mL) was added DIPEA (23 mg, 0.18 mmol), which was then heated to 70° C. and stirred for 6 h. After cooling to room temperature, the reaction mixture was purified by prep-HPLC to give the title compound 32 (12.6 mg, 31%).

MS m/z (ESI): 688 [M+1]

¹H NMR (400 MHz, CD₃OD) δ 8.33 (s, 1H), 7.70 (s, 1H), 7.22-6.71 (m, 4H), 5.39-5.10 (m, 3H), 4.49 (d, J=5.3 Hz, 1H), 3.95 (q, J=15.2 Hz, 2H), 3.67 (dd, J=14.2, 7.4 Hz, 1H), 3.54 (dd, J=13.9, 7.0 Hz, 2H), 3.42 (s, 3H), 1.35 (dd, J=72.9, 16.5 Hz, 4H), 0.87 (d, J=53.3 Hz, 3H), 0.68-0.14 (m, 12H).

### Example 22. N—((S)-1,1-dicyclopropyl-3-((4-((1S,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-1-ethyl-1H-pyrazole-5-carboxamide (Compound 37)

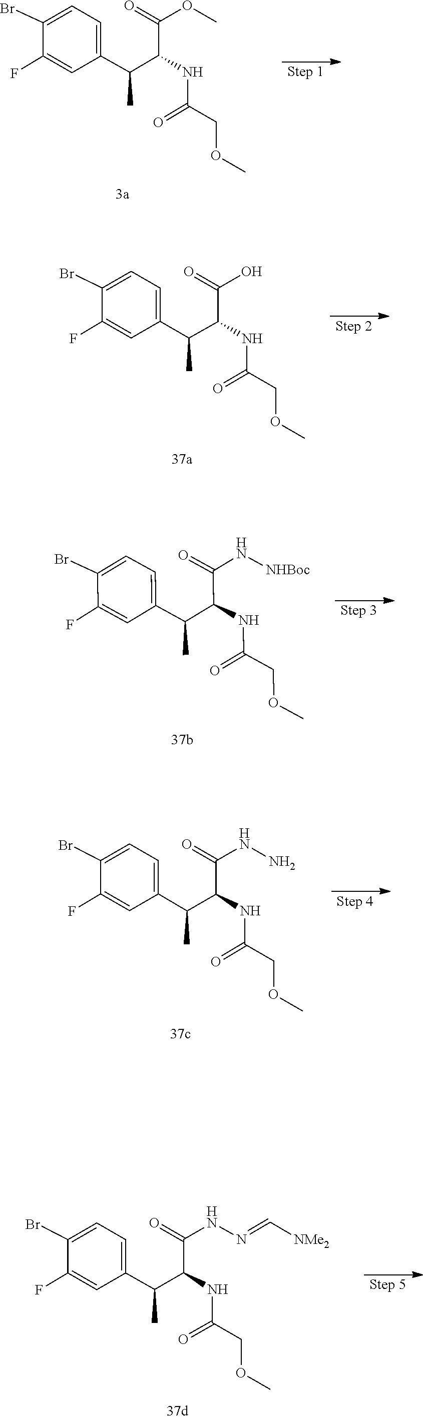 [SMILES: COCC(=O)N[C@@H](C(=O)OC)[C@@H](C)c1ccc(Br)c(F)c1 ; COCC(=O)N[C@@H](C(=O)O)[C@@H](C)c1ccc(Br)c(F)c1 ; COCC(=O)N[C@H](C(=O)NNC(=O)OC(C)(C)C)[C@@H](C)c1ccc(Br)c(F)c1 ; COCC(=O)N[C@H](C(=O)NN)[C@@H](C)c1ccc(Br)c(F)c1 ; COCC(=O)N[C@H](C(=O)N/N=C/N(C)C)[C@@H](C)c1ccc(Br)c(F)c1]

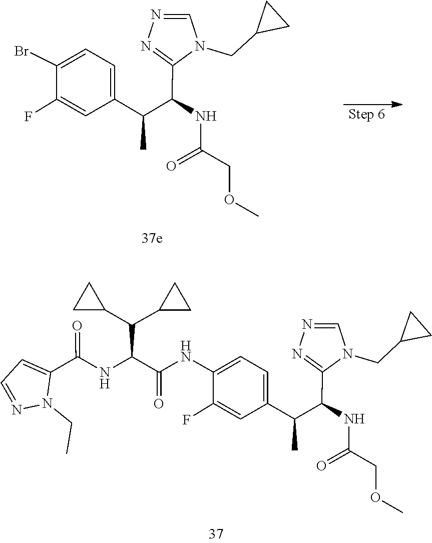 [SMILES: COCC(=O)N[C@H](c1nncn1CC1CC1)[C@@H](C)c1ccc(Br)c(F)c1 ; CCn1nccc1C(=O)N[C@H](C(=O)Nc1ccc([C@H](C)[C@H](NC(=O)COC)c2nncn2CC2CC2)cc1F)C(C1CC1)C1CC1]

### Step 1. (2R,3S)-3-(4-bromo-3-fluorophenyl)-2-(2-methoxyacetamido)butanoic acid (37a)

To a mixture of 3a (1.62 g, 4.5 mmol), THF (10 mL) and water (10 mL) was added LiOH·H₂O (206 mg, 4.9 mmol), which was then stirred for 1 h. The reaction mixture was added with dilute hydrochloric acid (1 N, 20 mL) and extracted with EtOAc (3×30 mL). The combined organic phase was dried over anhydrous Na₂SO₄ and filtered. The filtrate was concentrated to dryness, and the residue was purified by silica gel column chromatography (dichloromethane/methanol=100/0 to 30/1) to give the title compound 37a (1.55 g, 99%).

MS m/z (ESI): 348, 350 [M+1]

### Step 2. Tert-butyl 2-((2S,3S)-3-(4-bromo-3-fluorophenyl)-2-(2-methoxyacetamido)butanoyl)hydrazine-1-carboxylate (37b)

To a mixture of 37a (3.48 g, 10.0 mmol), tert-butyl carbazate (1.98 g, 15.0 mmol), HATU (6.84 g, 18.0 mmol) and DMF (40 mL) was added DIPEA (8.6 mL, 50.0 mmol), which was then stirred for 1 h. The residue was added with EtOAc (300 mL), washed with dilute hydrochloric acid (1 N, 100 mL) and saturated brine (3×100 mL). The organic phase was dried over anhydrous Na₂SO₄ and filtered. The filtrate was concentrated to dryness to give the title compound 37b (4.62 g, 99%). The crude product was directly used in the next step without further purification.

MS m/z (ESI): 462, 464 [M+1]

### Step 3. N-((2S,3S)-3-(4-bromo-3-fluorophenyl)-1-hydrazineyl-1-oxobutan-2-yl)-2-methoxyacetamide hydrochloride (37c)

A mixture of 37b (2.31 g, 5.0 mmol), hydrochloric acid in 1,4-dioxane (4 M, 10 mL) and methanol (10 mL) was stirred for 1 h. The reaction mixture was concentrated to dryness to give the title compound 37c (1.81 g, crude). The crude product was directly used in the next step without further purification.

MS m/z (ESI): 362, 364 [M+1]

### Step 4. N-((2S,3S)-3-(4-bromo-3-fluorophenyl)-1-(2-((E)-(dimethylamino)methylene)hydrazineyl)-1-oxobutan-2-yl)-2-methoxyacetamide (37d)

To a solution of 37c (crude, 1.81 g, 5 mmol) in acetonitrile (10 mL) was added DMF-DMA (1.3 mL, 10 mmol), which was then heated to 80° C. and stirred for 2 h. The crude reaction mixture was directly used in the next step without further purification.

MS m/z (ESI): 416, 418 [M+1]

### Step 5. N-((1S,2S)-2-(4-bromo-3-fluorophenyl)-1-(4-(cyclopropylmethyl)-4H-1,2,4-triazol-3-yl)propyl)-2-methoxyacetamide (37e)

To a reaction mixture from the previous step were added acetic acid (10 mL) and (aminomethyl)cyclopropane (2.2 mL, 25 mmol), which was then heated to 120° C. and stirred overnight. After cooling to room temperature, the reaction mixture was concentrated to dryness. The residue was purified by silica gel column chromatography (dichloromethane/methanol=100/0 to 9/1) and prep-HPLC in sequence to give the title compound 37e (180 mg, 8%).

MS m/z (ESI): 425, 427 [M+1]

### Step 6. N—((S)-1,1-dicyclopropyl-3-((4-((1S,2S)-1-(4-(cyclopropylmethyl)-4H-1,2,4-triazol-3-yl)-1-(2-methoxyacetamido)propan-2-yl)-2-fluorophenyl)amino)-3-oxopropan-2-yl)-1-ethyl-1H-pyrazole-5-carboxamide (37)

A mixture of 37e (85 mg, 0.2 mmol), is (87 mg, 0.3 mmol), Cs₂CO₃ (130 mg, 0.4 mmol), BrettPhos Pd G3 (18 mg, 0.02 mmol) and 1,4-dioxane (1 mL) was heated to 110° C. and stirred for 4 h. After cooling to room temperature, the reaction mixture was concentrated to dryness. The residue was purified by silica gel column chromatography (dichloromethane/methanol=100/0 to 19/1) and prep-HPLC in sequence to give the title compound 37 (43.3 mg, 34%).

MS m/z (ESI): 635 [M+1]

¹H NMR (400 MHz, DMSO-d₆) δ 9.88 (s, 1H), 8.86 (s, 1H), 8.39 (d, J=8.9 Hz, 1H), 8.23 (d, J=8.9 Hz, 1H), 7.76 (dd, J=10.9, 5.7 Hz, 1H), 7.48 (d, J=2.0 Hz, 1H), 7.30 (d, J=12.3 Hz, 1H), 7.16 (d, J=8.2 Hz, 1H), 6.98 (t, J=4.8 Hz, 1H), 5.35-5.29 (m, 2H), 4.94 (s, 1H), 4.53-4.44 (m, 2H), 3.97 (qd, J=14.4, 7.4 Hz, 2H), 3.68-3.46 (m, 3H), 2.97 (s, 3H), 1.29 (t, J=7.1 Hz, 3H), 1.23 (s, 1H), 1.11 (d, J=6.9 Hz, 3H), 0.96-0.87 (m, 1H), 0.85 (dd, J=12.1, 5.6 Hz, 1H), 0.75 (dd, J=17.2, 9.1 Hz, 1H), 0.56 (dd, J=7.8, 4.6 Hz, 2H), 0.45 (dd, J=8.2, 5.4 Hz, 2H), 0.43-0.35 (m, 2H), 0.33-0.26 (m, 2H), 0.26-0.18 (m, 2H), 0.18-0.12 (m, 1H).

### Example 23. N—((S)-1,1-dicyclopropyl-3-((4-((1R,2S)-1-(1-(cyclopropylmethyl)-1H-imidazol-2-yl)-1-(2-methoxyacetamido)propan-2-yl)-2-fluorophenyl)amino)-3-oxopropan-2-yl)-1-ethyl-1H-pyrazole-5-carboxamide (Compound 38)

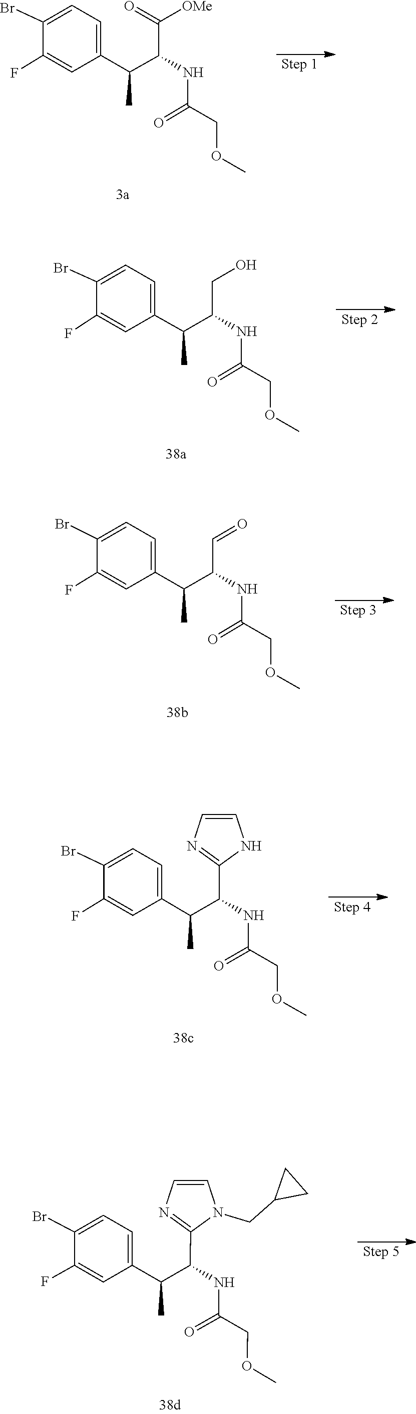 [SMILES: COCC(=O)N[C@@H](C(=O)OC)[C@@H](C)c1ccc(Br)c(F)c1 ; COCC(=O)N[C@@H](CO)[C@@H](C)c1ccc(Br)c(F)c1 ; COCC(=O)N[C@@H](C=O)[C@@H](C)c1ccc(Br)c(F)c1 ; COCC(=O)N[C@@H](c1ncc[nH]1)[C@@H](C)c1ccc(Br)c(F)c1 ; COCC(=O)N[C@@H](c1nccn1CC1CC1)[C@@H](C)c1ccc(Br)c(F)c1]

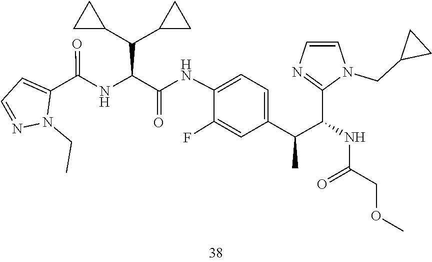 [SMILES: CCn1nccc1C(=O)N[C@H](C(=O)Nc1ccc([C@H](C)[C@@H](NC(=O)COC)c2nccn2CC2CC2)cc1F)C(C1CC1)C1CC1]

### Step 1. N-((2R,3S)-3-(4-bromo-3-fluorophenyl)-1-hydroxybutan-2-yl)-2-methoxyacetamide (38a)

To a solution of 3a (1.5 g, 4.1 mmol) in methanol (50 mL) was added NaBH₄ (1.57 g, 41.4 mmol), which was then stirred for 4 h. The reaction mixture was concentrated to dryness. The residue was added with dichloromethane (150 mL) and washed with a saturated aqueous NH₄Cl solution (50 mL) and saturated brine (50 mL). The organic phase was dried over anhydrous Na₂SO₄ and filtered. The filtrate was purified by prep-HPLC to give the title compound 38a (1.1 g, 80%).

MS m/z (ESI): 334, 336 [M+1]

### Step 2. N-((2R,3S)-3-(4-bromo-3-fluorophenyl)-1-oxobutan-2-yl)-2-methoxyacetamide (38b)

To a solution of 38a (1.1 g, 3.3 mmol) in acetonitrile (40 mL) was added IBX (1.38 g, 4.9 mmol), which was then heated to 80° C. and stirred for 4 h. After cooling to room temperature, the reaction mixture was concentrated to dryness. The residue was purified by silica gel column chromatography (PE/EtOAc=50/1 to 1/1) to give the title compound 38b (600 mg, 55%).

MS m/z (ESI): 332, 334 [M+1]

### Step 3. N-((1R,2S)-2-(4-bromo-3-fluorophenyl)-1-(1H-imidazol-2-yl)propyl)-2-methoxyacetamide (38c)

To a solution of 38b (600 mg, 1.8 mmol) in methanol (20 mL) were added glyoxal (40% aqueous solution, 4 mL) and ammonium hydroxide (25% aqueous solution, 7 mL), which was then stirred for 15 h. The reaction mixture was concentrated to dryness and the residue was purified by prep-HPLC to give the title compound 38c (400 mg, 60%).

MS m/z (ESI): 370, 372 [M+1]

### Step 4. N-((1R,2S)-2-(4-bromo-3-fluorophenyl)-1-(1-(cyclopropylmethyl)-1H-imidazol-2-yl)propyl)-2-methoxyacetamide (38d)

To a mixture of 38c (400 mg, 1.08 mmol), Cs₂CO₃ (1.05 g, 3.24 mmol) and DMF (12 mL) was added (bromomethyl)cyclopropane (365 mg, 2.7 mmol), which was then heated to 45° C. and stirred for 3 h. After cooling to room temperature, the reaction mixture was filtered. The filtrate was purified by prep-HPLC to give the title compound 38d (200 mg, 44%).

MS m/z (ESI): 424, 426 [M+1]

### Step 5. N—((S)-1,1-dicyclopropyl-3-((4-((1R,2S)-1-(1-(cyclopropylmethyl)-1H-imidazol-2-yl)-1-(2-methoxyacetamido)propan-2-yl)-2-fluorophenyl)amino)-3-oxopropan-2-yl)-1-ethyl-1H-pyrazole-5-carboxamide (38)

A mixture of 38d (85 mg, 0.2 mmol), is (87 mg, 0.3 mmol), Cs₂CO₃ (130 mg, 0.4 mmol), BrettPhos Pd G3 (18 mg, 0.02 mmol) and 1,4-dioxane (3 mL) was heated to 110° C. under microwave and stirred for 1.5 h. After cooling to room temperature, the reaction mixture was concentrated to dryness. The residue was purified by prep-HPLC to give the title compound 38 (33 mg, 26%).

MS m/z (ESI): 634 [M+1]

¹H NMR (400 MHz, CD₃OD) δ 7.76-7.68 (m, 1H), 7.50 (d, J=2.1 Hz, 1H), 6.99-6.86 (m, 4H), 6.83 (d, J=2.1 Hz, 1H), 5.25 (d, J=10.9 Hz, 1H), 4.92 (d, J=7.1 Hz, 1H), 4.53 (q, J=7.2 Hz, 2H), 3.94 (q, J=15.2 Hz, 2H), 3.53 (dd, J=14.3, 7.3 Hz, 1H), 3.45-3.35 (m, 5H), 1.50-1.33 (m, 6H), 0.91-0.72 (m, 4H), 0.56-0.15 (m, 12H).

### Example 24. N—((S)-1,1-dicyclopropyl-3-((2-fluoro-4-((1R,2S)-1-(1-(2-methoxyethyl)-1H-1,2,4-triazol-5-yl)-1-propionamidopropan-2-yl)phenyl)amino)-3-oxopropan-2-yl)-1-ethyl-1H-pyrazole-5-carboxamide (Compound 40)

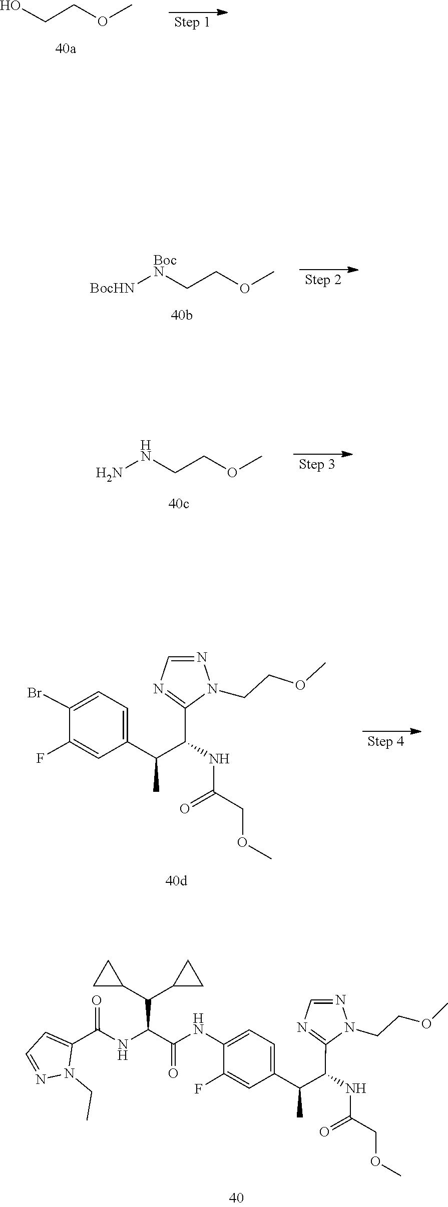

### Step 1. Di-tert-butyl 1-(2-methoxyethyl)hydrazine-1,2-dicarboxylate (40b)

To a mixture of 2-methoxyethan-1-ol 40a (11 g, 150 mmol), di-tert-butyl azodicarboxylate (23 g, 100 mmol) and THF (100 mL) was added PPh₃ (26 g, 100 mmol), which was then stirred for 2 h. The reaction mixture was concentrated to dryness and the residue was purified by silica gel column chromatography (PE/EtOAc=100/0 to 20/1) to give the title compound 40b (29 g, 99%).

### Step 2. (2-Methoxyethyl)hydrazine dihydrochloride (40c)

A solution of 40b (29 g, 100 mmol) in hydrochloric acid in 1,4-dioxane (4 M, 100 mL) was stirred overnight. The reaction mixture was added with EtOAc (100 mL) and filtered to give the title compound 40c (8.0 g, 28%). The crude product was directly used in the next step without further purification.

### Step 3. N-((1R,2S)-2-(4-bromo-3-fluorophenyl)-1-(1-(2-methoxyethyl)-1H-1,2,4-triazol-5-yl)propyl)-2-methoxyacetamide (40d)

To a solution of 19d (250 mg, 0.6 mmol) in acetic acid (5 mL) was added 40c (300 mg, 1.8 mmol), which was then heated to 80° C. and stirred for 2 h. After cooling to room temperature, the reaction mixture was concentrated to dryness. The residue was purified by prep-HPLC to give the title compound 40d (15 mg, 6%).

MS m/z (ESI): 429, 431 [M+1]

### Step 4. N—((S)-1,1-dicyclopropyl-3-((2-fluoro-4-((1R,2S)-1-(1-(2-methoxyethyl)-1H-1,2,4-triazol-5-yl)-1-propionamidopropan-2-yl)phenyl)amino)-3-oxopropan-2-yl)-1-ethyl-1H-pyrazole-5-carboxamide (40)

A mixture of 40d (15 mg, 0.035 mmol), is (11 mg, 0.035 mmol), Cs₂CO₃ (23 mg, 0.07 mmol), BrettPhos Pd G3 (4 mg, 0.0035 mmol) and 1,4-dioxane (0.5 mL) was heated to 110° C. and stirred overnight. After cooling to room temperature, the reaction mixture was purified by prep-HPLC to give the title compound 40 (12.6 mg, 57%).

MS m/z (ESI): 639 [M+1]

¹H NMR (400 MHz, DMSO-d₆) δ 9.82 (s, 1H), 8.36 (dd, J=8.9, 3.1 Hz, 2H), 7.80 (s, 1H), 7.67 (t, J=8.3 Hz, 1H), 7.47 (d, J=2.0 Hz, 1H), 7.06 (d, J=12.2 Hz, 1H), 7.00-6.89 (m, 2H), 5.34 (t, J=9.6 Hz, 1H), 4.91 (t, J=8.1 Hz, 1H), 4.47 (dt, J=9.3, 6.8 Hz, 2H), 4.11 (dtd, J=19.9, 14.4, 5.6 Hz, 2H), 3.86 (s, 2H), 3.61-3.36 (m, 4H), 3.28 (s, 3H), 3.12 (s, 3H), 1.28 (dd, J=13.9, 6.9 Hz, 6H), 1.03-0.64 (m, 2H), 0.54-0.03 (m, 8H).

The following intermediate was prepared according to steps 1 to 4 of Example 24, except that a different reagent was used instead of 2-methoxyethan-1-ol.

| | Reagent replacing 2- | MS m/z |
|---|---|---|
| Intermediate | methoxyethan-1-ol | (ESI) |
| N-((1R,2S)-2-(4-bromo-3-fluorophenyl)-1-(1-(2- | 2- | 442, 444 |
| (dimethylamino)ethyl)-1H-1,2,4-triazol-5-yl)propyl)-2- | (Dimethylamino)ethan- | [M + 1] |
| methoxyacetamide | 1-ol | |

Compound 52 was prepared according to the procedures for Example 24, except that a different reagent was used instead of 40a.

| | Reagent replacing | MS m/z |
|---|---|---|
| Compound | 40a | ESI) |
| N-((S)-1,1-dicyclopropyl-3-((2-fluoro-4-((1R,2S)-1-(2- | 2-(4- | 707 |
| methoxyacetamido)-1-(1-(2-(4-methylpiperazin-1-yl)ethyl)- | Methylpiperazin- | [M + 1] |
| 1H-1,2,4-triazol-5-yl)propan-2-yl)phenyl)amino)-3- | 1-yl)ethan-1-ol | |
| oxopropan-2-yl)-1-ethyl-1H-pyrazole-5-carboxamide (52) | | |

¹H NMR data of Compound 52 are shown below:

| Compound # | ¹H NMR |
|---|---|
| 52 | ¹H NMR (400 MHz, DMSO-d₆) δ 9.82 (s, 1H), 8.47 (d, J = 8.8 Hz, 1H), 8.36 |
| | (d, J = 8.8 Hz, 1H), 7.81 (s, 1H), 7.67 (s, 1H), 7.48 (d, J = 2.0 Hz, 1H), 7.10 |
| | (s, 1H), 6.95 (dd, J = 13.3, 5.6 Hz, 2H), 5.31 (d, J = 10.7 Hz, 1H), 4.90 (s, |
| | 1H), 4.57 (d, J = 7.0 Hz, 1H), 4.47 (d, J = 7.2 Hz, 2H), 4.11 (m, 4H), 3.88 (s, |
| | 2H), 3.30 (s, 3H), 2.00 (d, J = 7.7 Hz, 1H), 1.52 (d, J = 7.0 Hz, 2H), 1.29 (m, |
| | 15H), 0.74 (m, 2H), 0.25 (m, 8H). |

### Example 25. N—((S)-2-((4-((1R,2S)-1-(1-(cyclopropylmethyl)-1H-1,2,4-triazol-5-yl)-1-(2-methoxyacetamido)propan-2-yl)-2-fluorophenyl)amino)-1-((1R,4S)-4-methylcyclohexyl)-2-oxoethyl)-1-ethyl-1H-pyrazole-5-carboxamide (Compound 41)

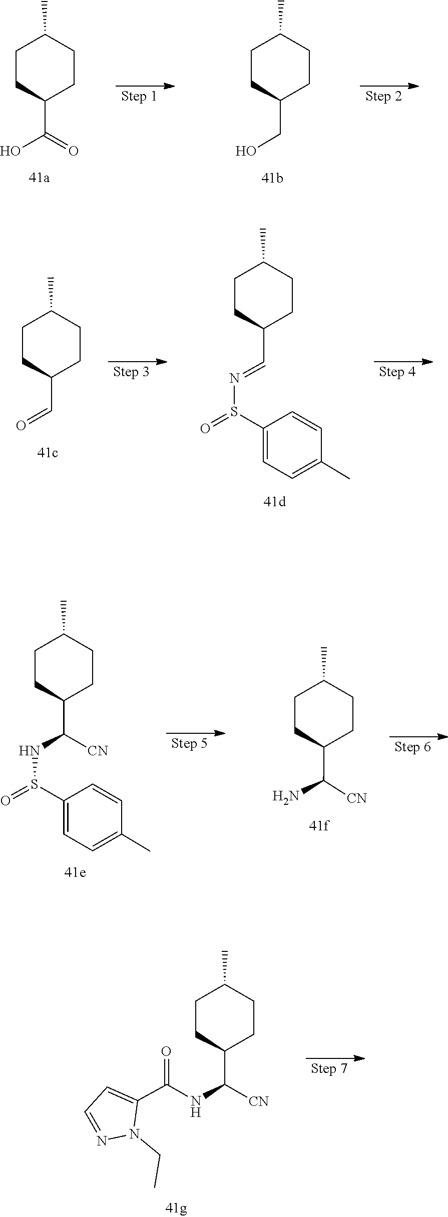

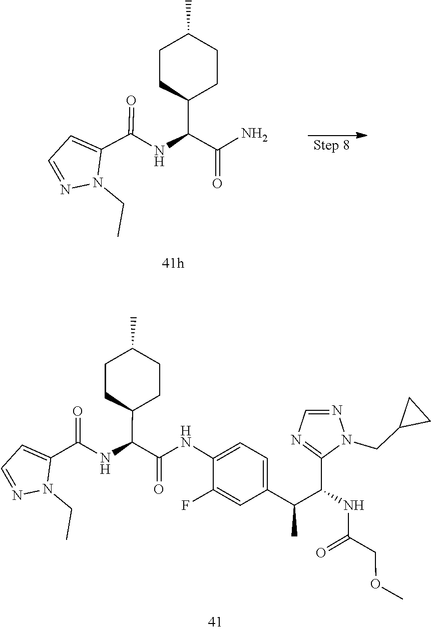 [SMILES: CCn1nccc1C(=O)N[C@H](C(N)=O)[C@H]1CC[C@H](C)CC1 ; CCn1nccc1C(=O)N[C@H](C(=O)Nc1ccc([C@H](C)[C@@H](NC(=O)COC)c2ncnn2CC2CC2)cc1F)[C@H]1CC[C@H](C)CC1]

### Step 1. ((1R,4R)-4-methylcyclohexyl)methanol (41b)

To a solution of (1R,4R)-4-methylcyclohexane-1-carboxylic acid 41a (8.5 g, 60 mmol) in THF (100 mL) at 0° C. was added LiAlH₄ (2.3 g, 60 mmol), which was then stirred for 15 h. The mixture was added with Na₂SO₄ 10H₂O (96.6 g) and water (3 mL), which was then stirred for 30 min. The reaction mixture was filtered and the filtrate was concentrated to dryness. The residue was purified by silica gel column chromatography (PE/EtOAc=100/0 to 5/1) to give the title compound 41b (5.0 g, 65%).

¹H NMR (400 MHz, CDCl₃) δ 3.41 (d, J=6.4 Hz, 2H), 2.32 (d, J=6.6 Hz, 1H), 1.83-1.65 (m, 4H), 1.47-1.22 (m, 2H), 1.00-0.82 (m, 7H).

### Step 2. (1R,4R)-4-methylcyclohexane-1-carbaldehyde (41c)

To a solution of 41b (4.8 g, 37.4 mmol) in dichloromethane (100 mL) at 0° C. was added Dess-Martin periodinane (23.8 g, 56.2 mmol), which was then stirred for 4 h. The mixture was concentrated to dryness and the residue was purified by silica gel column chromatography (PE/EtOAc=100/0 to 5/1) to give the title compound 41c (1.7 g, 36%).

¹H NMR (400 MHz, CDCl₃) δ 9.61 (d, J=1.6 Hz, 1H), 2.03-1.89 (m, 2H), 1.87-1.75 (m, 2H), 1.29-1.25 (m, 2H), 1.04-0.83 (m, 7H).

### Step 3. (S)-4-methyl-N-((E)-((1R,4S)-4-methylcyclohexyl)methylene)benzenesulfinamide (41d)

To a mixture of 41c (1.7 g, 13.5 mmol), (S)-4-methylbenzenesulfenamide (1.4 g, 9.0 mmol) and THF (10 mL) at 0° C. was added Ti(OEt)₄ (6.2 g, 27.0 mmol), which was then warmed to room temperature and stirred overnight. The reaction mixture was added with saturated brine (100 mL) and filtered. The filtrate was extracted with EtOAc (100 mL) and the organic phase was concentrated to dryness. The residue was purified by silica gel column chromatography (PE/EtOAc=100/0 to 5/1) to give the title compound 41d (1.7 g, 48%).

MS m/z (ESI): 264 [M+1]

### Step 4. (S)—N—((S)-cyano((1R,4S)-4-methylcyclohexyl)methyl)-4-methylbenzenesulfinamide (41e)

To a solution of 41d (1.70 g, 6.45 mmol) in hexanes (70 mL) at −40° C. were added trimethylsilyl cyanide (768 mg, 7.74 mmol) and CsF (1.18 g, 7.74 mmol), which was then stirred at −40° C. overnight. The reaction mixture was added with water (200 mL) and extracted with EtOAc (200 mL). The organic phase was concentrated to dryness and the residue was purified by silica gel column chromatography (PE/EtOAc=100/0 to 15/1) to give the title compound 41e (1.00 g, 53%).

### Step 5. (S)-2-amino-2-((1R,4S)-4-methylcyclohexyl)acetonitrile hydrochloride (41f)

A mixture of 41e (1.0 g, 3.4 mmol), hydrochloric acid in 1,4-dioxane (4 M, 5 mL) and methanol (20 mL) was stirred for 2 h. The reaction mixture was concentrated to dryness to give the title compound 41f (700 mg, crude). The crude product was directly used in the next step without further purification.

MS m/z (ESI): 153 [M+1]

### Step 6. N—((S-cyano((1R,4S)-4-methylcyclohexyl)methyl)-1-ethyl-1H-pyrazole-5-carboxamide (41g)

To a mixture of 41f (crude, 700 mg, 3.44 mmol), 1-ethyl-1H-pyrazole-5-carboxylic acid (964 mg, 6.88 mmol), HATU (1.96 g, 5.16 mmol) and DMF (10 mL) was added DIPEA (1.78 g, 13.76 mmol), which was then stirred for 1 h. The reaction mixture was purified by prep-HPLC to give the title compound 41g (640 mg, 68%).

MS m/z (ESI): 275 [M+1]

### Step 7. N—((S)-2-amino-1-((1R,4S)-4-methylcyclohexyl)-2-oxoethyl)-1-ethyl-1H-pyrazole-5-carboxamide (41h)

To a mixture of 41g (640 mg, 2.33 mmol), hydrogen peroxide (30% aqueous solution, 6 mL) and DMSO (20 mL) at 0° C. was added NaOH (19 mg, 0.47 mmol), which was then warmed to room temperature and stirred for 1 h. The reaction mixture was added with a saturated aqueous NH₄Cl solution (50 mL) and extracted with EtOAc (200 mL). The organic phase was concentrated to dryness and the residue was purified by silica gel column chromatography (dichloromethane/methanol=100/0 to 10/1) to give the title compound 41h (400 mg, 59%).

MS m/z (ESI): 293 [M+1]

### Step 8. N—((S)-2-((4-((1R,2S)-1-(1-(cyclopropylmethyl)-1H-1,2,4-triazol-5-yl)-1-(2-methoxyacetamido)propan-2-yl)-2-fluorophenyl)amino)-1-((1R,4S)-4-methylcyclohexyl)-2-oxoethyl)-1-ethyl-1H-pyrazole-5-carboxamide (41)

A mixture of 19e (21 mg, 0.05 mmol), 41h (29 mg, 0.1 mmol), Cs₂CO₃ (33 mg, 0.1 mmol), BrettPhos Pd G3 (11 mg, 0.013 mmol) and 1,4-dioxane (3 mL) was heated to 110° C. in microwave and stirred for 2 h. After cooling to room temperature, the reaction mixture was filtered. The filtrate was concentrated to dryness and the residue was purified by prep-HPLC to give the title compound 41 (9.8 mg, 31%).

MS m/z (ESI): 637 [M+1]

¹H NMR (400 MHz, CD₃OD) δ 8.38-8.21 (m, 1H), 7.84 (s, 1H), 7.72-7.62 (m, 1H), 7.48 (d, J=2.1 Hz, 1H), 7.05-6.91 (m, 1H), 6.84 (d, J=2.1 Hz, 1H), 5.40-5.30 (m, 1H), 4.57-4.41 (m, 3H), 3.95 (q, J=15.2 Hz, 2H), 3.79 (dd, J=14.4, 6.8 Hz, 1H), 3.69 (dd, J=14.4, 7.6 Hz, 1H), 3.54-3.35 (m, 4H), 1.93-1.69 (m, 5H), 1.46-1.12 (m, 9H), 1.02-0.77 (m, 6H), 0.48-0.20 (m, 4H).

Compound 42 was prepared according to the procedures of Example 25, except that a different reagent was used instead of ((1R,4R)-4-methylcyclohexyl)methanol.

| | | MS |
|---|---|---|
| | Reagent replacing ((1r,4r)-4- | m/z |
| Compound | methylcyclohexyl)methanol | (ESI) |
| N-((S)-1-cycloheptyl-2-((4-((1R,2S)-1-(1- | Cycloheptylmethanol | 637 |
| (cyclopropylmethyl)-1H-1,2,4-triazol-5-yl)-1-(2- | | [M + 1] |
| methoxyacetamido)propan-2-yl)-2- | | |
| fluorophenyl)amino)-2-oxoethyl)-1-ethyl-1H- | | |
| pyrazole-5-carboxamide (42) | | |

¹H NMR data of Compound 42 are shown below:

| Compound # | ¹H NMR |
|---|---|
| 42 | ¹H NMR (400 MHz, CD₃OD) δ 7.84 (s, 1H), 7.67 (td, J = 8.2, 5.0 Hz, 1H), |
| | 7.48 (d, J = 2.1 Hz, 1H), 7.03-6.91 (m, 2H), 6.82 (d, J = 2.1 Hz, 1H), 5.35 |
| | (d, J = 10.9 Hz, 1H), 4.53 (dt, J = 14.4, 7.7 Hz, 3H), 3.95 (q, J = 15.2 Hz, |
| | 2H), 3.79 (dd, J = 14.4, 6.8 Hz, 1H), 3.69 (dd, J = 14.4, 7.6 Hz, 1H), |
| | 3.53-3.37 (m, 4H), 2.12 (dd, J = 10.8, 6.4 Hz, 1H), 1.86-1.69 (m, 4H), 1.66-1.31 |
| | (m, 14H), 0.89 (d, J = 7.3 Hz, 1H), 0.48-0.21 (m, 4H). |

### Example 26. N—((S)-1,1-dicyclopropyl-3-((4-((1R,2S)-1-(1-(cyclopropylmethyl)-1H-1,2,4-triazol-5-yl)-1-(2-methoxyacetamido)propan-2-yl)-2-fluorophenyl)amino)-3-oxopropan-2-yl)-1-ethyl-1H-1,2,4-triazole-5-carboxamide (Compound 50)

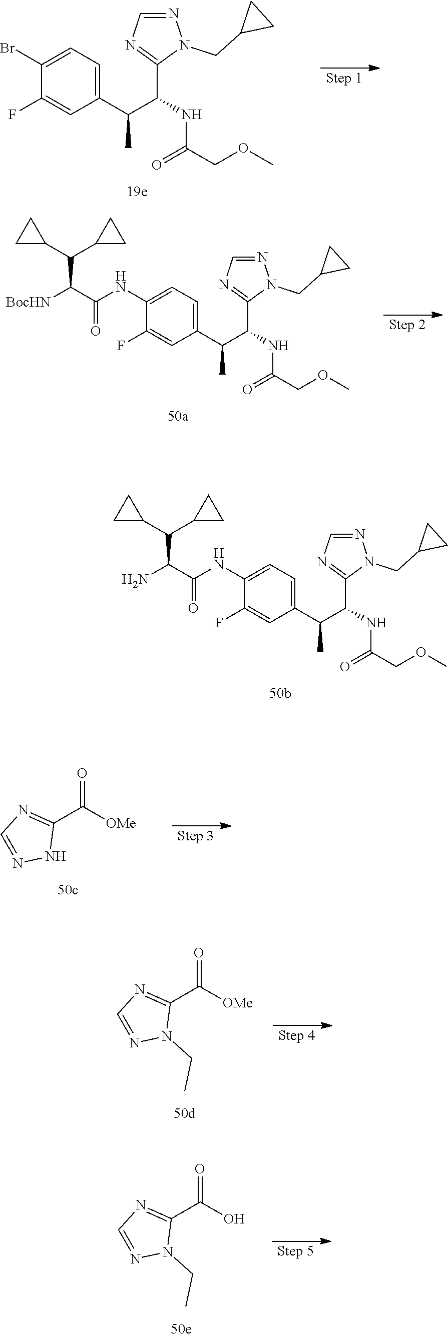

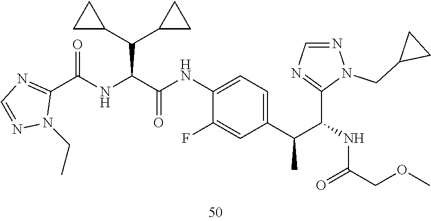 [SMILES: CCn1ncnc1C(=O)N[C@H](C(=O)Nc1ccc([C@H](C)[C@@H](NC(=O)COC)c2ncnn2CC2CC2)cc1F)C(C1CC1)C1CC1]

### Step 1. Tert-butyl ((S)-1,1-dicyclopropyl-3-((4-((1R,2S)-1-(1-(cyclopropylmethyl)-1H-1,2,4-triazol-5-yl)-1-(2-methoxyacetamido)propan-2-yl)-2-fluorophenyl)amino)-3-oxopropan-2-yl)carbamate (50a)

A mixture of 19e (64 mg, 0.15 mmol), 7d (60 mg, 0.23 mmol), Cs₂CO₃ (98 mg, 0.3 mmol), BrettPhos Pd G3 (27 mg, 0.03 mmol) and 1,4-dioxane (3 mL) was heated to 110° C. under microwave and stirred for 2 h. After cooling to room temperature, the reaction mixture was filtered and the filtrate was purified by silica gel column chromatography (dichloromethane/methanol=100/0 to 15/1) to give the title compound 50a (75 mg, 82%).

MS m/z (ESI): 613 [M+1]

### Step 2. (S)-2-amino-3,3-dicyclopropyl-N-(4-((1R,2S)-1-(1-(cyclopropylmethyl)-1H-1,2,4-triazol-5-yl)-1-(2-methoxyacetamido)propan-2-yl)-2-fluorophenyl)propenamide hydrochloride (50b)

A mixture of 50a (75 mg, 0.12 mmol), a solution of hydrochloric acid in 1,4-dioxane (4 M, 3 mL) and methanol (3 mL) was stirred for 2 h. The reaction mixture was concentrated to dryness to give the title compound 50b (60 mg, crude). The crude product was directly used in the next step without further purification.

MS m/z (ESI): 513 [M+1]

### Step 3. Methyl 1-ethyl-1H-1,2,4-triazole-5-carboxylate (50d)

To a mixture of methyl 1H-1,2,4-triazole-5-carboxylate 50c (6.36 g, 50 mmol), bromoethane (8.17 g, 75 mmol) and DMF (30 mL) was added K₂CO₃ (13.80 g, 100 mmol), which was then heated to 50° C. and stirred overnight. After cooling to room temperature, the reaction mixture was filtered, and the filtrate was purified by prep-HPLC to give the title compound 50d (1.0 g, 13%).

MS m/z (ESI): 156 [M+1]

### Step 4. 1-Ethyl-1H-1,2,4-triazole-5-carboxylic acid (50e)

To a solution of 50b (1.0 g, 6.4 mmol) in THF (10 mL) and water (10 mL) was added LiOH·H₂O (515 mg, 12.8 mmol), which was then stirred for 4 h. The reaction mixture was added with dilute hydrochloric acid (1 N, 20 mL) and the resulting mixture was concentrated to dryness to give the title compound 50e (1.4 g, crude). The crude product was directly used in the next step without further purification.

MS m/z (ESI): 142 [M+1]

### Step 5. N—((S)-1,1-dicyclopropyl-3-((4-((1R,2S)-1-(1-(cyclopropylmethyl)-1H-1,2,4-triazol-5-yl)-1-(2-methoxyacetamido)propan-2-yl)-2-fluorophenyl)amino)-3-oxopropan-2-yl)-1-ethyl-1H-1,2,4-triazole-5-carboxamide (50)

To a mixture of 50b (60 mg, 0.12 mmol), 50e (34 mg, 0.24 mmol), HATU (68 mg, 0.18 mmol) and DMF (3 mL) was added DIPEA (62 mg, 0.48 mmol), which was then stirred for 1 h. The reaction mixture was purified by prep-HPLC to give the title compound 50 (20.8 mg, 27%).

MS m/z (ESI): 636 [M+1]

¹H NMR (400 MHz, CD₃OD) δ 7.96 (s, 1H), 7.90 (s, 1H), 7.71 (t, J=8.3 Hz, 1H), 6.99 (dd, J=14.0, 4.1 Hz, 2H), 5.36 (d, J=11.0 Hz, 1H), 4.91 (d, J=6.0 Hz, 1H), 4.67 (q, J=7.1 Hz, 2H), 3.95 (q, J=15.2 Hz, 2H), 3.80 (dd, J=14.4, 6.8 Hz, 1H), 3.71 (dd, J=14.4, 7.6 Hz, 1H), 3.52-3.38 (m, 4H), 1.50-1.36 (m, 6H), 0.98-0.76 (m, 4H), 0.58-0.18 (m, 12H).

### Example 27. N—((S)-1-((4-((1R,2S)-1-(2-(azetidin-1-yl)acetamido)-1-(1-(cyclopropylmethyl)-1H-1,2,4-triazol-5-yl)propan-2-yl)-2-fluorophenyl)amino)-3,3-dicyclopropyl-1-oxopropan-2-yl)-1-ethyl-1H-pyrazole-5-carboxamide (Compound 51)

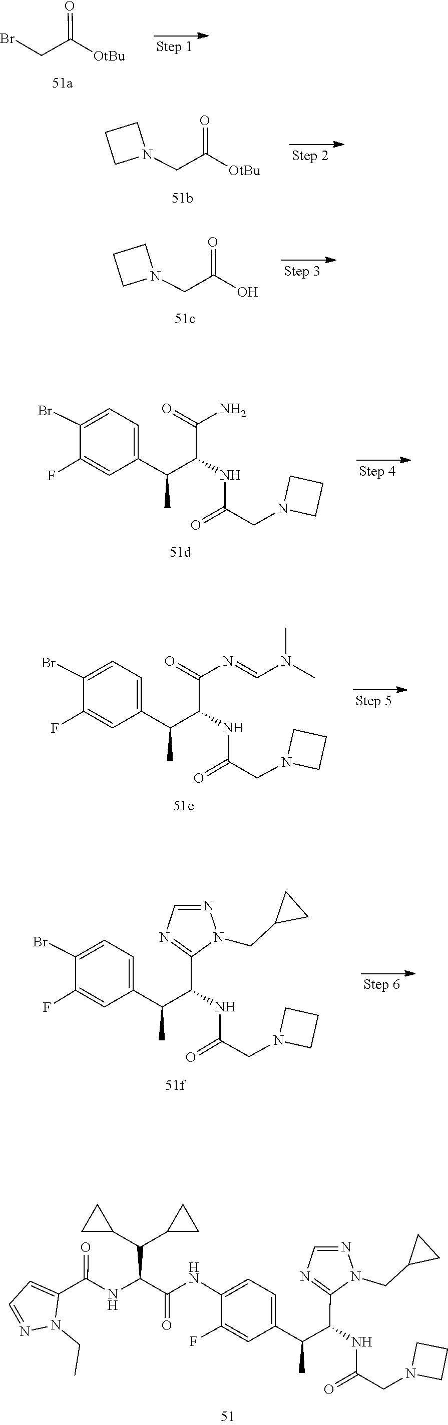

### Step 1. Tert-butyl 2-(azetidin-1-yl)acetate (51b)

To a mixture of tert-butyl 2-bromoacetate 51a (3.0 g, 32 mmol), azetidine hydrochloride (6.3 g, 32 mmol) and THF (100 mL) at 0° C. was added an aqueous NaOH solution (2 M, 30 mL), which was stirred for 1 h. The reaction mixture was added with saturated brine (100 mL) and extracted with dichloromethane (3×100 mL). The combined organic phase was dried over anhydrous Na₂SO₄ and filtered. The filtrate was concentrated to dryness to give the title compound 51b (5.0 g, 99%). The crude product was directly used in the next step without further purification.

MS m/z (ESI): 172 [M+1]

### Step 2. 2-(Azetidin-1-yl)acetic acid hydrochloride (51c)

A mixture of 51b (5.0 g, 30 mmol) and a solution of hydrochloric acid in 1,4-dioxane (4 M, 100 mL) was stirred overnight. The reaction mixture was filtered to give the title compound 51c (3.0 g, 66%). The crude product was directly used in the next step without further purification.

MS m/z (ESI): 116 [M+1]

### Step 3. (2R,3S)-2-(2-(azetidin-1-yl)acetamido)-3-(4-bromo-3-fluorophenyl)butanamide (51d)

To a mixture of 19b (500 mg, 1.8 mmol), 51c (545 mg, 3.6 mmol), HATU (1.3 g, 3.6 mmol) and DMF (10 mL) was added DIPEA (1.2 g, 9 mmol), which was then stirred for 1 h. The reaction mixture was added with saturated aqueous NH₄Cl (100 mL) and extracted with EtOAc (100 mL). The organic phase was concentrated to dryness and the residue was purified by silica gel column chromatography (dichloromethane/methanol=100/0 to 1/1) to give the title compound 51d (600 mg, 90%).

MS m/z (ESI): 372, 374 [M+1]

### Step 4. (2R,3S)-2-(2-(azetidin-1-yl)acetamido)-3-(4-bromo-3-fluorophenyl)-N-((E)-(dimethylamino)methylene)butanamide (51e)

A solution of 51d (600 mg, 1.8 mmol) in DMF-DMA (0.5 mL) was heated to 80° C. and stirred for 1 h. After cooling to room temperature, the reaction mixture was concentrated to dryness to give the title compound 51e (770 mg, crude). The crude product was directly used in the next step without further purification.

MS m/z (ESI): 427, 429 [M+1]

### Step 5. 2-(Azetidin-1-yl)-N-((1R,2S)-2-(4-bromo-3-fluorophenyl)-1-(1-(cyclopropylmethyl)-1H-1,2,4-triazol-5-yl)propyl)acetamide (51f)

To a solution of 51e (770 mg, 1.8 mmol) in acetic acid (10 mL) was added (cyclopropylmethyl)hydrazine dihydrochloride (633 mg, 3.6 mmol), which was then heated to 70° C. and stirred overnight. After cooling to room temperature, the reaction mixture was concentrated to dryness and the residue was purified by prep-HPLC to give the title compound 51f (90 mg, 11%).

MS m/z (ESI): 450, 452 [M+1]

### Step 6. N—((S)-1-((4-((1R,2S)-1-(2-(azetidin-1-yl)acetamido)-1-(1-(cyclopropylmethyl)-1H-1,2,4-triazol-5-yl)propan-2-yl)-2-fluorophenyl)amino)-3,3-dicyclopropyl-1-oxopropan-2-yl)-1-ethyl-1H-pyrazole-5-carboxamide (51)

A mixture of 51f (45 mg, 0.1 mmol), is (32 mg, 0.11 mmol), Cs₂CO₃ (65 mg, 0.2 mmol), BrettPhos Pd G3 (9 mg, 0.01 mmol) and 1,4-dioxane (0.5 mL) was heated to 110° C. and stirred for 4 h. After cooling to room temperature, the reaction mixture was filtered, and the filtrate was purified by prep-HPLC to give the title compound 51 (1.9 mg, 2%).

MS m/z (ESI): 660 [M+1]

¹H NMR (400 MHz, CD₃OD) δ 7.84 (s, 1H), 7.80-7.69 (m, 1H), 7.50 (s, 1H), 7.08-6.91 (m, 2H), 6.84 (s, 1H), 5.29 (d, J=10.8 Hz, 1H), 4.92 (d, J=6.9 Hz, 1H), 4.53 (dd, J=14.2, 7.0 Hz, 2H), 3.78 (dd, J=14.3, 6.6 Hz, 1H), 3.67 (dd, J=14.4, 7.6 Hz, 1H), 3.56 (d, J=27.6 Hz, 4H), 3.46-3.35 (m, 3H), 2.41-2.11 (m, 2H), 1.43 (d, J=6.8 Hz, 2H), 1.38 (t, J=7.1 Hz, 4H), 0.99-0.71 (m, 5H), 0.60-0.10 (m, 11H).

### Example 28. N—((S)-1-((4-((1R,2S)-1-(2-(azetidin-3-yloxy)acetamido)-1-(1-(cyclopropylmethyl)-1H-1,2,4-triazol-5-yl)propan-2-yl)-2-fluorophenyl)amino)-3,3-dicyclopropyl-1-oxopropan-2-yl)-4-methyl-1,2,5-oxadiazole-3-carboxamide (Compound 55)

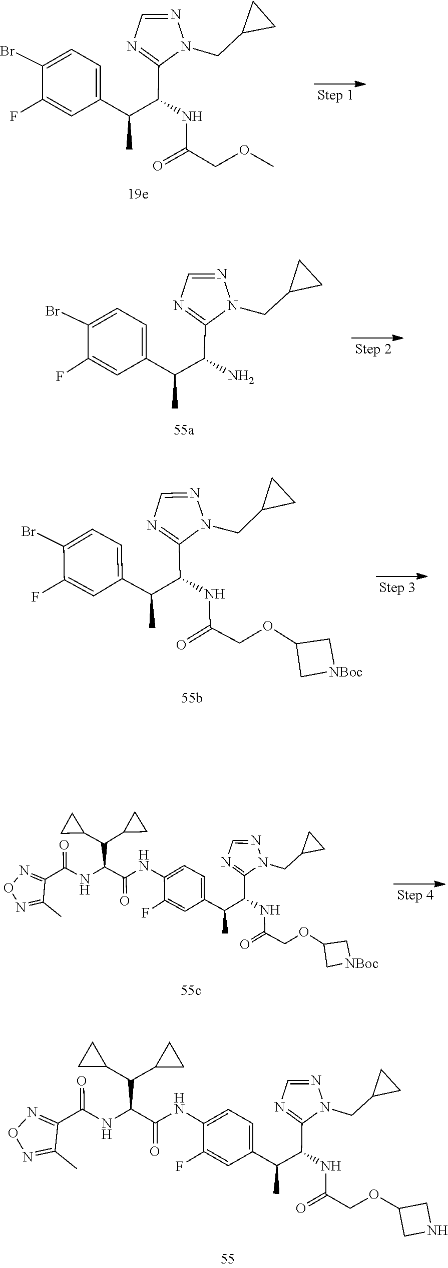 [SMILES: COCC(=O)N[C@@H](c1ncnn1CC1CC1)[C@@H](C)c1ccc(Br)c(F)c1 ; C[C@@H](c1ccc(Br)c(F)c1)[C@@H](N)c1ncnn1CC1CC1 ; C[C@@H](c1ccc(Br)c(F)c1)[C@@H](NC(=O)COC1CN(C(=O)OC(C)(C)C)C1)c1ncnn1CC1CC1 ; Cc1nonc1C(=O)N[C@H](C(=O)Nc1ccc([C@H](C)[C@@H](NC(=O)COC2CN(C(=O)OC(C)(C)C)C2)c2ncnn2CC2CC2)cc1F)C(C1CC1)C1CC1 ; Cc1nonc1C(=O)N[C@H](C(=O)Nc1ccc([C@H](C)[C@@H](NC(=O)COC2CNC2)c2ncnn2CC2CC2)cc1F)C(C1CC1)C1CC1]

### Step 1. (1R,2S)-2-(4-bromo-3-fluorophenyl)-1-(1-(cyclopropylmethyl)-1H-1,2,4-triazol-5-yl)propan-1-amine (55a)

A solution of 19e (2.1 g, 5 mmol) in dilute hydrochloric acid (6 N, 20 mL) was heated to 80° C. and stirred for 3 h. After cooling to room temperature, the reaction mixture was added with a saturated aqueous NaHCO₃ solution (200 mL) and extracted with dichloromethane (3×100 mL). The combined organic phase was dried over anhydrous Na₂SO₄ and filtered. The filtrate was concentrated to dryness to give the title compound 55a (1.8 g, 99%). The crude product was directly used in the next step without further purification.

MS m/z (ESI): 353, 355 [M+1]

### Step 2. Tert-butyl 3-(2-(((1R,2S)-2-(4-bromo-3-fluorophenyl)-1-(1-(cyclopropylmethyl)-1H-1,2,4-triazol-5-yl)propyl)amino)-2-oxoethoxy)azetidine-1-carboxylate (55b)

To a mixture of 55a (88 mg, 0.25 mmol), 2-((1-(tert-butoxycarbonyl)azetidin-3-yl)oxy)acetic acid (58 mg, 0.25 mmol), DMTMM·BF₄ (98 mg, 0.30 mmol) and acetonitrile (1 mL) was added TEA (1.7 mL, 12.5 mmol), which was then stirred for 1 h. The reaction mixture was concentrated to dryness. The residue was added with EtOAc (20 mL) then washed with dilute hydrochloric acid (2 N, 20 mL) and saturated brine (20 mL). The organic phase was dried over anhydrous Na₂SO₄ and filtered. The filtrate was concentrated to dryness and the residue was purified by silica gel column chromatography (dichloromethane/methanol=100/0 to 19/1) to give the title compound 55b (65 mg, 46%).

MS m/z (ESI): 566, 568 [M+1]

### Step 3. Tert-butyl 3-(2-(((1R,2S)-1-(1-(cyclopropylmethyl)-1H-1,2,4-triazol-5-yl)-2-(4-((S)-3,3-dicyclopropyl-2-(4-methyl-1,2,5-oxadiazole-3-carboxamido)propanamido)-3-fluorophenyl)propyl)amino)-2-oxoethoxy)azetidine-1-carboxylate (55c)

A mixture of 55b (57 mg, 0.1 mmol), (S)—N-(1-amino-3,3-dicyclopropyl-1-oxopropan-2-yl)-4-methyl-1,2,5-oxadiazole-3-carboxamide (28 mg, 0.1 mmol), Cs₂CO₃ (65 mg, 0.2 mmol), CuI (5.7 mg, 0.03 mmol), trans-N,N′-dimethylcyclohexane-1,2-diamine (8.5 mg, 0.06 mmol) and toluene (0.5 mL) was heated to 100° C. and stirred for 4 h. After cooling to room temperature, the reaction mixture was purified by silica gel column chromatography (dichloromethane/methanol=100/0 to 9/1) to give the title compound 55c (76 mg, 99%).

MS m/z (ESI): 764 [M+1]

### Step 4. N—((S)-1-((4-((1R,2S)-1-(2-(azetidin-3-yloxy)acetamido)-1-(1-(cyclopropylmethyl)-1H-1,2,4-triazol-5-yl)propan-2-yl)-2-fluorophenyl)amino)-3,3-dicyclopropyl-1-oxopropan-2-yl)-4-methyl-1,2,5-oxadiazole-3-carboxamide (55)

A mixture of 55c (76 mg, 0.1 mmol) and a solution of hydrochloric acid in 1,4-dioxane (4 M, 1 mL) was stirred for 2 h. The reaction mixture was concentrated to dryness and the residue was purified by prep-HPLC to give the title compound 55 (9 mg, 14%).

MS m/z (ESI): 664 [M+1]

¹H NMR (400 MHz, DMSO-d₆) δ 9.87 (s, 1H), 8.97 (s, 1H), 8.46 (d, J=8.2 Hz, 1H), 7.80 (s, 1H), 7.64 (s, 1H), 7.05 (d, J=11.4 Hz, 1H), 6.96 (d, J=7.2 Hz, 1H), 5.25 (s, 1H), 4.97 (s, 1H), 4.27 (s, 1H), 3.86 (s, 2H), 3.75 (d, J=7.0 Hz, 2H), 3.52-3.41 (m, 6H), 2.48 (d, J=3.4 Hz, 3H), 1.36-1.30 (m, 3H), 0.93 (d, J=7.3 Hz, 1H), 0.86 (d, J=4.2 Hz, 1H), 0.79 (d, J=4.8 Hz, 1H), 0.77-0.68 (m, 1H), 0.46 (d, J=7.3 Hz, 1H), 0.42-0.34 (m, 2H), 0.30 (d, J=8.9 Hz, 2H), 0.21 (dd, J=16.8, 11.7 Hz, 7H).

Compounds 57, 58, 60, 63, 64, 65, 66, 67, 70 and 75 were prepared according to steps 1 to 3 of the procedures for Example 28, except that different reagents were used instead of 2-((1-(tert-butoxycarbonyl)azetidin-3-yl)oxy)acetic acid.

| | Reagent replacing 2-((1-(tert- | MS |
|---|---|---|
| | butoxycarbonyl)azetidin-3- | m/z |
| Compound | yl)oxy)acetic acid | (ESI) |
| N-((S)-1,1-dicyclopropyl-3-((4-((1R,2S)-1-(1- | 2-Hydroxyacetic acid | 609 |
| (cyclopropylmethyl)-1H-1,2,4-triazol-5-yl)-1-(2- | | [M + 1] |
| hydroxyacetamido)propan-2-yl)-2- | | |
| fluorophenyl)amino)-3-oxopropan-2-yl)-4-methyl- | | |
| 1,2,5-oxadiazole-3-carboxamide (57) | | |
| N-((S)-1,1-dicyclopropyl-3-((4-((1R,2S)-1-(1- | 2-(Methoxy-d₃)acetic acid | 626 |
| (cyclopropylmethyl)-1H-1,2,4-triazol-5-yl)-1-(2- | | [M + 1] |
| (methoxy-d₃)acetamido)propan-2-yl)-2- | | |
| fluorophenyl)amino)-3-oxopropan-2-yl)-4-methyl- | | |
| 1,2,5-oxadiazole-3-carboxamide (58) | | |
| N-((S)-1-((4-((1R,2S)-1-(2- | 2-Cyclopropoxyacetic acid | 649 |
| cyclopropoxyacetamido)-1-(1- | | [M + 1] |
| (cyclopropylmethyl)-1H-1,2,4-triazol-5-yl)propan- | | |
| 2-yl)-2-fluorophenyl)amino)-3,3-dicyclopropyl-1- | | |
| oxopropan-2-yl)-4-methyl-1,2,5-oxadiazole-3- | | |
| carboxamide (60) | | |
| N-((S)-1,1-dicyclopropyl-3-((4-((1R,2S)-1-(1- | 2-Ethoxyacetic acid | 637 |
| (cyclopropylmethyl)-1H-1,2,4-triazol-5-yl)-1-(2- | | [M + 1] |
| ethoxyacetamido)propan-2-yl)-2- | | |
| fluorophenyl)amino)-3-oxopropan-2-yl)-4-methyl- | | |
| 1,2,5-oxadiazole-3-carboxamide (63) | | |
| N-((S)-1,1-dicyclopropyl-3-((4-((1R,2S)-1-(1- | 2-(Trifluoromethoxy)acetic acid | 677 |
| (cyclopropylmethyl)-1H-1,2,4-triazol-5-yl)-1-(2- | | [M + 1] |
| (trifluoromethoxy)acetamido)propan-2-yl)-2- | | |
| fluorophenyl)amino)-3-oxopropan-2-yl)-4-methyl- | | |
| 1,2,5-oxadiazole-3-carboxamide (64) | | |
| N-((S)-1,1-dicyclopropyl-3-((4-((1R,2S)-1-(1- | 2-(Difluoromethoxy)acetic acid | 659 |
| (cyclopropylmethyl)-1H-1,2,4-triazol-5-yl)-1-(2- | | [M + 1] |
| (difluoromethoxy)acetamido)propan-2-yl)-2- | | |
| fluorophenyl)amino)-3-oxopropan-2-yl)-4-methyl- | | |
| 1,2,5-oxadiazole-3-carboxamide (65) | | |
| N-((S)-1,1-dicyclopropyl-3-((4-((1R,2S)-1-(1- | Propionic acid | 607 |
| (cyclopropylmethyl)-1H-1,2,4-triazol-5-yl)-1- | | [M + 1] |
| propionamidopropan-2-yl)-2-fluorophenyl)amino)- | | |
| 3-oxopropan-2-yl)-4-methyl-1,2,5-oxadiazole-3- | | |
| carboxamide (66) | | |
| N-((S)-1-((4-((1R,2S)-1-acetamido-1-(1- | Acetic acid | 593 |
| (cyclopropylmethyl)-1H-1,2,4-triazol-5-yl)propan- | | [M + 1] |
| 2-yl)-2-fluorophenyl)amino)-3,3-dicyclopropyl-1- | | |
| oxopropan-2-yl)-4-methyl-1,2,5-oxadiazole-3- | | |
| carboxamide (67) | | |
| N-((S)-1-((4-((1R,2S)-1-(2-cyanoacetamido)-1-(1- | 2-Cyanoacetic acid | 618 |
| (cyclopropylmethyl)-1H-1,2,4-triazol-5-yl)propan- | | [M + 1] |
| 2-yl)-2-fluorophenyl)amino)-3,3-dicyclopropyl-1- | | |
| oxopropan-2-yl)-4-methyl-1,2,5-oxadiazole-3- | | |
| carboxamide (70) | | |
| N-((S)-1,1-dicyclopropyl-3-((4-((1R,2S)-1-(1- | Propanoic-d₅ acid | 612 |
| (cyclopropylmethyl)-1H-1,2,4-triazol-5-yl)-1- | | [M + 1] |
| (propanamido-2,2,3,3,3-d₅)propan-2-yl)-2- | | |
| fluorophenyl)amino)-3-oxopropan-2-yl)-4-methyl- | | |
| 1,2,5-oxadiazole-3-carboxamide (75) | | |

Compounds 61 and 76 were prepared according to step 3 of Example 28, except that different reagents were used instead of 55b.

| | | MS |
|---|---|---|
| | | m/z |
| Compound | Reagent replacing 55b | (ESI) |
| N-((S)-1,1-dicyclopropyl-3-((4-((1R,2S)-1-(1-(2- | N-((1R,2S)-2-(4-bromo-3- | 640 |
| (dimethylamino)ethyl)-1H-1,2,4-triazol-5-yl)-1-(2- | fluorophenyl)-1-(1-(2- | [M + 1] |
| methoxyacetamido)propan-2-yl)-2- | (dimethylamino)ethyl)-1H- | |
| fluorophenyl)amino)-3-oxopropan-2-yl)-4-methyl- | 1,2,4-triazol-5-yl)propyl)- | |
| 1,2,5-oxadiazole-3-carboxamide (61) | 2-methoxyacetamide | |
| N-((S)-1,1-dicyclopropyl-3-((4-((1R,2S)-1-(3- | 3-((1R,2S)-2-(4-bromo-3- | 648 |
| cyclopropyl-3-methylureido)-1-(1- | fluorophenyl)-1-(1- | [M + 1] |
| (cyclopropylmethyl)-1H-1,2,4-triazol-5-yl)propan-2- | (cyclopropylmethyl)-1H- | |
| yl)-2-fluorophenyl)amino)-3-oxopropan-2-yl)-4- | 1,2,4-triazol-5-yl)propyl)- | |
| methyl-1,2,5-oxadiazole-3-carboxamide (76) | 1-cyclopropyl-1- | |
| | methylurea | |

¹H NM/R data of compounds 57, 58, 60, 61, 63, 64, 65, 66, 67, 70, 75 and 76 are shown below:

| Compound # | ¹H NMR |
|---|---|
| 57 | ¹H NMR (400 MHz, CD₃OD) δ 7.84 (s, 1H), 7.73 (dd, J = 15.9, 8.0 Hz, 1H), |
| | 7.03-6.94 (m, 2H), 5.35 (t, J = 10.5 Hz, 1H), 4.98 (d, J = 6.6 Hz, 1H), 4.55 |
| | (s, 1H), 4.09-3.95 (m, 2H), 3.73 (ddd, J = 37.6, 14.5, 7.2 Hz, 2H), 3.49- |
| | 3.40 (m, 1H), 2.54 (s, 3H), 1.44 (d, J = 7.0 Hz, 3H), 0.89-0.74 (m, 3H), |
| | 0.55-0.36 (m, 6H), 0.33-0.20 (m, 6H). |
| 58 | ¹H NMR (400 MHz, CD₃OD) δ 7.83 (s, 1H), 7.73 (dd, J = 16.3, 7.7 Hz, 1H), |
| | 6.97 (s, 2H), 5.35 (d, J = 11.0 Hz, 1H), 4.98 (d, J = 6.6 Hz, 1H), 4.55 (s, |
| | 1H), 3.95 (d, J = 13.6 Hz, 2H), 3.77 (dd, J = 14.4, 6.8 Hz, 1H), 3.68 (dd, J = |
| | 14.4, 7.6 Hz, 1H), 3.45 (dq, J = 13.8, 6.9 Hz, 1H), 2.54 (s, 3H), 1.43 (d, J = |
| | 7.0 Hz, 3H), 0.98-0.77 (m, 4H), 0.56-0.20 (m, 11H). |
| 60 | ¹H NMR (400 MHz, DMSO-d₆) δ 9.86 (s, 1H), 8.98 (d, J = 8.9 Hz, 1H), |
| | 8.42 (d, J = 8.7 Hz, 1H), 7.79 (s, 1H), 7.64 (t, J = 8.3 Hz, 1H), 7.05 (dd, J = |
| | 12.1, 1.5 Hz, 1H), 6.96 (d, J = 8.4 Hz, 1H), 5.34-5.17 (m, 1H), 4.97 (dd, J = |
| | 8.8, 6.8 Hz, 1H), 3.94 (s, 2H), 3.75 (d, J = 7.1 Hz, 2H), 3.50-3.35 (m, |
| | 2H), 2.47 (s, 3H), 1.32 (d, J = 6.9 Hz, 3H), 1.01-0.68 (m, 4H), 0.56-0.51 |
| | (m, 2H), 0.50-0.14 (m, 14H). |
| 61 | ¹H NMR (400 MHz, DMSO-d₆) δ 9.88 (s, 1H), 8.99 (d, J = 9.0 Hz, 1H), |
| | 8.45 (d, J = 8.7 Hz, 1H), 7.79 (s, 1H), 7.67 (t, J = 8.3 Hz, 1H), 7.11-7.02 |
| | (m, 1H), 6.96 (d, J = 8.3 Hz, 1H), 5.44-5.17 (m, 1H), 4.98 (dd, J = 8.8, 6.8 |
| | Hz, 1H), 3.98 (tdd, J = 13.7, 11.0, 6.4 Hz, 2H), 3.87 (s, 2H), 3.54-3.43 (m, |
| | 1H), 3.29 (s, 3H), 2.47 (s, 3H), 2.42 (dd, J = 13.8, 6.3 Hz, 1H), 2.32-2.19 |
| | (m, 1H), 2.12 (s, 6H), 1.32 (d, J = 6.9 Hz, 3H), 0.95-0.65 (m, 3H), 0.53- |
| | 0.07 (m, 8H). |
| 63 | ¹H NMR (400 MHz, DMSO-d₆) δ 9.86 (s, 1H), 8.98 (d, J = 8.9 Hz, 1H), |
| | 8.33 (d, J = 8.8 Hz, 1H), 7.80 (s, 1H), 7.65 (t, J = 8.3 Hz, 1H), 7.05 (d, J = |
| | 12.1 Hz, 1H), 6.96 (d, J = 8.3 Hz, 1H), 5.53-5.15 (m, 1H), 4.97 (dd, J = |
| | 8.8, 6.8 Hz, 1H), 3.89 (s, 2H), 3.75 (d, J = 7.1 Hz, 2H), 3.53-3.36 (m, 3H), |
| | 2.47 (s, 3H), 1.32 (d, J = 6.9 Hz, 3H), 1.13 (t, J = 7.0 Hz, 3H), 1.01-0.64 |
| | (m, 4H), 0.51-0.13 (m, 12H). |
| 64 | ¹H NMR (400 MHz, DMSO-d₆) δ 9.86 (s, 1H), 9.06 (d, J = 8.5 Hz, 1H), |
| | 8.98 (d, J = 8.9 Hz, 1H), 7.81 (s, 1H), 7.65 (t, J = 8.3 Hz, 1H), 7.08 (dd, J = |
| | 12.2, 1.7 Hz, 1H), 6.97 (d, J = 8.4 Hz, 1H), 5.27 (dd, J = 10.4, 8.7 Hz, 1H), |
| | 4.97 (dd, J = 8.9, 6.7 Hz, 1H), 4.62 (q, J = 14.3 Hz, 2H), 3.80-3.69 (m, |
| | 2H), 3.47-3.36 (m, 1H), 2.47 (s, 3H), 1.34 (d, J = 6.9 Hz, 3H), 0.97-0.71 |
| | (m, 4H), 0.50-0.13 (m, 12H). |
| 65 | ¹H NMR (400 MHz, DMSO-d₆) δ 9.86 (s, 1H), 8.98 (d, J = 8.9 Hz, 1H), |
| | 8.85 (d, J = 8.6 Hz, 1H), 7.80 (s, 1H), 7.64 (t, J = 8.3 Hz, 1H), 7.06 (d, J = |
| | 12.2 Hz, 1H), 6.97 (d, J = 8.4 Hz, 1H), 6.72 (t, J = 75.0 Hz, 1H), 5.34-5.20 |
| | (m, 1H), 4.97 (dd, J = 8.8, 6.8 Hz, 1H), 4.43-4.28 (m, 2H), 3.75 (d, J = 7.1 |
| | Hz, 2H), 3.42 (td, J = 14.2, 7.2 Hz, 1H), 2.47 (s, 3H), 1.34 (d, J = 6.9 Hz, |
| | 3H), 0.99-0.68 (m, 4H), 0.51-0.11 (m, 12H). |
| 66 | ¹H NMR (400 MHz, DMSO-d₆) δ 9.86 (s, 1H), 8.98 (d, J = 8.9 Hz, 1H), |
| | 8.58 (d, J = 8.6 Hz, 1H), 7.78 (s, 1H), 7.64 (t, J = 8.3 Hz, 1H), 7.04 (d, J = |
| | 12.1 Hz, 1H), 6.96 (d, J = 8.4 Hz, 1H), 5.27-5.12 (m, 1H), 4.97 (dd, J = |
| | 8.8, 6.8 Hz, 1H), 3.76 (t, J = 6.9 Hz, 2H), 3.38 (dd, J = 10.3, 7.0 Hz, 1H), |
| | 2.47 (s, 3H), 2.14 (ddd, J = 41.7, 14.8, 7.5 Hz, 2H), 1.33 (d, J = 6.9 Hz, 3H), |
| | 0.98 (t, J = 7.6 Hz, 3H), 0.92 (dd, J = 8.7, 6.1 Hz, 1H), 0.86 (dd, J = 10.3, |
| | 6.1 Hz, 1H), 0.79 (dd, J = 8.8, 4.0 Hz, 1H), 0.73 (dd, J = 9.0, 7.1 Hz, 1H), |
| | 0.46 (dd, J = 11.7, 6.4 Hz, 1H), 0.42-0.34 (m, 2H), 0.33-0.26 (m, 2H), |
| | 0.26-0.09 (m, 7H). |
| 67 | ¹H NMR (400 MHz, DMSO-d₆) δ 9.85 (s, 1H), 8.98 (d, J = 9.1 Hz, 1H), |
| | 8.66 (d, J = 8.6 Hz, 1H), 7.78 (s, 1H), 7.71-7.46 (m, 1H), 7.11-7.01 (m, |
| | 1H), 6.95 (t, J = 7.4 Hz, 1H), 5.24-5.13 (m, 1H), 4.96 (t, J = 7.7 Hz, 1H), |
| | 3.83-3.64 (m, 2H), 2.47 (s, 3H), 2.00 (dd, J = 15.0, 7.2 Hz, 1H), 1.86 (s, |
| | 3H), 1.33 (d, J = 6.9 Hz, 3H), 0.97-0.66 (m, 4H), 0.54-0.09 (m, 12H). |
| 70 | ¹H NMR (400 MHz, DMSO-d₆) δ 9.86 (s, 1H), 9.13 (d, J = 8.5 Hz, 1H), |
| | 8.98 (d, J = 9.0 Hz, 1H), 7.81 (s, 1H), 7.75-7.52 (m, 1H), 7.08 (d, J = 12.1 |
| | Hz, 1H), 6.96 (d, J = 8.3 Hz, 1H), 5.35-5.09 (m, 1H), 4.97 (dd, J = 8.8, 6.8 |
| | Hz, 1H), 3.85-3.54 (m, 4H), 3.35 (s, 1H), 2.47 (s, 3H), 1.35 (d, J = 6.9 Hz, |
| | 3H), 1.03-0.61 (m, 4H), 0.59-0.06 (m, 12H). |
| 75 | ¹H NMR (400 MHz, DMSO-d₆) δ 9.85 (s, 1H), 8.97 (dd, J = 9.0, 4.2 Hz, |
| | 1H), 8.57 (d, J = 8.6 Hz, 1H), 7.78 (s, 1H), 7.63 (dd, J = 15.0, 6.7 Hz, 1H), |
| | 7.04 (dd, J = 12.2, 1.7 Hz, 1H), 6.99-6.91 (m, 1H), 5.21 (dd, J = 10.2, 8.9 |
| | Hz, 1H), 4.97 (dd, J = 8.9, 6.7 Hz, 1H), 3.76 (t, J = 7.0 Hz, 2H), 3.38 (dd, J = |
| | 10.4, 7.0 Hz, 1H), 2.47 (s, 3H), 1.33 (d, J = 7.0 Hz, 3H), 0.81 (ddd, J = |
| | 20.3, 16.1, 4.9 Hz, 4H), 0.51-0.11 (m, 12H). |
| 76 | ¹H NMR (400 MHz, DMSO-d₆) δ 9.87 (s, 1H), 8.99 (d, J = 9.0 Hz, 1H), |
| | 7.79 (s, 1H), 7.66 (t, J = 8.3 Hz, 1H), 6.99 (dd, J = 22.2, 10.3 Hz, 2H), 6.45 |
| | (d, J = 8.9 Hz, 1H), 5.09 (t, J = 9.5 Hz, 1H), 4.98 (dd, J = 8.8, 6.8 Hz, 1H), |
| | 3.73 (ddd, J = 38.3, 14.4, 7.1 Hz, 2H), 3.45 (dd, J = 10.0, 7.0 Hz, 1H), 2.74 |
| | (s, 3H), 2.48 (s, 3H), 1.37 (d, J = 6.9 Hz, 3H), 0.97-0.68 (m, 6H), 0.68- |
| | 0.12 (m, 15H). |

### Example 29. N—((S)-1,1-dicyclopropyl-3-((4-((1R,2S)-1-(1-(cyclopropylmethyl)-1H-1,2,4-triazol-5-yl)-1-(2-((1-methylazetidin-3-yl)oxy)acetamido)propan-2-yl)-2-fluorophenyl)amino)-3-oxopropan-2-yl)-4-methyl-1,2,5-oxadiazole-3-carboxamide (Compound 56)

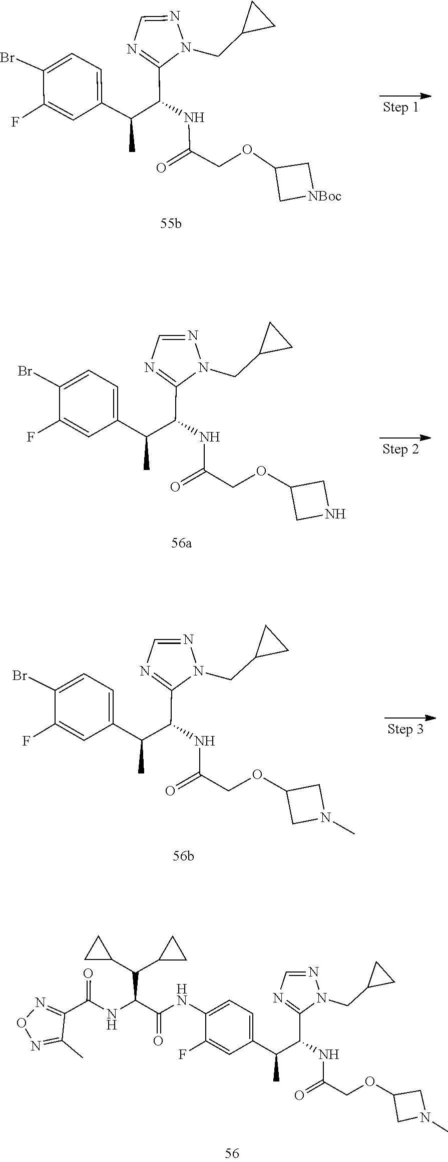 [SMILES: C[C@@H](c1ccc(Br)c(F)c1)[C@@H](NC(=O)COC1CN(C(=O)OC(C)(C)C)C1)c1ncnn1CC1CC1 ; C[C@@H](c1ccc(Br)c(F)c1)[C@@H](NC(=O)COC1CNC1)c1ncnn1CC1CC1 ; C[C@@H](c1ccc(Br)c(F)c1)[C@@H](NC(=O)COC1CN(C)C1)c1ncnn1CC1CC1 ; Cc1nonc1C(=O)N[C@H](C(=O)Nc1ccc([C@H](C)[C@@H](NC(=O)COC2CN(C)C2)c2ncnn2CC2CC2)cc1F)C(C1CC1)C1CC1]

### Step 1. 2-(Azetidin-3-yloxy)-N-((1R,2S)-2-(4-bromo-3-fluorophenyl)-1-(1-(cyclopropylmethyl)-1H-1,2,4-triazol-5-yl)propyl)acetamide hydrochloride (56a)

A solution of 55b (227 mg, 0.4 mmol) in a mixture of hydrochloric acid in 1,4-dioxane (4 M, 1 mL) and methanol (1 mL) was stirred for 1 h. The reaction mixture was concentrated to dryness to give the title compound 56a (187 mg, crude). The crude product was directly used in the next step without further purification.

MS m/z (ESI): 466, 468 [M+1]

### Step 2. N-((1R,2S)-2-(4-bromo-3-fluorophenyl)-1-(1-(cyclopropylmethyl)-1H-1,2,4-triazol-5-yl)propyl)-2-((1-methylazetidin-3-yl)oxy)acetamide (56b)

To a mixture of 56a (187 mg, 0.4 mmol), TEA (277 uL, 2.0 mmol), formaldehyde (37% aqueous solution, 49 uL, 0.6 mmol) and dichloromethane (2 mL) was added NaBH(OAc)₃ (127 mg, 0.6 mmol), which was then stirred for 3 h. The reaction mixture was concentrated to dryness and the residue was purified by prep-HPLC to give the title compound 56b (96 mg, 50%).

MS m/z (ESI): 480, 482 [M+1]

### Step 3. N—((S)-1,1-dicyclopropyl-3-((4-((1R,2S)-1-(1-(cyclopropylmethyl)-1H-1,2,4-triazol-5-yl)-1-(2-((1-methylazetidin-3-yl)oxy)acetamido)propan-2-yl)-2-fluorophenyl)amino)-3-oxopropan-2-yl)-4-methyl-1,2,5-oxadiazole-3-carboxamide (56)

A mixture of 56b (96 mg, 0.2 mmol), (S)—N-(1-amino-3,3-dicyclopropyl-1-oxopropan-2-yl)-4-methyl-1,2,5-oxadiazole-3-carboxamide (56 mg, 0.2 mmol), Cs₂CO₃ (99 mg, 0.3 mmol), CuI (11 mg, 0.06 mmol), trans-N,N′-dimethylcyclohexane-1,2-diamine (17 mg, 0.12 mmol) and toluene (1 mL) was heated to 100° C. and stirred for 4 h. After cooling to room temperature, the reaction mixture was purified by silica gel column chromatography (dichloromethane/methanol=100/0 to 7/3) and prep-HPLC to give the title compound 56 (29.8 mg, 22%).

MS m/z (ESI): 678 [M+1]

¹H NMR (400 MHz, DMSO-d₆) δ 9.87 (s, 1H), 8.99 (d, J=8.9 Hz, 1H), 8.48 (d, J=8.7 Hz, 1H), 7.81 (s, 1H), 7.66 (t, J=8.3 Hz, 1H), 7.06 (dd, J=12.1, 1.5 Hz, 1H), 6.97 (d, J=8.3 Hz, 1H), 5.31-5.18 (m, 1H), 4.98 (dd, J=8.8, 6.8 Hz, 1H), 4.03 (dd, J=11.6, 5.8 Hz, 1H), 3.87 (s, 2H), 3.76 (d, J=7.1 Hz, 2H), 3.45 (t, J=8.0 Hz, 3H), 2.83 (dd, J=14.2, 8.2 Hz, 2H), 2.48 (s, 3H), 2.21 (d, J=9.8 Hz, 3H), 1.34 (d, J=6.9 Hz, 3H), 1.01-0.91 (m, 1H), 0.88 (dd, J=12.5, 4.1 Hz, 1H), 0.84-0.78 (m, 1H), 0.74 (dd, J=9.0, 7.0 Hz, 1H), 0.53-0.43 (m, 1H), 0.43-0.35 (m, 2H), 0.32 (dd, J=11.3, 6.6 Hz, 2H), 0.28-0.12 (m, 7H).

### Example 30. N-((2S)-1,1-dicyclopropyl-3-((2-fluoro-4-((1R,2S)-1-(2-methoxyacetamido)-1-(5-(1-methylazetidin-2-yl)-1,3,4-thiadiazol-2-yl)propan-2-yl)phenyl)amino)-3-oxopropan-2-yl)-1-ethyl-1H-pyrazole-5-carboxamide (Compound 59)

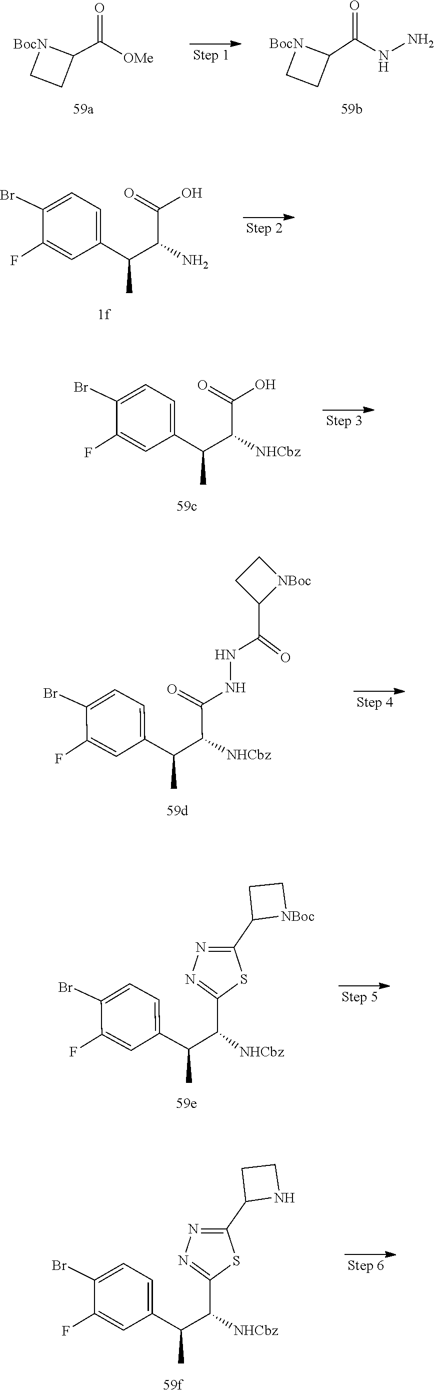

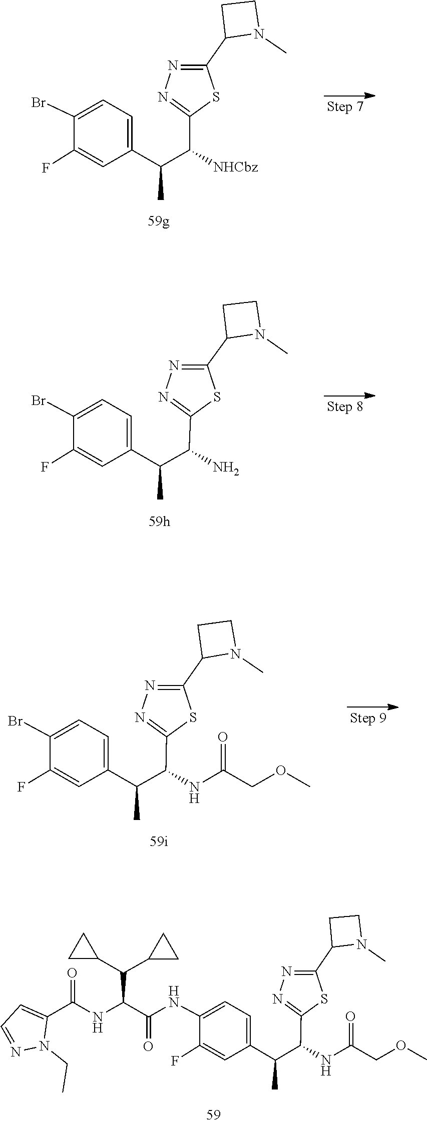 [SMILES: C[C@@H](c1ccc(Br)c(F)c1)[C@@H](NC(=O)OCc1ccccc1)c1nnc(C2CCN2C)s1 ; C[C@@H](c1ccc(Br)c(F)c1)[C@@H](N)c1nnc(C2CCN2C)s1 ; COCC(=O)N[C@@H](c1nnc(C2CCN2C)s1)[C@@H](C)c1ccc(Br)c(F)c1 ; CCn1nccc1C(=O)N[C@H](C(=O)Nc1ccc([C@H](C)[C@@H](NC(=O)COC)c2nnc(C3CCN3C)s2)cc1F)C(C1CC1)C1CC1]

### Step 1. Tert-butyl 2-(hydrazinecarbonyl)azetidine-1-carboxylate (59b)

To a solution of 1-(tert-butyl) 2-methyl azetidine-1,2-dicarboxylate 59a (430 mg, 2 mmol) in ethanol (5 mL) was added hydrazine hydrate (3 mL), which was then heated to reflux and stirred for 1 h. After cooling to room temperature, the reaction mixture was concentrated to dryness to give the title compound 59b (430 mg, 99%). The crude product was directly used in the next step without further purification.

MS m/z (ESI): 216 [M+1]

### Step 2. (2R,3S)-2-(((benzyloxy)carbonyl)amino)-3-(4-bromo-3-fluorophenyl)butanoic acid (59c)

To a mixture of 1f (600 mg, 2.2 mmol), NaOH (434 mg, 10.3 mmol), THF (5 mL) and water (5 mL) was added CbzCl (1.11 g, 6.5 mmol), which was then stirred for 30 min. The reaction mixture was concentrated to dryness and the residue was purified by prep-HPLC to give the title compound 59c (600 mg, 67%).

MS m/z (ESI): 410, 412 [M+1]

### Step 3. Tert-butyl 2-(2-((2R,3S)-2-(((benzyloxy)carbonyl)amino)-3-(4-bromo-3-fluorophenyl)butanoyl)hydrazine-1-carbonyl)azetidine-1-carboxylate (59d)

To a mixture of 59c (600 mg, 1.5 mmol), 59a (430 mg, 2 mmol), HATU (833 mg, 2.2 mmol) and DMF (10 mL) was added DIPEA (566 mg, 4.4 mmol), which was then stirred for 1 h. The reaction mixture was purified by prep-HPLC to give the title compound 59d (350 mg, 39%).

MS m/z (ESI): 607, 609 [M+1]

### Step 4. Tert-butyl 2-(5-((1R,2S)-1-(((benzyloxy)carbonyl)amino)-2-(4-bromo-3-fluorophenyl)propyl)-1,3,4-thiadiazol-2-yl)azetidine-1-carboxylate (59e)

To a solution of 59d (350 mg, 0.57 mmol) in THF (10 mL) was added Lawesson's reagent (350 mg, 0.86 mmol), which was then heated to 70° C. and stirred for 4 h. After cooling to room temperature, the reaction mixture was purified by prep-HPLC to give the title compound 59e (210 mg, 61%).

MS m/z (ESI): 605, 607 [M+1]

### Step 5. Benzyl ((1R,2S)-1-(5-(azetidin-2-yl)-1,3,4-thiadiazol-2-yl)-2-(4-bromo-3-fluorophenyl)propyl)carbamate hydrochloride (59f)

A solution of 59e (227 mg, 0.4 mmol) in a mixture of hydrochloric acid in 1,4-dioxane (4 M, 6 mL) and dichloromethane (6 mL) was stirred for 1 h. The reaction mixture was concentrated to dryness to give the title compound 59f (200 mg, 99%). The crude product was directly used in the next step without further purification.

MS m/z (ESI): 505, 507 [M+1]

### Step 6. Benzyl ((1R,2S)-2-(4-bromo-3-fluorophenyl)-1-(5-(1-methylazetidin-2-yl)-1,3,4-thiadiazol-2-yl)propyl)carbamate (59g)

To a mixture of 59f (200 mg, 0.35 mmol), polyformaldehyde (200 mg) and methanol (10 mL) was added NaBH₃CN (65 mg, 1.0 mmol), which was then heated 60° C. and stirred for 2 h. After cooling to room temperature, the reaction mixture was concentrated to dryness and the residue was purified by prep-HPLC to give the title compound 59g (110 mg, 61%).

MS m/z (ESI): 519, 521 [M+1]

### Step 7. (1R,2S)-2-(4-bromo-3-fluorophenyl)-1-(5-(1-methylazetidin-2-yl)-1,3,4-thiadiazol-2-yl)propan-1-amine (59h)

A solution of 59g (110 mg, 0.2 mmol) in TFA (6 mL) was heated to 90° C. and stirred for 3 h. After cooling to room temperature, the reaction mixture was concentrated to dryness and purified by prep-HPLC to give the title compound 59h (50 mg, 62%).

MS m/z (ESI): 385, 387 [M+1]

### Step 8. N-((1R,2S)-2-(4-bromo-3-fluorophenyl)-1-(5-(1-methylazetidin-2-yl)-1,3,4-thiadiazol-2-yl)propyl)-2-methoxyacetamide (59i)

To a mixture of 59h (50 mg, 0.13 mmol), 2-methoxyacetic acid (35 mg, 0.39 mmol), HATU (74 mg, 0.2 mmol) and DMF (3 mL) was added DIPEA (67 mg, 0.52 mmol), which was then stirred for 1 h. The reaction mixture was purified by prep-HPLC to give the title compound 59i (45 mg, 65%).

MS m/z (ESI): 457, 459 [M+1]

### Step 9. N-((2S)-1,1-dicyclopropyl-3-((2-fluoro-4-((1R,2S)-1-(2-methoxyacetamido)-1-(5-(1-methylazetidin-2-yl)-1,3,4-thiadiazol-2-yl)propan-2-yl)phenyl)amino)-3-oxopropan-2-yl)-1-ethyl-1H-pyrazole-5-carboxamide (59)

A mixture of 59i (45 mg, 0.1 mmol), is (57 mg, 0.2 mmol), Cs₂CO₃ (85 mg, 0.3 mmol), BrettPhos Pd G3 (24 mg, 0.03 mmol) and 1,4-dioxane (3 mL) was heated to 110° C. and stirred for 3 h. After cooling to room temperature, the reaction mixture was filtered. The filtrate was concentrated to dryness and the residue was purified by prep-HPLC to give the title compound 59 (4.3 mg, 5%).

MS m/z (ESI): 667 [M+1]

¹H NMR (400 MHz, CD₃OD) δ 7.81 (td, J=8.3, 2.1 Hz, 1H), 7.50 (d, J=2.1 Hz, 1H), 7.06 (d, J=11.8 Hz, 1H), 6.99 (d, J=3.5 Hz, 1H), 6.84 (d, J=2.1 Hz, 1H), 5.59 (dd, J=9.6, 3.2 Hz, 1H), 4.93 (d, J=7.0 Hz, 1H), 4.53 (q, J=7.2 Hz, 2H), 4.34 (t, J=8.1 Hz, 1H), 3.93 (dd, J=25.5, 10.2 Hz, 2H), 3.53 (t, J=7.0 Hz, 1H), 3.49-3.37 (m, 4H), 3.02 (dd, J=16.0, 8.2 Hz, 1H), 2.43 (dt, J=10.6, 7.8 Hz, 1H), 2.36-2.23 (m, 3H), 2.15 (dd, J=19.4, 9.3 Hz, 1H), 1.47-1.34 (m, 6H), 0.91-0.74 (m, 3H), 0.57-0.18 (m, 8H).

### Example 31. N—((S)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-1-(methyl-d₃)-1H-pyrazole-5-carboxamide (Compound 62)

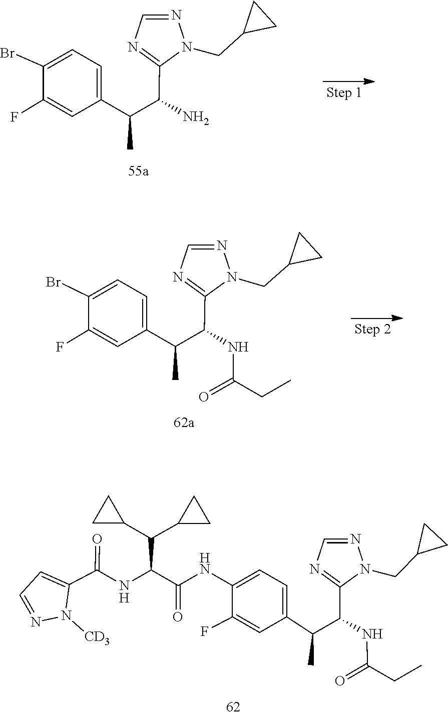 [SMILES: C[C@@H](c1ccc(Br)c(F)c1)[C@@H](N)c1ncnn1CC1CC1 ; CCC(=O)N[C@@H](c1ncnn1CC1CC1)[C@@H](C)c1ccc(Br)c(F)c1 ; [2H]C([2H])([2H])n1nccc1C(=O)N[C@H](C(=O)Nc1ccc([C@H](C)[C@@H](NC(=O)CC)c2ncnn2CC2CC2)cc1F)C(C1CC1)C1CC1]

### Step 1. N-((1R,2S)-2-(4-bromo-3-fluorophenyl)-1-(1-(cyclopropylmethyl)-1H-1,2,4-triazol-5-yl)propyl)propionamide (62a)

To a mixture of 55a (350 mg, 1.0 mmol), propionic acid (184 mg, 2.0 mmol), DMTMM·BF₄ (439 mg, 1.5 mmol) and acetonitrile (5 mL) was added TEA (200 mg, 2.0 mmol), which was then stirred for 1 h. The reaction mixture was concentrated to dryness and the residue was purified by silica gel column chromatography (dichloromethane/methanol=9/1) to give the title compound 62a (300 mg, 71%).

MS m/z (ESI): 409, 411 [M+1]

### Step 2. N—((S)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-1-(methyl-d₃)-1H-pyrazole-5-carboxamide (62)

A mixture of 62a (77 mg, 0.18 mmol), (S)—N-(1-amino-3,3-dicyclopropyl-1-oxopropan-2-yl)-1-(methyl-d₃)-1H-pyrazole-5-carboxamide (50 mg, 0.18 mmol), Cs₂CO₃ (118 mg, 0.36 mmol), CuI (10 mg, 0.05 mmol), trans-N,N′-dimethylcyclohexane-1,2-diamine (16 uL, 0.1 mmol) and toluene (1 mL) was heated to 110° C. and stirred for 3 h. After cooling to room temperature, the reaction mixture was purified by prep-HPLC to give the title compound 62 (65.8 mg, 58%).

MS m/z (ESI): 608 [M+1]

¹H NMR (400 MHz, DMSO-d₆) δ 9.81 (s, 1H), 8.57 (d, J=8.4 Hz, 1H), 8.35 (d, J=8.6 Hz, 1H), 7.77 (s, 1H), 7.65 (t, J=8.4 Hz, 1H), 7.46 (d, J=2.0 Hz, 1H), 7.10-6.99 (m, 2H), 6.95 (d, J=8.9 Hz, 1H), 5.25-5.16 (m, 1H), 4.91 (t, J=8.1 Hz, 1H), 3.74 (dd, J=17.8, 10.5 Hz, 2H), 2.14 (ddd, J=41.9, 14.9, 7.7 Hz, 3H), 1.33 (d, J=7.0 Hz, 3H), 0.98 (t, J=7.6 Hz, 3H), 0.74 (dd, J=23.5, 14.4 Hz, 4H), 0.26 (ddd, J=54.7, 36.5, 23.3 Hz, 12H).

Compound 43 was prepared according to the procedures of step 2 of Example 31, except that different a reagent was used instead of 62a.

| | | MS m/z |
|---|---|---|
| Compound | Reagent replacing 62a | (ESI) |
| N-((S)-1,1-dicyclopropyl-3-((4-((1R,2S)-1-(1- | N-((1R,2S)-2-(4-bromo-3- | 608 |
| (cyclopropylmethyl)-1H-1,2,4-triazol-3-yl)-1- | fluorophenyl)-1-(1- | [M + 1] |
| propionamidopropan-2-yl)-2-fluorophenyl)amino)-3- | (cyclopropylmethyl)-1H- | |
| oxopropan-2-yl)-1-(methyl-d₃)-1H-pyrazole-5- | 1,2,4-triazol-3- | |
| carboxamide (43) | yl)propyl)propionamide | |

Compounds 44 and 86 were prepared according to the procedures of Example 31, except that different reagents were used instead of (S)—N-(1-amino-3,3-dicyclopropyl-1-oxopropan-2-yl)-1-(methyl-d₃)-1H-pyrazole-5-carboxamide.

| | Reagent replacing (S)-N- | |
|---|---|---|
| | (1-amino-3,3- | |
| | dicyclopropyl-1- | |
| | oxopropan-2-yl)-1- | |
| | (methyl-d₃)-1H-pyrazole- | MS m/z |
| Compound | 5-carboxamide | (ESI) |
| N-((S)-1,1-dicyclopropyl-3-((4-((1R,2S)-1-(1- | (S)-N-(1-amino-3,3- | 633 |
| (cyclopropylmethyl)-1H-1,2,4-triazol-5-yl)-1- | dicyclopropyl-1- | [M + 1] |
| propionamidopropan-2-yl)-2-fluorophenyl)amino)-3- | oxopropan-2-yl)-1- | |
| oxopropan-2-yl)-1-isopropyl-1H-pyrazole-5- | isopropyl-1H-pyrazole-5- | |
| carboxamide (44) | carboxamide (4e) | |
| N-((S)-1,1-dicyclopropyl-3-((4-((1R,2S)-1-(1- | (S)-N-(1-amino-3,3- | 608 |
| (cyclopropylmethyl)-1H-1,2,4-triazol-5-yl)-1- | dicyclopropyl-1- | [M + 1] |
| propionamidopropan-2-yl)-2-fluorophenyl)amino)-3- | oxopropan-2-yl)-1- | |
| oxopropan-2-yl)-1-(methyl-d₃)-1H-pyrazole-3- | (methyl-d₃)-1H-pyrazole- | |
| carboxamide (86) | 3-carboxamide | |

Compounds 69 and 71 were prepared according to the procedures of Example 31, except that different reagents were used instead of propionic acid.

| | Reagent replacing | MS m/z |
|---|---|---|
| Compound | propionic acid | (ESI) |
| N-((S)-1,1-dicyclopropyl-3-((4-((1R,2S)-1-(1- | 2-Acetoxyacetic acid | 610 |
| (cyclopropylmethyl)-1H-1,2,4-triazol-5-yl)-1-(2- | | [M + 1] |
| hydroxyacetamido)propan-2-yl)-2- | | |
| fluorophenyl)amino)-3-oxopropan-2-yl)-1-(methyl- | | |
| d₃)-1H-pyrazole-5-carboxamide (69) | | |
| N-((S)-1,1-dicyclopropyl-3-((4-((1R,2S)-1-(1- | 2-(Methoxy-d₃)acetic acid | 627 |
| (cyclopropylmethyl)-1H-1,2,4-triazol-5-yl)-1-(2- | | [M + 1] |
| (methoxy-d₃)acetamido)propan-2-yl)-2- | | |
| fluorophenyl)amino)-3-oxopropan-2-yl)-1-(methyl- | | |
| d₃)-1H-pyrazole-5-carboxamide (71) | | |

¹H NMR data of compounds 43, 44, 69, 71 and 86 are shown below:

| Compound # | ¹H NMR |
|---|---|
| 43 | ¹H NMR (400 MHz, DMSO-d₆) δ 9.81 (s, 1H), 8.36 (d, J = 8.8 Hz, 1H), |
| | 8.32 (s, 1H), 8.18 (d, J = 9.3 Hz, 1H), 7.67 (t, J = 8.3 Hz, 1H), 7.46 (d, J = |
| | 2.0 Hz, 1H), 7.02 (d, J = 2.0 Hz, 1H), 6.91 (d, J = 12.3 Hz, 1H), 6.86 (d, J = |
| | 8.5 Hz, 1H), 5.13 (t, J = 8.9 Hz, 1H), 4.93 (t, J = 8.2 Hz, 1H), 3.87 (d, J = |
| | 7.1 Hz, 2H), 3.28-3.21 (m, 1H), 2.25-2.01 (m, 2H), 1.22 (d, J = 7.0 |
| | Hz, 3H), 1.07 (s, 1H), 0.96 (t, J = 7.6 Hz, 3H), 0.87 (s, 1H), 0.80 (s, 1H), |
| | 0.72 (dd, J = 17.4, 9.0 Hz, 1H), 0.43 (d, J = 7.9 Hz, 3H), 0.23 (d, J = 5.8 |
| | Hz, 8H), 0.16-0.08 (m, 1H). |
| 44 | ¹H NMR (400 MHz, DMSO-d₆) δ 9.78 (s, 1H), 8.57 (d, J = 8.5 Hz, 1H), |
| | 8.35 (d, J = 8.8 Hz, 1H), 7.78 (s, 1H), 7.65 (t, J = 8.3 Hz, 1H), 7.49 (d, J = |
| | 1.9 Hz, 1H), 7.03 (d, J = 12.1 Hz, 1H), 6.95 (d, J = 8.3 Hz, 1H), 6.89 (d, J = |
| | 1.9 Hz, 1H), 5.38 (dt, J = 13.1, 6.6 Hz, 1H), 5.25-5.07 (m, 1H), 4.89 (t, |
| | J = 8.1 Hz, 1H), 3.76 (qd, J = 14.4, 7.2 Hz, 2H), 3.37 (dd, J = 10.5, 7.0 Hz, |
| | 1H), 2.27-1.99 (m, 2H), 1.37 (d, J = 6.6 Hz, 3H), 1.33 (dd, J = 6.7, 4.1 Hz, |
| | 6H), 0.98 (t, J = 7.6 Hz, 3H), 0.93-0.65 (m, 4H), 0.47-0.09 (m, 12H). |
| 69 | ¹H NMR (400 MHz, DMSO-d₆) δ 9.82 (s, 1H), 8.36 (d, J = 8.8 Hz, 1H), |
| | 8.15 (d, J = 8.9 Hz, 1H), 7.79 (s, 1H), 7.66 (t, J = 8.3 Hz, 1H), 7.46 (d, J = |
| | 2.0 Hz, 1H), 7.03 (t, J = 7.1 Hz, 2H), 6.96 (d, J = 8.3 Hz, 1H), 5.47 (t, J = |
| | 6.0 Hz, 1H), 5.26 (t, J = 9.7 Hz, 1H), 4.91 (t, J = 8.2 Hz, 1H), 3.86 (dd, J = |
| | 8.3, 6.0 Hz, 2H), 3.74 (d, J = 7.1 Hz, 2H), 3.42 (d, J = 3.3 Hz, 1H), 1.33 (d, |
| | J = 6.9 Hz, 3H), 1.02-0.65 (m, 4H), 0.52-0.05 (m, 12H). |
| 71 | ¹H NMR (400 MHz, DMSO-d₆) δ 9.82 (s, 1H), 8.42 (d, J = 8.7 Hz, 1H), |
| | 8.36 (d, J = 8.9 Hz, 1H), 7.79 (s, 1H), 7.65 (t, J = 8.3 Hz, 1H), 7.46 (d, J = |
| | 2.0 Hz, 1H), 7.03 (dd, J = 12.7, 1.8 Hz, 2H), 6.95 (d, J = 8.4 Hz, 1H), 5.32- |
| | 5.16 (m, 1H), 4.91 (t, J = 8.2 Hz, 1H), 3.86 (s, 2H), 3.74 (d, J = 7.1 Hz, |
| | 2H), 3.44 (td, J = 13.6, 6.6 Hz, 1H), 1.33 (d, J = 6.9 Hz, 3H), 1.01-0.53 |
| | (m, 4H), 0.51-0.07 (m, 12H). |
| 86 | ¹H NMR (400 MHz, DMSO-d₆) δ 9.82 (s, 1H), 8.57 (d, J = 8.6 Hz, 1H), |
| | 7.84-7.74 (m, 2H), 7.69 (d, J = 9.4 Hz, 1H), 7.58 (t, J = 8.3 Hz, 1H), 7.03 |
| | (dd, J = 12.1, 1.6 Hz, 1H), 6.94 (d, J = 9.0 Hz, 1H), 6.64 (d, J = 2.3 Hz, 1H), |
| | 5.26-5.12 (m, 1H), 4.93 (dd, J = 9.4, 5.3 Hz, 1H), 3.76 (p, J = 7.1 Hz, 2H), |
| | 3.43-3.34 (m, 1H), 2.12 (ddt, J = 37.5, 15.1, 7.5 Hz, 2H), 1.32 (d, J = 6.9 |
| | Hz, 3H), 0.98 (t, J = 7.6 Hz, 3H), 0.94-0.79 (m, 2H), 0.76 (d, J = 8.6 Hz, |
| | 2H), 0.42-0.14 (m, 12H). |

### Example 32. N—((S)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-1-(1-methylazetidin-3-yl)-1H-pyrazole-5-carboxamide (Compound 73)

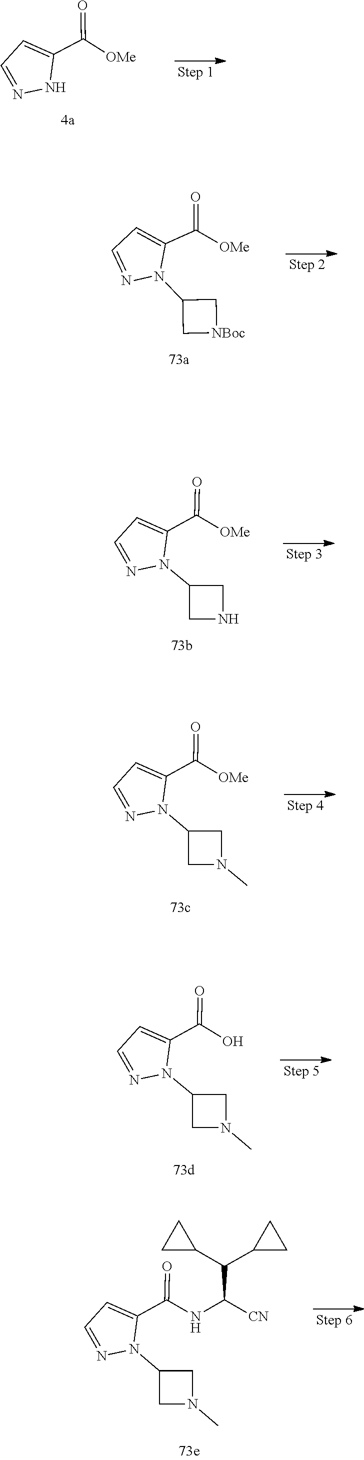 [SMILES: COC(=O)c1ccn[nH]1 ; COC(=O)c1ccnn1C1CN(C(=O)OC(C)(C)C)C1 ; COC(=O)c1ccnn1C1CNC1 ; COC(=O)c1ccnn1C1CN(C)C1 ; CN1CC(n2nccc2C(=O)O)C1 ; CN1CC(n2nccc2C(=O)N[C@H](C#N)C(C2CC2)C2CC2)C1]

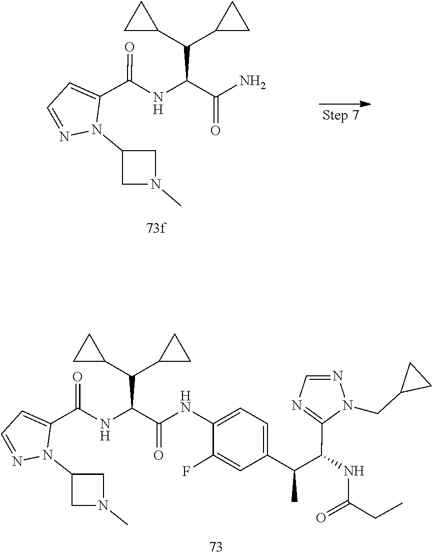 [SMILES: CN1CC(n2nccc2C(=O)N[C@H](C(N)=O)C(C2CC2)C2CC2)C1 ; CCC(=O)N[C@@H](c1ncnn1CC1CC1)[C@@H](C)c1ccc(NC(=O)[C@@H](NC(=O)c2ccnn2C2CN(C)C2)C(C2CC2)C2CC2)c(F)c1]

### Step 1. Methyl 1-(1-(tert-butoxycarbonyl)azetidin-3-yl)-1H-pyrazole-5-carboxylate (73a)

To a mixture of methyl 1H-pyrazole-5-carboxylate 4a (1.0 g, 7.9 mmol), tert-butyl 3-hydroxyazetidine-1-carboxylate (2.0 g, 11.9 mmol), PPh₃ (3.0 g, 11.9 mmol) and THF (30 mL) at 0° C. was added DEAD (2.0 g, 11.9 mmol), which was then stirred at 0° C. for 1 h. The reaction mixture was concentrated to dryness and the residue was purified by silica gel column chromatography (PE/EtOAc=4/1) to give the title compound 73a (1.6 g, 72%).

MS m/z (ESI): 282 [M+1]

### Step 2. Methyl 1-(azetidin-3-yl)-1H-pyrazole-5-carboxylate hydrochloride (73b)

A solution of 73a (1.6 g, 5.7 mmol) in a mixture of hydrochloric acid in 1,4-dioxane (4 M, 10 mL) and dichloromethane (30 mL) was stirred for 1 h. The reaction mixture was concentrated to dryness to give the title compound 73b (1.0 g, crude). The crude product was directly used in the next step without further purification.

MS m/z (ESI): 182 [M+1]

### Step 3. Methyl 1-(1-methylazetidin-3-yl)-1H-pyrazole-5-carboxylate (73c)

To a mixture of 73b (1.0 g, 5.5 mmol), formaldehyde (30% aqueous solution, 2.7 mL, 27.6 mmol) and methanol (20 mL) was added NaBH₃CN (1.7 g, 27.6 mmol), which was then stirred for 2 h. The reaction mixture was concentrated to dryness and the residue was purified by silica gel column chromatography (dichloromethane/methanol=10:1) to give the title compound 73c (820 mg, 76%).

MS m/z (ESI): 196 [M+1]

### Step 4. 1-(1-Methylazetidin-3-yl)-1H-pyrazole-5-carboxylic acid (73d)

To a solution of 73c (820 mg, 4.2 mmol) in THF (30 mL) and water (10 mL) was added LiOH·H₂O (353 mg, 8.4 mmol), which was then stirred for 2 h. The reaction mixture was concentrated to dryness and the residue was purified by prep-HPLC to give the title compound 73d (320 mg, 42%).

MS m/z (ESI): 182 [M+1]

### Step 5. (S)—N-(1-cyano-2,2-dicyclopropylethyl)-1-(1-methylazetidin-3-yl)-1H-pyrazole-5-carboxamide (73e)

To a mixture of 1q (265 mg, 1.8 mmol), 73d (320 mg, 1.8 mmol), HATU (1.0 g, 2.7 mmol) and DMF (20 mL) was added DIPEA (685 mg, 5.3 mmol), which was then stirred for 1 h. The reaction mixture was added with EtOAc (100 mL) and washed sequentially with a saturated aqueous NaHCO₃ solution (100 mL), dilute hydrochloric acid (1 M, 100 mL) and saturated brine (3×100 mL). The organic phase was concentrated to dryness and the residue was purified by silica gel column chromatography (dichloromethane/methanol=6/1) to give the title compound 73e (180 mg, 33%).

MS m/z (ESI): 314 [M+1]

### Step 6. (S)—N-(1-amino-3,3-dicyclopropyl-1-oxopropan-2-yl)-1-(1-methylazetidin-3-yl)-1H-pyrazole-5-carboxamide (73f)

To a mixture of 73e (180 mg, 0.6 mmol), hydrogen peroxide (30% aqueous solution, 1 mL) and DMSO (2 mL) at 0° C. was added NaOH (46 mg, 1.2 mmol), which was then stirred at 0° C. for 1 h. The reaction mixture was purified by prep-HPLC to give the title compound 73f (40 mg, 21%).

MS m/z (ESI): 332 [M+1]

### Step 7. N—((S)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-1-(1-methylazetidin-3-yl)-1H-pyrazole-5-carboxamide (73)

A mixture of 62a (54 mg, 0.16 mmol), 73f (40 mg, 0.21 mmol), Cs₂CO₃ (78 mg, 0.24 mmol), CuI (10 mg, 0.05 mmol), trans-N,N′-dimethylcyclohexane-1,2-diamine (14 mg, 0.10 mmol) and 1,4-dioxane (1 mL) was heated to 100° C. and stirred for 12 h. After cooling to room temperature, the reaction mixture was purified by prep-HPLC to give the title compound 73 (10.9 mg, 9%).

MS m/z (ESI): 660 [M+1]

¹H NMR (400 MHz, DMSO-d₆) δ 9.81 (s, 1H), 8.57 (d, J=8.6 Hz, 1H), 8.41 (d, J=8.8 Hz, 1H), 7.77 (s, 1H), 7.64 (t, J=8.3 Hz, 1H), 7.57 (d, J=2.0 Hz, 1H), 7.03 (dd, J=12.3, 1.6 Hz, 1H), 7.00 (d, J=2.0 Hz, 1H), 6.95 (d, J=8.5 Hz, 1H), 5.61-5.48 (m, 1H), 5.32 (t, J=4.7 Hz, 1H), 5.24-5.15 (m, 1H), 4.94-4.84 (m, 1H), 3.70 (ddd, J=21.7, 15.3, 7.5 Hz, 4H), 2.28 (s, 3H), 2.19 (dd, J=14.9, 7.6 Hz, 2H), 2.08 (dd, J=15.7, 8.4 Hz, 2H), 1.97-1.94 (m, 1H), 1.33 (d, J=6.9 Hz, 3H), 0.99 (d, J=7.6 Hz, 3H), 0.83-0.66 (m, 3H), 0.45-0.14 (m, 12H).

### Example 33. N—((S)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-4-methyl-1,2,5-thiadiazole-3-carboxamide (Compound 74)

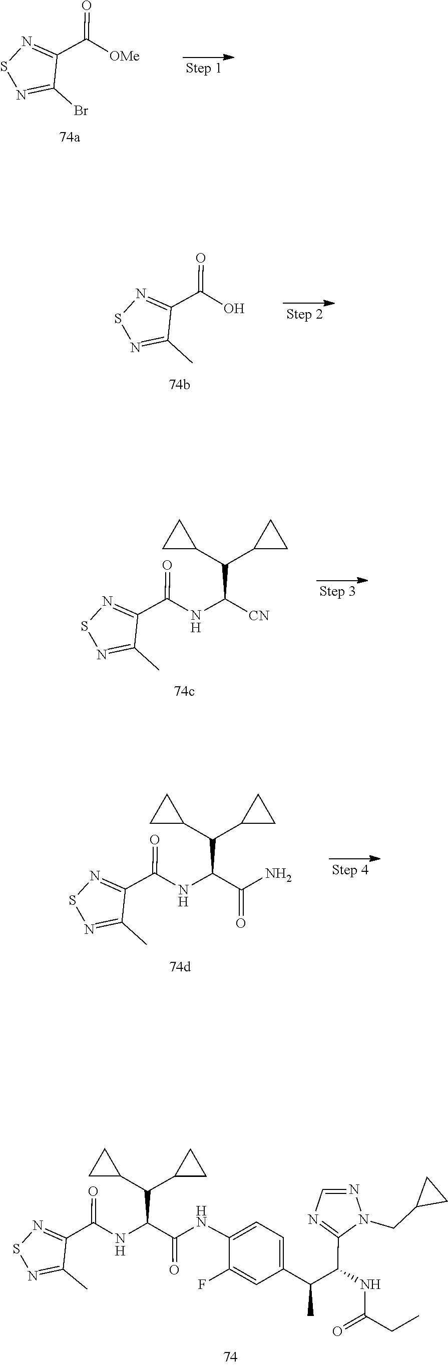

### Step 1. 4-Methyl-1,2,5-thiadiazole-3-carboxylic acid (74b)

A mixture of methyl 4-bromo-1,2,5-thiadiazole-3-carboxylate 74a (223 mg, 1.0 mmol), a solution of trimethylboroxine in THF (3.5 M, 0.6 mL), Cs₂CO₃ (650 mg, 2.0 mmol), Pd(dppf)Cl₂ (150 mg, 0.2 mmol), 1,4-dioxane (4 mL) and water (1 mL) was heated to reflux and stirred overnight. After cooling to room temperature, the reaction mixture was concentrated to dryness and the residue was purified by prep-HPLC to give the title compound 74b (40 mg, 28%).

MS m/z (ESI): 143 [M−1]

### Step 2. (S)—N-(1-cyano-2,2-dicyclopropylethyl)-4-methyl-1,2,5-thiadiazole-3-carboxamide (74c)

To a mixture of 1q (42 mg, 0.28 mmol), 74b (40 mg, 0.28 mmol), HATU (160 mg, 0.42 mmol) and DMF (1 mL) was added DIPEA (147 uL, 0.84 mmol), which was then stirred for 1 h. The reaction mixture was added with EtOAc (50 mL) and then washed sequentially with a saturated aqueous NaHCO₃ solution (50 mL), dilute hydrochloric acid (1 M, 50 mL) and saturated brine (50 mL). The organic phase was concentrated to dryness to give the title compound 74c (76 mg, 99%). The crude product was directly used in the next step without further purification.

MS m/z (ESI): 277 [M+1]

### Step 3. (S)—N-(1-amino-3,3-dicyclopropyl-1-oxopropan-2-yl)-4-methyl-1,2,5-thiadiazole-3-carboxamide (74d)

To a mixture of 74c (76 mg, 0.28 mmol), hydrogen peroxide (30% aqueous solution, 1 mL) and DMSO (1 mL) at 0° C. was added NaOH (2.2 mg, 0.06 mmol), which was then stirred at 0° C. for 1 h. The reaction mixture was added with EtOAc (50 mL) and then washed sequentially with saturated brine (50 mL). The organic phase was concentrated to dryness and the residue was purified by silica gel column chromatography (dichloromethane/methanol=100/0 to 10/1) to give the title compound 74d (79 mg, 99%).

MS m/z (ESI): 295 [M+1]

### Step 4. N—((S)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-4-methyl-1,2,5-thiadiazole-3-carboxamide (74)

A mixture of 62a (110 mg, 0.28 mmol), 74d (79 mg, 0.28 mmol), Cs₂CO₃ (182 mg, 0.56 mmol), CuI (14 mg, 0.07 mmol), trans-N,N′-dimethylcyclohexane-1,2-diamine (23 mg, 0.14 mmol) and toluene (1 mL) was heated to 100° C. and stirred overnight. After cooling to room temperature, the reaction mixture was purified by prep-HPLC to give the title compound 74 (25.4 mg, 15%).

MS m/z (ESI): 623 [M+1]

¹H NMR (400 MHz, DMSO-d₆) δ 9.85 (s, 1H), 8.57 (d, J=8.6 Hz, 1H), 8.41 (d, J=9.2 Hz, 1H), 7.77 (s, 1H), 7.61 (t, J=8.4 Hz, 1H), 7.04 (d, J=11.3 Hz, 1H), 6.95 (d, J=8.0 Hz, 1H), 5.25-5.15 (m, 1H), 4.94 (dd, J=8.6, 5.4 Hz, 1H), 3.84-3.70 (m, 2H), 3.42-3.35 (m, 1H), 2.71 (s, 3H), 2.25-2.03 (m, 2H), 1.33 (d, J=6.9 Hz, 3H), 0.98 (t, J=7.6 Hz, 3H), 0.94-0.71 (m, 4H), 0.49-0.12 (m, 12H).

### Example 34. N—((S)-2-((4-((1R,2S)-1-(1-(cyclopropylmethyl)-1H-1,2,4-triazol-5-yl)-1-propionamidopropan-2-yl)-2-fluorophenyl)amino)-1-(4-(difluoromethylene)cyclohexyl)-2-oxoethyl)-1-ethyl-1H-pyrazole-5-carboxamide (Compound 77)

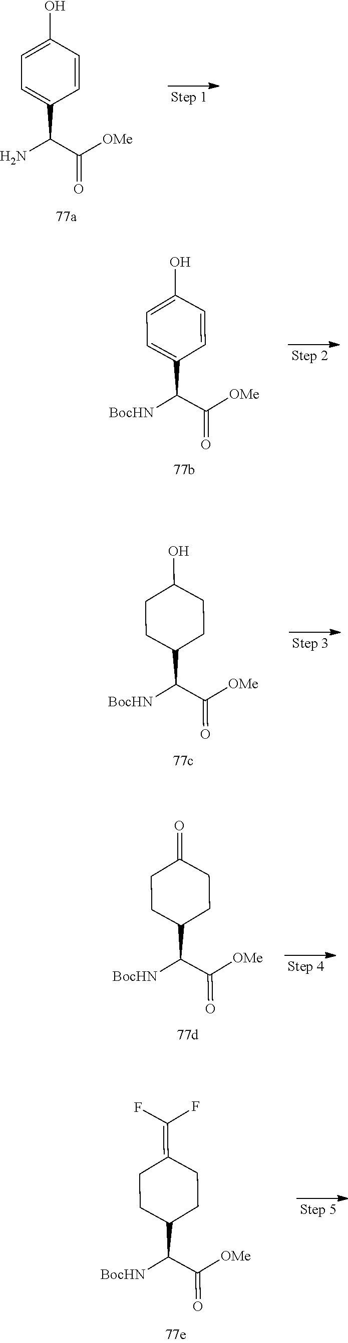 [SMILES: COC(=O)[C@@H](N)c1ccc(O)cc1 ; COC(=O)[C@@H](NC(=O)OC(C)(C)C)c1ccc(O)cc1 ; COC(=O)[C@@H](NC(=O)OC(C)(C)C)C1CCC(O)CC1 ; COC(=O)[C@@H](NC(=O)OC(C)(C)C)C1CCC(=O)CC1 ; COC(=O)[C@@H](NC(=O)OC(C)(C)C)C1CCC(=C(F)F)CC1]

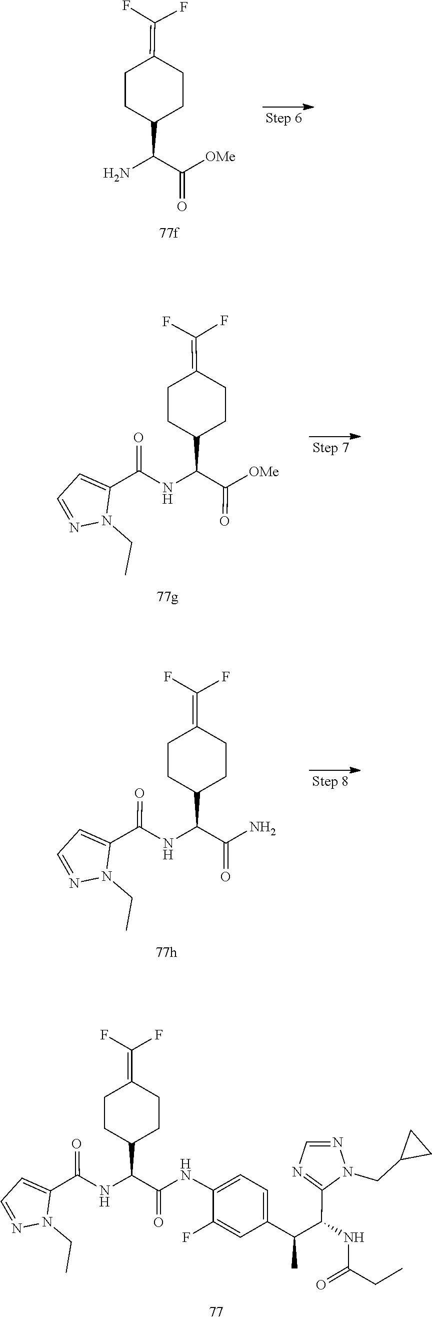 [SMILES: COC(=O)[C@@H](N)C1CCC(=C(F)F)CC1 ; CCn1nccc1C(=O)N[C@H](C(=O)OC)C1CCC(=C(F)F)CC1 ; CCn1nccc1C(=O)N[C@H](C(N)=O)C1CCC(=C(F)F)CC1 ; CCC(=O)N[C@@H](c1ncnn1CC1CC1)[C@@H](C)c1ccc(NC(=O)[C@@H](NC(=O)c2ccnn2CC)C2CCC(=C(F)F)CC2)c(F)c1]

### Step 1. Methyl (S)-2-((tert-butoxycarbonyl)amino)-2-(4-hydroxyphenyl)acetate (77b)

To a mixture of methyl (S)-2-amino-2-(4-hydroxyphenyl)acetate hydrochloride 77a (25.0 g, 115 mmol), Boc₂O (28.8 g, 132 mmol), 1,4-dioxane (200 mL) and water (400 mL) was added K₂CO₃ (31.8 g, 230 mmol) at 0° C., which was then stirred for 16 h. The reaction mixture was added EtOAc (300 mL) and washed with saturated brine (300 mL). The organic phase was dried over anhydrous Na₂SO₄ and filtered. The filtrate was concentrated to dryness and the residue was purified by silica gel column chromatography (PE/EtOAc=3/2) to give the title compound 77b (25.0 g, 77%).

MS m/z (ESI): 282 [M+1]

### Step 2. Methyl (2S)-2-((tert-butoxycarbonyl)amino)-2-(4-hydroxycyclohexyl)acetate (77c)

To a solution of 77b (15.0 g, 53 mmol) in acetic acid (100 mL) was added PtO₂ (1.82 g, 8 mmol), which was then heated to 50° C. and stirred for 48 h under hydrogen atmosphere. After cooling to room temperature, the reaction mixture was filtered. The filtrate was concentrated to dryness and the residue was purified by silica gel column chromatography (PE/EtOAc=2/1) to give the title compound 77c (7.3 g, 48%).

MS m/z (ESI): 288 [M+1]

### Step 3. Methyl (S)-2-((tert-butoxycarbonyl)amino)-2-(4-oxocyclohexyl)acetate (77d)

To a solution of oxalyl chloride (6.5 g, 50.8 mmol) in dichloromethane (160 mL) was added DMSO (7.9 g, 101.6 mmol) at −78° C., which was then stirred at −78° C. for 30 min. The reaction mixture was added a solution of 77c (7.3 g, 25.4 mmol) in dichloromethane (20 mL) at −78° C., which was then stirred at −78° C. for 1 h. The reaction mixture was added TEA (20.6 g, 203.2 mmol) at −78° C., which was then warmed to room temperature and stirred for 1 h. The reaction mixture was added dilute hydrochloric acid (1 M, 200 mL) and extracted with dichloromethane (300 mL). The organic phase was dried over anhydrous Na₂SO₄ and filtered. The filtrate was concentrated to dryness and the residue was purified by silica gel column chromatography (PE/EtOAc=2/1) to give the title compound 77d (6.0 g, 83%).

MS m/z (ESI): 286 [M+1]

### Step 4. Methyl (S)-2-((tert-butoxycarbonyl)amino)-2-(4-(difluoromethylene)cyclohexyl)acetate (77e)

To a mixture of 77d (6.0 g, 21 mmol), 2-((difluoromethyl)sulfonyl)pyridine (4.9 g, 25 mmol) and DMF (60 mL) was added potassium tert-butoxide (4.3 g, 38 mmol) at −48° C., which was then warmed to room temperature and stirred for 3 h. The reaction mixture was added with water (300 mL) and extracted with EtOAc (300 mL). The organic phase was dried over anhydrous Na₂SO₄ and filtered. The filtrate was concentrated to dryness and the residue was purified by silica gel column chromatography (PE/EtOAc=2/1) to give the title compound 77e (5.0 g, 74%).

MS m/z (ESI): 320 [M+1]

### Step 5. Methyl (S)-2-amino-2-(4-(difluoromethylene)cyclohexyl)acetate hydrochloride (77f)

A mixture of 77e (1.94 g, 6.0 mmol) and a solution of hydrochloric acid in 1,4-dioxane (4 M, 30 mL) was stirred for 1 h. The reaction mixture was concentrated to dryness to give the title compound 77f (1.55 g, 99%). The crude product was directly used in the next step without further purification.

MS m/z (ESI): 220 [M+1]

### Step 6. Methyl (S)-2-(4-(difluoromethylene)cyclohexyl)-2-(1-ethyl-1H-pyrazole-5-carboxamido)acetate (77g)

To a mixture of 77f (1.55 g, 6.0 mmol), 1-ethyl-1H-pyrazole-5-carboxylic acid (1.02 g, 7.3 mmol), HATU (2.78 g, 7.3 mmol) and DMF (15 mL) was added DIPEA (3.92 g, 30.4 mmol), which was then stirred for 1 h. The reaction mixture was added with water (100 mL) and extracted with EtOAc (100 mL). The organic phase was dried over anhydrous Na₂SO₄ and filtered. The filtrate was concentrated to dryness and the residue was purified by silica gel column chromatography (PE/EtOAc=10/1) to give the title compound 77g (2.00 g, 96%).

MS m/z (ESI): 342 [M+1]

### Step 7. (S)—N-(2-amino-1-(4-(difluoromethylene)cyclohexyl)-2-oxoethyl)-1-ethyl-1H-pyrazole-5-carboxamide (77h)

A mixture of 77f (710 mg, 2.1 mmol) and a solution of ammonia in methanol (7M, 50 mL) was heated to 60° C. and stirred for 24 h. After cooling to room temperature, the reaction mixture was concentrated to dryness to give the title compound 77h (550 mg, 81%). The crude product was directly used in the next step without further purification.

MS m/z (ESI): 327 [M+1]

### Step 8. N—((S)-2-((4-((1R,2S)-1-(1-(cyclopropylmethyl)-1H-1,2,4-triazol-5-yl)-1-propionamidopropan-2-yl)-2-fluorophenyl)amino)-1-(4-(difluoromethylene)cyclohexyl)-2-oxoethyl)-1-ethyl-1H-pyrazole-5-carboxamide (77)

A mixture of 62a (95 mg, 0.23 mmol), 77h (91 mg, 0.28 mmol), Cs₂CO₃ (151 mg, 0.46 mmol), CuI (13 mg, 0.07 mmol), trans-N,N′-dimethylcyclohexane-1,2-diamine (20 mg, 0.14 mmol) and toluene (4 mL) was heated to 100° C. and stirred for 6 h. After cooling to room temperature, the reaction mixture was filtered. The filtrate was concentrated to dryness and the residue was purified by prep-HPLC to give the title compound 77 (55 mg, 36%).

MS m/z (ESI): 655 [M+1]

¹H NMR (400 MHz, DMSO-d₆) δ 9.80 (s, 1H), 8.57 (d, J=8.6 Hz, 1H), 8.46 (d, J=8.1 Hz, 1H), 7.77 (s, 1H), 7.57 (td, J=8.2, 3.9 Hz, 1H), 7.47 (d, J=2.0 Hz, 1H), 7.07 (d, J=12.1 Hz, 1H), 6.99 (d, J=2.0 Hz, 1H), 6.93 (d, J=8.3 Hz, 1H), 5.23 (t, J=9.5 Hz, 1H), 4.56 (t, J=8.3 Hz, 1H), 4.45 (q, J=7.2 Hz, 2H), 3.86-3.73 (m, 2H), 3.39 (dd, J=10.4, 6.9 Hz, 1H), 2.39 (d, J=11.0 Hz, 2H), 2.24-2.03 (m, 2H), 2.02-1.64 (m, 5H), 1.35-1.07 (m, 8H), 0.95 (dt, J=12.6, 7.6 Hz, 4H), 0.41-0.20 (m, 4H).

### Example 35. N—((S)-2-((4-((1R,2S)-1-(1-(cyclopropylmethyl)-1H-1,2,4-triazol-5-yl)-1-propionamidopropan-2-yl)-2-fluorophenyl)amino)-1-(2,2-difluorospiro[3.5]nonan-7-yl)-2-oxoethyl)-1-ethyl-1H-pyrazole-5-carboxamide (Compound 78)

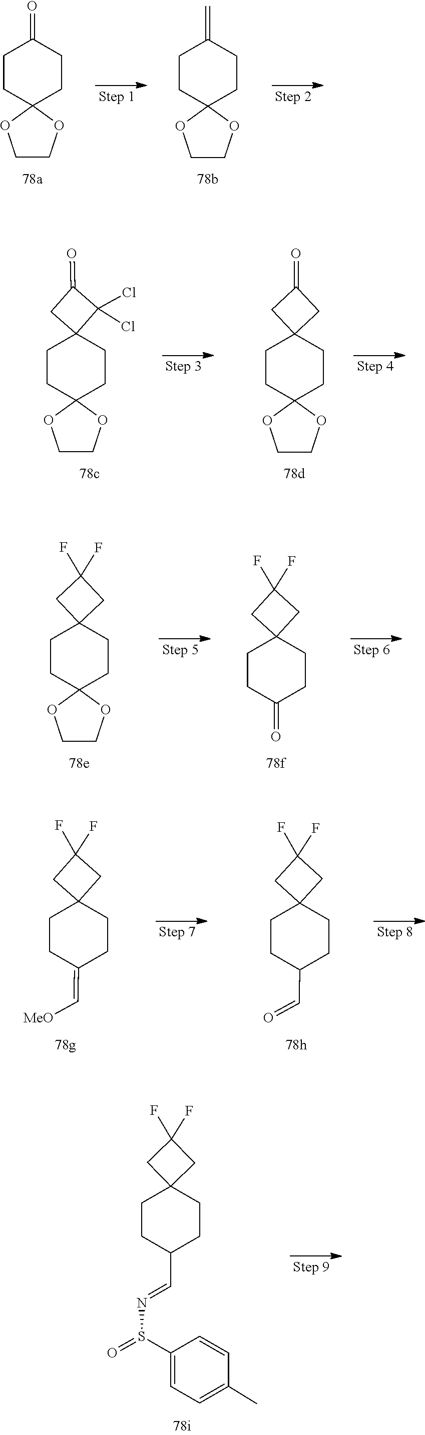

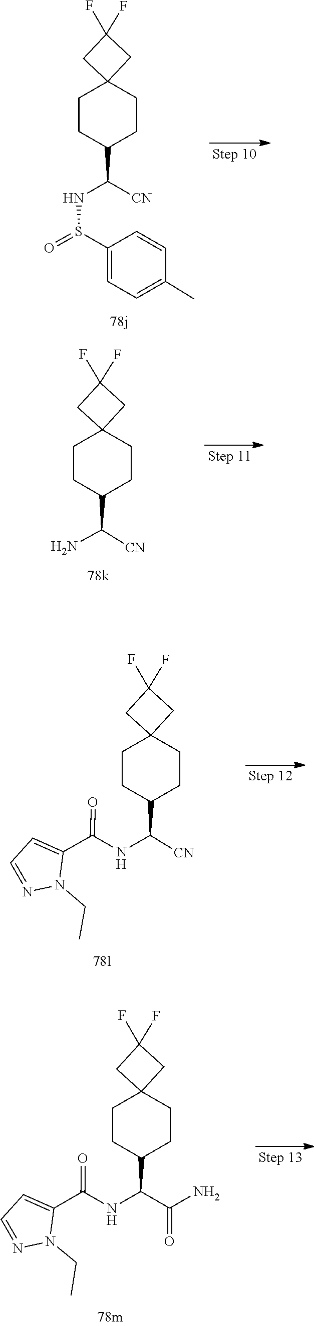

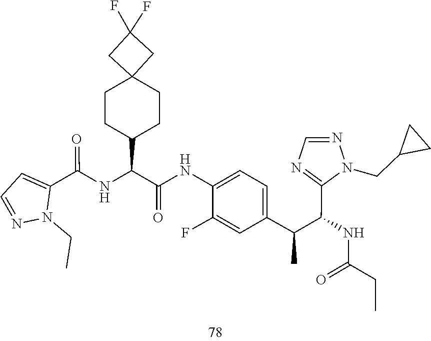 [SMILES: CCC(=O)N[C@@H](c1ncnn1CC1CC1)[C@@H](C)c1ccc(NC(=O)[C@@H](NC(=O)c2ccnn2CC)C2CCC3(CC2)CC(F)(F)C3)c(F)c1]

### Step 1. 8-Methylene-1,4-dioxaspiro[4.5]decane (78b)

To a suspension of methyltriphenylphosphonium bromide (53.6 g, 150 mmol) in THF (200 mL) at 0° C. was added potassium tert-butoxide (16.8 g, 150 mmol), which was then stirred for 1 h. The resulting mixture was added with 1,4-dioxaspiro[4.5]decan-8-one 78a (15.6 g, 100 mmol), which was then warmed to room temperature and stirred for 12 h. The reaction mixture was added with a saturated aqueous NH₄Cl solution (200 mL) and extracted with EtOAc (200 mL). The organic phase was dried over anhydrous Na₂SO₄ and filtered. The filtrate was concentrated to dryness and the residue was purified by silica gel column chromatography (PE/EtOAc=100/0 to 10/3) to give the title compound 78b (10.1 g, 66%).

### Step 2. 1,1-Dichloro-8,11-dioxadispiro[3.2.4⁷.2⁴]tridecan-2-one (78c)

To a suspension of zinc-copper couple (38.7 g, 300 mmol) in 1,2-dimethoxyethane (900 mL) at 0° C. was added 78b (15.4 g, 100 mmol) and 2,2,2-trichloroacetyl chloride (36.0 g, 200 mmol), which was then warmed to room temperature and stirred for 12 h. The reaction mixture was filtered. The filtrate was added with water (300 mL) and extracted with EtOAc (300 mL). The organic phase was dried over anhydrous Na₂SO₄ and filtered. The filtrate was concentrated to dryness and the residue was purified by silica gel column chromatography (PE/EtOAc=100/0 to 4/1) to give the title compound 78c (17.0 g, 57%).

### Step 3. 8,11-Dioxadispiro[3.2.4⁷.2⁴]tridecan-2-one (78d)

To a solution of 78c (1.5 g, 5.7 mmol) in methanol (50 mL) was added zinc (3.6 g, 56.8 mmol) and NH₄Cl (3.0 g, 56.8 mmol), which was then heated to 50° C. and stirred for 2 h. After cooling to room temperature, the reaction mixture was filtered. The filtrate was concentrated to dryness and the residue was purified by silica gel column chromatography (PE/EtOAc=100/0 to 5/1) to give the title compound 78d (820 mg, 75%).

### Step 4. 2,2-Difluoro-8,11-dioxadispiro[3.2.4⁷.2⁴]tridecane (78e)

To a solution of 78d (3.1 g, 15.8 mmol) in dichloromethane (30 mL) at 0° C. was added DAST (6.4 g, 39.5 mmol), which was then warmed to room temperature and stirred for 15 h. The reaction mixture was added with saturated NaHCO₃ solution (100 mL) and extracted with dichloromethane (100 mL). The organic phase was dried over anhydrous Na₂SO₄ and filtered.

The filtrate was concentrated to dryness and the residue was purified by silica gel column chromatography (PE/EtOAc=100/0 to 20/1) to give the title compound 78e (900 mg, 26%).

### Step 5. 2,2-Difluorospiro[3.5]nonan-7-one (78f)

A mixture of 78e (900 mg, 4.1 mmol), THF (6 mL) and dilute hydrochloric acid (6 M, 6 mL) was stirred for 1 h. The reaction mixture was concentrated to remove THF. The residue was added with a saturated aqueous NaHCO₃ solution (50 mL) and extracted with EtOAc (50 mL).

The organic phase was dried over anhydrous Na₂SO₄ and filtered. The filtrate was concentrated to dryness and the residue was purified by silica gel column chromatography (PE/EtOAc=100/0 to 30/1) to give the title compound 78f (700 mg, 97%).

### Step 6. 2,2-Difluoro-7-(methoxymethylene)spiro[3.5]nonane (78g)

To a suspension of (methoxymethyl)triphenylphosphonium chloride (1.93 g, 5.6 mmol) in THF (10 mL) at 0° C. was added a solution of KHMDS in THF (1.0 M, 5.4 mL), which was then stirred at 0° C. for 30 min. The reaction mixture was added with 78f (700 mg, 4.0 mmol), which was then warmed to room temperature and stirred for 30 min. The reaction mixture was added with saturated brine (100 mL) and extracted with EtOAc (100 mL). The organic phase was dried over anhydrous Na₂SO₄ and filtered. The filtrate was concentrated to dryness and the residue was purified by silica gel column chromatography (PE/EtOAc=100/0 to 50/1) to give the title compound 78g (500 mg, 62%).

### Step 7. 2,2-Difluorospiro[3.5]nonane-7-carbaldehyde (78h)

A mixture of 78g (500 mg, 2.5 mmol), THF (10 mL) and dilute hydrochloric acid (6 M, 10 mL) was stirred for 1 h. The reaction mixture was concentrated to dryness and the residue was purified by silica gel column chromatography (PE/EtOAc=100/0 to 20/1) to give the title compound 78h (300 mg, 65%).

### Step 8. (S,E)-N-((2,2-difluorospiro[3.5]nonan-7-yl)methylene)-4-methylbenzenesulfinamide (78i)

To a mixture of 78h (300 mg, 1.6 mmol), (S)-4-methylbenzenesulfenamide (247 mg, 1.6 mmol) and THF (20 mL) at 0° C. was added Ti(OEt)₄ (907 mg, 4.0 mmol), which was then warmed to room temperature and stirred for 15 h. The reaction mixture was added with saturated brine (100 mL) and filtered. The filtrate was extracted with EtOAc (100 mL). The organic phase was concentrated to dryness and the residue was purified by silica gel column chromatography (PE/EtOAc=100/0 to 10/1) to give the title compound 78i (350 mg, 68%).

MS m/z (ESI): 326 [M+1]

### Step 9. (S)—N—((S)-cyano(2,2-difluorospiro[3.5]nonan-7-yl)methyl)-4-methylbenzenesulfinamide (78j)

To a solution of 78i (350 mg, 1.1 mmol) in hexanes (15 mL) at −40° C. were added trimethylsilyl cyanide (128 mg, 1.3 mmol) and CsF (196 mg, 1.3 mmol), which was then warmed to room temperature and stirred for 15 h. The reaction mixture was added with water (50 mL) and extracted with EtOAc (50 mL). The organic phase was concentrated to dryness and the residue was purified by silica gel column chromatography (PE/EtOAc=100/0 to 10/1) to give the title compound 78j (190 mg, 50%).

MS m/z (ESI): 353 [M+1]

### Step 10. (S)-2-amino-2-(2,2-difluorospiro[3.5]nonan-7-yl)acetonitrile hydrochloride (78k)

A solution of 78j (140 mg, 0.4 mmol) in a mixture of hydrochloric acid in 1,4-dioxane (4M, 3 mL) and methanol (5 mL) was stirred for 1 h. The reaction mixture was concentrated to dryness. The residue was added with water (50 mL) and washed with EtOAc (50 mL). The aqueous layer was concentrated to dryness to give the title compound 78k (100 mg, crude). The crude product was directly used in the next step without further purification.

MS m/z (ESI): 215 [M+1]

### Step 11. (S)—N-(cyano(2,2-difluorospiro[3.5]nonan-7-yl)methyl)-1-ethyl-1H-pyrazole-5-carboxamide (78l)

To a mixture of 78k (100 mg, 0.4 mmol), 1-ethyl-1H-pyrazole-5-carboxylic acid (84 mg, 0.6 mmol), HATU (228 mg, 0.6 mmol) and DMF (3 mL) was added DIPEA (155 mg, 1.2 mmol), which was then stirred for 1 h. The reaction mixture was purified by prep-HPLC to give the title compound 781 (125 mg, 93%).

MS m/z (ESI): 337 [M+1]

### Step 12. (S)—N-(2-amino-1-(2,2-difluorospiro[3.5]nonan-7-yl)-2-oxoethyl)-1-ethyl-1H-pyrazole-5-carboxamide (78m)

To a mixture of 781 (125 mg, 0.37 mmol), hydrogen peroxide (30% aqueous solution, 0.6 mL) and DMSO (3 mL) at 0° C. was added NaOH (3 mg, 0.07 mmol), which was then warmed to room temperature and stirred for 2 h. The reaction mixture was added with water (20 mL) and filtered to give the title compound 78m (93 mg, 71%). The crude product was directly used in the next step without further purification.

MS m/z (ESI): 355 [M+1]

### Step 13. N—((S)-2-((4-((1R,2S)-1-(1-(cyclopropylmethyl)-1H-1,2,4-triazol-5-yl)-1-propionamidopropan-2-yl)-2-fluorophenyl)amino)-1-(2,2-difluorospiro[3.5]nonan-7-yl)-2-oxoethyl)-1-ethyl-1H-pyrazole-5-carboxamide (78)

A mixture of 62a (140 mg, 0.34 mmol), 78m (93 mg, 0.26 mmol), Cs₂CO₃ (169 mg, 0.52 mmol), CuI (20 mg, 0.10 mmol), trans-N,N′-dimethylcyclohexane-1,2-diamine (30 mg, 0.21 mmol), 1,4-dioxane (1 mL) and toluene (4 mL) was heated to 105° C. and stirred for 15 h. After cooling to room temperature, the reaction mixture was filtered, and the filtrate was concentrated to dryness. The residue was purified by silica gel column chromatography (dichloromethane/methanol=100/0 to 10/1) and by prep-HPLC to give the title compound 78 (77.4 mg, 44%).

MS m/z (ESI): 683 [M+1]

¹H NMR (400 MHz, CD₃OD) δ 7.81 (s, 1H), 7.73-7.62 (m, 1H), 7.48 (d, J=2.1 Hz, 1H), 7.07-6.92 (m, 2H), 6.83 (d, J=2.1 Hz, 1H), 5.28 (d, J=11.0 Hz, 1H), 4.57-4.43 (m, 3H), 3.82 (dd, J=14.4, 6.9 Hz, 1H), 3.72 (dd, J=14.4, 7.5 Hz, 1H), 3.46-3.37 (m, 1H), 2.38-2.14 (m, 6H), 1.79 (ddd, J=49.7, 22.4, 10.5 Hz, 5H), 1.56-1.39 (m, 5H), 1.36 (t, J=7.2 Hz, 3H), 1.29-1.08 (m, 5H), 0.92 (dd, J=12.9, 7.5 Hz, 1H), 0.48-0.34 (m, 2H), 0.27 (pd, J=9.2, 4.5 Hz, 2H).

### Example 36. N—((S)-1,1-dicyclopropyl-3-((4-((1R,2S)-1-(1-(2-(cyclopropylamino)ethyl)-1H-1,2,4-triazol-5-yl)-1-propionamidopropan-2-yl)-2-fluorophenyl)amino)-3-oxopropan-2-yl)-4-methyl-1,2,5-oxadiazole-3-carboxamide (Compound 79)

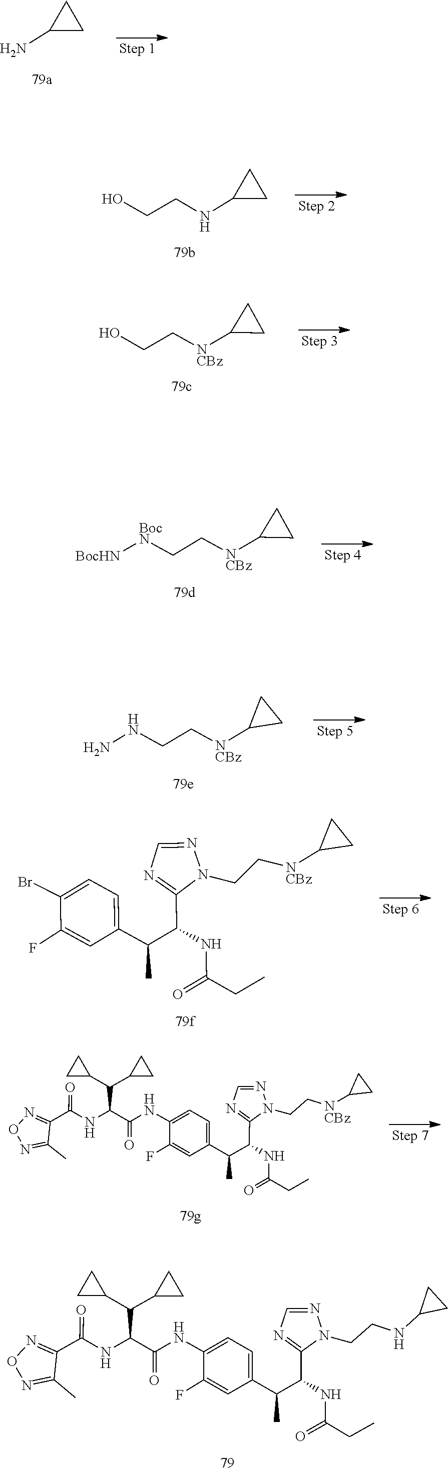

### Step 1. 2-(Cyclopropylamino)ethan-1-ol (79b)

To a solution of cyclopropanamine 79a (7.0 mL, 100 mmol) in ethanol (60 mL) was added 2-bromoethan-1-ol (6.2 g, 100 mmol), which was then heated to reflux and stirred overnight. After cooling to room temperature, the reaction mixture was concentrated to dryness to give the title compound 79b (5.0 g, 99%). The crude product was directly used in the next step without further purification.

MS m/z (ESI): 102 [M+1]

### Step 2. Benzyl cyclopropyl(2-hydroxyethyl)carbamate (79c)

To a mixture of 79b (5.0 g, 50 mmol), K₂CO₃ (13.8 g, 100 mmol), THF (100 mL) and H₂O (50 mL) was added CbzCl (10 mL, 75 mmol) at 0° C., which was then warmed to room temperature and stirred for 4 h. The reaction mixture was added with EtOAc (200 mL) and washed with saturated brine (200 mL). The organic phase was concentrated to dryness, and the residue was purified by silica gel column chromatography (PE/EA=1/0 to 2/1) to give the title compound 79c (2.0 g, 40%).

MS m/z (ESI): 236 [M+1]

### Step 3. Di-tert-butyl 1-(2-(((benzyloxy)carbonyl)(cyclopropyl)amino)ethyl)hydrazine-1,2-dicarboxylate (79d)

To a mixture of 79c (4.0 g, 17 mmol), di-tert-butyl azodicarboxylate (5.0 g, 22 mmol) and THF (50 mL) was added PPh₃ (5.7 g, 22 mmol), which was then heated to 60° C. and stirred for 4 h. After cooling to room temperature, the reaction mixture was concentrated to dryness and the residue was purified by silica gel column chromatography (PE/EtOAc=100/0 to 2/1) to give the title compound 79d (4.0 g, 52%).

MS m/z (ESI): 450 [M+1]

### Step 4. Benzyl cyclopropyl(2-hydrazineylethyl)carbamate hydrochloride (79e)

A mixture of 79d (4.0 g, 9.0 mmol) and a solution of hydrochloric acid in 1,4-dioxane (4 M, 20 mL) was heated to 60° C. and stirred for 2 h. After cooling to room temperature, the reaction mixture was concentrated to dryness to give the title compound 79e (1.5 g, 58%). The crude product was directly used in the next step without further purification.

MS m/z (ESI): 250 [M+1]

### Step 5. Benzyl (2-(5-((1R,2S)-2-(4-bromo-3-fluorophenyl)-1-propionamidopropyl)-1H-1,2,4-triazol-1-yl)ethyl)(cyclopropyl)carbamate (79f)

To a solution of (2R,3S)-3-(4-bromo-3-fluorophenyl)-N-((E)-(dimethylamino)methylene)-2-propionamidobutanamide (1.9 g, 5.0 mmol) in acetic acid (20 mL) was added 79e (1.5 g, 5.0 mmol), which was then heated to 90° C. and stirred overnight. After cooling to room temperature, the reaction mixture was concentrated to dryness and the residue was purified by prep-HPLC to give the title compound 79f (400 mg, 15%).

MS m/z (ESI): 572, 574 [M+1]

### Step 6. Benzyl cyclopropyl(2-(5-((1R,2S)-2-(4-((S)-3,3-dicyclopropyl-2-(4-methyl-1,2,5-oxadiazole-3-carboxamido)propanamido)-3-fluorophenyl)-1-propionamidopropyl)-1H-1,2,4-triazol-1-yl)ethyl)carbamate (79g)

A mixture of 79f (400 mg, 0.69 mmol), (S)—N-(1-amino-3,3-dicyclopropyl-1-oxopropan-2-yl)-4-methyl-1,2,5-oxadiazole-3-carboxamide (194 mg, 0.90 mmol), Cs₂CO₃ (455 mg, 1.40 mmol), CuI (35 mg, 0.18 mmol), trans-N,N′-dimethylcyclohexane-1,2-diamine (56 uL, 0.35 mmol) and toluene (2.5 mL) was heated to 100° C. and stirred overnight. After cooling to room temperature, the reaction mixture was purified by silica gel column chromatography (dichloromethane/methanol=100/0 to 9/1) to give the title compound 79g (400 mg, 75%).

MS m/z (ESI): 770 [M+1]

### Step 7. N—((S)-1,1-dicyclopropyl-3-((4-((1R,2S)-1-(1-(2-(cyclopropylamino)ethyl)-1H-1,2,4-triazol-5-yl)-1-propionamidopropan-2-yl)-2-fluorophenyl)amino)-3-oxopropan-2-yl)-4-methyl-1,2,5-oxadiazole-3-carboxamide (79)

A mixture of 79g (400 mg, 0.5 mmol) and Pd/C (10%, 200 mg) in ethanol (2 mL) was stirred under hydrogen atmosphere (30 psi) for 2 h. The reaction mixture was filtered, and the filtrate was concentrated to dryness. The residue was purified by prep-HPLC to give the title compound 79 (108 mg, 33%).

MS m/z (ESI): 636 [M+1]

¹H NMR (400 MHz, DMSO-d₆) δ 9.88 (s, 1H), 8.98 (d, J=8.9 Hz, 1H), 8.56 (d, J=8.5 Hz, 1H), 7.79 (s, 1H), 7.68 (t, J=8.3 Hz, 1H), 7.04 (d, J=12.0 Hz, 1H), 6.94 (d, J=8.4 Hz, 1H), 5.25-5.16 (m, 1H), 5.02-4.94 (m, 1H), 4.04-3.88 (m, 2H), 3.38 (td, J=13.9, 6.8 Hz, 1H), 2.71 (dt, J=13.6, 6.9 Hz, 1H), 2.55 (dd, J=12.4, 6.8 Hz, 2H), 2.47 (s, 3H), 2.14 (ddq, J=40.7, 15.1, 7.6 Hz, 2H), 1.99 (dd, J=6.4, 3.2 Hz, 1H), 1.33 (d, J=6.9 Hz, 3H), 0.98 (t, J=7.6 Hz, 3H), 0.91-0.67 (m, 3H), 0.51-0.10 (m, 12H).

### Example 37. N—((S)-1,1-dicyclopropyl-3-((4-((1R,2S)-1-(1-(2-(cyclopropylamino)ethyl)-1H-1,2,4-triazol-3-yl)-1-propionamidopropan-2-yl)-2-fluorophenyl)amino)-3-oxopropan-2-yl)-4-methyl-1,2,5-oxadiazole-3-carboxamide (Compound 80)

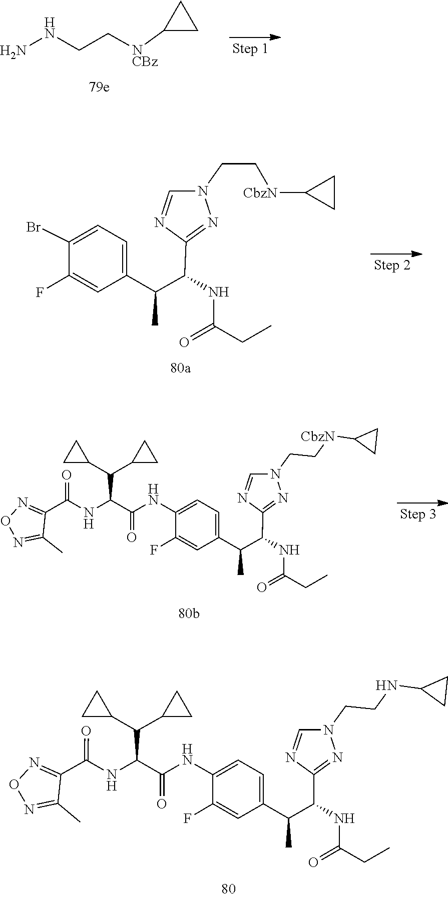

### Step 1. Benzyl (2-(3-((1R,2S)-2-(4-bromo-3-fluorophenyl)-1-propionamidopropyl)-1H-1,2,4-triazol-1-yl)ethyl)(cyclopropyl)carbamate (80a)

To a solution of (2R,3S)-3-(4-bromo-3-fluorophenyl)-N-((E)-(dimethylamino)methylene)-2-propionamidobutanamide (1.9 g, 5.0 mmol) in acetic acid (20 mL) was added 79e (1.5 g, 5.0 mmol), which was then heated to 90° C. and stirred overnight. After cooling to room temperature, the reaction mixture was concentrated to dryness and the residue was purified by prep-HPLC to give the title compound 80a (40 mg, 2%).

MS m/z (ESI): 572, 574 [M+1]

### Step 2. Benzyl cyclopropyl(2-(3-((1R,2S)-2-(4-((S)-3,3-dicyclopropyl-2-(4-methyl-1,2,5-oxadiazole-3-carboxamido)propanamido)-3-fluorophenyl)-1-propionamidopropyl)-1H-1,2,4-triazol-1-yl)ethyl)carbamate (80b)

A mixture of 80a (40 mg, 0.07 mmol), (S)—N-(1-amino-3,3-dicyclopropyl-1-oxopropan-2-yl)-4-methyl-1,2,5-oxadiazole-3-carboxamide (23 mg, 0.10 mmol), Cs₂CO₃ (46 mg, 0.14 mmol), CuI (3.5 mg, 0.02 mmol), trans-N,N′-dimethylcyclohexane-1,2-diamine (6 uL, 0.04 mmol) and toluene (1 mL) was heated to 100° C. and stirred overnight. After cooling to room temperature, the reaction mixture was purified by silica gel column chromatography (dichloromethane/methanol=100/0 to 9/1) to give the title compound 80b (40 mg, 74%).

MS m/z (ESI): 770 [M+1]

### Step 3. N—((S)-1,1-dicyclopropyl-3-((4-((1R,2S)-1-(1-(2-(cyclopropylamino)ethyl)-1H-1,2,4-triazol-3-yl)-1-propionamidopropan-2-yl)-2-fluorophenyl)amino)-3-oxopropan-2-yl)-4-methyl-1,2,5-oxadiazole-3-carboxamide (80)

A mixture of 80b (40 mg, 0.05 mmol) and Pd/C (10%, 40 mg) in ethanol (2 mL) was stirred under hydrogen atmosphere (30 psi) for 2 h. The reaction mixture was filtered, and the filtrate was concentrated to dryness. The residue was purified by prep-HPLC to give the title compound 80 (7 mg, 21%).

MS m/z (ESI): 636 [M+1]

¹H NMR (400 MHz, DMSO-d₆) δ 9.87 (s, 1H), 8.98 (d, J=8.8 Hz, 1H), 8.29 (s, 1H), 8.16 (d, J=9.4 Hz, 1H), 7.67 (t, J=8.3 Hz, 1H), 6.96 (d, J=12.1 Hz, 1H), 6.87 (d, J=6.7 Hz, 1H), 5.32 (s, 1H), 5.14 (t, J=8.7 Hz, 1H), 4.99 (s, 1H), 4.08 (s, 2H), 2.54 (s, 1H), 2.48 (s, 3H), 2.20-2.07 (m, 2H), 2.00 (dd, J=14.4, 6.8 Hz, 3H), 1.20 (d, J=7.1 Hz, 3H), 0.95 (t, J=7.6 Hz, 3H), 0.78-0.68 (m, 3H), 0.49-0.05 (m, 12H).

The following intermediate was prepared according to step 1 of Example 37, except that a different reagent was used instead of 79e.

| | | MS |
|---|---|---|
| | | m/z |
| Intermediate | Reagent replacing 79e | (ESI) |
| N-((1R,2S)-2-(4-bromo-3-fluorophenyl)-1-(1- | (Cyclopropylmethyl)hydrazine | 409, |
| (cyclopropylmethyl)-1H-1,2,4-triazol-3- | hydrochloride | 411 |
| yl)propyl)propionamide | | [M + 1] |

Compound 81 was prepared according to the procedures of steps 1 to 2 of Example 37, except that a different reagent was used instead of 79e.

| | | MS m/z |
|---|---|---|
| Compound | Reagent replacing 79e | (ESI) |
| N-((S)-1,1-dicyclopropyl-3-((4-((1R,2S)-1- | (Cyclopropylmethyl)hydrazine | 607 |
| (1-(cyclopropylmethyl)-1H-1,2,4-triazol-3- | hydrochloride | [M + 1] |
| yl)-1-propionamidopropan-2-yl)-2- | | |
| fluorophenyl)amino)-3-oxopropan-2-yl)-4- | | |
| methyl-1,2,5-oxadiazole-3-carboxamide | | |
| (81) | | |

¹H NMR data of Compound 81 are shown below:

| Compound # | ¹H NMR |
|---|---|
| 81 | ¹H NMR (400 MHz, DMSO-d₆) δ 9.62 (s, 1H), 8.75 (d, J = 9.0 Hz, 1H), 8.09 |
| | (s, 1H), 7.95 (d, J = 9.4 Hz, 1H), 7.43 (t, J = 8.3 Hz, 1H), 6.69 (d, J = 12.3 Hz, |
| | 1H), 6.64 (d, J = 7.5 Hz, 1H), 4.90 (t, J = 8.9 Hz, 1H), 4.75 (d, J = 15.6 Hz, |
| | 1H), 3.64 (d, J = 7.1 Hz, 2H), 3.02 (dd, J = 15.0, 7.4 Hz, 1H), 2.24 (s, 3H), 2.02- |
| | 1.80 (m, 2H), 0.99 (d, J = 7.1 Hz, 3H), 0.85 (dt, J = 11.9, 5.8 Hz, 1H), 0.73 |
| | (t, J = 7.6 Hz, 3H), 0.69-0.46 (m, 3H), 0.20 (d, J = 8.0 Hz, 3H), 0.17-0.11 |
| | (m, 1H), 0.07 (d, J = 4.0 Hz, 1H), 0.05-−0.10 (m, 7H). |

### Example 38. N—((S)-1,1-dicyclopropyl-3-((4-((1R,2S)-1-(3-(cyclopropylmethyl)pyridin-2-yl)-1-propionamidopropan-2-yl)-2-fluorophenyl)amino)-3-oxopropan-2-yl)-1-ethyl-1H-pyrazole-5-carboxamide (Compound 82)

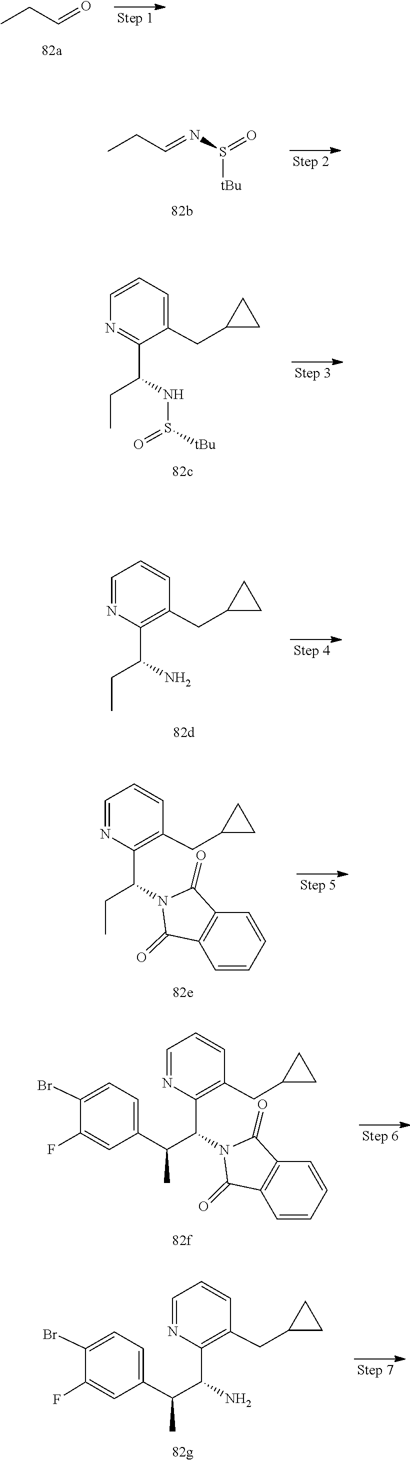

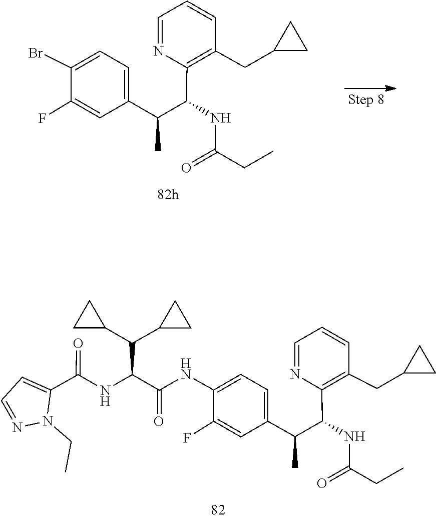 [SMILES: CCC(=O)N[C@@H](c1ncccc1CC1CC1)[C@@H](C)c1ccc(Br)c(F)c1 ; CCC(=O)N[C@@H](c1ncccc1CC1CC1)[C@@H](C)c1ccc(NC(=O)[C@@H](NC(=O)c2ccnn2CC)C(C2CC2)C2CC2)c(F)c1]

### Step 1. (S,E)-2-methyl-N-propylidenepropane-2-sulfinamide (82b)

To a mixture of propionaldehyde 82a (7.0 mL, 100 mmol), copper sulfate (16.0 g, 100 mmol) and dichloromethane (80 ml) was added (S)-2-methylpropane-2-sulfinamide (6.0 g, 50 mmol), which was then stirred for 16 h. The reaction mixture was filtered, and the filtrate was concentrated to dryness. The residue was purified by silica gel column chromatography (PE/EtOAc=9/1 to 4/1) to give the title compound 82b (7.0 g, 87%).

MS m/z (ESI): 162 [M+1]

### Step 2. (S)—N—((R)-1-(3-(cyclopropylmethyl)pyridin-2-yl)propyl)-2-methylpropane-2-sulfinamide (82c)

To a solution of 28c (4.2 g, 20 mmol) in THF (80 mL) at −60° C. was added a solution of n-BuLi in hexanes (2.5 M, 12 mL), which was then stirred at −60° C. for 30 min. The reaction mixture was added with 82b (4.8 g, 30 mmol) at −60° C., which was then stirred at −60° C. for 30 min. The reaction mixture was added with a saturated aqueous NH₄Cl solution (100 mL) and extracted with EtOAc (200 mL). The organic phase was washed with saturated brine (150 mL) and concentrated to dryness. The residue was purified by silica gel column chromatography (PE/EtOAc=3/2 to 0/1) to give the title compound 82c (800 mg, 14%).

MS m/z (ESI): 295 [M+1]

### Step 3. (R)-1-(3-(cyclopropylmethyl)pyridin-2-yl)propan-1-amine dihydrochloride (82d)

A solution of 82c (800 mg, 2.7 mmol) in a mixture of hydrochloric acid in 1,4-dioxane (4 M, 5 mL) and methanol (5 mL) was stirred for 1 h. The reaction mixture was concentrated to dryness to give the title compound 82d (500 mg, crude). The crude product was directly used in the next step without further purification.

MS m/z (ESI): 191 [M+1]

### Step 4. (R)-2-(1-(3-(cyclopropylmethyl)pyridin-2-yl)propyl)isoindoline-1,3-dione (82e)

To a solution of 82d (510 mg, 2.7 mmol) in toluene (11 mL) were added phthalic anhydride (600 mg, 4.1 mmol) and TEA (1.1 mL, 8.1 mmol), which was then heated to reflux and stirred for 2 h. After cooling to room temperature, the reaction mixture was added with a saturated aqueous NaHCO₃ solution (100 mL) and extracted with EtOAc (100 mL). The organic phase was concentrated to dryness, and the residue was purified by silica gel column chromatography (PE/EtOAc=4/1 to 3/2) to give the title compound 82e (800 mg, 92%).

MS m/z (ESI): 321 [M+1]

### Step 5. 2-((1R,2S)-2-(4-bromo-3-fluorophenyl)-1-(3-(cyclopropylmethyl)pyridin-2-yl)propyl)isoindoline-1,3-dione (82f)

A mixture of 82e (800 mg, 2.5 mmol), 1-bromo-2-fluoro-4-iodobenzene (1.1 g, 3.7 mmol), silver acetate (1.3 g, 7.5 mmol), palladium pivalate (77 mg, 0.25 mmol), TFA (0.3 mL, 3.7 mmol) and 1,1,1,3,3,3-hexafluoropropan-2-ol (20 mL) was heated to 120° C. and stirred for 12 h. After cooling to room temperature, the reaction mixture was filtered, and the filtrate was concentrated to dryness. The residue was purified by silica gel column chromatography (PE/EtOAc=9/1 to 3/7) to give the title compound 82f (300 mg, 24%).

MS m/z (ESI): 493, 495 [M+1]

### Step 6. (1R,2S)-2-(4-bromo-3-fluorophenyl)-1-(3-(cyclopropylmethyl)pyridin-2-yl)propan-1-amine dihydrochloride (82g)

To a solution of 82f (150 mg, 0.3 mmol) in ethanol (2 mL) was added hydrazine hydrate (40 uL, 0.7 mmol), which was then heated to reflux and stirred for 4 h. After cooling to room temperature, the reaction mixture was filtered. The filtrate was concentrated to dryness to give the title compound 82g (110 mg, crude). The crude product was directly used in the next step without further purification.

MS m/z (ESI): 363, 365 [M+1]

### Step 7. N-((1R,2S)-2-(4-bromo-3-fluorophenyl)-1-(3-(cyclopropylmethyl)pyridin-2-yl)propyl)propionamide (82h)

To a mixture of 82g (110 mg, crude, 0.3 mmol), propionyl chloride (60 uL, 0.7 mmol) and THF (20 mL) at 0° C. was added an aqueous NaOH solution (2 M, 300 uL), which was then stirred at 0° C. for 2 h. The reaction mixture was added with EtOAc (50 mL) and washed with a saturated aqueous NaHCO₃ solution (50 mL). The organic phase was concentrated to dryness and the residue was purified by silica gel column chromatography (PE/EtOAc=9/1 to 3/7) to give the title compound 82h (80 mg, 63%).

MS m/z (ESI): 419, 421 [M+1]

### Step 8. N—((S)-1,1-dicyclopropyl-3-((4-((1R,2S)-1-(3-(cyclopropylmethyl)pyridin-2-yl)-1-propionamidopropan-2-yl)-2-fluorophenyl)amino)-3-oxopropan-2-yl)-1-ethyl-1H-pyrazole-5-carboxamide (82)

A mixture of 82h (80 mg, 0.19 mmol), is (70 mg, 0.24 mmol), Cs₂CO₃ (130 mg, 0.40 mmol), CuI (11 mg, 0.06 mmol), trans-N,N′-dimethylcyclohexane-1,2-diamine (20 uL, 0.13 mmol) and toluene (0.8 mL) was heated to 90° C. and stirred for 3 h. After cooling to room temperature, the reaction mixture was purified by prep-HPLC to give the title compound 82 (22.3 mg, 18%).

MS m/z (ESI): 629 [M+1]

¹H NMR (400 MHz, DMSO-d₆) δ 9.73 (s, 1H), 8.43 (d, J=3.2 Hz, 1H), 8.34 (d, J=9.0 Hz, 1H), 8.20 (d, J=9.2 Hz, 1H), 7.59 (t, J=8.3 Hz, 1H), 7.48 (d, J=8.0 Hz, 2H), 7.12 (dd, J=7.7, 4.6 Hz, 1H), 6.96 (d, J=1.9 Hz, 1H), 6.77 (t, J=9.8 Hz, 2H), 5.32-5.26 (m, 1H), 4.88 (t, J=8.2 Hz, 1H), 4.46 (dd, J=14.6, 7.3 Hz, 2H), 3.37 (dd, J=16.8, 6.6 Hz, 1H), 2.20 (dd, J=14.7, 7.5 Hz, 1H), 2.04 (ddd, J=21.3, 12.3, 7.4 Hz, 4H), 1.31 (d, J=7.0 Hz, 3H), 1.29-1.25 (m, 3H), 0.96 (t, J=7.6 Hz, 3H), 0.77-0.68 (m, 3H), 0.35 (d, J=5.6 Hz, 3H), 0.27 (dd, J=8.8, 5.0 Hz, 2H), 0.18 (dt, J=11.4, 5.7 Hz, 3H), 0.12-0.04 (m, 2H), −0.02-−0.10 (m, 2H).

### Example 39. N—((R)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-1-(methyl-d₃)-1H-pyrazole-5-carboxamide (Compound 85)

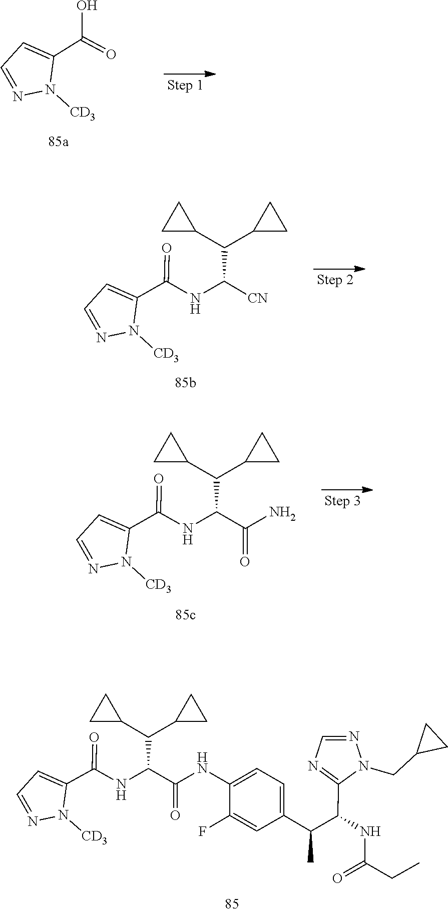 [SMILES: [2H]C([2H])([2H])n1nccc1C(=O)O ; [2H]C([2H])([2H])n1nccc1C(=O)N[C@@H](C#N)C(C1CC1)C1CC1 ; [2H]C([2H])([2H])n1nccc1C(=O)N[C@@H](C(N)=O)C(C1CC1)C1CC1 ; [2H]C([2H])([2H])n1nccc1C(=O)N[C@@H](C(=O)Nc1ccc([C@H](C)[C@@H](NC(=O)CC)c2ncnn2CC2CC2)cc1F)C(C1CC1)C1CC1]

### Step 1. (R)—N-(1-cyano-2,2-dicyclopropylethyl)-1-(methyl-d₃)-1H-pyrazole-5-carboxamide (85b)

To a mixture of (R)-2-amino-3,3-dicyclopropylpropanenitrile hydrochloride (400 mg, 2.7 mmol), 1-(methyl-d₃)-1H-pyrazole-5-carboxylic acid 85a (410 mg, 3.2 mmol), HATU (1.5 mg, 3.9 mmol) and DMF (10 mL) was added DIPEA (1.0 g, 7.7 mmol), which was then stirred for 1 h. The reaction mixture was added with EtOAc (100 mL) and then washed sequentially with a saturated aqueous NaHCO₃ solution (100 mL), dilute hydrochloric acid (1 M, 100 mL) and saturated brine (100 mL). The organic phase was concentrated to dryness, and the residue was purified by silica gel column chromatography (PE/EtOAc=4/1) to give the title compound 85b (650 mg, 93%).

MS m/z (ESI): 262 [M+1]

### Step 2. (R)—N-(1-amino-3,3-dicyclopropyl-1-oxopropan-2-yl)-1-(methyl-d₃)-1H-pyrazole-5-carboxamide (85c)

To a mixture of 85b (650 mg, 2.5 mmol), hydrogen peroxide (30% aqueous solution, 3 mL) and DMSO (10 mL) at 0° C. was added NaOH (500 mg, 12.5 mmol), which was then stirred at 0° C. for 1 h. The reaction mixture was added with water (100 mL) and extracted with EtOAc (100 mL). The organic phase was dried over anhydrous Na₂SO₄ and filtered. The filtrate was concentrated to dryness to give the title compound 85c (500 mg, 72%). The crude product was directly used in the next step without further purification.

MS m/z (ESI): 280 [M+1]

### Step 3. N—((R)-1,1-dicyclopropyl-3-((4-((1R,2S)-1-(1-(cyclopropylmethyl)-1H-1,2,4-triazol-5-yl)-1-propionamidopropan-2-yl)-2-fluorophenyl)amino)-3-oxopropan-2-yl)-1-(methyl-d₃)-1H-pyrazole-5-carboxamide (85)

A mixture of 85c (290 mg, 0.7 mmol), 62a (200 mg, 0.72 mmol), Cs₂CO₃ (470 mg, 1.4 mmol), CuI (41 mg, 0.2 mmol), trans-N,N′-dimethylcyclohexane-1,2-diamine (61 mg, 0.4 mmol) and toluene (3 mL) was heated to 110° C. and stirred overnight. After cooling to room temperature, the reaction mixture was purified by prep-HPLC to give the title compound 85 (215.7 mg, 44%).

MS m/z (ESI): 608 [M+1]

¹H NMR (400 MHz, DMSO-d₆) δ 9.81 (s, 1H), 8.57 (d, J=8.6 Hz, 1H), 8.34 (d, J=8.9 Hz, 1H), 7.77 (s, 1H), 7.62 (t, J=8.3 Hz, 1H), 7.46 (d, J=2.0 Hz, 1H), 7.04 (dd, J=14.6, 1.9 Hz, 2H), 6.93 (d, J=9.8 Hz, 1H), 5.20 (dd, J=10.2, 8.9 Hz, 1H), 5.00-4.84 (m, 1H), 3.75 (qd, J=14.3, 7.1 Hz, 2H), 3.37 (dd, J=10.3, 7.0 Hz, 1H), 2.21-2.04 (m, 2H), 1.33 (d, J=6.9 Hz, 3H), 0.98 (t, J=7.6 Hz, 3H), 0.95-0.78 (m, 3H), 0.71 (dd, J=17.1, 8.9 Hz, 1H), 0.45-0.09 (m, 12H).

Compound 84 was prepared according to the procedures of Example 39, except that a different reagent was used instead of 85a.

| | | MS m/z |
|---|---|---|
| Compound | Reagent replacing 85a | (ESI) |
| N-((R)-1,1-dicyclopropyl-3-((4-((1R,2S)-1- | 4-Methyl-1,2,5-oxadiazole-3- | 607 |
| (1-(cyclopropylmethyl)-1H-1,2,4-triazol-5- | carboxylic acid | [M + 1] |
| yl)-1-propionamidopropan-2-yl)-2- | | |
| fluorophenyl)amino)-3-oxopropan-2-yl)-4- | | |
| methyl-1,2,5-oxadiazole-3-carboxamide | | |
| (84) | | |

¹H NMR data of Compound 84 are shown below:

| Compound # | ¹H NMR |
|---|---|
| 84 | ¹H NMR (400 MHz, DMSO-d₆) δ 9.85 (s, 1H), 8.97 (d, J = 9.0 Hz, 1H), 8.58 |
| | (d, J = 8.6 Hz, 1H), 7.78 (s, 1H), 7.61 (t, J = 8.3 Hz, 1H), 7.06 (d, J = 12.1 Hz, |
| | 1H), 6.94 (d, J = 8.3 Hz, 1H), 5.28-5.16 (m, 1H), 4.96 (dd, J = 8.8, 6.7 Hz, |
| | 1H), 3.86-3.66 (m, 2H), 3.38 (dd, J = 10.4, 7.0 Hz, 1H), 2.47 (s, 3H), 2.26- |
| | 2.02 (m, 2H), 1.33 (d, J = 6.9 Hz, 3H), 0.98 (t, J = 7.6 Hz, 3H), 0.95-0.65 (m, |
| | 4H), 0.52-0.43 (m, 1H), 0.37 (dd, J = 11.5, 5.9 Hz, 2H), 0.34-0.28 (m, 2H), |
| | 0.28-0.11 (m, 7H). |

### Example 40. N—((S)-1,1-dicyclopropyl-3-((4-((1S,2S)-1-(1-(cyclopropylmethyl)-1H-1,2,4-triazol-5-yl)-1-propionamidopropan-2-yl)-2-fluorophenyl)amino)-3-oxopropan-2-yl)-1-(methyl-d₃)-1H-pyrazole-5-carboxamide (Compound 87)

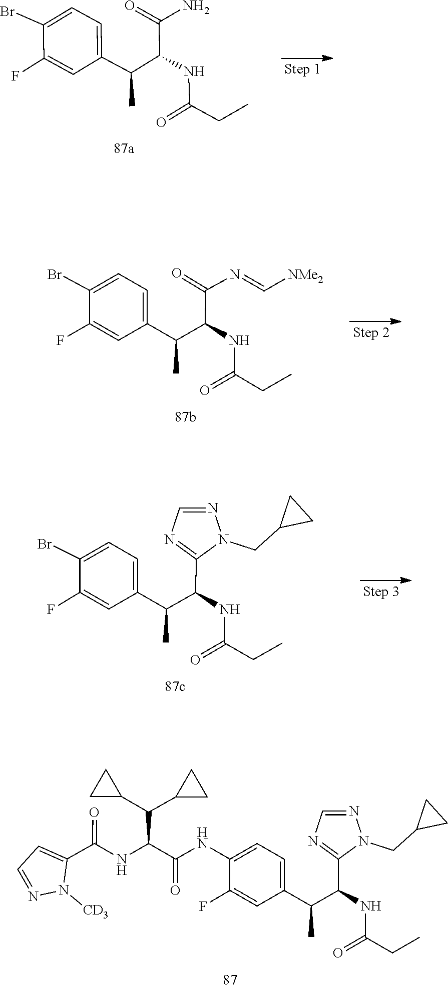 [SMILES: CCC(=O)N[C@@H](C(N)=O)[C@@H](C)c1ccc(Br)c(F)c1 ; CCC(=O)N[C@H](C(=O)/N=C/N(C)C)[C@@H](C)c1ccc(Br)c(F)c1 ; CCC(=O)N[C@H](c1ncnn1CC1CC1)[C@@H](C)c1ccc(Br)c(F)c1 ; [2H]C([2H])([2H])n1nccc1C(=O)N[C@H](C(=O)Nc1ccc([C@H](C)[C@H](NC(=O)CC)c2ncnn2CC2CC2)cc1F)C(C1CC1)C1CC1]

### Step 1. (2R,3S)-3-(4-bromo-3-fluorophenyl)-N-((E)-(dimethylamino)methylene)-2-(2-methoxyacetamido)butanamide (87b)

A solution of (2R,3S)-3-(4-bromo-3-fluorophenyl)-2-propionamidobutanamide 87a (2.6 g, 7.8 mmol) in DMF-DMA (30 mL) was heated to 90° C. and stirred overnight. After cooling to room temperature, the reaction mixture was concentrated to dryness to give the title compound 87b (3.0 g, 99%). The crude product was directly used in the next step without further purification.

MS m/z (ESI): 386, 388 [M+1]

### Step 2. N-((1S,2S)-2-(4-bromo-3-fluorophenyl)-1-(1-(cyclopropylmethyl)-1H-1,2,4-triazol-5-yl)propyl)propionamide (87c)

To a solution of 87b (3.0 g, 7.7 mmol) in acetic acid (36 mL) was added (cyclopropylmethyl)hydrazine hydrochloride (1.4 g, 11.0 mmol), which was then heated to 90° C. and stirred for 3 h. After cooling to room temperature, the reaction mixture was concentrated to dryness and the residue was purified by prep-HPLC to give the title compound 87c (640 mg, 20%).

MS m/z (ESI): 409, 411 [M+1]

### Step 3. N—((S)-1,1-dicyclopropyl-3-((4-((1S,2S)-1-(1-(cyclopropylmethyl)-1H-1,2,4-triazol-5-yl)-1-propionamidopropan-2-yl)-2-fluorophenyl)amino)-3-oxopropan-2-yl)-1-(methyl-d₃)-1H-pyrazole-5-carboxamide (87)

A mixture of 87c (293 mg, 0.7 mmol), (S)—N-(1-amino-3,3-dicyclopropyl-1-oxopropan-2-yl)-1-(methyl-d₃)-1H-pyrazole-5-carboxamide (200 mg, 0.72 mmol), Cs₂CO₃ (470 mg, 1.4 mmol), CuI (41 mg, 0.2 mmol), trans-N,N′-dimethylcyclohexane-1,2-diamine (61 mg, 0.4 mmol) and toluene (3 mL) was heated to 110° C. and stirred overnight. After cooling to room temperature, the reaction mixture was purified by prep-HPLC to give the title compound 87 (259.6 mg, 52%).

MS m/z (ESI): 608 [M+1]

¹H NMR (400 MHz, DMSO-d₆) δ 9.88 (s, 1H), 8.34 (dd, J=31.3, 9.0 Hz, 2H), 7.95 (s, 1H), 7.77 (t, J=8.3 Hz, 1H), 7.47 (d, J=2.0 Hz, 1H), 7.27 (d, J=12.4 Hz, 1H), 7.13 (d, J=9.8 Hz, 1H), 7.05 (d, J=2.1 Hz, 1H), 5.42-5.15 (m, 1H), 5.08-4.83 (m, 1H), 4.12 (dd, J=7.2, 2.2 Hz, 2H), 3.49-3.37 (m, 1H), 1.81 (tt, J=7.9, 4.1 Hz, 2H), 1.26 (t, J=7.5 Hz, 1H), 1.02 (d, J=6.9 Hz, 3H), 0.92-0.71 (m, 3H), 0.66 (t, J=7.6 Hz, 3H), 0.53-0.13 (m, 12H).

Compound 83 was prepared according to the procedures of Example 40, except that a different reagent was used instead of (S)—N-(1-amino-3,3-dicyclopropyl-1-oxopropan-2-yl)-1-(methyl-d₃)-1H-pyrazole-5-carboxamide.

| | Reagent replacing (S)-N-(1-amino- | |
|---|---|---|
| | 3,3-dicyclopropyl-1-oxopropan-2- | |
| | yl)-1-(methyl-d₃)-1H-pyrazole-5- | MS m/z |
| Compound | carboxamide | (ESI) |
| N-((S)-1,1-dicyclopropyl-3-((4-((1S,2S)-1- | (S)-N-(1-amino-3,3-dicyclopropyl-1- | 607 |
| (1-(cyclopropylmethyl)-1H-1,2,4-triazol-5- | oxopropan-2-yl)-4-methyl-1,2,5- | [M + 1] |
| yl)-1-propionamidopropan-2-yl)-2- | oxadiazole-3-carboxamide | |
| fluorophenyl)amino)-3-oxopropan-2-yl)-4- | | |
| methyl-1,2,5-oxadiazole-3-carboxamide | | |
| (83) | | |

¹H NMR data of Compound 83 are shown below:

| Compound # | ¹H NMR |
|---|---|
| 83 | ¹H NMR (400 MHz, DMSO-d₆) δ 9.93 (s, 1H), 9.01 (d, J = 9.0 Hz, 1H), 8.31 |
| | (d, J = 9.0 Hz, 1H), 7.95 (s, 1H), 7.76 (t, J = 8.3 Hz, 1H), 7.28 (d, J = 12.3 Hz, |
| | 1H), 7.15 (d, J = 7.8 Hz, 1H), 5.27 (t, J = 9.9 Hz, 1H), 5.02 (dd, J = 8.9, 6.8 |
| | Hz, 1H), 4.12 (d, J = 6.6 Hz, 2H), 3.41 (dt, J = 13.7, 6.9 Hz, 1H), 2.49 (s, 3H), |
| | 1.88-1.72 (m, 2H), 1.26 (s, 1H), 1.02 (d, J = 6.9 Hz, 3H), 0.78 (dd, J = 19.3, |
| | 10.3 Hz, 3H), 0.66 (t, J = 7.6 Hz, 3H), 0.58-0.45 (m, 3H), 0.45-0.36 (m, |
| | 3H), 0.35-0.09 (m, 6H). |

### Example 41. N—((S)-1-((4-((1R,2S)-1-([1,2,4]triazolo[4,3-a]pyrazin-3-yl)-1-(2-methoxyacetamido)propan-2-yl)-2-fluorophenyl)amino)-3,3-dicyclopropyl-1-oxopropan-2-yl)-1-ethyl-1H-pyrazole-5-carboxamide (Compound 88)

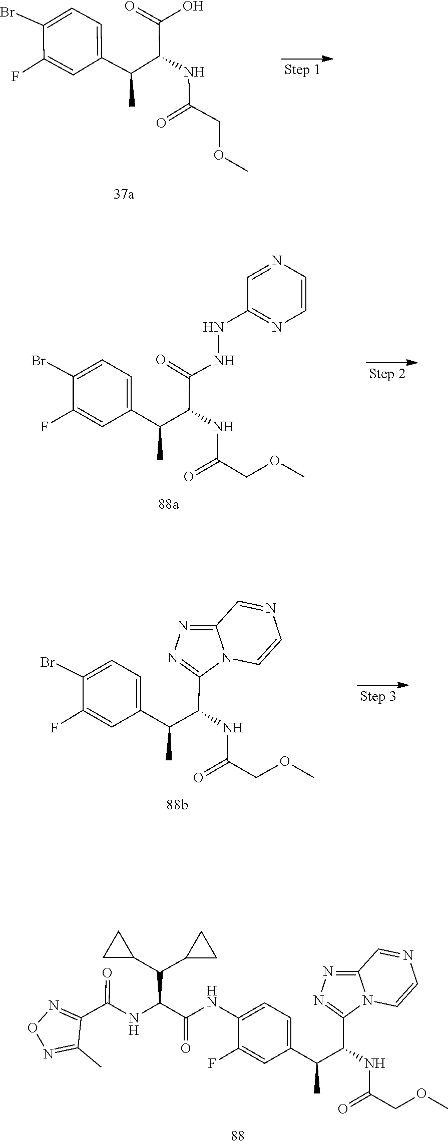 [SMILES: COCC(=O)N[C@@H](C(=O)O)[C@@H](C)c1ccc(Br)c(F)c1 ; COCC(=O)N[C@@H](C(=O)NNc1cnccn1)[C@@H](C)c1ccc(Br)c(F)c1 ; COCC(=O)N[C@@H](c1nnc2cnccn12)[C@@H](C)c1ccc(Br)c(F)c1 ; COCC(=O)N[C@@H](c1nnc2cnccn12)[C@@H](C)c1ccc(NC(=O)[C@@H](NC(=O)c2nonc2C)C(C2CC2)C2CC2)c(F)c1]

### Step 1. N-((2R,3S)-3-(4-bromo-3-fluorophenyl)-1-oxo-1-(2-(pyrazin-2-yl)hydrazineyl)butan-2-yl)-2-methoxyacetamide (88a)

To a mixture of 37a (1.00 g, 2.87 mmol), 2-hydrazinopyrazine (316 mg, 2.87 mmol), DMTMM·BF₄ (846 mg, 2.87 mmol) and acetonitrile (20 mL) was added TEA (290 mg, 2.87 mmol), which was then stirred for 1 h. The reaction mixture was concentrated to dryness and the residue was purified by silica gel column chromatography (dichloromethane/methanol=100/0 to 30/1) to give the title compound 88a (980 mg, 78%).

MS m/z (ESI): 440, 442 [M+1]

### Step 2. N-((1R,2S)-1-([1,2,4]triazolo[4,3-a]pyrazin-3-yl)-2-(4-bromo-3-fluorophenyl)propyl)-2-methoxyacetamide (88b)

To a solution of 88a (560 mg, 1.27 mmol) in THF (20 mL) at 0° C. was added Burgess reagent (1.21 g, 5.08 mmol), which was warmed to room temperature and stirred for 5 h. The reaction mixture was then further heated to 90° C. and stirred for 12 h. After cooling to room temperature, the reaction mixture was concentrated to dryness and the residue was purified by prep-HPLC to give the title compound 88b (90 mg, 17%).

MS m/z (ESI): 422, 424 [M+1]

### Step 3. N—((S)-1-((4-((1R,2S)-1-([1,2,4]triazolo[4,3-a]pyrazin-3-yl)-1-(2-methoxyacetamido)propan-2-yl)-2-fluorophenyl)amino)-3,3-dicyclopropyl-1-oxopropan-2-yl)-1-ethyl-1H-pyrazole-5-carboxamide (88)

A mixture of 88b (90 mg, 0.21 mmol), is (73 mg, 0.25 mmol), Cs₂CO₃ (205 mg, 0.63 mmol), Pd₂(dba)₃ (29 mg, 0.032 mmol), XantPhos (37 mg, 0.064 mmol) and 1,4-dioxane (3 mL) was heated to 100° C. under microwave and stirred for 1.5 h. After cooling to room temperature, the reaction mixture was filtered. The filtrate was purified by silica gel column chromatography (dichloromethane/methanol=100/0 to 40/1) and prep-HPLC in sequence to give the title compound 88 (11.9 mg, 9%).

MS m/z (ESI): 632 [M+1]

¹H NMR (400 MHz, DMSO-d₆) δ 9.72 (s, 1H), 9.24 (d, J=1.5 Hz, 1H), 8.81 (d, J=8.0 Hz, 1H), 8.54 (dd, J=4.9, 1.5 Hz, 1H), 8.33 (d, J=8.8 Hz, 1H), 7.87 (d, J=4.8 Hz, 1H), 7.56 (t, J=8.3 Hz, 1H), 7.46 (d, J=2.0 Hz, 1H), 7.14 (d, J=12.2 Hz, 1H), 6.96 (t, J=4.9 Hz, 2H), 5.84-5.70 (m, 1H), 4.86 (t, J=8.1 Hz, 1H), 4.45 (tt, J=13.4, 6.7 Hz, 2H), 3.87 (s, 2H), 3.75 (td, J=12.1, 5.1 Hz, 1H), 3.28 (s, 3H), 2.07-1.91 (m, 1H), 1.42 (d, J=6.8 Hz, 3H), 1.26 (t, J=7.2 Hz, 3H), 0.84-0.65 (m, 2H), 0.48-0.03 (m, 8H).

### Biological Experiments

### Example 42. Competitive Inhibition of Human IL-17AA and IL-17RA Binding

Recombinant IL-17AA (His-tag, Catalog Number 12047-H07B) and IL-17RA (hFc-tag, Catalog Number 10895-H02H) were purchased from Sino Biological. In the experiment, 10 μL of IL-17AA (1 nM) or IL-17RA (1 nM) was added to 10 μL of anti-6His-Tb (0.4 nM, Revvity, Catalog Number 61HISTLF) or anti-human IgG-d2 (1 μg/mL, anti-human IgG-d2, Revvity, Catalog Number 61HFCDAF), respectively and incubated at room temperature for 1 hour. Compounds were serially diluted starting from 10 μM. 5 μL of each compound solution was incubated with 5 μL of a mixture of IL-17AA and anti-6His-Tb at room temperature for 2 hours. After pre-incubation, to which was added 10 μL of a mixture of IL-17RA and anti-human IgG-d2, and incubation was continued for additional 20 minutes. Subsequently, a microplate reader (Envision, PerkinElmer) was used to detect fluorescence intensity at 615 nm and 665 nm, and the ratio of intensity thereof indicates the degree of IL-17AA and IL-17RA interaction. The ratio of intensity in the absence of tested compound was the highest value of binding between IL-17AA and IL-17RA, and the ratio of intensity in the absence of IL-17AA was the lowest value.

Therefore, the percentage of inhibition of tested compound was calculated as

$$Percentage of inhibition = 100 - \frac{( \frac{Fluorescence @ 665 nm}{Fluorescence @ 615 nm} - lowest value )}{( highest value - lowest value )} \times 100$$

Experimental results were analyzed using XLfit (IDBS) for 4-parameter nonlinear fitting analysis, to provide the IC₅₀ value for each tested compound (Table 1).

### Example 43. Inhibition of IL-17AA-Induced IL-17 Signaling Pathway

Interleukin-17 (IL-17) reporter cell line (HEK-Blue IL-17, InvivoGen, Catalog Number hkb-i117) is created by genetically engineering human IL-17RA/IL-17RC heterodimeric receptor and downstream adapter protein ACT1 into human embryonic kidney 293 cells (HEK293). These cells also express secreted embryonic alkaline phosphatase (SEAP) reporter gene induced by NF-κB and AP-1. Therefore, IL-17AA can activate NF-κB and AP-1 signaling pathways inside the cells by binding to IL-17RA/RC on cell membrane, leading to expression of SEAP protein. After a colorimetric reaction between SEAP protein and Quanti-Blue solution, the absorbance at 620 nm is measured using a microplate reader (BioTek H1M, Agilent, Catalog Number SH1M-SN) to determine IL-17AA-activated signaling pathway.

In the experiment, HEK-Blue IL-17 cells were cultured at 37° C. under 5% CO₂ conditions using the recommended medium from InvivoGen's instruction. Compounds were serially diluted starting from 10 μM. 4 μL of each compound solution was added to 32 μL of cells (10,000 cells) at 37° C. for 1 hour. After pre-incubation, 4 μL of IL-17AA with a final concentration of 2.5 ng/mL (Sino Biological, Catalog Number 12047-HNAS) was added to the cells, and incubation was continued for additional 20 hours. After incubation, 5 μL of cell culture supernatant was added to 45 μL of Quanti-Blue for the colorimetric reaction. The group without tested compound had the highest absorbance at 620 nm, and the group without IL-17AA had the lowest absorbance at 620 nm.

Therefore, the percentage of inhibition of tested compound was calculated as

$$Percentage of inhibition = 100 - \frac{( OD 620 nm - lowest value )}{( highest value - lowest value )} \times 100$$

Experimental results were analyzed using XLfit (IDBS) for 4-parameter nonlinear fitting analysis to provide the IC₅₀ value for each tested compound (Table 1).

### Example 44. Inhibition of IL-17AF-Induced IL-17 Signaling Pathway

The same experimental procedure to Example 42 was followed where IL-17AF was used instead of IL-17AA. The results are summarized in Table 1.

TABLE 1

| | IC₅₀ for Inhibiting | IC₅₀ for Inhibiting | IC₅₀ for Inhibiting |
|---|---|---|---|
| | IL-AA/IL-RA Binding | IL-AA Signaling | IL-AF Signaling |
| Compound No. | (nM) | (nM) | (nM) |
| 1 | 27 | 13 | 49 |
| 2 | 12 | 50 | 99 |
| 3 | 7.3 | 4.1 | 25 |
| 4 | 10 | 17 | 20 |
| 6 | 66 | 146 | 516 |
| 7 | 5.2 | 3.9 | 8.9 |
| 8 | 13 | 35 | 73 |
| 9 | 16 | 31 | 133 |
| 10 | 109 | 277 | 1041 |
| 11 | 13 | 44 | 92 |
| 12 | 49 | 91 | 320 |
| 13 | 40 | 67 | 216 |
| 14 | 2.9 | 2.3 | 9.0 |
| 15 | 172 | 1115 | 2132 |
| 16 | 6.3 | 6.3 | 13 |
| 17 | 442 | 85 | 251 |
| 18 | 17 | 14 | 51 |
| 19 | 4.5 | 2.6 | 4.0 |
| 20 | 20 | 14 | 50 |
| 21 | 2.2 | 1.8 | 3.7 |
| 22 | 13 | 28 | 46 |
| 23 | 21 | 25 | 91 |
| 24 | 3.2 | 2.5 | 10 |
| 25 | 57 | 115 | 313 |
| 26 | 0.8 | 2.0 | 4.5 |
| 27 | 27 | 39 | 102 |
| 28 | 7.8 | 8.4 | 24 |
| 29 | 0.9 | 2.0 | 3.2 |
| 30 | 23 | 32 | 66 |
| 31 | 11 | 6.9 | 13 |
| 32 | 2.8 | 5.7 | 8.6 |
| 33 | 7.9 | 3.1 | 7.7 |
| 34 | 2.6 | 2.2 | 3.2 |
| 35 | 5.2 | 4.5 | 8.6 |
| 36 | 3.7 | 2.0 | 2.2 |
| 37 | 125 | 74 | 232 |
| 38 | 7.5 | 2.9 | 7.0 |
| 39 | 2.2 | 1.8 | 2.6 |
| 40 | 7.4 | 2.6 | 8.6 |
| 41 | 4.9 | 11 | 16 |
| 42 | 4.9 | 5.1 | 11 |
| 43 | 9.4 | 16 | 27 |
| 44 | 1.9 | 4.4 | 29 |
| 45 | 153 | 194 | 1214 |
| 46 | 29 | 124 | 159 |
| 47 | 2.0 | 0.92 | 2.2 |
| 48 | 41 | 13 | 202 |
| 49 | 28 | 93 | 87 |
| 50 | 5.6 | 7.9 | 26 |
| 51 | 50 | 178 | 211 |
| 52 | 182 | 2215 | 2726 |
| 53 | 7.4 | 5.6 | 16 |
| 54 | 3.0 | 3.6 | 16 |
| 55 | 40 | 142 | 210 |
| 56 | 13 | 78 | 145 |
| 57 | 6.8 | 95 | 88 |
| 58 | 1.4 | 5.0 | 3.8 |
| 59 | 235 | 946 | 1547 |
| 60 | 1.7 | 3.3 | 3.9 |
| 61 | 12 | 39 | 74 |
| 62 | 2.6 | 6.5 | 18.4 |
| 63 | 2.5 | 4.0 | 8.3 |
| 64 | 1.1 | 3.6 | 5.9 |
| 65 | 0.9 | 2.8 | 3.6 |
| 66 | 1.1 | 3.3 | 8.4 |
| 67 | 1.1 | 8.0 | 13 |
| 68 | 4.5 | 5.6 | 6.4 |
| 69 | 13 | 42 | 74 |
| 70 | 3.4 | 7.5 | 7.3 |
| 71 | 5.3 | 9.6 | 13 |
| 72 | 1.0 | 1.9 | 8.5 |
| 73 | 174 | 153 | 180 |
| 74 | 5.4 | 31 | 61 |
| 75 | 1.1 | 6.1 | 8.1 |
| 76 | 6.5 | 0.7 | 1.5 |
| 77 | 5.6 | 11 | 32 |
| 78 | 48 | 376 | 517 |
| 79 | 6.0 | 19 | 36 |
| 80 | 18 | 32 | 49 |
| 81 | 4.5 | 9.4 | 16 |
| 82 | 5.6 | 10 | 8.4 |
| 83 | 21 | 45 | 39 |
| 84 | 31 | 63 | 85 |
| 85 | 165 | 539 | 810 |
| 86 | 23 | 143 | 228 |
| 87 | 1978 | >2000 | >2000 |
| 88 | 23 | 42 | 98 |

### Example 45. In Vivo Efficacy Study in Rat Collagen Induced Arthritis (CIA) Model

Bovine type II collagen solution (Chondrex) was emulsified with an equal volume of incomplete Freund's adjuvant (IFA, Sigma) to achieve an emulsion with a collagen concentration of 1 mg/mL. Female Lewis rats were immunized with the emulsion (200 μL per rat) through subcutaneous injection at the base of tail. After seven days from the initial immunization, rats received a booster injection of the emulsion (100 μL per rat). Each paw of the rat was evaluated and scored on a 0-4 scale based on arthritis severity, and the arthritis score of each rat was the sum of scores from four paws. When the arthritis scores reached 2-3, rats were randomized into four groups and dosed once a day via oral gavage (PO) with vehicle, compounds 35, 49 and 66 for 14 days, respectively. The arthritis score was assessed every other day. Efficacy was calculated by [1−(T14−T0)/(V14−V0)]×100%, where T14 and V14 are the average arthritis scores of the treatment group and the vehicle group on Day 14 of measurement, respectively, while T0 and V0 are the average arthritis scores of the treatment group and the vehicle group on Day 0, respectively. Statistical significance of difference in arthritis scores between the treatment and vehicle groups was analyzed using one-way ANOVA followed by Dunnett's test, and P<0.05 was considered statistically significant.

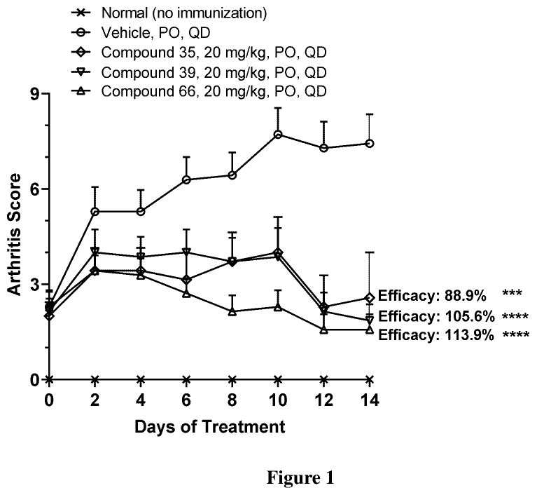

The efficacy results are shown in FIG. 1, demonstrating that compounds 35, 39 and 66 completely suppressed arthritis disease progression at 20 mpk PO QD in the rat CIA model with the P values are <<0.001, <0.0001 and <0.0001, respectively.

What is claimed is:

1. A compound according to Formula (IIb), 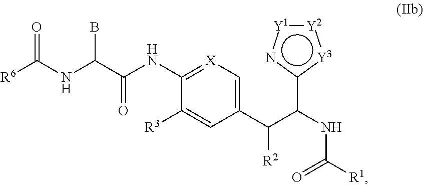
or a pharmaceutically acceptable salt thereof,
wherein:
B is C₁₋₆ alkyl or C₃₋₁₀ cycloalkyl, where one or more hydrogens of the alkyl and cycloalkyl are optionally substituted by C₃₋₁₀ cycloalkyl, C₁₋₆ alkyl, C₁₋₂ alkenyl or halogen, where one or more hydrogens of the alkyl, cycloalkyl and alkenyl are further optionally substituted by halogen or C₁₋₆ alkyl;
X is CH or N;
Y¹ is N or CR³¹;
Y² is N, NR³² or CR³¹;
Y³ is N, NR³², O or S;
R³¹ is H, halogen or C₁₋₆ alkyl;
R³² is C₁₋₆ alkyl, C₃₋₆ cycloalkyl or 4- to 6-membered heterocyclyl, where one or more hydrogens of the alkyl, cycloalkyl and heterocyclyl are optionally substituted by C₃₋₆ cycloalkyl, halogen, C₁₋₆ alkyl, 4- to 6-membered heterocyclyl, —OR^{b} or —NR¹³R¹⁴, where one or more hydrogens of cycloalkyl and heterocyclyl are further optionally substituted by halogen or C₁₋₆ alkyl;
R¹ is C₁₋₆ alkyl, where one or more hydrogens of the alkyl are optionally substituted by halogen, D, CN, 4- to 6-membered heterocyclyl, —OR^{d} or —NR¹⁵R¹⁶.
R² is methyl or cyclopropyl;
R³ is H, F or —OCH₃;
R⁶ is 5-membered heteroaryl containing one to three heteroatoms selected from N, O and S, where one or more hydrogens of the heteroaryl are optionally substituted by C₁₋₆ alkyl, fluorinated C₁₋₆ alkyl, deuterated C₁₋₆ alkyl or C₃₋₆ cycloalkyl;
R^{d} is H, C₁₋₆ alkyl, C₃₋₆ cycloalkyl or 4- to 6-membered heterocyclyl, where one or more hydrogens of the alkyl, cycloalkyl and heterocyclyl are optionally substituted by F, D, C₁₋₆ alkyl or C₃₋₆ cycloalkyl; and
R^{b}, R¹³, R¹⁴, R¹⁵ and R¹⁶ are independently selected from H, C₁₋₆ alkyl and C₃₋₆ cycloalkyl.

2. The compound of claim 1 selected from a group consisting of: 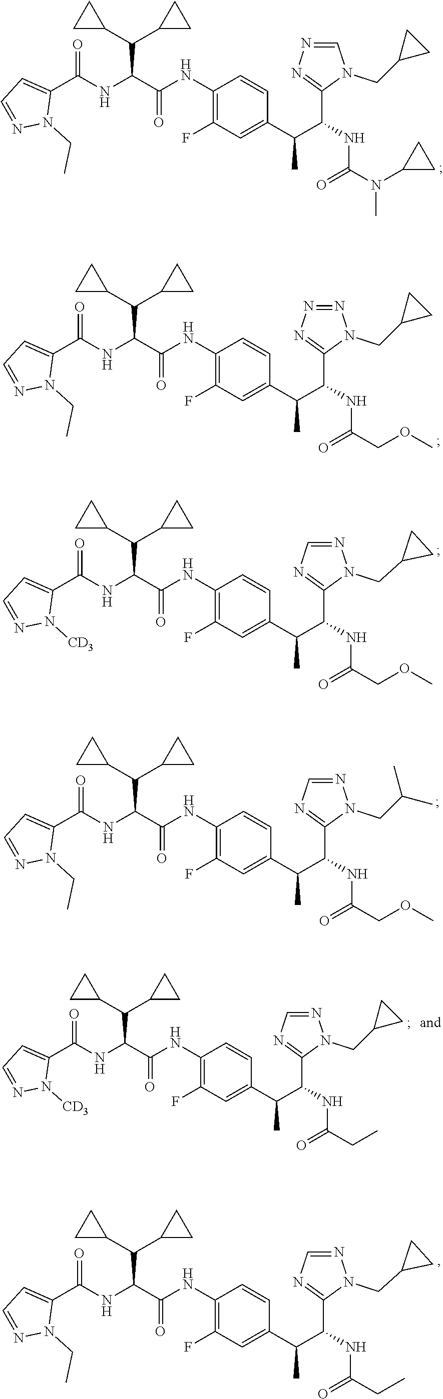
or a pharmaceutically acceptable salt, or a stable isotope derivative thereof, or a stereoisomer thereof.

3. A compound having the following structure: 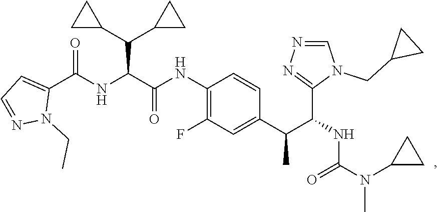 [SMILES: CCn1nccc1C(=O)N[C@H](C(=O)Nc1ccc([C@H](C)[C@@H](NC(=O)N(C)C2CC2)c2nncn2CC2CC2)cc1F)C(C1CC1)C1CC1]
or a pharmaceutically acceptable salt, or a stable isotope derivative thereof, or a stereoisomer thereof.

4. A compound having the following structure: 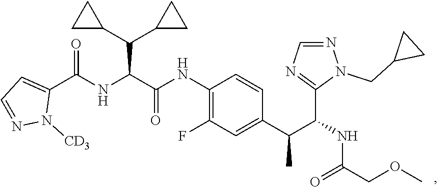 [SMILES: [2H]C([2H])([2H])n1nccc1C(=O)N[C@H](C(=O)Nc1ccc([C@H](C)[C@@H](NC(=O)COC)c2ncnn2CC2CC2)cc1F)C(C1CC1)C1CC1]
or a pharmaceutically acceptable salt, or a stable isotope derivative thereof, or a stereoisomer thereof.

5. A compound having the following structure: 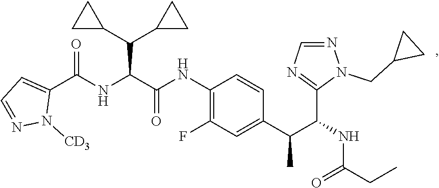 [SMILES: [2H]C([2H])([2H])n1nccc1C(=O)N[C@H](C(=O)Nc1ccc([C@H](C)[C@@H](NC(=O)CC)c2ncnn2CC2CC2)cc1F)C(C1CC1)C1CC1]
or a pharmaceutically acceptable salt, or a stable isotope derivative thereof, or a stereoisomer thereof.

6. A pharmaceutical composition comprising a compound according to claim 1, or pharmaceutically acceptable salts, or stable isotope derivatives, or stereoisomers thereof, and a pharmaceutically acceptable carrier or excipient thereof.

7. A method for treating a disease mediated by IL-17, comprising administering to a patient in need thereof a therapeutically effective amount of the compound of claim 1, or pharmaceutically acceptable salts, or stable isotope derivatives, or stereoisomers thereof.
